(19) United States
(12) Patent Application Publication
(10) Pub. No.: US 2026/0325471 A1
(43) Pub. Date: Oct. 22, 2026

(54) METHODS, SYSTEMS, AND COMPOSITIONS FOR DIAGNOSING AND TREATING BIOENERGETIC INSUFFICIENCY

(71) Applicant: Senovia Biosciences LLC, Florence, AL (US)

(72) Inventor: Joel R. Anderson, II, Florence, AL (US)

(21) Appl. No.: 19/647,204

(22) Filed: Apr. 14, 2026

Related U.S. Application Data
(60) Provisional application No. 63/788,356, filed on Apr. 14, 2025.
(60) Provisional application No. 63/845,009, filed on Jul. 16, 2025.
(60) Provisional application No. 63/920,856, filed on Nov. 19, 2025.

(51) Int. Cl. A61K 31/19; A61K 31/22; A61K 45/06; G01N 33/64

(52) U.S. Cl. CPC A61K 31/19; A61K 31/22; A61K 45/06; G01N 33/64

(57) ABSTRACT

Disclosed are methods, systems, compositions, and kits for diagnosing, staging, monitoring, and treating conditions associated with bioenergetic insufficiency in a subject or biological system. The disclosure is grounded in the Unified Theory of Bioenergetic Sufficiency (UTBS), which defines cellular health based on maintaining a Metabolic Sufficiency Ratio (M), the ratio of effective energy generation rate (E) to total energy requirement rate (R), above a critical threshold (M=E/R >M_crit). As well as, Metabolic-Epigenetic-Redox Immune Therapy (MERIT), a new therapeutic class of pharmacological, treat-to-target ketone body therapies for diseases characterized by metabolic, redox, epigenetic, or immune dysregulation.

### CROSS-REFERENCE TO RELATED APPLICATIONS

This application claims priority to and the benefit of U.S. Provisional Patent Application No. 63/788,356, filed Apr. 14, 2025, U.S. Provisional Patent Application No. 63/845,009, filed Jul. 16, 2025, and U.S. Provisional Patent Application No. 63/920,856, filed Nov. 19, 2025, the contents of each of which are hereby incorporated by reference in their entirety.

### FIELD OF THE INVENTION

The present disclosure relates generally to the field of cellular metabolism and bioenergetics. More specifically, it relates to methods, systems, compositions, and kits for diagnosing, staging, monitoring, and treating conditions characterized by bioenergetic insufficiency in cells, tissues, or organs, based on the framework of the Unified Theory of Bioenergetic Sufficiency (UTBS). In particular, the disclosure pertains to methods of treating Metabolic-Epigenetic-Redox-Immune (MERIT) responsive conditions through the targeted pharmacological induction and maintenance of circulating ketone body elevation sufficient to engage downstream target-engagement mechanisms including histone β-hydroxybutyrylation, inflammasome suppression, G-protein coupled receptor signaling, redox homeostasis modulation, and circadian rhythm alignment.

The present disclosure additionally relates to Metabolic-Epigenetic-Redox Immune Therapy (MERIT), a new therapeutic class of pharmacological, treat-to-target ketone body therapies for diseases characterized by metabolic, redox, epigenetic, or immune dysregulation. In particular, provided herein are methods, compositions, and systems for inducing and controlling Therapeutic Titratable Ketosis (TTK)—a drug mediated, diet-independent, programmable elevation of blood ketone levels—as a platform modality to treat a broad spectrum of disorders (oncology, neurological, psychiatric, metabolic, inflammatory, rare genetic, cardiovascular, etc.) that share underlying metabolic, redox, or epigenetic dysregulation (MRED). The disclosure also encompasses combination therapies with standard-of-care drugs, diagnostic and monitoring tools (e.g., continuous ketone monitors and algorithms), and treat-to-target regimens aimed at achieving specific metabolic and molecular endpoints (e.g., NAD⁺/NADH ratio correction, histone acylation marks, inflammasome inhibition) using exogenous ketone compositions. This approach is distinct from dietary ketosis or nutritional supplements; instead, MERIT positions therapeutic ketonemia as a platform medical modality akin to CAR-T cells or RNA interference, wherein controlled ketone biology is harnessed to correct complex dysregulated states across multiple disease axes. The disclosure further encompasses the use of MERIT-state biomarkers to select subjects for treatment, to guide dose titration, and to predict therapeutic response. The disclosure is not limited to any particular mechanism of ketosis induction nor to any particular disease category, but rather encompasses the broad principle that the therapeutic efficacy of the ketotic state is determined by the circulating ketone body exposure and duration of target engagement, independent of whether the ketotic state is induced through dietary intervention, pharmacological agents, or any combination thereof.

### BACKGROUND

Chronic non-communicable diseases impose a staggering and escalating burden on global health systems. Conditions such as neurodegenerative disorders, including Alzheimer's disease (AD) and Parkinson's disease (PD); cardiovascular diseases, notably heart failure (HF); metabolic disorders like type 2 diabetes (T2D) and metabolic syndrome (MetS); and other debilitating states such as traumatic brain injury (TBI) and cachexia, affect millions worldwide and represent major unmet medical needs. Prevailing medical approaches frequently address these complex conditions in isolation, focusing research and therapeutic development on disease-specific pathological hallmarks. Examples include targeting amyloid-beta plaques or neurofibrillary tangles in AD, managing cardiac remodeling and contractile dysfunction in HF, or addressing specific substrate utilization defects, such as impaired brain glucose uptake (glucose hypometabolism) in AD or defects in myocardial fatty acid oxidation (FAO) observed in certain types of HF.

While valuable, this fragmented perspective, often constrained by traditional medical specialties (e.g., neurology, cardiology, endocrinology), may fail to recognize and address fundamental, shared upstream mechanisms that could underlie these seemingly disparate conditions. The frequent clinical observation of disease co-occurrence, such as the increased risk of AD in patients with HF or the strong interrelation between HF, T2D, and MetS, strongly suggests the existence of systemic vulnerabilities and common pathophysiological pathways that transcend organ-specific boundaries. Focusing solely on downstream pathological manifestations or isolated metabolic defects, like glucose hypometabolism measured by fluorodeoxyglucose-positron emission tomography (FDG-PET) or specific FAO impairments, provides an incomplete picture. Such approaches often neglect the broader context of cellular energy balance, the dynamic interplay between multiple fuel sources, and the capacity of cells and tissues to adapt to metabolic stress. Consequently, many current treatments remain primarily symptom-focused or target downstream events, potentially failing to correct the root cause driving disease initiation and progression. This fragmented approach is further compounded by what may be termed the “Target-First Medicine” paradigm: the identification of a molecular target, the selection of a chemical entity to modulate that target, and the advancement of that entity through preclinical and clinical development based on target engagement metrics, without treating the metabolic state of the subject as a therapeutically actionable dimension. This paradigm has left a trail of failed programs-drugs with robust target engagement that nonetheless failed to show clinical benefit in Phase II or Phase III; drugs that showed efficacy in a subset of patients and total failure in others without a clear mechanistic explanation-when in fact those drugs may have been evaluated in the wrong metabolic state.

Furthermore, impaired energy metabolism is a common pathological feature across many rare (or “orphan”) diseases, particularly in neurological and metabolic disorders. In these conditions, cells are often unable to adequately utilize glucose or fatty acids for ATP production, leading to energy deficits. For example, GLUT1 Deficiency Syndrome (GLUT1-DS) involves a defect in glucose transport into the brain, causing seizures and developmental delay due to chronic cerebral energy deprivation. Pyruvate dehydrogenase complex deficiency (PDHc-D) is another rare metabolic encephalopathy where blocked pyruvate utilization leads to lactic acidosis and neurological damage. Many mitochondrial disorders (e.g. Leigh syndrome, MELAS) and fatty acid oxidation disorders (FAODs) similarly manifest as an “energy crisis” in tissues, resulting in organ dysfunction or acute life-threatening metabolic decompensation. In such patients, alternative energetic substrates like ketone bodies can play a lifesaving role by bypassing the metabolic block and providing fuel to vital organs.

There exists, therefore, a compelling need for a unifying conceptual framework capable of integrating the understanding of metabolic dysfunction across diverse chronic diseases. Such a framework should provide a common language and set of principles to explain how disturbances in cellular energy balance contribute to pathology in different tissues. It must account for the utilization of, and competition between, multiple energy substrates (including glucose, fatty acids, ketone bodies, lactate, and amino acids), the complex regulation of fuel partitioning, tissue-specific energy demands and vulnerabilities, and the mechanisms by which chronic metabolic stress leads to progressive cellular damage and functional decline. The Unified Theory of Bioenergetic Sufficiency (UTBS), which synthesizes and extends foundational concepts from prior theoretical work, is proposed to fulfill this need. UTBS offers a comprehensive paradigm centered on the principle of maintaining cellular energy supply relative to demand, thereby providing a foundation for developing integrated diagnostic, preventative, and therapeutic strategies targeting fundamental bioenergetic failure across a wide spectrum of human diseases. This approach moves beyond addressing isolated substrate defects, such as glucose hypometabolism, to encompass the overall energy state of the cell or tissue, irrespective of the specific fuel pathway initially compromised. Ketone bodies-β-hydroxybutyrate (BHB) and acetoacetate (AcAc)—exert therapeutic effects through mechanisms wholly independent of energy metabolism. These mechanisms operate through five orthogonal pillars: Metabolic (NAD⁺/NADH ratio, mitochondrial function, ATP kinetics), Epigenetic (histone β-hydroxybutyrylation (Kbhb), HDAC inhibition), Redox (ROS suppression, Nrf2 activation, oxidative-stress buffering), Immune (NLRP3 inflammasome suppression, T-cell metabolic reprogramming, barrier function), and Signaling (HCAR2/GPR109A activation, FGF21 induction, circadian synchronization). Together these constitute the Metabolic-Epigenetic-Redox-Immune Therapy (MERIT) pillar framework. Critically, none of these mechanisms require that the subject exhibit glucose hypometabolism or any particular baseline metabolic phenotype-they require only that circulating ketone body elevation be present and sustained above a threshold sufficient to engage the relevant target-engagement mechanisms.

Additionally, ketogenic therapies have traditionally been fragmented in scope and implementation. The ketogenic diet (high-fat, low-carbohydrate regimen) has been used for a century to treat epilepsy and more recently explored in metabolic diseases and cancer, but it is difficult to adhere to and not drug-standardized. The therapeutic effects of ketosis have been recognized since the ketogenic diet's introduction in the 1920s, and observational and interventional studies have since documented its efficacy across neurological disorders (including Parkinson's disease, Alzheimer's disease, and traumatic brain injury), oncological conditions, inflammatory and autoimmune diseases, metabolic disorders, and psychiatric conditions including depression and treatment-resistant mood disorders. Despite this evidence base spanning decades, ketogenic diet therapies have never achieved widespread clinical adoption outside of epilepsy, for well-established reasons: non-compliance due to dietary burden and palatability constraints; inability to titrate dose; contraindication in populations with swallowing disorders, malabsorption, or severe systemic illness; poor scalability in resource-limited settings; and the fundamental impossibility of deploying dietary ketosis as an acute adjunct to existing pharmacological regimens without wholesale lifestyle overhaul. These limitations constitute the “dietary ceiling”—the outer bound beyond which dietary ketosis cannot penetrate into mainstream clinical practice. Exogenous ketones (ketone esters, ketone salts, medium-chain triglycerides) have emerged as dietary supplements to elevate blood ketone bodies-chiefly β-hydroxybutyrate (BHB) and acetoacetate (AcAc)—-independent of diet. Prior efforts have targeted narrow applications: for example, performance and endurance enhancement via ketone monoesters, weight management and cognitive benefits via ketone drinks, lowering blood glucose in diabetes by ketone supplementation, and neuroprotection or seizure control via ketone esters and salts. Despite promising early results, these approaches are limited in several ways:

Fragmented Indications: Existing exogenous ketone therapies are typically developed for single conditions (e.g. one for muscle performance, another for anxiety or epilepsy). They do not recognize or leverage the commonality of metabolic, redox, and epigenetic dysregulation across many diseases.

Lack of Treat-to-Target Control: the state of the art teaches raising ketone levels, but not precise control to therapeutic windows. For instance, ketone ester use in performance aimed for ~2-6 mM BHB acutely, and “nutritional ketosis” is broadly defined as 0.5-5 mM, but no existing therapy explicitly measures and titrates ketones to maintain a target range over time as a primary treatment paradigm. There is no closed-loop or algorithmic closing in the prior art (with rare recent exceptions as in an experimental D-BHB system that includes measuring ketones and glucose).

Limited Mechanistic Scope: The mechanistic rationale in existing ketone therapies is often one dimensional (e.g., providing an alternative fuel to glucose, or stimulating GABA for seizures). However, ketone bodies have multi-axis effects: they can alter cellular redox state, act as signaling metabolites (binding GPCRs like HCAR2, inhibiting HDACs, modifying histones via β hydroxybutyrylation/acetoacetylation), reduce inflammation (NLRP3 inflammasome inhibition), enhance mitochondrial function, etc. Prior approaches do not coordinate all these effects. No current therapy treats the entirety of a “metabolic-redox-epigenetic immune” state.

Composition Constraints: Many exogenous ketone formulations rely on 1,3-butanediol (BD) esters (e.g., (R)-3-hydroxybutyl (R)-3-hydroxybutyrate monoester) or racemic mixtures and salt loads. BD-containing esters raise BHB effectively, but BD is an alcohol that requires hepatic metabolism; high doses can cause side effects (e.g., intoxication and acidosis at extreme doses) and may impose a metabolic burden. BHB-only approaches (without AcAc) have inherent disadvantages: BHB is the reduced form of a ketone body and must convert to AcAc by consuming NAD⁺ (producing NADH), which in a reductively-stressed cell can worsen NADH/NAD⁺ imbalance rather than relieve it. BHB-only formulations thus may not optimally address redox dysregulation. Likewise, BHB's anticonvulsant effects may be limited compared to ketosis that includes acetoacetate; some studies suggest AcAc or the act of ketogenesis itself might contribute unique neuroprotective effects. Furthermore, certain signaling and epigenetic effects require the presence of specific ketone metabolites: e.g., lysine acetoacetylation (Kacac) requires acetoacetyl-CoA availability; a BHB-only therapy might not increase Kacac, potentially missing epigenetic reprogramming benefits. In summary, prior art compositions are either BD-heavy or AcAc deficient, whereas the present disclosure recognizes the advantage of BD-free, dual-ketone (AcAc+BHB) strategies to simultaneously optimize safety, redox balance, and signaling breadth. Additional prior modalities for inducing ketosis each carry disqualifying clinical limitations: ketone monoester compounds, while capable of inducing robust circulating ketosis, cause nausea, vomiting, and diarrhea in 20-40% of subjects at effective doses, have poor palatability, produce rapid Cmax spikes followed by rapid clearance (half-life approximately 20-30 minutes), and are extraordinarily expensive per dose; ketone salts are limited by the sodium, potassium, or calcium load ceiling that prevents administration of sufficient quantities to achieve meaningful and sustained BHB levels; 1,3-butanediol is associated with intoxication-like side effects at therapeutic doses; MCT oil induces only mild ketosis (typically 0.5-1.5 mM BHB) with GI tolerability issues; SGLT2 inhibitors induce only modest ketosis (0.5-1 mM) as a secondary pharmacological effect; and direct intravenous infusion of BHB or AcAc, while controllable, is wholly impractical outside of intensive care unit settings and cannot be deployed as a chronic outpatient therapeutic modality.

Given these gaps, there is a need for a unified therapeutic platform that treats disease as a dysregulated metabolic state and uses controllable ketosis as the treatment. There is an unmet clinical need for a pharmacological, oral, titratable, sustained, well-tolerated, diet-independent, ambulatory means of inducing and maintaining a ketotic state sufficient to engage MERIT mechanisms and gate drug efficacy —one that (1) induces and maintains circulating BHB levels ≥0.5 mM for extended durations; (2) is orally bioavailable and suitable for chronic closing; (3) is titratable within a therapeutic window to match pharmacokinetic and pharmacodynamic parameters to individual patient needs; (4) does not require any dietary restriction or metabolic phenotype change on the part of the subject; (5) is suitable for use in hospitalized, frail, and ambulatory populations alike; (6) is compatible with rapid deployment in acute clinical contexts and concurrent administration with other pharmacological agents; and (7) provides a degree of safety and tolerability consistent with chronic use in diverse patient populations. No existing modality meets all seven criteria simultaneously. The present disclosure addresses this need by introducing MERIT—a comprehensive framework wherein diseases of diverse etiology are managed by correcting their common metabolic/redox/epigenetic abnormalities through titrated ketone therapy. By defining treatable “MERIT states” (e.g. a target NAD⁺/NADH ratio, a target level of histone hydroxybutyrylation, a target inflammasome activity reduction, etc.) and controlling ketone closing to achieve those states, this disclosure transcends the fragmented prior art.

A further critical conceptual shift embedded in this disclosure is the decoupling of therapeutic ketosis from glucose hypometabolism. Prior art on ketogenic diet therapy and fasting has often framed the therapeutic benefit of ketosis as consequent upon a glucose hypometabolic state —a reduced cerebral metabolic rate of glucose (CMRg1c), altered glycolytic flux, or a whole-body shift toward oxidative phosphorylation. This framing, while mechanistically accurate for dietary ketosis and fasting, has become an unwarranted constraint on the scope of ketone-based therapy. The MERIT pillar mechanisms-BHB-mediated histone modification (Kbhb), NLRP3 suppression, HCAR2 signaling, and circadian clock resetting-—are not dependent on a glucose hypometabolic state; they are dependent on circulating BHB elevation. A subject on an unrestricted, carbohydrate-rich diet will still experience histone β-hydroxybutyrylation and NLRP3 suppression if circulating BHB is sufficiently elevated. The present disclosure therefore encompasses the treatment of MERIT-responsive conditions independent of whether the subject exhibits glucose hypometabolism, independent of whether the subject's glucose metabolism is altered, and independent of any particular baseline metabolic phenotype, dramatically expanding the scope of therapeutic application and eliminating a major constraint on clinical accessibility.

Further, the present disclosure provides compositions and single-disease treatments, as well as the concept of therapeutic ketonemia as a programmable, indication-agnostic modality-covering multi-axis mechanism-based uses, combination treatments with existing drugs, diagnostic-guided closing, and system implementations. This is supported by convergent clinical evidence demonstrating that metabolic state acts as a gating variable for drug efficacy. Studies have shown that the same immunotherapy agent at identical doses can yield markedly different response rates depending on the metabolic state of the subject, with ketogenic-diet animals bearing immunologically cold tumors achieving complete response rates several-fold higher than standard-diet controls, mediated by NLRP3-dependent remodeling of the tumor microenvironment and Kbhb-driven epigenetic reprogramming of T-cells. Similarly, targeted oncological agents that cause adverse metabolic feedback in standard metabolic conditions have been shown to be rescued to full clinical benefit when administered in the context of an induced ketotic state. Multiple large clinical analyses in checkpoint inhibitor oncology have further documented that metabolic state accounts for up to 30% of the variance in immunotherapy response, with metabolically healthy subjects showing response rates up to 3.5-fold higher than metabolically dysregulated subjects. These findings collectively support the principle that pharmacological induction of a MERIT-competent ketotic state can function as a drug-efficacy gating mechanism, independent of diet and independent of baseline glucose metabolism.

### SUMMARY

The present disclosure provides methods, systems, compositions, and kits derived from the principles of the Unified Theory of Bioenergetic Sufficiency (UTBS) for the assessment and therapeutic modulation of the bioenergetic state of biological systems, including cells, tissues, organs, or whole subjects.

In one aspect, the disclosure provides methods for diagnosing, assessing, or monitoring a state of energy insufficiency or bioenergetic deficit in a subject or biological sample. These methods comprise measuring one or more parameters indicative of the Metabolic Sufficiency Ratio (M), defined as the ratio of effective energy generation rate (E) to total energy requirement rate (R) (M=E/R). Such parameters can include, but are not limited to, levels, concentrations, uptake rates, or metabolic fluxes of energy substrates (e.g., glucose, fatty acids, ketone bodies like β-hydroxybutyrate (BHB), lactate, amino acids); levels of metabolic intermediates; levels of high-energy phosphates (e.g., adenosine triphosphate (ATP), phosphocreatine (PCr)) or their ratios (e.g., PCr/ATP); oxygen consumption rates; heat production; expression levels or activities of substrate transporters (e.g., Glucose Transporters (GLUTs), Monocarboxylate Transporters (MCTs)); activities of key metabolic enzymes; indices of metabolic flexibility (e.g., respiratory quotient (RQ) changes in response to metabolic challenges); or integrated biomarker scores reflecting overall metabolic stress or vulnerability (e.g., the Metabolic Vulnerability Index (MVX)). These measurements may optionally be performed using techniques such as positron emission tomography (PET) with appropriate tracers (e.g., ¹⁸F-FDG for glucose, 11C-acetoacetate for ketones), magnetic resonance spectroscopy (MRS) (e.g., 31P-MRS for PCr/ATP, 1H-MRS for lactate/ketones), mass spectrometry- or NMR-based metabolomics, indirect calorimetry, or biochemical assays on biological samples (e.g., blood, plasma, tissue biopsies) using commercially available kits (e.g., luciferase-based ATP assays, enzymatic BHB assays).

In another aspect, methods for determining a metabolic stage or assessing the severity of bioenergetic insufficiency in a subject are provided herein. These methods involve correlating the measured M=E/R-related parameters or derived biomarker profiles with predefined stages that reflect the degree of bioenergetic sufficiency or deficit, such as the illustrative UTBS Stages 0 through 4 described herein. This staging can be used for prognosis, risk stratification, or guiding therapeutic decisions.

In a further aspect, methods for treating, preventing, or delaying the progression of a condition associated with energy insufficiency are provided herein. In some embodiments, the conditions include, but are not limited to, neurodegenerative diseases (e.g., Alzheimer's disease, Parkinson's disease), cardiovascular diseases (e.g., heart failure), metabolic disorders (e.g., type 2 diabetes, metabolic syndrome), acute brain injury (e.g., TBI), wasting syndromes (e.g., cachexia), and age-related functional decline. In some embodiments, the conditions include orphan diseases selected from the group consisting of GLUT1 deficiency syndrome, Pyruvate Dehydrogenase Complex Deficiency (PDHc-D), SLC13A5 epileptic encephalopathy, PCDH19-Related Epilepsy, Dravet syndrome, Lennox-Gastaut syndrome, infantile spasms, CDKL5 Deficiency Disorder, Doose syndrome (myoclonic-astatic epilepsy), Mitochondrial Complex I Deficiencies, Leigh syndrome, MELAS, MERRF, Friedreich's ataxia, juvenile ALS, multiple acyl-CoA dehydrogenase deficiency (MADD), Very Long-Chain Acyl-CoA Dehydrogenase Deficiency (VLCAD), Long-Chain 3-Hydroxyacyl-CoA Dehydrogenase Deficiency/Trifunctional Protein Deficiency (LCHAD/TFP), medium-chain acyl-CoA dehydrogenase deficiency (MCAD deficiency), Carnitine Palmitoyltransferase II Deficiency (CPT-II), HMG-COA Lyase Deficiency, Barth syndrome, LMNA-related dilated cardiomyopathy, Duchenne muscular dystrophy-associated cardiomyopathy, spinal muscular atrophy type II, mtDNA Depletion Syndromes, early-onset familial Alzheimer's or Parkinson's disease, Febrile Infection-Related Epilepsy Syndrome (FIRES), Glioblastoma Multiforme (GBM), Diffuse Intrinsic Pontine Glioma (DIPG), Aicardi-Goutières Syndrome (AGS), SCN8A-Related Epilepsy (SCN8A), Tuberous Sclerosis Complex (TSC), Rasmussen Encephalitis (RE), IDH-mutant High-Grade Glioma (HGG), Atypical Teratoid Rhabdoid Tumors (ATRT), Rett Syndrome (RTT), Angelman Syndrome (AS), Lafora Disease (LD), Frontotemporal Dementia (FTD), Acute Necrotizing Encephalopathy (ANEC), Autoimmune Limbic Encephalitis (ALE), Focal Cortical Dysplasia (FCD), Hypothalamic Hamartoma (HH), Neuronal Ceroid Lipofuscinoses (NCLs), Unverricht-Lundborg Disease (EPM1), Sturge-Weber Syndrome (SWS), Hemimegalencephaly (HME), and Krabbe Disease (KD).

In some embodiments, the condition associated with energy insufficiency is characterized by one or more symptoms selected from the group consisting of cardiomyopathy, seizures, hypoketotic hypoglycemia, lactic acidosis, ataxia, cognitive decline, cognitive impairment, developmental delay, exercise intolerance, failure to thrive, hypoglycemia, intracranial pressure, lethargy, metabolic crises, multiple seizure types, muscle weakness, myoclonic-astatic seizures in childhood, neurodegeneration, neurodevelopmental delay, neuropathy, recurrent non-ketotic hypoglycemia, recurrent rhabdomyolysis, refractory epilepsy, refractory seizures, super-refractory status epilepticus, and tumor progression.

These therapeutic methods comprise administering to a subject in need thereof a therapeutically effective amount of an agent or intervention designed to increase the Metabolic Sufficiency Ratio (M). This increase in M can be achieved by increasing the effective energy generation rate (E)—for example, by providing alternative energy substrates like ketone bodies (e.g., via ketone esters like D-β-hydroxybutyrate-(R)-1,3-butanediol, salts, or medium-chain triglycerides (MCTs)), lactate precursors, or specific amino acids; by enhancing the efficiency of energy production (e.g., using mitochondrial modulators like NAD⁺ precursors); or by improving the utilization of primary fuels- or by decreasing the total energy requirement rate (R)—for example, by reducing inflammation, oxidative stress, or excessive workload.

In another aspect, compositions, including pharmaceutical compositions and nutritional formulations, comprising one or more therapeutic agents capable of modulating the M=E/R ratio are provided herein. Examples of such agents include ketone bodies, ketone precursors (e.g., ketone esters, ketone salts, MCTs), lactate precursors, specific amino acids or their derivatives, agents that enhance mitochondrial function or efficiency (e.g., NAD⁺ precursors, Coenzyme Q10), agents that enhance metabolic flexibility, or agents that reduce energy demand (e.g., anti-inflammatory agents).

In a further aspect, systems for diagnosing, monitoring, or guiding the treatment of energy insufficiency are provided herein. Such systems typically comprise one or more measurement devices (e.g., a PET scanner adapted for specific metabolic tracers, an MRS system, a biomarker assay platform utilizing kits like) capable of acquiring data related to M=E/R parameters, and a processor operably connected to the measurement device(s). The processor is configured with instructions or algorithms, potentially based on the mathematical formulation of UTBS or trained using machine learning, to process the acquired data, calculate or estimate M or related indices (e.g., PCr/ATP ratio, MVX score), determine a metabolic stage, predict outcomes, simulate therapeutic effects, or recommend or control a therapeutic intervention.

In another aspect, kits for use in diagnosing, monitoring, or treating energy insufficiency according to the UTBS framework are provided herein. Such kits may comprise reagents for measuring one or more M=E/R-related biomarkers (e.g., antibodies, enzymes, standards for assays like ATP, BHB, or lactate, labeled substrates for imaging or flux analysis), or a supply of a therapeutic agent targeting M=E/R modulation (e.g., ketone esters), optionally packaged with instructions for use in assessing metabolic status, determining a UTBS stage, or administering the therapy based on the principles disclosed herein.

The disclosure explicitly encompasses embodiments wherein the diagnosed or treated energy insufficiency involves glucose hypometabolism as a specific contributing factor, while maintaining a primary focus and broader scope covering energy insufficiency arising from defects in any primary fuel pathway, excessive energy demand, impaired metabolic flexibility, or any combination thereof leading to an overall imbalance represented by M<M_crit.

Further, MERIT (Metabolic-Epigenetic-Redox Immune Therapy) is defined herein as a therapeutic approach that simultaneously modulates metabolism, redox homeostasis, epigenetic regulation, and immune/inflammatory status by means of controlled exposure to ketone bodies. The disclosure recognizes that a wide array of diseases—from cancer to neurodegeneration to autoimmune disorders-share a common denominator: Metabolic, Redox, or Epigenetic Dysregulation (MRED). As used herein, “MRED” refers to a pathological state in which cellular energy metabolism, redox balance (e.g., NADH/NAD⁺ ratio, reactive oxygen species), and/or epigenetic markers (e.g., histone acetylation or acylation status) are aberrant, leading to downstream dysfunction such as immune activation or suppressed cellular resilience. By treating this underlying state, rather than just disease-specific endpoints, MERIT provides a unified therapeutic strategy across indications.

The disclosure posits MERIT as a platform modality akin to other paradigm-shifting therapies (CAR-T cell therapy, RNA interference, PROTACs)—i.e., a modality that can be retargeted to various diseases by adjusting the therapeutic program (in this case, the ketone closing and targeting regimen). This stands in contrast to viewing ketone bodies as mere metabolic fuel or general dietary supplement. In effect, therapeutic ketonemia becomes a drug-controlled state that can be achieved, measured, and maintained for clinical benefit.

Therapeutic Titratable Ketosis (TTK)—Treat-to-Target Ketonemia: Described herein is Therapeutic Titratable Ketosis (TTK), a treat-to-target platform for achieving drug-grade ketosis —defined, sustained levels of blood ketone bodies—in a manner that is tunable and programmable by the clinician. TTK is essentially the “control engine” of MERIT, comprising methods and tools to:
a. Induce ketosis pharmacologically without reliance on carbohydrate restriction. This is achieved via novel exogenous ketone compositions (e.g., ketone esters, salts, precursors or prodrugs), formulated to be 1,3-butanediol-free and enriched in acetoacetate precursors (to provide both BHB and AcAc in vivo). The compositions can be oral, enteral, or parenteral, and are designed for high purity of the biologically active enantiomer (D-BHB, if applicable) and optimal tolerability.
b. Measure ketosis continuously or intermittently, using devices such as blood BHB/AcAc sensors (including continuous ketone monitors, microneedle patch sensors, etc.), and to track related metabolic markers (glucose, lactate, etc.).
c. Adjust closing in real-time based on feedback to hit specified targets—e.g., maintain blood BHB between 2-4 mM and AcAc between 1-2 mM for 12 hours a day, or achieve a BHB:AcAc ratio of approximately 1:1. This feedback-controlled closing (algorithmic or manual) ensures the patient remains in the therapeutic window (avoiding too low to be ineffective, and too high to risk ketoacidosis). TTK thus transforms ketosis from a static outcome of a diet into a dynamic, prescription-controlled state. For example, rather than simply administering a fixed ketone dose, a TTK protocol may involve: (a) administering an initial ketone infusion or drink, (b) measuring blood BHB and AcAc after 30 minutes, (c) if levels are below target, administering an additional booster dose or adjusting the infusion rate, (d) continuously monitoring via a sensor, and (e) automatically modulating a ketone delivery pump to sustain the target range. This “administer-measure-adjust” cycle is repeated to maintain therapeutic ketonemia. Importantly, such closed-loop or semi-closed-loop control of ketone therapy is a novel aspect, differentiating from prior art that delivered ketones in fixed dose boluses without ongoing titration. It also aligns the treatment closely with individual patient needs (personalized ketosis closing).

In another aspect provided herein is a method of treating a metabolic-epigenetic-redox-immune (MERIT) responsive condition in a subject, comprising administering to the subject a therapeutically effective amount of a ketogenic agent selected from the group consisting of: (a) dietary restriction in the form of a ketogenic diet; (b) fasting selected from intermittent fasting and prolonged fasting; (c) an exogenous ketone monoester comprising (R)-3-hydroxybutyryl-(R)-3-hydroxybutyrate or an analog thereof; (d) an exogenous ketone salt selected from sodium β-hydroxybutyrate, potassium β-hydroxybutyrate, calcium β-hydroxybutyrate, sodium acetoacetate, potassium acetoacetate, and combinations thereof; (e)_{1,3}-butanediol or a derivative thereof; (f) a medium-chain triglyceride selected from C6, C8, C10, and C12 triglycerides; (g) a ketone-carrier conjugate comprising a polyol linked via a linker to one or more ketone bodies; (h) an SGLT2 inhibitor selected from empagliflozin, dapagliflozin, canagliflozin, ertugliflozin, and combinations thereof; (i) any combination of (a)-(h); in an amount sufficient to induce and maintain in the subject a circulating β-hydroxybutyrate concentration of at least 0.5 mM for a duration of at least 7 days, thereby engaging metabolic state remodeling, epigenetic chromatin deacetylation, redox rebalancing, and immune tolerance; wherein the method does not require that the subject exhibit glucose hypometabolism prior to, during, or after said administration, and is independent of dietary macronutrient restriction beyond that inherent to the ketogenic agent itself.

In another aspect provided herein is a method of increasing the therapeutic efficacy of a second pharmaceutical agent in a subject having a MERIT-responsive condition, comprising inducing in the subject a ketotic metabolic state by administering a ketogenic agent selected from the group consisting of a ketogenic diet, intermittent or prolonged fasting, an exogenous ketone monoester, an exogenous ketone salt, 1,3-butanediol or a derivative thereof, a medium-chain triglyceride, a ketone-carrier conjugate, an SGLT2 inhibitor, or any combination thereof, in an amount sufficient to achieve and maintain a circulating β-hydroxybutyrate concentration of at least 0.5 mM for a duration of at least 7 days; subsequently administering to the subject said second pharmaceutical agent in a dose and schedule that would be therapeutically ineffective or sub-efficacious in the absence of the ketotic state; wherein the second pharmaceutical agent exhibits greater therapeutic efficacy, as measured by tumor response rate, disease remission rate, symptom reduction, or biomarker improvement, when administered during or immediately following the ketotic state compared to historical baseline or control populations receiving said second agent without concomitant ketotic state induction; wherein the method does not require that the subject exhibit glucose hypometabolism, and is independent of dietary restriction of the subject.

In another aspect provided herein is a method of treating an immune-inflammatory condition characterized by dysregulation of NLRP3 inflammasome-mediated inflammation, in a subject not required to exhibit glucose hypometabolism, comprising administering a ketogenic agent selected from the group consisting of a ketogenic diet, intermittent or prolonged fasting, an exogenous ketone monoester, an exogenous ketone salt, 1,3-butanediol or a derivative thereof, a medium-chain triglyceride, a ketone-carrier conjugate, an SGLT2 inhibitor, or any combination thereof, in an amount sufficient to induce and maintain a circulating β-hydroxybutyrate concentration of at least 0.5 mM for at least 7 days; wherein the ketotic state suppresses NLRP3 inflammasome activation, as evidenced by reduced circulating IL-18, IL-18, or IL-6 levels; and wherein the method does not require that the subject exhibit glucose hypometabolism, and is independent of dietary macronutrient restriction beyond that inherent to the ketogenic agent itself.

In another aspect provided herein is a method of treating a dermatologic condition characterized by inflammatory infiltrate and dysregulated cutaneous immunity, in a subject not required to exhibit glucose hypometabolism, comprising administering a ketogenic agent selected from the group consisting of a ketogenic diet, intermittent or prolonged fasting, an exogenous ketone monoester, an exogenous ketone salt, 1,3-butanediol or a derivative thereof, a medium-chain triglyceride, a ketone-carrier conjugate, an SGLT2 inhibitor, or any combination thereof, in an amount sufficient to achieve and maintain a circulating β-hydroxybutyrate concentration of at least 1.0 mM for at least 14 days; wherein the ketotic state remodels cutaneous immune cell function and reduces inflammatory cytokine production; measuring clinical endpoints including lesion erythema, induration, extent of affected body surface area, and pruritus intensity; and wherein the method does not require that the subject exhibit glucose hypometabolism, and is independent of dietary restriction of the subject.

In another aspect provided herein is a method of treating a psychiatric or neurologic condition mediated at least in part by reduced nicotinamide adenine dinucleotide (NAD⁺) signaling, histone deacetylase (HDAC) dysregulation, or metabolic insufficiency in the central nervous system, in a subject not required to exhibit glucose hypometabolism, comprising administering a ketogenic agent selected from the group consisting of a ketogenic diet, intermittent or prolonged fasting, an exogenous ketone monoester, an exogenous ketone salt, 1,3-butanediol or a derivative thereof, a medium-chain triglyceride, a ketone-carrier conjugate, an SGLT2 inhibitor, or any combination thereof, in an amount sufficient to induce and maintain a circulating β-hydroxybutyrate concentration of at least 1.0 mM for at least 7 days; wherein the circulating and central nervous system BHB acts as a substrate for NAD⁺-dependent signaling, inhibits HDAC activity via histone acetylation, and enhances mitochondrial function in neuronal and glial cells; measuring clinical endpoints including symptom severity score, functional outcome measure, and mood or cognition assessment; and wherein the method does not require that the subject exhibit glucose hypometabolism, and is independent of dietary restriction of the subject.

In another aspect provided herein is a method of treating a bacterial or viral infection, including chronic infection or sepsis, in a subject not required to exhibit glucose hypometabolism, comprising administering a ketogenic agent selected from the group consisting of a ketogenic diet, intermittent or prolonged fasting, an exogenous ketone monoester, an exogenous ketone salt, 1,3-butanediol or a derivative thereof, a medium-chain triglyceride, a ketone-carrier conjugate, an SGLT2 inhibitor, or any combination thereof, in an amount sufficient to induce and maintain a circulating β-hydroxybutyrate concentration of at least 0.5 mM for a duration dependent on infection type and severity; wherein the ketotic state suppresses NLRP3 inflammasome-mediated pathologic inflammation while preserving or enhancing adaptive immune response (T cell and B cell function); co-administering an antimicrobial agent (antibiotic or antiviral) specific to the pathogen; measuring endpoints including pathogen load (viral titer, bacterial colony—forming units), inflammatory biomarkers (IL-1β, TNF-α, IL-6), organ function (creatinine, transaminases, coagulation parameters), and clinical outcome (survival, ventilator-free days, ICU-free days); and wherein the method does not require that the subject exhibit glucose hypometabolism, and is independent of dietary restriction of the subject.

In another aspect provided herein is a method of treating an oncologic indication via immune checkpoint inhibitor therapy combined with metabolic state manipulation, in a subject not required to exhibit glucose hypometabolism, comprising inducing a ketotic metabolic state by administering a ketogenic agent selected from the group consisting of a ketogenic diet, intermittent or prolonged fasting, an exogenous ketone monoester, an exogenous ketone salt, 1,3-butanediol or a derivative thereof, a medium-chain triglyceride, a ketone-carrier conjugate, an SGLT2 inhibitor, or any combination thereof, in an amount sufficient to achieve and maintain a circulating β-hydroxybutyrate concentration of at least 1.0 mM; maintaining said ketotic state for a period of 7-14 days prior to the first dose of immune checkpoint inhibitor therapy; subsequently administering an immune checkpoint inhibitor selected from anti-PD-1, anti-PD-L1, anti-CTLA-4, or combinations thereof, maintaining concurrent ketotic state induction throughout the duration of checkpoint inhibitor therapy; measuring clinical endpoints including objective response rate, duration of response, progression-free survival, and overall survival; wherein the ketotic state remodels tumor-associated immune dysfunction, reduces tumor-infiltrating regulatory T cells and myeloid-derived suppressor cells, enhances tumor-infiltrating effector T cell function, and increases tumor immunogenicity; and wherein the method does not require that the subject exhibit glucose hypometabolism, and is independent of dietary restriction of the subject.

In another aspect provided herein is a method of selecting and monitoring subjects for ketosis-based therapeutic intervention, comprising measuring in a biological sample obtained from the subject at least one MERIT biomarker selected from the group consisting of: (i) circulating β-hydroxybutyrate concentration; (ii) urinary ketone concentration; (iii) adipose tissue transcriptomic signature indicating dysregulated energy metabolism; (iv) peripheral blood mononuclear cell (PBMC) NAD⁺/NADH ratio; (v) NLRP3 inflammasome activity, as measured by circulating IL-1β, IL-18, or caspase-1 activity; (vi) HCAR2 signaling activity in immune cell populations; (vii) histone deacetylase (HDAC) activity; (viii) a composite metabolic vulnerability index derived from a multiplex biomarker panel; comparing the measured biomarker value(s) to an established threshold or reference range; selecting for ketosis-based therapeutic intervention any subject whose biomarker value(s) indicate dysregulation of metabolic state, epigenetic tone, redox balance, or immune function; wherein the method enables identification of MERIT-responsive subjects independent of traditional disease classification; and wherein the method does not require that the subject exhibit glucose hypometabolism and is independent of dietary restriction of the subject.

In another aspect provided herein is a method of monitoring therapeutic response to a ketogenic agent in a subject with a MERIT-responsive condition, comprising measuring baseline target-engagement biomarkers in said subject selected from the group consisting of NLRP3 inflammasome activity, HCAR2 signaling activity, PBMC NAD⁺/NADH ratio, HDAC activity, circulating metabolic substrate ratios, and epigenetic chromatin histone acetylation state; initiating ketogenic agent administration in an amount sufficient to induce and maintain a circulating β-hydroxybutyrate concentration of at least 0.5 mM; measuring on-treatment target-engagement biomarkers at defined timepoints (days 3, 7, 14, 28); continuing ketogenic agent administration if target-engagement biomarkers demonstrate the intended mechanistic effect; adjusting ketogenic agent dose or switching to an alternative ketogenic agent if target-engagement biomarkers fail to demonstrate expected engagement; measuring clinical outcome biomarkers (disease-specific endpoints) at days 7, 14, 28, and 90 to assess therapeutic efficacy; wherein target-engagement biomarker-guided adjustment optimizes therapeutic response independent of empirical dose escalation alone; and wherein the method does not require that the subject exhibit glucose hypometabolism, and is independent of dietary restriction of the subject.

In another aspect provided herein is a kit for MERIT-responsive subject selection and therapeutic monitoring, comprising: (a) a ketogenic agent selected from the group consisting of a ketogenic diet, intermittent or prolonged fasting, an exogenous ketone monoester, an exogenous ketone salt, 1,3-butanediol or a derivative thereof, a medium-chain triglyceride, a ketone-carrier conjugate, an SGLT2 inhibitor, or any combination thereof; (b) one or more MERIT biomarker assay(s) selected from the group consisting of circulating β-hydroxybutyrate concentration, urinary ketone concentration, adipose tissue transcriptomic signature, PBMC NAD⁺/NADH ratio, NLRP3 inflammasome activity, HCAR2 signaling activity, HDAC activity, and a composite metabolic vulnerability index, in a form suitable for point-of-care or laboratory measurement; (c) a reference table or nomogram relating biomarker values to MERIT-responsive phenotype and ketosis-based intervention eligibility; (d) written instructions for subject selection, ketogenic agent closing, on-treatment biomarker monitoring at defined intervals (days 3, 7, 14, 28, 90), and dose adjustment criteria; (e) optionally, a portable meter or assay cartridge for measuring circulating β-hydroxybutyrate in a clinic or home setting; wherein the kit is configured for use with subjects who do not exhibit glucose hypometabolism, and is independent of dietary restriction of the subject.

In another aspect provided herein is a pharmaceutical composition for enhancing the therapeutic efficacy of a second pharmaceutical agent via concomitant metabolic state modulation, comprising: (a) a ketogenic agent selected from the group consisting of an exogenous ketone monoester comprising (R)-3-hydroxybutyryl-(R)-3-hydroxybutyrate, an exogenous ketone salt selected from sodium, potassium, or calcium β-hydroxybutyrate, a ketone-carrier conjugate comprising a polyol linked to one or more ketone bodies, 1,3-butanediol or a derivative thereof, a medium-chain triglyceride, or an SGLT2 inhibitor selected from empagliflozin, dapagliflozin, canagliflozin, or ertugliflozin; (b) a second pharmaceutical agent selected from the group consisting of an immune checkpoint inhibitor (anti-PD-1, anti-PD-L1, anti-CTLA-4), a kinase inhibitor (PI3K, MEK, JAK, or tyrosine kinase inhibitor), a chemotherapeutic agent, a psychiatric or neurologic agent (SSRI, SNRI, mood stabilizer, antiepileptic), an antimicrobial agent (antibiotic or antiviral), or a biologic immune-modulating agent; and optionally a pharmaceutically acceptable excipient, carrier, or vehicle; formulated for co-administration in amounts sufficient to (1) induce and maintain a circulating β-hydroxybutyrate concentration of at least 0.5 mM, and (2) achieve therapeutic concentrations of the second agent at the site of action; wherein the composition is formulated to provide synergistic or super-additive therapeutic benefit via state-dependent enhancement of second agent efficacy; and wherein the composition does not require that the subject exhibit glucose hypometabolism, and is independent of dietary restriction of the subject.

In another aspect provided herein is a kit for treating a MERIT-responsive condition via combined ketosis induction and therapeutic intervention, comprising: (a) a ketogenic agent in a form suitable for oral or intravenous administration, in a unit-dose or multi-dose presentation; (b) a second pharmaceutical agent selected from the group consisting of an immune checkpoint inhibitor, a kinase inhibitor, a chemotherapeutic agent, a psychiatric or neurologic agent, an antimicrobial agent, or a biologic immune-modulating agent, in a therapeutic unit-dose; (c) a MERIT biomarker assay or point-of-care measurement device selected from the group consisting of circulating β-hydroxybutyrate concentration, urinary ketone concentration, adipose tissue transcriptomic signature, PBMC NAD⁺/NADH ratio, NLRP3 inflammasome activity, HCAR2 signaling activity, HDAC activity, and a composite metabolic vulnerability index; (d) written instructions comprising subject selection criteria based on MERIT biomarker profile, ketogenic agent closing and administration route and frequency, timing of second agent administration relative to ketogenic agent initiation, on-treatment biomarker monitoring schedule and decision rules, clinical outcome assessment timepoints and target endpoints, and safety monitoring parameters and dose adjustment algorithms; and (e) optionally a patient adherence tracking tool, such as a mobile application or paper diary; wherein the kit is configured for use with subjects who do not exhibit glucose hypometabolism, and is independent of dietary restriction of the subject.

In another aspect provided herein is a therapeutic combination product comprising a ketogenic agent and a diagnostic biomarker measurement component, integrated for use in identifying and treating MERIT-responsive subjects, comprising: (a) a pharmaceutical composition containing a ketogenic agent selected from the group consisting of a ketogenic diet, intermittent or prolonged fasting, an exogenous ketone monoester, an exogenous ketone salt, 1,3-butanediol or a derivative thereof, a medium-chain triglyceride, a ketone-carrier conjugate, an SGLT2 inhibitor, or any combination thereof; (b) an integrated diagnostic device or assay capable of measuring a MERIT biomarker selected from the group consisting of circulating β-hydroxybutyrate concentration, urinary ketone concentration, adipose tissue transcriptomic signature, PBMC NAD⁺/NADH ratio, NLRP3 inflammasome activity, HCAR2 signaling activity, HDAC activity, and a composite metabolic vulnerability index, in a real-time or near-real-time fashion (results available within 5-60 minutes); (c) a computational algorithm or display interface that correlates the measured biomarker value(s) to subject MERIT-responsiveness and provides a recommendation for ketogenic agent initiation, dose, duration, or discontinuation; (d) written instructions for use, including subject selection, biomarker measurement procedure, interpretation, and ketogenic agent administration guidance; wherein the combination product enables bedside or clinic-based MERIT phenotyping and real-time therapeutic guidance; and wherein the composition does not require that the subject exhibit glucose hypometabolism, and is independent of dietary restriction of the subject.

In another aspect provided herein is a method of manufacturing a fixed-dose combination pharmaceutical composition comprising a ketogenic agent and a second therapeutic agent, comprising mixing a therapeutically effective amount of a ketogenic agent selected from the group consisting of a ketogenic diet, intermittent or prolonged fasting, an exogenous ketone monoester, an exogenous ketone salt, 1,3-butanediol or a derivative thereof, a medium-chain triglyceride, a ketone-carrier conjugate, an SGLT2 inhibitor, or any combination thereof, with a therapeutically effective amount of a second agent selected from the group consisting of an immune checkpoint inhibitor, a kinase inhibitor, a chemotherapeutic agent, a psychiatric or neurologic agent, an antimicrobial agent, or a biologic immune-modulating agent; incorporating the mixture into a pharmaceutically acceptable carrier, excipient, or vehicle; forming unit-dose forms (oral liquids, suspensions, tablets, capsules, sachets, or vials); quantitatively assaying the final composition to verify target β-hydroxybutyrate equivalent concentration (molar basis) and second agent concentration; packaging the unit-dose forms with labeling and instructions specifying use for MERIT-responsive condition treatment; wherein the fixed-dose combination is manufactured to ensure stoichiometric equivalence across unit doses, enabling predictable bioavailability and pharmacodynamic target engagement; and wherein the method does not require that the subject exhibit glucose hypometabolism, and is independent of dietary restriction of the subject.

In another aspect provided herein is a pharmaceutical composition in a form suitable for oral administration to a human subject, comprising: (a) a ketogenic agent selected from the group consisting of an exogenous ketone monoester, an exogenous ketone salt, a ketone-carrier conjugate, 1,3-butanediol, or a medium-chain triglyceride; (b) a taste-masking agent selected from the group consisting of microcrystalline cellulose, silica, citric acid, or a proprietary taste-masking polymer; (c) a bioavailability enhancer selected from the group consisting of permeation enhancers, surfactants, or lipid-based formulation excipients; (d) optionally a coloring agent approved for food or pharmaceutical use; (e) optionally a sweetening agent; formulated as an oral liquid, suspension, powder sachet, tablet, or capsule; wherein the composition is formulated to optimize palatability and oral bioavailability of the ketogenic agent, enabling consistent achievement of circulating β-hydroxybutyrate concentrations of at least 0.5 mM within 60-120 minutes of administration.

In another aspect provided herein is a method of treating a MERIT-responsive condition in a subject, comprising administering a ketogenic agent in an amount sufficient to achieve a target ratio of circulating β-hydroxybutyrate to acetoacetate (BHB:AcAc) in the range of about 1:1 to about 5:1; monitoring the BHB:AcAc ratio by measuring both circulating BHB and circulating AcAc; and adjusting the dose or composition of the ketogenic agent to maintain the target BHB:AcAc ratio for a duration sufficient to engage at least one of histone β-hydroxybutyrylation, NLRP3 inflammasome suppression, or HCAR2 signaling; wherein the method does not require that the subject exhibit glucose hypometabolism; and wherein the method is independent of dietary restriction of the subject.

In another aspect provided herein is a method of treating a MERIT-responsive condition in a subject, comprising determining a circadian phase of the subject by measuring at least one circadian biomarker selected from the group consisting of cortisol awakening response, core body temperature nadir timing, melatonin onset, and clock gene expression level of at least one of BMAL1, PER2, CRY1, and CLOCK; administering a ketogenic agent at a time of day selected to align peak circulating β-hydroxybutyrate concentration with a target circadian phase of the subject; wherein the target circadian phase is selected to maximize engagement of at least one MERIT pillar mechanism; wherein the method does not require that the subject exhibit glucose hypometabolism; and wherein the method is independent of dietary restriction of the subject.

In another aspect provided herein is a method of monitoring and optimizing therapeutic ketosis in a subject being treated for a MERIT-responsive condition, comprising measuring circulating glucose concentration and circulating β-hydroxybutyrate concentration in the subject; calculating a Glucose-Ketone Index (GKI) according to the formula: GKI=[glucose (mg/dL)]/([BHB (mmol/L)]×18.01); comparing the calculated GKI to a target GKI threshold; and adjusting the dose of a ketogenic agent administered to the subject to achieve or maintain a GKI at or below the target GKI threshold; wherein the method does not require that the subject exhibit glucose hypometabolism; and wherein the method is independent of dietary restriction of the subject.

In another aspect provided herein is a kit comprising a dual-analyte point-of-care meter for measuring glucose and β-hydroxybutyrate; instructions for calculating a Glucose-Ketone Index (GKI); and a reference card listing indication-specific target GKI thresholds for MERIT-responsive conditions; wherein use of the kit does not require that the subject exhibit glucose hypometabolism; and wherein use of the kit is independent of dietary restriction of the subject.

In another aspect provided herein is a method of selecting a subject for ketosis-based therapy for a MERIT-responsive condition, comprising measuring a baseline GKI in the subject; determining that the baseline GKI is above a threshold indicative of metabolic dysregulation for the subject's MERIT-responsive condition; and administering a ketogenic agent to the subject to reduce the GKI to at or below the indication-specific target threshold; wherein the method does not require that the subject exhibit glucose hypometabolism; and wherein the method is independent of dietary restriction of the subject.

In another aspect provided herein is a method of treating a MERIT-responsive condition in a subject, comprising: (a) determining whether the subject's condition is characterized as fuel-driven (where the primary mechanism is energy substrate delivery to dysfunctional mitochondria) or signal-driven (where the primary mechanism is activation of specific signaling pathways at defined concentration thresholds); (b) if fuel-driven, selecting a closing regimen of a ketogenic agent comprising high single-dose or infrequent (once to twice daily) administration to maximize total circulating β-hydroxybutyrate area-under-the-curve (AUC); (c) if signal-driven, selecting a closing regimen of a ketogenic agent comprising lower, frequent (three to four times daily) administration to maintain circulating β-hydroxybutyrate within a target concentration window (0.5-2.0 mM) for ≥2.6 hours daily; (d) administering the selected closing regimen; and (e) monitoring for therapeutic response; wherein the method does not require that the subject exhibit glucose hypometabolism; and wherein the method is independent of dietary restriction of the subject.

In another aspect provided herein is a method of optimizing pharmacologically-induced ketosis to simultaneously engage multiple beneficial mechanisms while avoiding off-target mechanisms, comprising: (a) identifying a subject with a MERIT-responsive condition where multiple beneficial mechanisms require engagement; (b) selecting a ketogenic agent and dose to maintain circulating β-hydroxybutyrate in a therapeutic optimization corridor of 0.5-2.0 mM for maximal duration per day, wherein the corridor simultaneously engages NLRP3 inflammasome suppression, HCAR2 signaling, and Kbhb histone modification while remaining below the class I HDAC engagement threshold of approximately 2.4 mM; (c) administering the selected ketogenic agent; and (d) measuring circulating BHB and at least one mechanism-specific biomarker to confirm multi-mechanism engagement; wherein the method does not require that the subject exhibit glucose hypometabolism; and wherein the method is independent of dietary restriction of the subject.

In another aspect provided herein is a method of treating a MERIT-responsive condition in a subject, comprising: (a) classifying the subject's condition into one of the following categories: (i) sepsis or systemic infection; (ii) autoimmune or inflammatory disease; (iii) oncology with immune-exclusion phenotype; (iv) neurodegenerative disease; (v) psychiatric or mood disorder; (vi) cardiac disease; (vii) dermatologic disease; or (viii) kidney disease; (b) selecting an indication-specific target circulating β-hydroxybutyrate range based on the condition category: 0.5-1.0 mM for sepsis; 1.0-2.0 mM for autoimmune disease; 1.5-3.0 mM for oncology; 0.5-1.5 mM for neurodegeneration; 0.5-1.5 mM for psychiatry; 0.5-1.0 mM for cardiac disease; 0.5-1.5 mM for dermatology; or 0.5-1.0 mM for kidney disease; (c) administering a ketogenic agent in an amount to achieve the indication-specific target; and (d) measuring circulating BHB at intervals and adjusting dose to maintain the target range; wherein the method does not require that the subject exhibit glucose hypometabolism; and wherein the method is independent of dietary restriction of the subject.

In another aspect provided herein is a method of treating an autoinflammatory disease in a subject, comprising: (a) identifying a subject with an autoinflammatory condition selected from familial Mediterranean fever, cryopyrin-associated periodic syndromes (FCAS, Muckle-Wells syndrome, NOMID), gout, or other monogenic autoinflammatory syndromes where NLRP3 inflammasome hyperactivation is the validated pathophysiologic mechanism; (b) administering a ketogenic agent in an amount sufficient to induce and maintain circulating β-hydroxybutyrate in the range 1.0-2.0 mM for at least 2.6 hours per day, while remaining below 2.4 mM; (c) measuring circulating BHB and NLRP3 inflammasome activation at intervals; and (d) adjusting dose to maintain the specified BHB time-window; wherein the method does not require that the subject exhibit glucose hypometabolism; and wherein the method is independent of dietary restriction of the subject.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a bar graph showing dose-dependent effects of acetoacetate (AcAc) on neuronal viability in Alzheimer's disease (AD) neurons. Neurons were treated with amyloid beta (Aβ1-42) and increasing concentrations of AcAc (approximately 0.1 mM, 0.5 mM, 1 mM, 10 mM, and 100 mM). Cell viability was measured using a luminescence-based live/dead assay (CellTiter-Glo). Increasing concentrations of AcAc progressively improve viability relative to untreated AD neurons exposed to Aβ, approaching levels observed in healthy control neurons.

FIG. 2 is a bar graph showing dose-dependent effects of acetoacetate (AcAc) on reactive oxygen species (ROS) levels in AD neurons. Neurons were treated with amyloid beta (Aβ1-42) and increasing concentrations of AcAc (approximately 0.1 mM to 100 mM). ROS levels were measured using MitoSOX staining. Increasing concentrations of AcAc reduce MitoSOX fluorescence intensity relative to untreated AD neurons, indicating a dose-dependent reduction in oxidative stress.

FIG. 3 is a bar graph showing effects of multiple ketone bodies, including D-β-hydroxybutyrate (D-BHB), L-β-hydroxybutyrate (L-BHB), and acetoacetate (AcAc), on ROS levels in AD neurons under amyloid beta stimulation. Ketone treatment reduces ROS relative to untreated AD neurons, demonstrating a class-wide effect of ketones on oxidative stress reduction.

FIG. 4 is a set of fluorescence microscopy images showing mitochondrial structure and oxidative stress in neurons under amyloid beta-induced conditions. Neurons were treated with 1 μM amyloid beta (Aβ1-42) in the presence or absence of β-hydroxybutyrate (β-OHB) at concentrations of approximately 250 μM and 500 μM. MitoTracker staining (green) indicates mitochondrial structure and integrity, while MitoSOX staining (red) indicates mitochondrial reactive oxygen species (ROS) production. Ketone-treated cells exhibit reduced MitoSOX signal intensity and improved mitochondrial morphology relative to amyloid-treated cells without ketone treatment, indicating reduced oxidative stress and preservation of mitochondrial integrity.

FIG. 5 is a bar graph quantifying mitochondrial oxidative stress in neurons under amyloid beta-induced conditions. Neurons were treated with 1 μM amyloid beta (Aβ1-42) in the presence or absence of β-hydroxybutyrate (β-OHB) at concentrations of approximately 250 μM and 500 μM. Reactive oxygen species (ROS) levels were measured using MitoSOX staining and normalized to MitoTracker signal. Treatment with β-OHB reduced MitoSOX fluorescence intensity relative to amyloid-treated cells without ketone treatment, indicating reduced mitochondrial oxidative stress. Lower values correspond to reduced oxidative stress.

FIG. 6 is a bar graph showing dose-dependent effects of acetoacetate (AcAc) on intracellular ATP levels in Alzheimer's disease (AD) neurons. Neurons were treated with 1 μM amyloid beta (Aβ1-42) and increasing concentrations of AcAc (approximately 0.1 mM, 0.5 mM, 1 mM, 10 mM, and 100 mM). Intracellular ATP levels were measured using an ATP-dependent luminescence assay and are presented relative to healthy control neurons. AD neurons exhibit reduced ATP levels relative to healthy controls, while treatment with increasing concentrations of AcAc results in a dose-dependent increase in ATP levels, indicating restoration of cellular energy metabolism.

FIG. 7 is a bar graph showing neuronal viability under basal and amyloid beta-stimulated conditions in AD neurons. Neurons were treated with 1 μM amyloid beta (Aβ1-42) in the presence or absence of ketone bodies, including D-β-hydroxybutyrate (D-BHB), L-β-hydroxybutyrate (L-BHB), and acetoacetate (AcAc), at approximately 5 mM for 14 days. Cell viability was measured using a CellTiter-Glo Live/Dead assay. Amyloid beta exposure reduces viability relative to basal conditions, while ketone treatment restores viability to varying extents.

FIG. 8 is a bar graph showing apoptosis levels in AD neurons as measured by Annexin V staining. Neurons were treated with 1 μM amyloid beta (Aβ1-42) in the presence or absence of ketone bodies (D-BHB, L-BHB, and AcAc) at approximately 5 mM for 14 days. The percentage of Annexin V-positive cells is increased following amyloid beta exposure and reduced following ketone treatment, indicating decreased apoptotic signaling.

FIG. 9 is a graph showing oxygen consumption rate (OCR) over time in AD neurons as measured by a Seahorse XF Mito Stress Test. Neurons were treated with ketone bodies (D-BHB, L-BHB, and AcAc). OCR was measured across sequential additions of mitochondrial modulators, including oligomycin, FCCP, and rotenone/antimycin A. Ketone-treated neurons exhibit increased mitochondrial respiration relative to untreated AD neurons.

FIG. 10 is a bar graph showing ATP-linked respiration in AD neurons. Neurons were treated with ketone bodies (D-BHB, L-BHB, and AcAc) and ATP concentration was measured. Ketone-treated neurons exhibit increased ATP relative to untreated AD neurons.

FIG. 11 is a bar graph showing basal respiratory capacity in AD neurons. Neurons were treated with ketone bodies (D-BHB, L-BHB, and AcAc). Basal respiration was determined using Seahorse XF Mito Stress Test analysis. Ketone-treated neurons exhibit increased basal respiratory capacity relative to untreated AD neurons, indicating improved mitochondrial function.

FIG. 12 is a bar graph showing neuronal viability in Parkinson's disease (PD) neurons. Neurons were treated with ketone bodies (D-BHB, L-BHB, and AcAc). Cell viability was measured using a CellTiter-Glo Live/Dead assay. PD neurons exhibit reduced viability relative to healthy controls, and ketone treatment improves viability, with AcAc demonstrating a comparatively strong effect.

FIG. 13 is a bar graph showing apoptosis levels in PD neurons as measured by Annexin V staining. Neurons were treated with ketone bodies (D-BHB, L-BHB, and AcAc). PD neurons exhibit increased percentages of Annexin V-positive cells relative to healthy controls, and ketone treatment reduces apoptotic signaling.

FIG. 14 is a bar graph showing reactive oxygen species (ROS) levels in PD neurons as measured by MitoSOX staining. Neurons were treated with ketone bodies (D-BHB, L-BHB, and AcAc). PD neurons exhibit elevated ROS levels relative to healthy controls, and ketone treatment reduces ROS levels to varying extents.

FIG. 15 is a graph showing oxygen consumption rate (OCR) over time in PD neurons as measured by a Seahorse XF Mito Stress Test. Neurons were treated with ketone bodies (D-BHB, L-BHB, and AcAc). OCR was measured across sequential additions of mitochondrial modulators, including oligomycin, FCCP, and rotenone/antimycin A. Ketone-treated neurons exhibit increased mitochondrial respiration relative to untreated PD neurons.

FIG. 16 is a bar graph showing ATP production in PD neurons. Neurons were treated with ketone bodies (D-BHB, L-BHB, and AcAc). ATP-linked respiration was derived from Seahorse XF Mito Stress Test measurements. Ketone treatment increases ATP production relative to untreated PD neurons.

FIG. 17 is a bar graph showing basal respiratory capacity in PD neurons. Neurons were treated with ketone bodies (D-BHB, L-BHB, and AcAc). Basal respiration was determined using Seahorse XF Mito Stress Test analysis. Ketone treatment increases basal respiratory capacity relative to untreated PD neurons.

FIG. 18 is a bar graph showing uptake of amyloid beta (Aβ1-42) by microglia treated with ketone esters. Microglia were treated with ketone esters and exposed to fluorescently labeled Aβ1-42 for approximately 1 hour. Following acid wash to remove non-internalized amyloid, intracellular fluorescence was measured. Increased fluorescence intensity indicates enhanced internalization of amyloid beta in ketone-treated microglia relative to untreated controls.

FIG. 19 is a bar graph showing clearance of amyloid beta (Aβ1-42) by microglia following ketone treatment. Microglia were treated with ketone esters and exposed to fluorescently labeled Aβ1-42 for approximately 24 hours. Residual extracellular amyloid beta was quantified by fluorescence analysis. Reduced fluorescence intensity indicates decreased residual amyloid beta and enhanced clearance relative to untreated controls.

FIG. 20 is a line graph showing the relative kinetics of amyloid beta (Aβ1-42) clearance in microglia over time. Microglia were treated with acetoacetate (AcAc) and exposed to amyloid beta, and clearance was monitored over a time course of approximately 0 to 30 hours. Ketone-treated microglia exhibit enhanced uptake at early time points and accelerated clearance relative to vehicle-treated controls, resulting in improved overall amyloid clearance kinetics.

FIG. 21 displays a graph demonstrating the mean time-plasma BHB concentration following a representative compound of Formula (A), TrehaAce, PO closing in mice.

### DETAILED DESCRIPTION

### Overview of Bioenergetic Sufficiency and UTBS Framework

The present disclosure is predicated on the Unified Theory of Bioenergetic Sufficiency (UTBS), a comprehensive framework designed to explain the fundamental role of cellular energy balance in health and disease. UTBS posits that the ability of a cell, tissue, or organ to maintain its structure, function, and resilience is critically dependent on achieving and sustaining a state of bioenergetic sufficiency. This state is quantitatively represented by the Metabolic Sufficiency Ratio (M), defined as the ratio of the effective rate of energy generation (E) to the total rate of energy requirement (R), i.e., M=E/R.

The central hypothesis underpinning UTBS and the present disclosure is that optimal cellular and tissue health requires M to be maintained above a certain critical threshold (M_crit), which may be tissue-specific. A state where M≥M_crit signifies bioenergetic sufficiency, allowing cells to meet their energy demands for basal function, activity, stress responses, and repair. Conversely, chronic disease states are proposed to arise, or be significantly exacerbated, when M persistently falls below this critical threshold (M<M_crit). Such a decline into energy insufficiency can be triggered by various factors, including: (1) impaired uptake or utilization of primary energy substrates (e.g., glucose, fatty acids); (2) inadequate supply of necessary fuels; (3) defects in mitochondrial function leading to inefficient energy conversion; (4) pathologically elevated energy demands (e.g., due to chronic inflammation, oxidative stress, or excessive workload); or (5) impaired metabolic flexibility, hindering the ability to adapt fuel utilization to changing conditions.

A key aspect of UTBS is its potential to serve as a unifying paradigm, providing a common mechanistic explanation for a wide array of seemingly disparate pathologies. Conditions such as Alzheimer's disease (AD) and Parkinson's disease (PD), heart failure (HF), type 2 diabetes (T2D) and metabolic syndrome (MetS), traumatic brain injury (TBI), cachexia, and aspects of the aging process can all be viewed through the lens of underlying bioenergetic failure in specific vulnerable tissues or systemically. Furthermore, there exist many orphan conditions that can also be viewed through this lens. These include, but are not limited to refractory epilepsies (e.g., Dravet Syndrome, Lennox-Gastaut Syndrome, GLUT1 Deficiency Syndrome, CDKL5 Deficiency Disorder, PCDH19-Related Epilepsy, and Infantile Spasms) and Mito- and Neuro-degenerative conditions (e.g., Friedreich Ataxia, Leigh Syndrome, MELAS Spectrum Disorders, Mitochondrial Complex I Deficiency, mtDNA Depletion Syndromes, and Mitochondrial Parkinson's Disease. UTBS integrates and extends concepts from foundational works, providing a structured approach to diagnose, stage, and treat these conditions by targeting the fundamental M=E/R balance.

The present disclosure encompasses several inventive concepts, each contributing to the field-defining scope of MERIT:

A. Ketone-Driven “MERIT States” as Treatable Targets: Introduced herein is the concept that disease associated dysregulated states (e.g., high NADH/NAD⁺ ratio, chronic inflammasome activation, hypoacetylation of histones, hyperexcitability of neurons, etc.) are themselves targets for therapy, and that controllable ketosis is the means to treat those states. In other words, rather than focusing only on a disease's end-symptoms, MERIT therapy targets the underlying MRED state. For instance, cancer and neurodegenerative diseases both often feature a Warburg-like metabolic shift (high glycolysis, impaired mitochondrial oxidation) and reductive stress (excess NADH). MERIT therapy would treat “Warburg/redox dysregulation” via ketosis, thereby impacting both diseases, albeit with different clinical outcomes. This is a broad reframing: a method of treating any MRED-characterized disease by inducing a specified ketone state. No prior therapy has claimed the general treatment of “a disease of metabolic/redox dysregulation” with ketones as provided herein with one unified approach.

B. NADH Drain via Acetoacetate (Redox Correction): A hallmark mechanistic innovation is leveraging acetoacetate as an “NADH sink”. In normal mitochondria, the enzyme D-β-hydroxybutyrate dehydrogenase (BDH1) interconverts BHB and AcAc, using NAD⁺/NADH as co-substrate. By providing exogenous AcAc (or precursors that yield AcAc intracellularly), the reaction consumes excess NADH (converting it to NAD⁺) while reducing AcAc to BHB. This NADH drain relieves reductive stress—a condition where NADH builds up relative to NAD⁺, impairing metabolism and signaling. Reductive stress is implicated in diverse conditions (ischemia, heart failure, Alzheimer's, cancer cells' metabolic profile, etc.). The disclosure recognizes AcAc therapy as a general remedy for reductive stress: for example, treating a patient with mitochondrial dysfunction by giving an AcAc donor to oxidize NADH and restore NAD⁺ levels. This is in stark contrast to prior ketone therapies that predominantly elevate BHB (which actually pushes the equilibrium toward NADH production). Our approach can normalize markers like the blood lactate/pyruvate (L/P) ratio and the β-hydroxybutyrate: acetoacetate ratio (BHB:AcAc) in favor of a more oxidized state (higher AcAc relative to BHB, indicating higher NAD⁺/NADH). The present methods of treating diseases by shifting these ratios—e.g., “administering a ketone composition to reduce the BHB:AcAc ratio to ≤1:1 in a subject, thereby alleviating a reductive stress condition”. This aspect is novel; existing literature notes ketones affect NAD⁺/NADH but no therapeutic regimen has been built around titrating a redox ratio. Presented herein are not only compositions rich in AcAc or precursors (like 1,3-butanediol diacetoacetate, AcAc salts, AcAc polymers), but also the treat-to-target method of adjusting dose until NAD⁺ increases or NADH decreases to a desired level in cells or blood.

C. Epigenetic Modulation via Ketones (HDAC Inhibition and Histone Acylation): The disclosure also targets epigenetic dysregulation using ketosis. BHB is known to be a class I HDAC inhibitor at millimolar concentrations, leading to increased histone acetylation at genes for stress resistance and FOXO3, etc., Additionally, metabolites BHB and AcAc can form novel histone post-translational modifications: lysine β-hydroxybutyrylation (Kbhb) and lysine acetoacetylation (Kacac), respectively, which have been detected during fasting/ketosis and regulate gene expression. MERIT therapy aims to treat conditions of epigenetic imbalance (e.g., repression of beneficial genes or hypermethylation syndromes) by elevating these ketone driven marks. For example, certain neurodevelopmental or neurodegenerative disorders involve aberrant HDAC activation (e.g., HDAC2 overactivity in Alzheimer's disease or cognitive impairment). A MERIT treatment might raise blood BHB to ~3 mM for several hours a day to inhibit HDAC2 in neurons, thereby reactivating silenced genes (like BDNF or antioxidant enzymes). Similarly, in certain cancers or congenital epigenetic syndromes (Rett syndrome, Kabuki syndrome, etc.), boosting histone acetylation or introducing Kbhb/Kacac marks could correct gene expression patterns. The disclosure presents methods such as: administering a ketone composition to achieve a ≥X % increase in histone H3K9ac and H3K14bhb in target tissues, or to reduce HDAC activity by Y %, as a way to treat disease. Notably, no prior clinical therapy deliberately uses a metabolite to modify chromatin marks in vivo.

D. Immunometabolic Reprogramming (Inflammasome and Immune Cell Modulation): The MERIT platform extends to the immune system: many chronic diseases have an immunometabolic component—e.g., overactive innate immunity (inflammasomes like NLRP3 driving IL-1B release in inflammatory diseases, or T-cell exhaustion in cancer and chronic infections due to metabolic insufficiency). Ketone bodies are herein used as immunometabolic modulators. BHB has been found to inhibit the NLRP3 inflammasome assembly in macrophages, thereby reducing IL-1β and IL-18 secretion, especially under acidic conditions. The disclosure leverages this for treating NLRP3-driven disorders (e.g., gout, rheumatoid arthritis, autoinflammatory syndromes, possibly neuroinflammation in Alzheimer's). MERIT therapy can maintain ketone levels such that immune cells are exposed to ~5 mM BHB, enough to dampen inflammasome activation. Likewise, ketone metabolism increases the available acetyl-CoA in mitochondria, supporting T cell oxidative phosphorylation and thereby potentially reversing T-cell exhaustion in tumors (improving responsiveness to checkpoint inhibitors) or in chronic viral infections. Described herein are methods of treating an inflammatory or immune dysfunction disease by achieving a ketone state that improves immune biomarkers (such as lowered IL-1β, increased T-cell NAD⁺, increased cytokine IL-2 production, or enhanced memory T-cell formation). In some embodiments, a method of treating an NLRP3-associated inflammatory disease (e.g., gout or CAPS) comprising administering an effective dose of a ketone composition to maintain BHB>2 mM for at least 8 hours per day, thereby reducing NLRP3 inflammasome activity and alleviating symptoms. In some embodiments, a method of improving cancer immunotherapy response comprising inducing therapeutic ketosis (GKI (glucose: ketone index)≤2.0) during anti-PD-1 therapy, to reprogram T-cell metabolism and enhance anti-tumor immunity.

E. Closed-Loop MERIT Control Systems: In addition to compositions and methods, the disclosure includes systems and algorithms for implementing MERIT therapy safely and effectively. This includes continuous ketone monitors (CKM) and software that computes closing adjustments—for example, a wearable sensor that transmits BHB levels to a pump controlling ketone infusion, analogous to a “ketone pancreas.” It also includes multi-sensor approaches, where an “M-score”—a composite index of metabolic/redox/epigenetic biomarkers—is calculated and kept within a target range. The system might incorporate a computer program that uses inputs like blood BHB, AcAc, glucose, lactate, breath acetone, and even gene expression markers, to recommend or automatically administer the next ketone dose. The disclosure thus anticipates a future where MERIT therapy is delivered via smart devices (possibly as an at-home kit containing a ketone formulation, a meter, and instructions/algorithms for titration). In some embodiments, a system comprises: (i) a ketone delivery device containing a therapeutic ketone composition, (ii) a sensor for real-time monitoring of ketone level in the patient, and (iii) a controller programmed to adjust the delivery device output to maintain a predetermined ketone level or ratio. In some embodiments, kits comprise package a ketone agent with a compatible monitoring tool and closing guidelines for specific indications (e.g., a “MERIT kit for bipolar disorder” with instructions to achieve a target BHB level shown to correlate with mood stabilization).

### Definitions

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligo-or polynucleotide chemistry and hybridization described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

The articles “a” and “an” are used herein to refer to one or to more than one (i.e. at least one) of the grammatical objects of the article.

### Chemical Definitions

Definitions of specific functional groups and chemical terms are described in more detail below.

In some embodiments, compounds described herein comprise one or more asymmetric centers, and thus exist in various isomeric forms, e.g., enantiomers and/or diastereomers. For example, in some embodiments, the compounds described herein are in the form of an individual enantiomer, diastereomer or geometric isomer, or are in the form of a mixture of stereoisomers, including racemic mixtures and mixtures enriched in one or more stereoisomer. In some embodiments, isomers are isolated from mixtures by methods known to those skilled in the art, including chiral high pressure liquid chromatography (HPLC) and the formation and crystallization of chiral salts; or preferred isomers are prepared by asymmetric syntheses. The disclosure additionally encompasses compounds described herein as individual isomers substantially free of other isomers, and alternatively, as mixtures of various isomers.

In some embodiments, compounds described herein also comprise one or more isotopic substitutions. For example, in some embodiments, H is in any isotopic form, including ¹H, ²H (D or deuterium), and ³H (T or tritium); C is any isotopic form, including ¹²C, ¹³C, and ¹⁴C; O is in any isotopic form, including ¹⁶O and ¹⁸O; F is in any isotopic form, including ¹⁸F and ¹⁹F; and the like.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₆, C₄₋₅, and C₅₋₆ alkyl.

As used herein, “alkyl” refers to a radical of a straight-chain or branched saturated hydrocarbon group, e.g., having 1 to 20 carbon atoms (“C₁-C₂₀ alkyl”). In some embodiments, an alkyl group has 1 to 10 carbon atoms (“C₁-C₁₀ alkyl”). In some embodiments, an alkyl group has 1 to 9 carbon atoms (“C₁-C₉ alkyl”). In some embodiments, an alkyl group has 1 to 8 carbon atoms (“C₁-C₈ alkyl”). In some embodiments, an alkyl group has 1 to 7 carbon atoms (“C₁-C₇ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). Examples of C₁-C₆ alkyl groups include methyl, ethyl, propyl, isopropyl, butyl, isobutyl, pentyl, hexyl, and the like.

As used herein, “alkylene,” refers to a divalent radical of an alkyl group. When a range or number of carbons is provided for a particular “alkylene” group, it is understood that the range or number refers to the range or number of carbons in the linear carbon divalent chain. In some embodiments, “alkylene” group is substituted or unsubstituted with one or more substituents as described herein.

As used herein, “aryl” refers to a radical of a monocyclic or polycyclic (e.g., bicyclic or tricyclic)₄n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”).

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In some embodiments, in heteroaryl groups that contain one or more nitrogen atoms, the point of attachment is a carbon or nitrogen atom, as valency permits.

In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively.

The term “cycloalkyl” refers to a monovalent saturated cyclic, bicyclic, or bridged cyclic (e.g., adamantyl) hydrocarbon group of 3-12, 3-10, 3-8, 4-8, or 4-6 carbons, referred to herein, e.g., as “C₃-C₁₀cycloalkyl,” derived from a cycloalkane. Exemplary cycloalkyl groups include, but are not limited to, cyclohexanes, cyclopentanes, cyclobutanes and cyclopropanes.

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, and sulfur (“3-10 membered heterocyclyl”). In some embodiments, in heterocyclyl groups that contain one or more nitrogen atoms, the point of attachment is a carbon or nitrogen atom, as valency permits. In some embodiments, a heterocyclyl group is either be monocyclic (“monocyclic heterocyclyl”) or a fused, bridged or spiro ring system such as a bicyclic system (“bicyclic heterocyclyl”), and is saturated or is partially unsaturated. In some embodiments, heterocyclyl bicyclic ring systems include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more phenyl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” are used interchangeably.

In some embodiments, a heterocyclyl group is a 3-10 membered non-aromatic ring system having ring carbon atoms and 1-4 ring heteroatoms, wherein each heteroatom is independently selected from nitrogen, oxygen, and sulfur (“3-10 membered heterocyclyl”). In some embodiments, a heterocyclyl group is a 3-7 membered non-aromatic ring system having ring carbon atoms and 1-4 ring heteroatoms, wherein each heteroatom is independently selected from nitrogen, oxygen, and sulfur (“3-7 membered heterocyclyl”). In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

“Hetero” when used to describe a compound or a group present on a compound means that one or more carbon atoms in the compound or group have been replaced by a nitrogen, oxygen, or sulfur heteroatom. In some embodiments, hetero is applied to any of the hydrocarbyl groups described above such as alkyl, e.g., heteroalkyl; cycloalkyl, e.g., heterocyclyl; aryl, e.g., heteroaryl; and the like having from 1 to 5, and particularly from 1 to 3 heteroatoms.

The terms “halo” and “halogen” as used herein refer to an atom selected from fluorine (fluoro, —F), chlorine (chloro, —Cl), bromine (bromo, —Br), and iodine (iodo, —I). In certain embodiments, the halo group is either fluoro or chloro.

The term “haloalkyl” includes mono, poly, and perhaloalkyl groups substituted with one or more halogen atoms where the halogens are independently selected from fluorine, chlorine, bromine, and iodine.

The term “oxo” as used herein refers to ═O.

The term “hydroxyl” as used herein refers to-OH.

In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms are substituted or unsubstituted as valency permits, and include primary, secondary, tertiary, and quarternary nitrogen atoms.

These and other exemplary substituents are described in more detail in the Detailed Description, Examples, and Claims. The disclosure is not intended to be limited in any manner by the above exemplary listing of substituents.

### Other Definitions

As used herein, the term “about” when modifying a numeric value means within a reasonable range of the stated value, typically +10% unless otherwise specified. This term accounts for normal experimental or biological variability around the precise value.

As used herein, “pharmaceutically acceptable excipient” refers to any substance in a pharmaceutical formulation other than the active pharmaceutical ingredient(s). Exemplary pharmaceutical excipients include those that aid the manufacturing process; protect, support or enhance stability; increase bioavailability; or increase patient acceptability. They may also assist in product identification or enhance the overall safety or function of the product during storage or use.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” “individual” and “subject” are used interchangeably herein. None of the terms require the supervision of medical personnel.

The terms “disease”, “disorder”, and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition.

As used herein, and unless otherwise specified, a “therapeutically effective amount” or an “effective amount” of a compound is an amount sufficient to provide a therapeutic benefit in the treatment of a disease, disorder or condition, or to delay or minimize one or more symptoms associated with the disease, disorder or condition. A therapeutically effective amount or an effective amount of a compound means an amount of therapeutic agent, alone or in combination with other therapies, which provides a therapeutic benefit in the treatment of the disease, disorder or condition. In some embodiments, the term “therapeutically effective amount” or “effective amount” encompasses an amount that improves overall therapy, reduces or avoids symptoms or causes of disease or condition, or enhances the therapeutic efficacy of another therapeutic agent.

As used herein, the term “ketone body”, “exogenous ketone” or “exogenous ketone body” means a compound or species which is a ketone or a ketone body precursor, that is, a compound or species which is a precursor to a ketone and which may be converted or metabolized to a ketone. Ketone bodies are produced when fatty acids levels are raised in the body and are metabolized by the body for energy. Ketone bodies have been disclosed as being suitable for reducing the levels of free fatty acids circulating in the plasma of a subject and that ingestion of ketone bodies can lead to various clinical benefits, including an enhancement of cognitive performance and treatment of cardiovascular conditions, diabetes and treatment of mitochondrial dysfunction disorders and in treating muscle fatigue and impairment. Examples of ketone bodies include, but are not limited to, beta-hydroxybutyrate (“BHB”) and acetoacetate (“AcAc”).

For consistency, the terms “administering” and “delivering” (with respect to providing a therapeutic composition to a subject) are used interchangeably herein. Likewise, terms like “exogenous ketone therapy,” “ketone composition,” or “ketone formulation” refer to the use of one or more ketone compounds (or precursors) administered to the subject as described.

For clarity and consistency throughout this specification, the following terms are defined as follows:

Bioenergetic Sufficiency refers to a metabolic state within a biological system (e.g., cell, tissue, organ, organism) where the effective rate of energy generation (E) is adequate to meet or exceed the total rate of energy requirement (R). Quantitatively, this state is characterized by a Metabolic Sufficiency Ratio (M) equal to or greater than 1 (M≥1), or more specifically, equal to or greater than the tissue-specific critical threshold (M≥ M_crit). This state allows for optimal function, maintenance, repair, and resilience to stress.

Energy Insufficiency/Bioenergetic Deficit/Metabolic Failure refers to a metabolic state where the effective rate of energy generation (E) is inadequate to meet the total rate of energy requirement (R). Quantitatively, this state is characterized by M<1, or more specifically, M<M_crit. This state is considered the central pathophysiological condition addressed by the present disclosure, leading to cellular stress, impaired function, accumulation of damage (entropy), and ultimately, disease manifestation or progression. Metabolic Sufficiency Ratio (M) refers to a dimensionless ratio defined as M =E/R, representing the core quantitative metric of bioenergetic health within the UTBS framework. It provides a universal measure of the balance between energy supply and demand.

Effective Energy Generation (E) represents the net rate at which usable energy, primarily in the form of ATP, is synthesized and made available to the cell or tissue from all utilized metabolic fuels. It is a function of the rate of uptake or availability of each substrate i (U_i), the proportion of that substrate directed towards energy-yielding oxidative pathways (governed by Partition Factor PF_i), and the efficiency with which each substrate is converted to ATP(η_i, reflecting pathway stoichiometry and mitochondrial coupling efficiency, e.g., P/O ratio). Mathematically, E can be represented as the sum over all contributing substrates: E=Σ_i (PF_i*U_i*η_i), where i includes glucose, fatty acids, ketone bodies, lactate, amino acids, etc.

Energy Requirement (R) represents the total rate of ATP consumption required by the cell or tissue to sustain all its functions. It includes components for basal metabolic processes (R_basal, e.g., maintaining ion gradients, basal biosynthesis), responding to stressors (R_stress, e.g., energy costs of inflammation, oxidative damage repair, unfolded protein response), performing tissue-specific work (R_activity, e.g., neuronal firing, muscle contraction, active transport), and cellular repair and maintenance (R_repair, e.g., protein turnover, DNA repair). R can be dynamic and increase significantly under pathological conditions.

Substrate Relativity refers to a core principle of UTBS stating that when the utilization of a primary or preferred fuel substrate is impaired (reducing its contribution to E), cells and tissues attempt to compensate and maintain M by dynamically upregulating the uptake and oxidation of alternative, available fuels. The metabolic importance of secondary fuels (e.g., ketones, lactate) thus increases relative to the failing primary pathway. This principle underlies the rationale for therapeutic strategies using alternative fuels.

Partition Factors (PFs) refers to a dimensionless variables (0≤PF_i≤1) representing the fraction of an available substrate i (after uptake) that is directed towards a specific metabolic fate, such as oxidation for ATP production (PF_i_ox), storage (e.g., glycogen, triglycerides), or biosynthesis. PFs are dynamically regulated by hormonal signals, substrate availability, cellular energy status (related to M), and genetic factors, determining fuel selection and allocation.

Metabolic Flexibility is defined within UTBS as the capacity of a biological system to dynamically and appropriately adjust its substrate Partition Factors (PFs) in response to changing physiological conditions (e.g., feeding vs. fasting, rest vs. exercise) or metabolic challenges, in order to maintain bioenergetic sufficiency (M). Metabolic inflexibility, the failure of this adaptive capacity, is a hallmark of conditions like insulin resistance and MetS and contributes to energy insufficiency.

Critical Metabolic Sufficiency Threshold (M_crit) refers to the specific value of the Metabolic Sufficiency Ratio (M) below which a particular cell type or tissue begins to exhibit functional impairment or enters a state of pathological stress. M_crit is tissue-specific and depends on the tissue's minimum energy requirement for essential function (R min functional) relative to its maximum potential energy generation capacity (E_max_capacity), potentially modeled as M_crit,j=R_min_functional,j/E_max_capacity,j for tissue j. Tissues with high intrinsic energy demands or limited compensatory capacity have a higher M_crit, making them more vulnerable to systemic metabolic stress.

Energy Rescue refers to therapeutic strategies specifically designed to restore or improve the Metabolic Sufficiency Ratio (M) in states of energy insufficiency. This is typically achieved by interventions that increase effective energy generation (E) or decrease excessive energy requirements (R).

Entropy Accumulation, within the UTBS framework, links chronic energy insufficiency (persistently low M) to the principles of the second law of thermodynamics. It posits that maintaining the highly ordered state of living systems requires continuous energy input (high M). When M is insufficient, the system's ability to counteract the natural tendency towards disorder (entropy) is compromised, leading to an accelerated rate of entropy generation (σ). This manifests physiologically as accumulating cellular damage, dysfunction, aging, and the progression of chronic disease. Improving M is thus viewed as a strategy to slow entropy accumulation.

β-Hydroxybutyrate (BHB) refers to a ketone body and primary energy metabolite used in this therapy. It exists in D and L enantiomers; D-βHB is the biologically active form readily metabolized to acetoacetate and then to acetyl-CoA. Blood BHB is measured in millimolar (mM). Nutritional ketosis is typically 0.5-5 mM BHB. In this document “BHB” usually refers to D-βHB unless specified.

Acetoacetate (AcAc) refers to another ketone body, chemically a 4-carbon keto-acid. It is in equilibrium with BHB (BHB ↔AcAc via dehydrogenase). AcAc is less stable (prone to decarboxylate). Some esters release AcAc directly; others release BHB which partially converts to AcAc. Therapeutically both serve as fuels.

Ketone Bodies, collectively BHB, AcAc, and acetone (the last is a minor byproduct). They are produced by the liver during fat metabolism and used by tissues as energy. Exogenous ketone therapy delivers these without the need for fat breakdown.

Orphan Disease refers to a disease affecting <200,000 people in the USA (or meeting region-specific prevalence criteria). Often genetic, rare, and may have no current effective therapies due to small market. Examples given span metabolic, neurologic, and neuromuscular categories.

Impaired Energy Metabolism is a general term for any condition where cells cannot effectively generate ATP from the usual substrates (glucose, fats). This could be due to enzyme deficiencies, transporter defects, mitochondrial dysfunction, etc. Consequences include energy crisis, cell damage, and organ dysfunction.

Genus (of compounds) refers to a broad class of chemical compounds covered by a description or formula. Here, the genus is ketone esters/analogs that elevate ketones. Representative species are individual examples within that genus.

GLUT1-DS is Glucose transporter type 1 deficiency syndrome. A rare genetic epilepsy caused by insufficient glucose transport into the brain. Abbreviations: Glut1 (gene/protein), DS for deficiency syndrome.

PDHc Deficiency is Pyruvate dehydrogenase complex deficiency. Causes a buildup of pyruvate/lactate and lack of acetyl-CoA. Often X-linked (PDHA1 gene) or others; leads to lactic acidosis and neuro symptoms.

MELAS, MERRF are Mitochondrial syndromes (MELAS=Mitochondrial Encephalomyopathy, Lactic Acidosis, Stroke-like episodes; MERRF=Myoclonic Epilepsy with Ragged Red Fibers). Caused by mtDNA mutations, energy failure in brain/muscle.

Leigh Syndrome is a severe neurological disorder (often infantile) with lesions in brainstem/basal ganglia due to various mitochondrial defects (can be nuclear or mtDNA genes, e.g., NDUFS4). Also called Subacute Necrotizing Encephalopathy.

FAOD refers to Fatty Acid Oxidation Disorder. A class of metabolic diseases where breakdown of fatty acids is impaired (e.g., VLCAD, MCAD, LCHAD deficiencies). Characterized by hypoketotic hypoglycemia, muscle breakdown, cardiomyopathy during fasting or stress.

Triheptanoin (C₇) is a triglyceride of three heptanoic acid chains. Used as an anaplerotic substrate in FAOD. Yields ketone bodies and TCA cycle substrates upon metabolism. Now approved as a therapy for LC-FAOD (branded Dojolvi).

Ketogenic Diet (KD) refers to a high-fat, low-carb diet that induces ketosis. Used as therapy for epilepsy and some metabolic conditions. Variants include classic KD, MCT diet, Modified Atkins Diet (MAD), etc.

NAD⁺/NADH refers to nicotinamide adenine dinucleotide, oxidized and reduced forms. Key redox couple in metabolism. NAD⁺ is required for glycolysis and TCA cycle dehydrogenases; NADH is produced and feeds electrons to the ETC. The ratio NAD⁺/NADH affects metabolic flux and signaling (sirtuins, etc.). A higher NAD⁺ (or higher NAD⁺/NADH) is generally a sign of healthy metabolism.

Lactate is the end product of anaerobic glycolysis. Builds up when pyruvate isn't fully metabolized in mitochondria. Elevated lactic acid is a marker of mitochondrial dysfunction or PDH block. Reducing lactate via ketones is beneficial for conditions like PDH deficiency.

PID Controller or Proportional-Integral-Derivative controller refers to a common algorithm in closed-loop systems that adjusts outputs to minimize error in a measured variable. In our context, it reads ketone level (error=difference from target) and adjusts pump rate accordingly. The parameters (P, I, D gains) are tuned for stable control.

MCT or Medium-Chain Triglyceride are often C8 and C10 fats that are ketogenic because they are rapidly oxidized to ketones. Used in some modified ketogenic diets.

HDAC inhibitor or Histone deacetylase inhibitor includes BHB which is a mild HDAC inhibitor, influencing gene expression (like elevating antioxidant genes). Mentioned for neuroprotective signaling.

BDNF or Brain-Derived Neurotrophic Factor refers to a protein that supports neuron survival and plasticity. Ketones have been observed to increase BDNF levels in some contexts, which could benefit neurodegenerative diseases.

Liver and muscle enzymes such as aspartate aminotransferase (AST), alanine aminotransferase (ALT), creatine kinase (CK). ALT and CK are mentioned as injury markers in examples.

EEG or Electroencephalography, measures brain electrical activity, used to detect seizures. EEG improvements re referred to in Example 4.

“Metabolic dysregulation” refers to an abnormal state of cellular or organismal metabolism. It may include impaired glucose utilization, mitochondrial dysfunction, a shift toward glycolytic (Warburg) metabolism, altered ATP production, or dysregulated anabolic/catabolic pathways. Indicators of metabolic dysregulation can include elevated lactate, low ATP levels, altered respiratory quotient, insulin resistance, or aberrant fuel substrate usage. For example, cancer cells and activated immune cells often exhibit metabolic dysregulation by favoring glycolysis over oxidative phosphorylation.

“Redox dysregulation” refers to an imbalance in the cellular redox couples, such as NADH/NAD⁺, NADPH/NADP⁺, or glutathione (GSH/GSSG), leading to either reductive stress (excess reducing equivalents, high NADH relative to NAD⁺) or oxidative stress (excess ROS, depleted NADPH). Redox dysregulation often manifests as high lactate: pyruvate ratio, high β-hydroxybutyrate: acetoacetate (BHB:AcAc) ratio (reflecting a low NAD⁺/NADH in mitochondria), accumulation of ROS, and oxidative damage. It can result in impaired enzyme function (since many dehydrogenases and sirtuins require NAD⁺) and activation of redox-sensitive pathological pathways.

“Epigenetic dysregulation” refers to aberrations in the patterns of gene expression control that are not due to DNA sequence changes but due to chromatin modifications and related mechanisms. This includes abnormal histone modifications (hypoacetylation or hyperacetylation, abnormal methylation patterns), DNA methylation changes, or altered chromatin structure leading to gene silencing or overexpression of pathogenic genes. Epigenetic dysregulation is implicated in cancer (silencing tumor suppressors), neurodevelopmental disorders (mutations in chromatin regulators leading to improper gene expression), and aging-related decline (accumulation of repressive chromatin marks). Markers of epigenetic dysregulation could be, for example, low levels of histone acetylation at promoters of neuroplasticity genes, or a global increase in repressive marks like H3K9me3

“Immune dysregulation” in the context of MERIT refers to abnormal immune system activity that stems at least in part from underlying metabolic or redox disturbances. It can mean chronic inflammation (inflammasome activation, elevated cytokines), autoimmunity, or immune suppression/exhaustion (e.g., T cells in tumors becoming non-functional). Often, immune dysregulation is tied to metabolic state: for instance, macrophages in a pro-inflammatory state rely on glycolysis and have high NADH, which can be corrected by metabolic interventions. In this disclosure, immune dysregulation is addressed indirectly by correcting metabolic/redox drivers or directly via ketone signaling (e.g., BHB acting on immune cell receptors or inflammasome components).

“Disease characterized by metabolic, redox, or epigenetic dysregulation (MRED)” means any disease or condition wherein one or more of the following is a feature of its pathology: abnormal cellular metabolism, abnormal redox balance, or abnormal epigenetic/chromatin state. This is an intentionally broad category. It includes, for example: metabolic diseases (diabetes, obesity, metabolic syndrome-metabolic dysregulation obvious), neurological diseases (Alzheimer's, Parkinson's—have deficits in glucose uptake/metabolism in brain, redox stress, and epigenetic changes in neurons), psychiatric illnesses (schizophrenia, bipolar-evidence of mitochondrial dysfunction and epigenetic changes), cancers (nearly all cancers rewire metabolism (Warburg effect) and have redox and epigenetic aberrations), rare genetic disorders of metabolism or chromatin (e.g. mitochondrial myopathies, Rett syndrome, Kabuki syndrome—by definition involve these axes), cardiovascular diseases (heart failure involves metabolic shift and redox stress), inflammatory/autoimmune diseases (often involving immune cell metabolic shifts and redox inflammatory signaling), and so on. A disease can be characterized by MRED even if it's not traditionally labeled a metabolic disease—it suffices that at least one of those dysregulated processes plays a significant role in the disease pathology or symptoms.

“Metabolic-Epigenetic-Redox Immune Therapy (MERIT)” is a therapeutic approach as described in this patent that targets MRED aspects of disease using ketone bodies. Reference to “MERIT therapy”, implie a treatment regimen that induces a controlled ketotic state in the patient in order to correct metabolic, redox, epigenetic, and consequent immune abnormalities. MERIT is thus an umbrella term for the approach, which could be delivered via various compositions but always with the idea of multi-axis correction by ketones.

“Therapeutic Titratable Ketosis (TTK)” is the state of controllable, adjustable ketonemia induced in a patient for therapeutic benefit. “Therapeutic ketonemia” means blood ketone body concentrations elevated to a level sufficient to have pharmacological effects (typically >0.5 mM BHB, often 1-6 mM range, which is above normal fed levels but below diabetic ketoacidosis levels). “Titratable” emphasizes that the level is not incidental but is set as a target and actively regulated (like one would titrate blood pressure to a target range using meds). TTK encompasses both the process (the treat-to-target method) and the outcome state. Reference to “achieving TTK” in a patient mean the desired ketone level window has been achieved. TTK can be further described by parameters like “time-in-range”, meaning the percentage of a 24-hour period that the patient's ketone levels remain within the target window. For instance, 50% time-in-range might mean 12 hours/day in therapeutic ketosis. TTK also may involve programmed variation—e.g., cyclic ketosis (intermittent fasting mimicking) vs sustained ketosis, depending on what's optimal for the condition.

“Therapeutic ketonemia” means the presence of ketone bodies in the bloodstream at concentrations that confer a therapeutic effect. Typically, reference to β-hydroxybutyrate (BHB) and acetoacetate (AcAc) as the primary ketone bodies (acetone is also a ketone body but mostly a byproduct). In context, this term implies that ketone levels are elevated intentionally (through exogenous supply) to within a safe yet efficacious range (for example, 1-5 mM BHB). It is distinguished from nutritional ketosis by the fact that it is induced by administered agents and can be attained even with normal carbohydrate intake, and distinguished from ketoacidosis by being controlled (e.g., pH maintained, levels not excessively high such as >10 mM).

“BHB:AcAc ratio” or “β-hydroxybutyrate: acetoacetate ratio” refers to the molar ratio of BHB to AcAc in a given compartment (often measured in blood plasma). This ratio is a well-known proxy for the mitochondrial NAD⁺/NADH ratio in the liver: a high BHB:AcAc implies a high NADH/NAD⁺ (more reduced state), whereas a lower ratio implies a more oxidized state. In healthy nutritional ketosis, BHB:AcAc might range roughly from 1:1 up to 4:1, whereas in diabetic ketoacidosis it skews higher (due to extreme redox changes). In this disclosure, modulation of the BHB:AcAc ratio is used as a therapeutic lever. For example, “AKBR” (Arterial Ketone Body Ratio) is sometimes used in literature to denote [AcAc]/[BHB]; herein BHB:AcAc is used for consistency but conceptually one could use either. A BHB:AcAc ratio of ~1:1 or 2:1 is targeted for certain therapies, as opposed to >5:1 in uncontrolled states. Acceptable ranges wherein the therapy maintains a BHB:AcAc ratio between 0.8 and 3.0″ etc.

“AKBR” specifically stands for Arterial Ketone Body Ratio, traditionally defined as [acetoacetate]/[β hydroxybutyrate] in arterial blood. It's used clinically as an indicator of liver mitochondrial redox state (with low AKBR indicating a reduced state often seen in shock). Herein mention of AKBR, an increase in AKBR means shifting toward more AcAc (more NAD⁺ available). Since AKBR is the inverse of BHB:AcAc, controlling AKBR is essentially the same information as controlling the ratio, just inverted. Focus on BHB:AcAc is used for simplicity, but either term could be used.

“NADH drain” refers to the therapeutic mechanism of consuming NADH to form NAD⁺. Typically, this is via the reaction AcAc+NADH→BHB+NAD⁺ (catalyzed by BDH1). Sometimes also called “NAD⁺ regeneration”. Reference to a therapy “drains NADH”, it is meant that it pulls the excess reducing equivalents out by converting them along with AcAc into BHB, thereby relieving reductive stress.

“Kbhb” and “Kacac” refer to specific histone lysine acylation marks: Kbhb is lysine β hydroxybutyrylation, a post-translational modification where a β-hydroxybutyryl group (derivable from BHB-CoA) is attached to the epsilon-amino group of a lysine on a histone or other protein. Kacac is lysine acetoacetylation, attachment of an acetoacetyl group (derivable from acetoacetyl COA) to a lysine. These modifications can alter chromatin in ways analogous to or distinct from acetylation. They often mark active gene regions (e.g., high Kbhb was found at active gene promoters during starvation). In this document, reference to “increase Kbhb or Kacac”, it is meant that increasing the prevalence of those histone marks in target cells, presumably to affect gene expression. For example, raising cellular BHB might increase histone Kbhb at promoters of oxidative stress resistance genes, thereby enhancing their transcription.

“HDAC2 hyperactivity” is a shorthand for conditions in which histone deacetylase 2 (or other HDACs similarly) are over-active or over-expressed, leading to pathological hypoacetylation of histones and gene repression. Mention of HDAC2 specifically because it's implicated in memory impairment and other pathology. MERIT can counter HDAC2 hyperactivity by direct inhibition by BHB or by increasing acetyl-CoA (substrate for HATs) or via Kbhb that functionally may prevent HDAC binding on chromatin.

“M-score” (MERIT score or metabolic score) is a composite index comprising multiple measurable biomarkers reflecting the metabolic, redox, epigenetic, and immune state of a patient. For instance, an M-score could be calculated from a weighted combination of: NAD⁺/NADH ratio, GSH/GSSG ratio, blood BHB level, lactate level, histone acetylation level in PBMCs, IL-6 and IL-1β levels, etc. A high M-score might indicate a healthy balanced state, whereas a low M-score indicates severe dysregulation. In practice, the M-score is used to guide therapy: e.g., only patients below a threshold M-score get the treatment, or the dose is titrated until the M-score improves by a certain amount. It's analogous to an APGAR score or MELD score but for metabolic/redox/epigenetic health. Since it's not a standard measure, it is defined operationally in examples (e.g., “M-score= [NAD⁺](μM)+10*[acetylation (% of histone H3K9)]−[IL-6](pg/mL)” as a toy formula).

“Time-in-range” for ketone or ratio windows refers to the percentage of time a patient's measured value stays within a predefined target range. Borrowed from diabetes glucose management, where “time in range” is a metric, herein it applies to ketone therapy. For example, if the target BHB is 2-4 mM, and over 24 hours the patient's BHB was between 2 and 4 mM for 12 hours, that's 50% time—in range or ≥50% time-in-range at 1-5 mM BHB over at least 7 days of therapy. Herein it will often specify that effective therapy requires maintaining the ketones in range for a minimum duration (since brief spikes might not recapitulate all gene expression changes or immune effects). Time-in-range can also apply to ratios (e.g., maintaining BHB:AcAc between 1:1 and 4:1 for X % of the day). It underscores the concept of continuous or frequent closing vs one-off closing.

“MERIT-Responsive Condition” refers to any disease, disorder, or pathological state characterized by dysregulation of one or more of the Metabolic, Epigenetic, Redox, or Immune (MERIT) pillars, or by dysregulation of circadian or endocrine signaling pathways that are influenced by ketone body metabolism. Examples include, without limitation, oncological conditions in which T-cell metabolic state gates immunotherapeutic efficacy; autoimmune and inflammatory diseases characterized by NLRP3 inflammasome hyperactivation; infectious diseases and sepsis-associated organ dysfunction driven by dysregulated innate immunity; neurodegenerative diseases characterized by glial activation and neuroinflammation; neuropsychiatric conditions characterized by epigenetic dysregulation or bioenergetic dysfunction; metabolic diseases including diabetes and obesity; cardiovascular diseases associated with inflammation and metabolic dysfunction; and aging-related disorders and biological senescence. The critical feature is that the condition is responsive to the pharmacological induction and maintenance of a ketotic state, independent of dietary restriction, glucose hypometabolism, or any particular baseline metabolic phenotype. A MERIT-responsive condition is defined functionally by responsiveness, not by etiology.

“Metabolic-Epigenetic-Redox-Immune (MERIT) Dysregulation” refers to abnormal function in one or more of five orthogonal pillars: (1) Metabolic dysregulation, characterized by mitochondrial dysfunction, abnormal NAD⁺/NADH ratios, impaired oxidative phosphorylation, or altered substrate utilization; (2) Epigenetic dysregulation, characterized by abnormal histone acetylation, aberrant histone modification patterns, dysregulated expression of epigenetic writers and erasers (histone acetyltransferases, histone deacetylases), or impaired chromatin remodeling; (3) Redox dysregulation, characterized by oxidative stress, impaired ROS scavenging, dysregulation of Nrf2-mediated antioxidant response, or altered NAD⁺/NADH and NADPH/NADP+ ratios; (4) Immune dysregulation, characterized by NLRP3 inflammasome hyperactivation, altered T-cell or B-cell metabolic state, impaired barrier function (intestinal, blood-brain, or mucosal), aberrant innate immune signaling, or dysregulated cytokine production; and (5) Signaling dysregulation, including abnormal circadian rhythm, dysregulated FGF21 signaling, impaired HCAR2/GPR109A signaling, or impaired stress-response hormone signaling. A MERIT-responsive condition exhibits dysregulation in one or more of these pillars and is ameliorated by pharmacological restoration of MERIT homeostasis through ketosis induction.

“Ketogenic Agent” refers to any pharmacological, dietary, or compositional means that results in the elevation of circulating ketone bodies (β-hydroxybutyrate and/or acetoacetate) to a therapeutically meaningful level. The genus of ketogenic agents includes, without limitation: (a) dietary interventions (ketogenic diet, low-carbohydrate high-fat diet, carbohydrate-restricted diet); (b) fasting regimens (intermittent fasting, time-restricted feeding, fasting-mimicking diet, prolonged fasting); (c) ketone monoester compounds (e.g., (R)-3-hydroxybutyl acetoacetate, R,S-1,3-butanediol acetoacetate esters, and related compounds); (d) ketone salts and their derivatives (sodium β-hydroxybutyrate, potassium β-hydroxybutyrate, calcium β-hydroxybutyrate, magnesium β-hydroxybutyrate, and related salts); (e)_{1,3}-butanediol and its metabolites; (f) medium-chain triglycerides (MCT oil, hexanoic acid, octanoic acid, decanoic acid, and related compounds); (g) ketone-carrier conjugates (prodrugs of ketone bodies linked to carrier molecules, peptides, lipids, or carbohydrate moieties that enhance bioavailability, tolerability, or kinetics); (h) free ketone body infusates (intravenous, intra-arterial, or direct tissue administration of β-hydroxybutyrate or acetoacetate); (i) SGLT2 inhibitors and related sodium-glucose transporter modulators; (j) FGF21 agonists or direct FGF21 administration; (k) nicotinamide adenine dinucleotide (NAD⁺) boosters (NMN, NR, NA, or precursors thereof) that enhance NAD⁺/NADH ratios and fatty acid oxidation; (1) peroxisome proliferator-activated receptor (PPAR) agonists that enhance mitochondrial biogenesis and fatty acid oxidation; (m) direct mitochondrial function enhancers or mitochondrial biogenesis activators; (n) any pharmacological agent that increases hepatic ketone production or reduces hepatic ketone utilization; and (o) any combination of the foregoing. The genus is open-ended and non-limiting. Any compound, composition, diet, regimen, or intervention that achieves elevation of circulating ketone bodies to a therapeutically meaningful level falls within the definition of ketogenic agent unless expressly excluded from a specific claim.

“Ketotic State” refers to a physiological condition characterized by elevation of circulating ketone bodies (β-hydroxybutyrate and acetoacetate, individually or in combination) to a level sufficient to engage downstream target-engagement mechanisms including histone β-hydroxybutyrylation, NLRP3 inflammasome suppression, HCAR2/GPR109A signaling, circadian rhythm modulation, or mitochondrial bioenergetic optimization. In functional terms, a ketotic state is defined as a circulating β-hydroxybutyrate concentration of at least 0.5 millimolar (mM) when measured under fasting or post-absorptive conditions, or equivalent concentration of acetoacetate, or total ketone body concentration (BHB+AcAc) meeting or exceeding the 0.5 mM threshold. For purposes of sustained therapeutic effect, the ketotic state should be maintained for a duration of at least several hours per day (sufficient for meaningful target engagement) and preferably for the majority of the 24-hour day. The specific circulating BHB target may vary depending on the indication, the particular MERIT pillar being engaged, and individual pharmacogenetic factors; ranges of 0.5-5 mM BHB are typical for most MERIT-responsive indications, with specific target ranges determined through biomarker-guided dose titration. Importantly, the definition of ketotic state encompasses the total ketone body exposure (BHB+AcAc, including both endogenously produced and exogenously delivered ketone bodies) and is not limited to any particular means of ketosis induction.

“Target Engagement” refers to the measurable biochemical or biophysical interaction between a circulating ketone body (or its metabolites) and a downstream molecular target, resulting in a quantifiable change in cellular or tissue-level function. Target-engagement readouts include, without limitation: (a) histone β-hydroxybutyrylation (Kbhb) measured by mass spectrometry, immunoassay, or high-resolution proteomics; (b) histone acetylation measured by immunohistochemistry, flow cytometry, or Western blotting; (c) NLRP3 inflammasome activation status measured by caspase-1 activity, pro-IL-1β cleavage, or multiplex cytokine assay; (d) NAD⁺/NADH ratio measured by fluorescence spectroscopy or mass spectrometry; (e) histone deacetylase activity measured by enzymatic assay; (f) circulating cytokine levels (IL-1β, IL-6, TNF-α, IL-10, IL-18) measured by ELISA or multiplex immunoassay; (g) T-cell metabolic phenotype (measured by metabolic flux analysis, extracellular acidification rate, or oxygen consumption rate); (h) circulating FGF21 levels; (i) circadian rhythm markers (BMAL1, PER2, Clock gene expression); (j) ROS production or antioxidant enzyme activity; or (k) any other biochemical marker indicative of target engagement in a MERIT pillar. Target engagement is a quantitative measure of the degree to which the ketotic state has engaged the downstream mechanisms responsible for therapeutic efficacy.

“State-Dependent Drug Efficacy” refers to the phenomenon in which the clinical efficacy of a therapeutic agent (a kinase inhibitor, monoclonal antibody, receptor agonist, cytokine modulator, or other pharmacological agent) is significantly modulated by the metabolic state of the subject receiving the agent. In the context of the present disclosure, state-dependent efficacy refers specifically to the situation in which a drug that shows poor or null efficacy in subjects with dysregulated MERIT (e.g., metabolically dysregulated subjects, obese subjects, subjects with elevated insulin, subjects with systemic inflammation) shows substantially improved efficacy when the same drug is administered to subjects in whom the ketotic state has been pharmacologically induced and maintained. State-dependent efficacy is not merely a pharmacokinetic effect (altered absorption, distribution, or elimination of the drug); rather, it is a pharmacodynamic effect in which the metabolic state of the target tissue or immune cell population gates the ability of the drug to engage its target and produce a therapeutic response. The induction of a ketotic state prior to or concurrent with drug administration can convert non-responders to responders, increase the magnitude of response in partial responders, or restore efficacy to drugs that had previously failed in unselected populations.

“Pharmacologically Induced Ketosis” refers to the elevation of circulating ketone bodies through the administration of a pharmacological agent (a ketogenic agent as defined above), in contrast to dietary or behavioral ketosis achieved through dietary restriction, fasting, or other lifestyle modification. Pharmacologically induced ketosis encompasses oral agents (ketone salts, ketone monoesters, ketone-carrier conjugates, SGLT2 inhibitors, NAD⁺ boosters, PPAR agonists), parenteral agents (intravenous or intra-arterial ketone infusates), and transdermal or other routes. Critically, pharmacologically induced ketosis is independent of dietary restriction: a subject receiving a pharmacological ketogenic agent need not alter their diet, need not fast, and need not exhibit any baseline metabolic phenotype change to achieve a therapeutic ketotic state. This is the defining feature that distinguishes pharmacological ketosis from dietary or fasting-induced ketosis.

“Treat-to-Target” refers to a therapeutic approach in which the dose and frequency of administration of a ketogenic agent are titrated (increased, decreased, or adjusted in timing) to achieve a predefined target biomarker readout. In the context of the present disclosure, treat-to-target approaches may aim to achieve a target circulating BHB level (e.g., 1-2 mM), a target degree of histone β-hydroxybutyrylation in peripheral blood cells (e.g., ≥50% of total histones bearing the Kbhb mark), a target degree of NLRP3 inflammasome suppression (e.g., ≥60% reduction in caspase-1 activity), a target NAD⁺/NADH ratio (e.g., >1.5-fold elevation), or a composite MERIT biomarker index. Treat-to-target closing is analogous to target-engagement-guided closing of kinase inhibitors (where dose is titrated to achieve a target degree of phosphorylation suppression) or therapeutic drug monitoring in immunosuppressive therapy. Treat-to-target approaches enable individualization of ketogenic agent closing, accommodation of pharmacogenetic variability, and optimization of the balance between efficacy and tolerability.

“Independent of Dietary Restriction” means that the methods, compositions, and uses described herein do not require the subject to undergo any change in dietary intake, macronutrient composition, caloric intake, or eating pattern. A subject receiving a pharmacological ketogenic agent may maintain an unrestricted diet (including ad libitum carbohydrate, protein, fat, and caloric intake) and may achieve a therapeutic ketotic state through the pharmacological agent alone. This contrasts with dietary ketogenic therapy, which requires a sustained reduction in carbohydrate intake and a corresponding increase in fat intake to maintain therapeutic ketosis. Independence from dietary restriction is a critical feature enabling clinical accessibility, scalability, and compliance.

“Does Not Require Glucose Hypometabolism” means that the methods, compositions, and uses described herein are not limited to subjects exhibiting reduced cerebral metabolic rate of glucose (CMRg1c), reduced whole-body glucose oxidation, elevated blood glucose, hyperglycemia, glucose intolerance, insulin resistance, or any other particular glucose metabolic phenotype. A subject may have a normal or elevated glucose metabolic rate, may be insulin sensitive, and may have optimal glucose homeostasis while still achieving therapeutic benefit from a pharmacologically induced ketotic state. The MERIT mechanisms engaged by ketosis (histone β-hydroxybutyrylation, NLRP3 suppression, HCAR2 signaling, circadian synchronization) are independent of glucose metabolic state and operate through non-energy mechanisms. This decoupling from glucose hypometabolism dramatically expands the scope of therapeutic application and removes a major constraint on clinical accessibility that had limited prior dietary ketogenic therapies.

“MERIT Biomarker” refers to a measurable biochemical, immunological, biophysical, or imaging parameter that reflects the functional status of one or more of the five MERIT pillars or that predicts responsiveness to pharmacologically induced ketosis. MERIT biomarkers include, without limitation: (a) Metabolic biomarkers: circulating β-hydroxybutyrate concentration, acetoacetate concentration, total ketone body concentration, NAD⁺/NADH ratio, NAD⁺/NADP+ ratio, lactate/pyruvate ratio, metabolic rate measured by indirect calorimetry, ATP/ADP ratio, free fatty acid concentration, fractional fatty acid oxidation; (b) Epigenetic biomarkers: histone acetylation (lysine acetylation on histone H3 and H4), histone β-hydroxybutyrylation (Kbhb), global histone methylation patterns, histone deacetylase activity (HDAC1-11 isoform-specific activities), bromodomain protein engagement, transcriptional output of histone-acetylation-dependent genes; (c) Redox biomarkers: reactive oxygen species (ROS) production measured by fluorescence or electron paramagnetic resonance, oxidative stress markers (malondialdehyde, protein carbonyls, 8-oxo-deoxyguanosine), antioxidant enzyme activity (catalase, superoxide dismutase, glutathione peroxidase), glutathione (GSH/GSSG ratio), Nrf2 nuclear translocation and activity; (d) Immune biomarkers: NLRP3 inflammasome activation (caspase-1 activity, ASC speck formation, NLRP3 protein-protein interaction measured by immunoprecipitation or proximity ligation assay), circulating cytokines (IL-1β, IL-18, IL-6, TNF-α, IL-10, interferon-Y), T-cell phenotype and metabolic state (CD4+vs CD8+ ratio, T-regulatory cell frequency, T-cell memory phenotype, T-cell glucose uptake and extracellular acidification rate), barrier function markers (zonula occludens-1, claudins, intestinal permeability measured by FITC-dextran flux or lactulose/mannitol ratio, occludin); (e) Signaling and circadian biomarkers: circulating FGF21, HCAR2/GPR109A engagement (measured by receptor internalization, pathway activation, or downstream effector phosphorylation), circadian rhythm markers (BMAL1, CLOCK, PER1, PER2, CRY1, CRY2 gene expression or protein abundance), melatonin secretion, cortisol circadian amplitude; and (f) Integrated MERIT biomarker panels: composite indices integrating multiple biomarkers from different pillars (e.g., a weighted sum of normalized Kbhb, NLRP3 suppression, NAD⁺/NADH elevation, and T-cell metabolic shift) to provide a comprehensive MERIT activation score (MVX). MERIT biomarkers are used for patient selection (enriching for subjects most likely to benefit from ketogenic agent treatment), response prediction (predicting which subjects will achieve therapeutic benefit), dose titration (guiding the adjustment of ketogenic agent dose to achieve target biomarker readouts), and mechanism-of-action validation (confirming that ketosis is engaging intended downstream targets).

These definitions are intended to clarify but not limit the scope-many terms like metabolic dysregulation or therapeutic ketosis should be read broadly in light of the description.

### Exemplary Compounds and Compositions as Ketogenic Agents

### Compounds

Disclosed herein, in certain embodiments, the ketogenic agent is a compound of Formula (A):

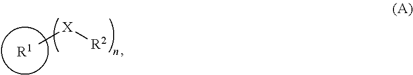

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH—or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and n is an integer from 1 to 50, wherein n represents the total number of ketone body moieties attached to R¹, and wherein n can be less than or equal to the total number of available attachment sites on R^{1.}

As generally defined above R¹ is a polyol, amino acid, or a derivative thereof. In certain embodiments, R¹ is a polyol. A polyol is a compound containing two or more hydroxy (OH) groups. In certain embodiments, R¹ is a polyol which is capable of delivering about 1 g to 50 g or more of ketone bodies to a subject diagnosed with a condition associated with energy insufficiency. In certain embodiments, R¹ is a polyol which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a condition associated with energy insufficiency. In certain embodiments, R¹ is a polyol which is capable of delivering about 1 g to 5 g or more of ketone bodies to a subject diagnosed with a condition associated with energy insufficiency. In certain embodiments, R¹ is a polyol which is capable of delivering about 5 g to 10 g or more of ketone bodies to a subject diagnosed with a condition associated with energy insufficiency. In certain embodiments, R¹ is a polyol which is capable of delivering about 10 g to 15 g or more of ketone bodies to a subject diagnosed with a condition associated with energy insufficiency. In certain embodiments, R¹ is a polyol which is capable of delivering about 15 g to 20 g or more of ketone bodies to a subject diagnosed with a condition associated with energy insufficiency. In certain embodiments, R¹ is a polyol which is capable of delivering about 20 g to 25 g or more of ketone bodies to a subject diagnosed with a condition associated with energy insufficiency. In certain embodiments, R¹ is a polyol which is capable of delivering about 25 g to 30 g or more of ketone bodies to a subject diagnosed with a condition associated with energy insufficiency. In certain embodiments, R¹ is a polyol which is capable of delivering about 30 g to about 35 g or more of ketone bodies to a subject diagnosed with a condition associated with energy insufficiency. In certain embodiments, R¹ is a polyol which is capable of delivering about 35 g to 40 g or more of ketone bodies to a subject diagnosed with a condition associated with energy insufficiency. In certain embodiments, R¹ is a polyol which is capable of delivering about 40 g to 45 g or more of ketone bodies to a subject diagnosed with a condition associated with energy insufficiency. In certain embodiments, R¹ is a polyol which is capable of delivering or about 45 g to 50 g or more of ketone bodies to a subject diagnosed with a condition associated with energy insufficiency.

In certain embodiments, R¹ is a polyol which is capable of delivering about 1 g or more of ketone bodies to a subject diagnosed with a condition associated with energy insufficiency. In certain embodiments, R¹ is a polyol which is capable of delivering about 5 g or more of ketone bodies to a subject diagnosed with a condition associated with energy insufficiency. In certain embodiments, R¹ is a polyol which is capable of delivering about 10 g or more of ketone bodies to a subject diagnosed with a condition associated with energy insufficiency. In certain embodiments, R¹ is a polyol which is capable of delivering about 15 g or more of ketone bodies to a subject diagnosed with a condition associated with energy insufficiency. In certain embodiments, R¹ is a polyol which is capable of delivering about 20 g or more of ketone bodies to a subject diagnosed with a condition associated with energy insufficiency. In certain embodiments, R¹ is a polyol which is capable of delivering about 25 g or more of ketone bodies to a subject diagnosed with a condition associated with energy insufficiency. In certain embodiments, R¹ is a polyol which is capable of delivering about 30 g or more of ketone bodies to a subject diagnosed with a condition associated with energy insufficiency. In certain embodiments, R¹ is a polyol which is capable of delivering about 35 g or more of ketone bodies to a subject diagnosed with a condition associated with energy insufficiency. In certain embodiments, R¹ is a polyol which is capable of delivering about 40 g or more of ketone bodies to a subject diagnosed with a condition associated with energy insufficiency. In certain embodiments, R¹ is a polyol which is capable of delivering or about 45 g or more of ketone bodies to a subject diagnosed with a condition associated with energy insufficiency. In certain embodiments, R¹ is a polyol which is capable of delivering or about 50 g or more of ketone bodies to a subject diagnosed with a condition associated with energy insufficiency.

In certain embodiments, R¹ is a carbohydrate. In certain embodiments, R¹ is a carbohydrate which is capable of delivering about 1 g to 50 g or more of ketone bodies to a subject diagnosed with a condition associated with energy insufficiency. In certain embodiments, R¹ is a carbohydrate which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a condition associated with energy insufficiency. In certain embodiments, R¹ is a carbohydrate which is capable of delivering about 1 g to 5 g or more of ketone bodies to a subject diagnosed with a condition associated with energy insufficiency. In certain embodiments, R¹ is a carbohydrate which is capable of delivering about 5 g to 10 g or more of ketone bodies to a subject diagnosed with a condition associated with energy insufficiency. In certain embodiments, R¹ is a carbohydrate which is capable of delivering about 10 g to 15 g or more of ketone bodies to a subject diagnosed with a condition associated with energy insufficiency. In certain embodiments, R¹ is a carbohydrate which is capable of delivering about 15 g to 20 g or more of ketone bodies to a subject diagnosed with a condition associated with energy insufficiency. In certain embodiments, R¹ is a carbohydrate which is capable of delivering about 20 g to 25 g or more of ketone bodies to a subject diagnosed with a condition associated with energy insufficiency. In certain embodiments, R¹ is a carbohydrate which is capable of delivering about 25 g to 30 g or more of ketone bodies to a subject diagnosed with a condition associated with energy insufficiency. In certain embodiments, R¹ is a carbohydrate which is capable of delivering about 30 g to about 35 g or more of ketone bodies to a subject diagnosed with a condition associated with energy insufficiency. In certain embodiments, R¹ is a carbohydrate which is capable of delivering about 35 g to 40 g or more of ketone bodies to a subject diagnosed with a condition associated with energy insufficiency. In certain embodiments, R¹ is a carbohydrate which is capable of delivering about 40 g to 45 g or more of ketone bodies to a subject diagnosed with a condition associated with energy insufficiency. In certain embodiments, R¹ is a carbohydrate which is capable of delivering or about 45 g to 50 g or more of ketone bodies to a subject diagnosed with a condition associated with energy insufficiency.

In certain embodiments, R¹ is a carbohydrate which is capable of delivering about 1 g or more of ketone bodies to a subject diagnosed with a condition associated with energy insufficiency. In certain embodiments, R¹ is a carbohydrate which is capable of delivering about 5 g or more of ketone bodies to a subject diagnosed with a condition associated with energy insufficiency. In certain embodiments, R¹ is a carbohydrate which is capable of delivering about 10 g or more of ketone bodies to a subject diagnosed with a condition associated with energy insufficiency. In certain embodiments, R¹ is a carbohydrate which is capable of delivering about 15 g or more of ketone bodies to a subject diagnosed with a condition associated with energy insufficiency. In certain embodiments, R¹ is an carbohydrate which is capable of delivering about 20 g or more of ketone bodies to a subject diagnosed with a condition associated with energy insufficiency. In certain embodiments, R¹ is a carbohydrate which is capable of delivering about 25 g or more of ketone bodies to a subject diagnosed with a condition associated with energy insufficiency. In certain embodiments, R¹ is an carbohydrate which is capable of delivering about 30 g or more of ketone bodies to a subject diagnosed with a condition associated with energy insufficiency. In certain embodiments, R¹ is a carbohydrate which is capable of delivering about 35 g or more of ketone bodies to a subject diagnosed with a condition associated with energy insufficiency. In certain embodiments, R¹ is a carbohydrate which is capable of delivering about 40 g or more of ketone bodies to a subject diagnosed with a condition associated with energy insufficiency. In certain embodiments, R¹ is a carbohydrate which is capable of delivering or about 45 g or more of ketone bodies to a subject diagnosed with a condition associated with energy insufficiency. In certain embodiments, R¹ is a carbohydrate which is capable of delivering or about 50 g or more of ketone bodies to a subject diagnosed with a condition associated with energy insufficiency.

In certain embodiments, R¹ is an amino acid or a derivative thereof. In certain embodiments, R¹ is a amino acid or a derivative thereof which is capable of delivering about 1 g to 50 g or more of ketone bodies to a subject diagnosed with a condition associated with energy insufficiency. In certain embodiments, R¹ is a amino acid or a derivative thereof which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a condition associated with energy insufficiency. In certain embodiments, R¹ is a amino acid or a derivative thereof which is capable of delivering about 1 g to 5 g or more of ketone bodies to a subject diagnosed with a condition associated with energy insufficiency. In certain embodiments, R¹ is a amino acid or a derivative thereof which is capable of delivering about 5 g to 10 g or more of ketone bodies to a subject diagnosed with a condition associated with energy insufficiency. In certain embodiments, R¹ is a amino acid or a derivative thereof which is capable of delivering about 10 g to 15 g or more of ketone bodies to a subject diagnosed with a condition associated with energy insufficiency. In certain embodiments, R¹ is a amino acid or a derivative thereof which is capable of delivering about 15 g to 20 g or more of ketone bodies to a subject diagnosed with a condition associated with energy insufficiency. In certain embodiments, R¹ is a amino acid or a derivative thereof which is capable of delivering about 20 g to 25 g or more of ketone bodies to a subject diagnosed with a condition associated with energy insufficiency. In certain embodiments, R¹ is a amino acid or a derivative thereof which is capable of delivering about 25 g to 30 g or more of ketone bodies to a subject diagnosed with a condition associated with energy insufficiency. In certain embodiments, R¹ is a amino acid or a derivative thereof which is capable of delivering about 30 g to about 35 g or more of ketone bodies to a subject diagnosed with a condition associated with energy insufficiency. In certain embodiments, R¹ is a amino acid or a derivative thereof which is capable of delivering about 35 g to 40 g or more of ketone bodies to a subject diagnosed with a condition associated with energy insufficiency. In certain embodiments, R¹ is a amino acid or a derivative thereof which is capable of delivering about 40 g to 45 g or more of ketone bodies to a subject diagnosed with a condition associated with energy insufficiency. In certain embodiments, R¹ is a amino acid or a derivative thereof which is capable of delivering or about 45 g to 50 g or more of ketone bodies to a subject diagnosed with a condition associated with energy insufficiency.

In certain embodiments, R¹ is a amino acid or a derivative thereof which is capable of delivering about 1 g or more of ketone bodies to a subject diagnosed with a condition associated with energy insufficiency. In certain embodiments, R¹ is a amino acid or a derivative thereof which is capable of delivering about 5 g or more of ketone bodies to a subject diagnosed with a condition associated with energy insufficiency. In certain embodiments, R¹ is a amino acid or a derivative thereof which is capable of delivering about 10 g or more of ketone bodies to a subject diagnosed with a condition associated with energy insufficiency. In certain embodiments, R¹ is a amino acid or a derivative thereof which is capable of delivering about 15 g or more of ketone bodies to a subject diagnosed with a condition associated with energy insufficiency. In certain embodiments, R¹ is an amino acid or a derivative thereof which is capable of delivering about 20 g or more of ketone bodies to a subject diagnosed with a condition associated with energy insufficiency. In certain embodiments, R¹ is a amino acid or a derivative thereof which is capable of delivering about 25 g or more of ketone bodies to a subject diagnosed with a condition associated with energy insufficiency. In certain embodiments, R¹ is an amino acid or a derivative thereof which is capable of delivering about 30 g or more of ketone bodies to a subject diagnosed with a condition associated with energy insufficiency. In certain embodiments, R¹ is a amino acid or a derivative thereof which is capable of delivering about 35 g or more of ketone bodies to a subject diagnosed with a condition associated with energy insufficiency. In certain embodiments, R¹ is a amino acid or a derivative thereof which is capable of delivering about 40 g or more of ketone bodies to a subject diagnosed with a condition associated with energy insufficiency. In certain embodiments, R¹ is a amino acid or a derivative thereof which is capable of delivering or about 45 g or more of ketone bodies to a subject diagnosed with a condition associated with energy insufficiency. In certain embodiments, R¹ is a amino acid or a derivative thereof which is capable of delivering or about 50 g or more of ketone bodies to a subject diagnosed with a condition associated with energy insufficiency.

In certain embodiments, R¹ is selected from the group consisting of trehalose, inositol, ribose, inulin, creatine, glycine, carnitine, and taurine. In certain embodiments, R¹ is selected from the group consisting of trehalose, myo-inositol, D-ribose, inulin, creatine, glycine, carnitine, taurine, glycerol, allulose, aspartic acid, and carnosine. In certain embodiments, R¹ is selected from the group consisting of trehalose, myo-inositol, D-ribose, inulin, creatine, glycine, carnitine, and taurine. In certain embodiments, R¹ is D-ribose. In certain embodiments, R¹ is inulin. In certain embodiments, R¹ is creatine. In certain embodiments, R¹ is glycine. In certain embodiments, R¹ is carnitine. In certain embodiments, R¹ is taurine. In certain embodiments, R¹ is glycerol. In certain embodiments, R¹ is allulose. In certain embodiments, R¹ is aspartic acid. In certain embodiments, R¹ is carnosine.

In certain preferred embodiments, R¹ is trehalose, myo-inositol, or D-ribose. In certain embodiments, R¹ is myo-inositol or trehalose. In certain embodiments, R¹ is myo-inositol. In certain embodiments, R¹ is trehalose.

As generally defined above, X is a covalent linker joining selected from the group consisting of —O—C(O)—, —C(O)—O—, —C(O)—NH—, —NH—C(O)—, —C(O)—S—, —S—C(O)—, —O—C(O)—O—, —O—C(O)—NH—or —NH—C(O)—O—, —O—P(O₂)—O—, —O—SO₂—O—, —C═N—, —C═N—NH—, —O—, —NH—, —S—, and —S—S—. In certain embodiments, X is —O—C(O)—. In certain embodiments, X is —C(O)—O—. In certain embodiments, X is —C(O)—NH—. In certain embodiments, X is —NH—C(O)—. In certain embodiments, X is —C(O)—S—. In certain embodiments, X is —S—C(O)—. In certain embodiments, X is —O—C(O)—O—. In certain embodiments, X is —O—C(O)—NH—or —NH—C(O)—O—. In certain embodiments, X is —O—P(O₂)—O—. In certain embodiments, X is —O—SO₂—O—. In certain embodiments, X is —C═N—. In certain embodiments, X is —C═N—NH—. In certain embodiments, X is —O—. In certain embodiments, X is —NH—. In certain embodiments, X is —S—. In certain embodiments, X is —S—S—. In preferred embodiments, X is —O—or —NH—.

As generally defined above R² is a ketone body moiety selected from β-hydroxybutyryl (BHB), acetoacetyl (AcAc), or a derivative thereof. In certain embodiments, R² is β-hydroxybutyryl (BHB) or a derivative thereof. In certain embodiments, R² is β-hydroxybutyryl (BHB). In certain embodiments, R² is a acetoacetyl (AcAc) or a derivative thereof. In certain embodiments, R² is a acetoacetyl (AcAc). In certain embodiments, R² is

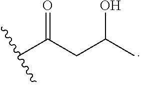 [SMILES: *C(=O)CC(C)O]

In certain embodiments, R² is

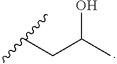 [SMILES: *CC(C)O]

In certain embodiments, R² is

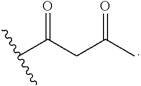 [SMILES: *C(=O)CC(C)=O]

In certain embodiments, R² is

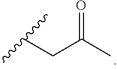 [SMILES: *CC(C)=O]

As generally defined above, n is an integer from 1 to 50. In certain embodiments, n is an integer from 1 to 20. In specific embodiments, n is 1, 2, 3, 4, 5, 6, 7, 8, 9, 10, 11, 12, 13, 14, 15, 16, 17, 18, 19, or 20. In certain embodiments n is 1. In certain embodiments n is 2. In certain embodiments n is 3. In certain embodiments n is 4. In certain embodiments n is 5. In certain embodiments n is 6. In certain embodiments n is 7. In certain embodiments n is 8. In certain embodiments n is 9. In certain embodiments n is 10. In certain embodiments nis 11. In certain embodiments n is 12. In certain embodiments n is 13. In certain embodiments n is 14. In certain embodiments n is 15. In certain embodiments n is 16. In certain embodiments n is 17. In certain embodiments n is 18. In certain embodiments n is 19. In certain embodiments n is 20. In certain embodiments, n is an integer which is equal to the number of hydroxy groups in R¹

In certain embodiments, the compound is compound of Formula (A):

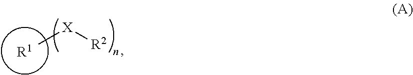

or a pharmaceutically acceptable salt or solvate thereof, wherein
R¹ is selected from the group consisting of trehalose, myo-inositol, D-ribose, inulin, creatine, glycine, carnitine, and taurine;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH—or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In certain embodiments, the compound is compound of Formula (A):

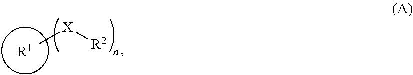

or a pharmaceutically acceptable salt or solvate thereof, wherein
R¹ is selected from the group consisting of trehalose, myo-inositol, D-ribose, inulin, creatine, glycine, carnitine, and taurine;
X is —O—or —NH—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In certain embodiments, the compound is compound of Formula (A):

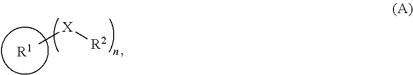

or a pharmaceutically acceptable salt or solvate thereof, wherein
R¹ is trehalose, myo-inositol, or D-ribose;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH—or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 8, wherein n represents the total number of ketone body moieties attached to R¹, and wherein n can be less than or equal to the total number of available attachment sites on R¹.

In certain embodiments, the compound is compound of Formula (A):

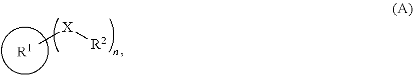

or a pharmaceutically acceptable salt or solvate thereof, wherein R¹ is trehalose, myo-inositol, or D-ribose;
X is —O—C(O)—;
R² is a ketone body moiety selected from β-hydroxybutyryl, acetoacetyl, or a derivative thereof; and
n is an integer from 1 to 8, wherein n represents the total number of ketone body moieties attached to R¹, and wherein n can be less than or equal to the total number of available attachment sites on R¹.

In certain embodiments, the compound is a compound of Table 1, or a pharmaceutically acceptable salt thereof. It is understood that each of these compounds may exist in partially esterified/amidated forms where not all available hydroxyl or amine groups are conjugated to ketone bodies. Additionally, mixed conjugates are contemplated wherein some attachment sites contain β-hydroxybutyrate moieties while others contain acetoacetate moieties on the same carrier molecule.

TABLE 1
List of compounds.

| Compound | | |
|---|---|---|
| No. | Name | Structure |
| 1 | Trehalose-BHB Ester (TrehaBHB) | 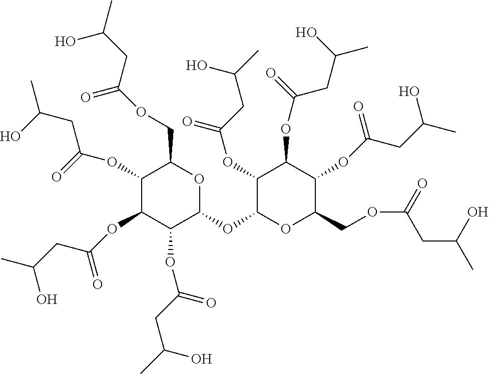 [SMILES: CC(O)CC(=O)OC[C@H]1O[C@H](O[C@H]2O[C@H](COC(=O)CC(C)O)[C@@H](OC(=O)CC(C)O)[C@H](OC(=O)CC(C)O)[C@H]2OC(=O)CC(C)O)[C@H](OC(=O)CC(C)O)[C@@H](OC(=O)CC(C)O)[C@@H]1OC(=O)CC(C)O] |
| 2 | Trehalose- Acetoacetate Ester (TrehaAce) | 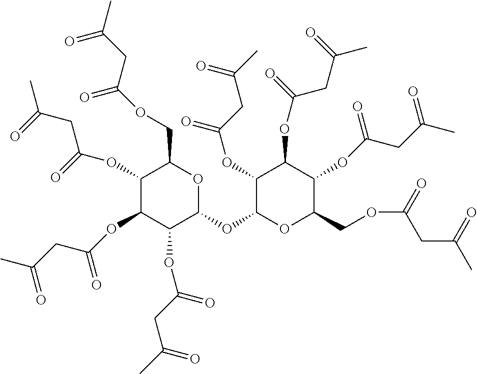 [SMILES: CC(=O)CC(=O)OC[C@H]1O[C@H](O[C@H]2O[C@H](COC(=O)CC(C)=O)[C@@H](OC(=O)CC(C)=O)[C@H](OC(=O)CC(C)=O)[C@H]2OC(=O)CC(C)=O)[C@H](OC(=O)CC(C)=O)[C@@H](OC(=O)CC(C)=O)[C@@H]1OC(=O)CC(C)=O] |
| 3 | Inositol-BHB Ester (InosBHB) | 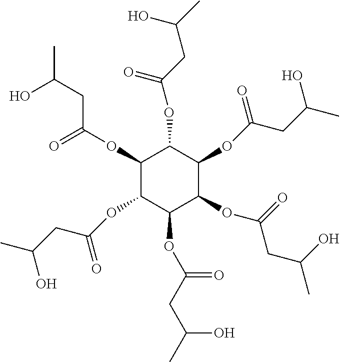 [SMILES: CC(O)CC(=O)O[C@H]1[C@H](OC(=O)CC(C)O)[C@@H](OC(=O)CC(C)O)[C@H](OC(=O)CC(C)O)[C@@H](OC(=O)CC(C)O)[C@H]1OC(=O)CC(C)O] |
| 4 | D-Ribose-BHB Ester (RiboBHB) | 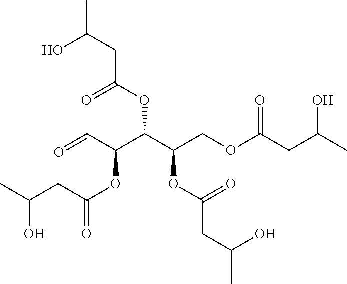 [SMILES: CC(O)CC(=O)OC[C@@H](OC(=O)CC(C)O)[C@@H](OC(=O)CC(C)O)[C@H](C=O)OC(=O)CC(C)O] |
| 5 | Inulin-BHB Ester (FibraBHB) | 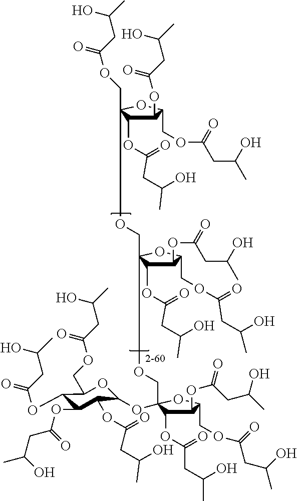 |
| 6 | Creatine-BHB Ester (CreaBHB) | 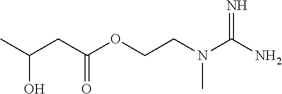 |
| 7 | Glycine- Acetoacetate Amide (GlyAce) | 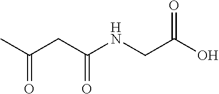 |
| 8 | Carnitine- Acetoacetate Ester (CarnAce) | 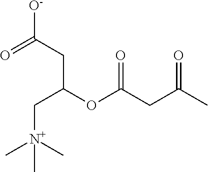 [SMILES: CC(=O)CC(=O)OC(CC(=O)[O-])C[N+](C)(C)C] |
| 9 | Taurine- Acetoacetate Amide (TaurAce) | 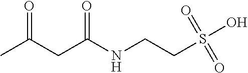 |
| 10 | Inositol- Acetoacetate Ester (InosAce) | 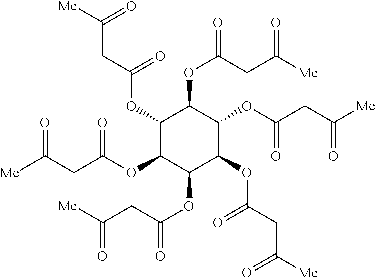 [SMILES: CC(=O)CC(=O)O[C@H]1[C@H](OC(=O)CC(C)=O)[C@@H](OC(=O)CC(C)=O)[C@H](OC(=O)CC(C)=O)[C@@H](OC(=O)CC(C)=O)[C@H]1OC(=O)CC(C)=O] |
| 11 | Glycerol- Acetoacetate Ester (GlycAce) | 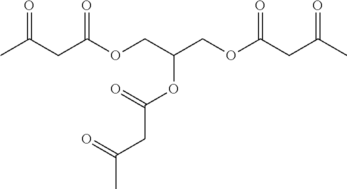 [SMILES: CC(=O)CC(=O)OCC(COC(=O)CC(C)=O)OC(=O)CC(C)=O] |
| 12 | Allulose- Acetoacetate Ester (AllAce) | 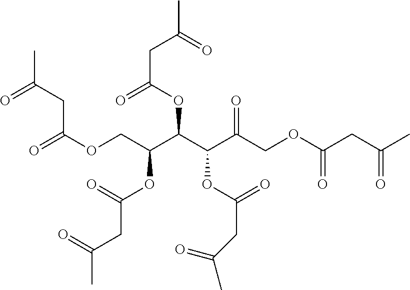 [SMILES: CC(=O)CC(=O)OCC(=O)[C@H](OC(=O)CC(C)=O)[C@H](OC(=O)CC(C)=O)[C@H](COC(=O)CC(C)=O)OC(=O)CC(C)=O] |
| 13 | Aspartic Acid- Acetoacetate Ester (AspAce) | 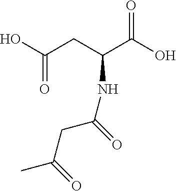 |
| 14 | Glycerol- BHB Ester (GlycBHB) | 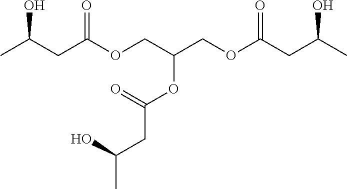 [SMILES: C[C@H](O)CC(=O)OCC(COC(=O)C[C@@H](C)O)OC(=O)C[C@@H](C)O] |
| 15 | Carnosine-BHB Ester (CarnosBHB) | 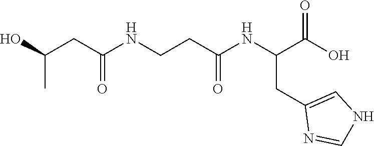 [SMILES: C[C@@H](O)CC(=O)NCCC(=O)NC(Cc1c[nH]cn1)C(=O)O] |

The compounds of Formula (A) described herein offer certain advantages over simpler ketone supplements (such as monomeric ketone esters or salts). By attaching multiple ketone body moieties (R²) to a single polyol backbone (R¹), a single compound can deliver a higher payload of ketone equivalents per molecule, potentially increasing the efficiency of raising blood ketone levels. Additionally, the use of a polyol backbone can impart favorable pharmacokinetic properties—for instance, as the conjugate is metabolized, it may release ketone bodies gradually, acting as a built-in sustained-release mechanism. This could help maintain therapeutically relevant ketone concentrations over a longer duration compared to a rapid spike from a simple ketone salt. In some embodiments, such multi-ketone conjugates may also improve tolerability or palatability: by distributing the ketone load across a larger molecule, peak gastrointestinal irritation or the strong taste associated with free ketones or salts might be reduced. While the invention is not bound by theory, these potential benefits illustrate the innovative nature of the polyol-based ketone conjugates, which go beyond conventional monomeric ketone compounds.

### Compositions

Another aspect of the invention provides ketogenic agents which are compositions comprising one or more compounds of Formula (A). In certain embodiments, the comprises at least two compounds from Table 1.

In certain embodiments, the composition comprises Compound 1 and Compound 2. In certain embodiments, the composition comprises Compound 1 and Compound 3. In certain embodiments, the composition comprises Compound 1 and Compound 4. In certain embodiments, the composition comprises Compound 1 and Compound 5. In certain embodiments, the composition comprises Compound 1 and Compound 6. In certain embodiments, the composition comprises Compound 1 and Compound 7. In certain embodiments, the composition comprises Compound 1 and Compound 8. In certain embodiments, the composition comprises Compound 1 and Compound 9. In certain embodiments, the composition comprises Compound 1 and Compound 10. In certain embodiments, the composition comprises Compound 1 and Compound 11. In certain embodiments, the composition comprises Compound 1 and Compound 12. In certain embodiments, the composition comprises Compound 1 and Compound 13. In certain embodiments, the composition comprises Compound 1 and Compound 14. In certain embodiments, the composition comprises Compound 1 and Compound 15.

In certain embodiments, the composition comprises Compound 2 and Compound 3. In certain embodiments, the composition comprises Compound 2 and Compound 4. In certain embodiments, the composition comprises Compound 2 and Compound 5. In certain embodiments, the composition comprises Compound 2 and Compound 6. In certain embodiments, the composition comprises Compound 2 and Compound 7. In certain embodiments, the composition comprises Compound 2 and Compound 8. In certain embodiments, the composition comprises Compound 2 and Compound 9. In certain embodiments, the composition comprises Compound 2 and Compound 10. In certain embodiments, the composition comprises Compound 2 and Compound 11. In certain embodiments, the composition comprises Compound 2 and Compound 12. In certain embodiments, the composition comprises Compound 2 and Compound 13. In certain embodiments, the composition comprises Compound 2 and Compound 14. In certain embodiments, the composition comprises Compound 2 and Compound 15.

In certain embodiments, the composition comprises Compound 3 and Compound 4. In certain embodiments, the composition comprises Compound 3 and Compound 5. In certain embodiments, the composition comprises Compound 3 and Compound 6. In certain embodiments, the composition comprises Compound 3 and Compound 7. In certain embodiments, the composition comprises Compound 3 and Compound 8. In certain embodiments, the composition comprises Compound 3 and Compound 9. In certain embodiments, the composition comprises Compound 3 and Compound 10. In certain embodiments, the composition comprises Compound 3 and Compound 11. In certain embodiments, the composition comprises Compound 3 and Compound 12. In certain embodiments, the composition comprises Compound 3 and Compound 13. In certain embodiments, the composition comprises Compound 3 and Compound 14. In certain embodiments, the composition comprises Compound 3 and Compound 15.

In certain embodiments, the composition comprises Compound 4 and Compound 5. In certain embodiments, the composition comprises Compound 4 and Compound 6. In certain embodiments, the composition comprises Compound 4 and Compound 7. In certain embodiments, the composition comprises Compound 4 and Compound 8. In certain embodiments, the composition comprises Compound 4 and Compound 9. In certain embodiments, the composition comprises Compound 4 and Compound 10. In certain embodiments, the composition comprises Compound 4 and Compound 11. In certain embodiments, the composition comprises Compound 4 and Compound 12. In certain embodiments, the composition comprises Compound 4 and Compound 13. In certain embodiments, the composition comprises Compound 4 and Compound 14. In certain embodiments, the composition comprises Compound 4 and Compound 15.

In certain embodiments, the composition comprises Compound 5 and Compound 6. In certain embodiments, the composition comprises Compound 5 and Compound 7. In certain embodiments, the composition comprises Compound 5 and Compound 8. In certain embodiments, the composition comprises Compound 5 and Compound 9. In certain embodiments, the composition comprises Compound 5 and Compound 10. In certain embodiments, the composition comprises Compound 5 and Compound 11. In certain embodiments, the composition comprises Compound 5 and Compound 12. In certain embodiments, the composition comprises Compound 5 and Compound 13. In certain embodiments, the composition comprises Compound 5 and Compound 14. In certain embodiments, the composition comprises Compound 5 and Compound 15.

In certain embodiments, the composition comprises Compound 6 and Compound 7. In certain embodiments, the composition comprises Compound 6 and Compound 8. In certain embodiments, the composition comprises Compound 6 and Compound 9. In certain embodiments, the composition comprises Compound 6 and Compound 10. In certain embodiments, the composition comprises Compound 6 and Compound 11. In certain embodiments, the composition comprises Compound 6 and Compound 12. In certain embodiments, the composition comprises Compound 6 and Compound 13. In certain embodiments, the composition comprises Compound 6 and Compound 14. In certain embodiments, the composition comprises Compound 6 and Compound 15.

In certain embodiments, the composition comprises Compound 7 and Compound 8. In certain embodiments, the composition comprises Compound 7 and Compound 9. In certain embodiments, the composition comprises Compound 7 and Compound 10. In certain embodiments, the composition comprises Compound 7 and Compound 11. In certain embodiments, the composition comprises Compound 7 and Compound 12. In certain embodiments, the composition comprises Compound 7 and Compound 13. In certain embodiments, the composition comprises Compound 7 and Compound 14. In certain embodiments, the composition comprises Compound 7 and Compound 15.

In certain embodiments, the composition comprises Compound 8 and Compound 9. In certain embodiments, the composition comprises Compound 8 and Compound 10. In certain embodiments, the composition comprises Compound 8 and Compound 11. In certain embodiments, the composition comprises Compound 8 and Compound 12. In certain embodiments, the composition comprises Compound 8 and Compound 13. In certain embodiments, the composition comprises Compound 8 and Compound 14. In certain embodiments, the composition comprises Compound 8 and Compound 15.

In certain embodiments, the composition comprises Compound 9 and Compound 10. In certain embodiments, the composition comprises Compound 9 and Compound 11. In certain embodiments, the composition comprises Compound 9 and Compound 12. In certain embodiments, the composition comprises Compound 9 and Compound 13. In certain embodiments, the composition comprises Compound 9 and Compound 14. In certain embodiments, the composition comprises Compound 9 and Compound 15.

In certain embodiments, the composition comprises Compound 10 and Compound 11. In certain embodiments, the composition comprises Compound 10 and Compound 12. In certain embodiments, the composition comprises Compound 10 and Compound 13. In certain embodiments, the composition comprises Compound 10 and Compound 14. In certain embodiments, the composition comprises Compound 10 and Compound 15.

In certain embodiments, the composition comprises Compound 11 and Compound 12. In certain embodiments, the composition comprises Compound 11 and Compound 13. In certain embodiments, the composition comprises Compound 11 and Compound 14. In certain embodiments, the composition comprises Compound 11 and Compound 15.

In certain embodiments, the composition comprises Compound 12 and Compound 13. In certain embodiments, the composition comprises Compound 12 and Compound 14. In certain embodiments, the composition comprises Compound 12 and Compound 15.

In certain embodiments, the composition comprises Compound 13 and Compound 14. In certain embodiments, the composition comprises Compound 13 and Compound 15.

In certain embodiments, the composition comprises Compound 14 and Compound 15.

In certain embodiments, the composition comprises at least two compounds of Formula (A). In certain embodiments, each compound of Formula (A) has the same R¹ and X.

As generally defined above R¹ is a polyol, amino acid, or a derivative thereof. In certain embodiments, R¹ is a polyol. A polyol is a compound containing two or more hydroxy (OH) groups.

In certain embodiments, R¹ is a polyol which is capable of delivering about 1 g to 50 g or more of ketone bodies to a subject diagnosed with a condition associated with energy insufficiency. In certain embodiments, R¹ is a polyol which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a condition associated with energy insufficiency. In certain embodiments, R¹ is a polyol which is capable of delivering about 1 g to 5 g or more of ketone bodies to a subject diagnosed with a condition associated with energy insufficiency. In certain embodiments, R¹ is a polyol which is capable of delivering about 5 g to 10 g or more of ketone bodies to a subject diagnosed with a condition associated with energy insufficiency. In certain embodiments, R¹ is a polyol which is capable of delivering about 10 g to 15 g or more of ketone bodies to a subject diagnosed with a condition associated with energy insufficiency. In certain embodiments, R¹ is a polyol which is capable of delivering about 15 g to 20 g or more of ketone bodies to a subject diagnosed with a condition associated with energy insufficiency. In certain embodiments, R¹ is a polyol which is capable of delivering about 20 g to 25 g or more of ketone bodies to a subject diagnosed with a condition associated with energy insufficiency. In certain embodiments, R¹ is a polyol which is capable of delivering about 25 g to 30 g or more of ketone bodies to a subject diagnosed with a condition associated with energy insufficiency. In certain embodiments, R¹ is a polyol which is capable of delivering about 30 g to about 35 g or more of ketone bodies to a subject diagnosed with a condition associated with energy insufficiency. In certain embodiments, R¹ is a polyol which is capable of delivering about 35 g to 40 g or more of ketone bodies to a subject diagnosed with a condition associated with energy insufficiency. In certain embodiments, R¹ is a polyol which is capable of delivering about 40 g to 45 g or more of ketone bodies to a subject diagnosed with a condition associated with energy insufficiency. In certain embodiments, R¹ is a polyol which is capable of delivering or about 45 g to 50 g or more of ketone bodies to a subject diagnosed with a condition associated with energy insufficiency.

In certain embodiments, R¹ is a polyol which is capable of delivering about 1 g or more of ketone bodies to a subject diagnosed with a condition associated with energy insufficiency. In certain embodiments, R¹ is a polyol which is capable of delivering about 5 g or more of ketone bodies to a subject diagnosed with a condition associated with energy insufficiency. In certain embodiments, R¹ is a polyol which is capable of delivering about 10 g or more of ketone bodies to a subject diagnosed with a condition associated with energy insufficiency. In certain embodiments, R¹ is a polyol which is capable of delivering about 15 g or more of ketone bodies to a subject diagnosed with a condition associated with energy insufficiency. In certain embodiments, R¹ is a polyol which is capable of delivering about 20 g or more of ketone bodies to a subject diagnosed with a condition associated with energy insufficiency. In certain embodiments, R¹ is a polyol which is capable of delivering about 25 g or more of ketone bodies to a subject diagnosed with a condition associated with energy insufficiency. In certain embodiments, R¹ is a polyol which is capable of delivering about 30 g or more of ketone bodies to a subject diagnosed with a condition associated with energy insufficiency. In certain embodiments, R¹ is a polyol which is capable of delivering about 35 g or more of ketone bodies to a subject diagnosed with a condition associated with energy insufficiency. In certain embodiments, R¹ is a polyol which is capable of delivering about 40 g or more of ketone bodies to a subject diagnosed with a condition associated with energy insufficiency. In certain embodiments, R¹ is a polyol which is capable of delivering or about 45 g or more of ketone bodies to a subject diagnosed with a condition associated with energy insufficiency. In certain embodiments, R¹ is a polyol which is capable of delivering or about 50 g or more of ketone bodies to a subject diagnosed with a condition associated with energy insufficiency.

In certain embodiments, R¹ is a carbohydrate. In certain embodiments, R¹ is a carbohydrate which is capable of delivering about 1 g to 50 g or more of ketone bodies to a subject diagnosed with a condition associated with energy insufficiency. In certain embodiments, R¹ is a carbohydrate which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a condition associated with energy insufficiency. In certain embodiments, R¹ is a carbohydrate which is capable of delivering about 1 g to 5 g or more of ketone bodies to a subject diagnosed with a condition associated with energy insufficiency. In certain embodiments, R¹ is a carbohydrate which is capable of delivering about 5 g to 10 g or more of ketone bodies to a subject diagnosed with a condition associated with energy insufficiency. In certain embodiments, R¹ is a carbohydrate which is capable of delivering about 10 g to 15 g or more of ketone bodies to a subject diagnosed with a condition associated with energy insufficiency. In certain embodiments, R¹ is a carbohydrate which is capable of delivering about 15 g to 20 g or more of ketone bodies to a subject diagnosed with a condition associated with energy insufficiency. In certain embodiments, R¹ is a carbohydrate which is capable of delivering about 20 g to 25 g or more of ketone bodies to a subject diagnosed with a condition associated with energy insufficiency. In certain embodiments, R¹ is a carbohydrate which is capable of delivering about 25 g to 30 g or more of ketone bodies to a subject diagnosed with a condition associated with energy insufficiency. In certain embodiments, R¹ is a carbohydrate which is capable of delivering about 30 g to about 35 g or more of ketone bodies to a subject diagnosed with a condition associated with energy insufficiency. In certain embodiments, R¹ is a carbohydrate which is capable of delivering about 35 g to 40 g or more of ketone bodies to a subject diagnosed with a condition associated with energy insufficiency. In certain embodiments, R¹ is a carbohydrate which is capable of delivering about 40 g to 45 g or more of ketone bodies to a subject diagnosed with a condition associated with energy insufficiency. In certain embodiments, R¹ is a carbohydrate which is capable of delivering or about 45 g to 50 g or more of ketone bodies to a subject diagnosed with a condition associated with energy insufficiency.

In certain embodiments, R¹ is a carbohydrate which is capable of delivering about 1 g or more of ketone bodies to a subject diagnosed with a condition associated with energy insufficiency. In certain embodiments, R¹ is a carbohydrate which is capable of delivering about 5 g or more of ketone bodies to a subject diagnosed with a condition associated with energy insufficiency. In certain embodiments, R¹ is a carbohydrate which is capable of delivering about 10 g or more of ketone bodies to a subject diagnosed with a condition associated with energy insufficiency. In certain embodiments, R¹ is a carbohydrate which is capable of delivering about 15 g or more of ketone bodies to a subject diagnosed with a condition associated with energy insufficiency. In certain embodiments, R¹ is an carbohydrate which is capable of delivering about 20 g or more of ketone bodies to a subject diagnosed with a condition associated with energy insufficiency. In certain embodiments, R¹ is a carbohydrate which is capable of delivering about 25 g or more of ketone bodies to a subject diagnosed with a condition associated with energy insufficiency. In certain embodiments, R¹ is an carbohydrate which is capable of delivering about 30 g or more of ketone bodies to a subject diagnosed with a condition associated with energy insufficiency. In certain embodiments, R¹ is a carbohydrate which is capable of delivering about 35 g or more of ketone bodies to a subject diagnosed with a condition associated with energy insufficiency. In certain embodiments, R¹ is a carbohydrate which is capable of delivering about 40 g or more of ketone bodies to a subject diagnosed with a condition associated with energy insufficiency. In certain embodiments, R¹ is a carbohydrate which is capable of delivering or about 45 g or more of ketone bodies to a subject diagnosed with a condition associated with energy insufficiency. In certain embodiments, R¹ is a carbohydrate which is capable of delivering or about 50 g or more of ketone bodies to a subject diagnosed with a condition associated with energy insufficiency.

In certain embodiments, R¹ is an amino acid or a derivative thereof. In certain embodiments, R¹ is a amino acid or a derivative thereof which is capable of delivering about 1 g to 50 g or more of ketone bodies to a subject diagnosed with a condition associated with energy insufficiency. In certain embodiments, R¹ is a amino acid or a derivative thereof which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a condition associated with energy insufficiency. In certain embodiments, R¹ is a amino acid or a derivative thereof which is capable of delivering about 1 g to 5 g or more of ketone bodies to a subject diagnosed with a condition associated with energy insufficiency. In certain embodiments, R¹ is a amino acid or a derivative thereof which is capable of delivering about 5 g to 10 g or more of ketone bodies to a subject diagnosed with a condition associated with energy insufficiency. In certain embodiments, R¹ is a amino acid or a derivative thereof which is capable of delivering about 10 g to 15 g or more of ketone bodies to a subject diagnosed with a condition associated with energy insufficiency. In certain embodiments, R¹ is a amino acid or a derivative thereof which is capable of delivering about 15 g to 20 g or more of ketone bodies to a subject diagnosed with a condition associated with energy insufficiency. In certain embodiments, R¹ is a amino acid or a derivative thereof which is capable of delivering about 20 g to 25 g or more of ketone bodies to a subject diagnosed with a condition associated with energy insufficiency. In certain embodiments, R¹ is a amino acid or a derivative thereof which is capable of delivering about 25 g to 30 g or more of ketone bodies to a subject diagnosed with a condition associated with energy insufficiency. In certain embodiments, R¹ is a amino acid or a derivative thereof which is capable of delivering about 30 g to about 35 g or more of ketone bodies to a subject diagnosed with a condition associated with energy insufficiency. In certain embodiments, R¹ is a amino acid or a derivative thereof which is capable of delivering about 35 g to 40 g or more of ketone bodies to a subject diagnosed with a condition associated with energy insufficiency. In certain embodiments, R¹ is a amino acid or a derivative thereof which is capable of delivering about 40 g to 45 g or more of ketone bodies to a subject diagnosed with a condition associated with energy insufficiency. In certain embodiments, R¹ is a amino acid or a derivative thereof which is capable of delivering or about 45 g to 50 g or more of ketone bodies to a subject diagnosed with a condition associated with energy insufficiency.

In certain embodiments, R¹ is a amino acid or a derivative thereof which is capable of delivering about 1 g or more of ketone bodies to a subject diagnosed with a condition associated with energy insufficiency. In certain embodiments, R¹ is a amino acid or a derivative thereof which is capable of delivering about 5 g or more of ketone bodies to a subject diagnosed with a condition associated with energy insufficiency. In certain embodiments, R¹ is a amino acid or a derivative thereof which is capable of delivering about 10 g or more of ketone bodies to a subject diagnosed with a condition associated with energy insufficiency. In certain embodiments, R¹ is a amino acid or a derivative thereof which is capable of delivering about 15 g or more of ketone bodies to a subject diagnosed with a condition associated with energy insufficiency. In certain embodiments, R¹ is an amino acid or a derivative thereof which is capable of delivering about 20 g or more of ketone bodies to a subject diagnosed with a condition associated with energy insufficiency. In certain embodiments, R¹ is a amino acid or a derivative thereof which is capable of delivering about 25 g or more of ketone bodies to a subject diagnosed with a condition associated with energy insufficiency. In certain embodiments, R¹ is an amino acid or a derivative thereof which is capable of delivering about 30 g or more of ketone bodies to a subject diagnosed with a condition associated with energy insufficiency. In certain embodiments, R¹ is a amino acid or a derivative thereof which is capable of delivering about 35 g or more of ketone bodies to a subject diagnosed with a condition associated with energy insufficiency. In certain embodiments, R¹ is a amino acid or a derivative thereof which is capable of delivering about 40 g or more of ketone bodies to a subject diagnosed with a condition associated with energy insufficiency. In certain embodiments, R¹ is a amino acid or a derivative thereof which is capable of delivering or about 45 g or more of ketone bodies to a subject diagnosed with a condition associated with energy insufficiency. In certain embodiments, R¹ is a amino acid or a derivative thereof which is capable of delivering or about 50 g or more of ketone bodies to a subject diagnosed with a condition associated with energy insufficiency.

In certain embodiments, R¹ is selected from the group consisting of trehalose, inositol, ribose, inulin, creatine, glycine, carnitine, and taurine. In certain embodiments, R¹ is selected from the group consisting of trehalose, myo-inositol, D-ribose, inulin, creatine, glycine, carnitine, taurine, glycerol, allulose, aspartic acid, and carnosine. In certain embodiments, R¹ is selected from the group consisting of trehalose, myo-inositol, D-ribose, inulin, creatine, glycine, carnitine, and taurine.

In certain embodiments, R¹ is selected from the group consisting of trehalose, inositol, ribose, inulin, creatine, glycine, carnitine, and taurine. In certain embodiments, R¹ is selected from the group consisting of trehalose, myo-inositol, D-ribose, inulin, creatine, glycine, carnitine, taurine, glycerol, allulose, aspartic acid, and carnosine. In certain embodiments, R¹ is selected from the group consisting of trehalose, myo-inositol, D-ribose, inulin, creatine, glycine, carnitine, and taurine.

In certain preferred embodiments, R¹ is trehalose, myo-inositol, or D-ribose. In certain embodiments, R¹ is myo-inositol or trehalose. In certain embodiments, R¹ is myo-inositol. In certain embodiments, R¹ is trehalose. In certain embodiments, R¹ is D-ribose. In certain embodiments, R¹ is inulin. In certain embodiments, R¹ is creatine. In certain embodiments, R¹ is glycine. In certain embodiments, R¹ is carnitine. In certain embodiments, R¹ is taurine. In certain embodiments, R¹ is glycerol. In certain embodiments, R¹ is allulose. In certain embodiments, R¹ is aspartic acid. In certain embodiments, R¹ is carnosine.

As generally defined above, X is a covalent linker joining selected from the group consisting of —O—C(O)—, —C(O)—O—, —C(O)—NH—, —NH—C(O)—, —C(O)—S—, —S—C(O)—, —O—C(O)—O—, —O—C(O)—NH—or —NH—C(O)—O—, —O—P(O₂)—O—, —O—SO₂—O—, —C═N—, —C═N—NH—, —O—, —NH—, —S—, and —S—S—. In certain embodiments, X is —O—C(O)—. In certain embodiments, X is —C(O)—O—. In certain embodiments, X is —C(O)—NH—. In certain embodiments, X is —NH—C(O)—. In certain embodiments, X is —C(O)—S—. In certain embodiments, X is —S—C(O)—. In certain embodiments, X is —O—C(O)—O—. In certain embodiments, X is —O—C(O)—NH—or —NH—C(O)—O—. In certain embodiments, X is —O—P(O₂)—O—. In certain embodiments, X is —O—SO₂—O—. In certain embodiments, X is —C—N—. In certain embodiments, X is —C═N—NH—. In certain embodiments, X is —O—. In certain embodiments, X is —NH—. In certain embodiments, X is —S—. In certain embodiments, X is —S—S—. In preferred embodiments, X is —O—or —NH—.

In certain preferred embodiments, R¹ is trehalose, myo-inositol, or D-ribose. In certain embodiments, R¹ is myo-inositol or trehalose. In certain embodiments, R¹ is myo-inositol. In certain embodiments, R¹ is trehalose. In certain embodiments, R¹ is D-ribose. In certain embodiments, R¹ is inulin. In certain embodiments, R¹ is creatine. In certain embodiments, R¹ is glycine. In certain embodiments, R¹ is carnitine. In certain embodiments, R¹ is taurine. In certain embodiments, R¹ is glycerol. In certain embodiments, R¹ is allulose. In certain embodiments, R¹ is aspartic acid. In certain embodiments, R¹ is carnosine.

In certain embodiments, the composition comprises at least two unique compounds of Formula (A). In certain embodiments, the composition comprises one unique compound of Formula (A) is:

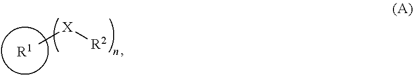

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH—or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl or a derivative thereof; and
n is an integer from 1 to 50, wherein n represents the total number of ketone body moieties attached to R¹, and wherein n can be less than or equal to the total number of available attachment sites on R¹, and
wherein the other unique compound of Formula (A) is:

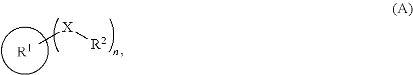

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH—or —NH—C(O)—O—, —O—P(O₂)—O—, —O—SO₂—O—, —C═N—, —C═N—NH—, —O—, —NH—, —S—, and —S—S—;
R² is a ketone body moiety selected from acetoacetyl (AcAc) or a derivative thereof; and
n is an integer from 1 to 50, wherein n represents the total number of ketone body moieties attached to R¹, and wherein n can be less than or equal to the total number of available attachment sites on R¹.

The compounds of Formula (A) may be formulated as pharmaceutical compositions by combining the compound with pharmaceutically acceptable carriers, excipients, and/or diluents. Such compositions may be formulated for oral, enteral, parenteral, or intravenous administration.

For oral administration, the compounds may be formulated as tablets, capsules, powders, solutions, suspensions, or emulsions. For parenteral administration, the compounds may be formulated as solutions, suspensions, or emulsions in aqueous or oil vehicles.

### Therapeutic Applications and Advantages

The compounds and compositions of the present disclosure provide significant therapeutic advantages for treating condition associated with energy insufficiencys:

Enhanced Bioavailability: By conjugating ketone bodies to carrier molecules via stable linkers (e.g., ester or amide bonds), the compounds and compositions exhibit improved absorption, extended circulation time, and enhanced tissue penetration compared to free ketone bodies.

Increased Ketone Payload: In certain embodiments, the multi-esterification design allows delivery of multiple ketone moieties per carrier molecule, maximizing the therapeutic effect while minimizing excipient load.

Targeted Delivery: Specific carrier molecules can preferentially direct ketones to tissues most affected by glucose hypometabolism. For example, trehalose-based conjugates may have enhanced blood-brain barrier penetration for neurological conditions.

Controlled Release: The linkers (e.g., the ester or amide bonds) are enzymatically hydrolyzed in vivo, providing sustained release of ketone bodies over time rather than the sharp peaks and troughs seen with direct ketone salt administration.

Synergistic Effects: The carrier molecules themselves often have complementary therapeutic effects. For example, inositol supports membrane stabilization and signaling, while creatine enhances cellular energy reserves.

Reduced Side Effects: The conjugate formulations minimize gastrointestinal distress and electrolyte imbalances commonly associated with ketone salt administration.

Dosing Convenience: The stable solid formulations allow for convenient oral dosing, improving patient compliance compared to liquid formulations or dietary interventions.

### Mechanisms of Action

The compounds and compositions of the present disclosure work through multiple complementary mechanisms:

Metabolic Bypass: The released ketone bodies provide an alternative energy substrate that bypasses the impaired glucose metabolism pathways seen in conditions like Alzheimer's disease, Parkinson's disease, and heart failure.

Enhanced Energetic Efficiency: Ketone bodies produce more ATP per oxygen molecule compared to glucose (2.5 vs 2.25 ATP/O), providing energetic advantages in oxygen-limited or mitochondrially compromised tissues.

Reduced Oxidative Stress: Ketone metabolism produces fewer reactive oxygen species than glucose or fatty acid oxidation, reducing oxidative damage in affected tissues.

Signaling Effects: Beyond serving as energy substrates, ketone bodies (particularly β-hydroxybutyrate) activate beneficial signaling pathways, including those related to mitochondrial biogenesis, antioxidant defense, and epigenetic regulation through histone deacetylase inhibition.

Neuroprotection: In neurodegenerative conditions, ketone bodies provide neuroprotection through multiple mechanisms, including reduced excitotoxicity, enhanced GABA signaling, and stabilization of neuronal membrane potentials.

Cardiac Protection: In heart failure, ketone bodies improve cardiac efficiency, reduce fibrosis, and enhance calcium handling in cardiomyocytes.

### The Metabolic Sufficiency Ratio (M=E/R) and its Components

The Metabolic Sufficiency Ratio, M=E/R, serves as the central organizing principle of UTBS and the foundation for the diagnostic and therapeutic methods of the present disclosure. Understanding the factors that determine E and R is crucial for applying this framework.

The Effective Energy Generation Rate (E) represents the numerator of the ratio and reflects the capacity of the system to produce usable energy (ATP). E is a composite term determined by several factors for each potential fuel substrate i:

Substrate Uptake/Availability (U_i): This depends on the concentration of the substrate in the circulation or local environment and the capacity and kinetics of specific transporter proteins responsible for its entry into the cell. Examples include Glucose Transporters (GLUTs) for glucose, Monocarboxylate Transporters (MCTs) for ketones and lactate, and transporters like FAT/CD36 for fatty acids. The expression and activity of these transporters can be dynamically regulated, influencing substrate availability. Techniques like PET can measure substrate uptake rates in vivo (e.g., FDG for glucose, 11C-acetoacetate for ketones).

Partitioning (PF_i): Once inside the cell, substrates can be directed to various pathways. The Partition Factor (PF_i) determines the fraction channeled towards oxidative pathways that yield ATP(e.g., glycolysis followed by TCA cycle and OXPHOS for glucose; beta-oxidation followed by TCA/OXPHOS for fatty acids; ketolysis followed by TCA/OXPHOS for ketones). Other fates include storage (glycogen, lipids) or biosynthesis. PFs are dynamically controlled (see below).

Efficiency (η_i): This reflects the amount of ATP generated per mole of substrate oxidized or per mole of oxygen consumed during its oxidation. It depends on the stoichiometry of the specific metabolic pathway and the efficiency of mitochondrial oxidative phosphorylation (OXPHOS), often related to the P/O ratio (ATP produced per oxygen atom reduced), Mitochondrial integrity, the functional status of the electron transport chain (ETC) complexes (e.g., Complex I defects in PD), and the degree of mitochondrial coupling influence η_i. Some substrates, like ketone bodies, may offer higher ATP yield per unit of oxygen consumed compared to others under certain conditions, suggesting a potential efficiency advantage.

The Total Energy Requirement Rate (R) represents the denominator of the ratio and reflects the total energy demand or ATP consumption rate of the system. R is also a composite term:

Basal Metabolism (R_basal): The energy required for essential housekeeping functions, such as maintaining ion gradients across membranes, basic protein synthesis and turnover, and other fundamental cellular processes.

Stress Response (R_stress): The additional energy expenditure incurred when cells respond to stressors like inflammation (mediated by cytokines like IL-6, TNF-α), oxidative damage, unfolded protein accumulation (UPR), infection, or hypoxia. Repairing damage and mounting defensive responses are energetically costly.

Activity-Related Demand (R_activity): The energy required for the specific functions of a given tissue, such as neuronal firing and neurotransmission in the brain, muscle contraction in the heart and skeletal muscle, active transport in the kidney, or complex signaling processes.

Repair and Maintenance (R_repair): The energy needed for ongoing repair of molecular damage (e.g., DNA repair) and the turnover and maintenance of cellular structures. It is critical to recognize that both E and R are dynamic variables. Pathological states can simultaneously decrease the capacity for energy generation (e.g., mitochondrial dysfunction reducing E) and increase energy demand (e.g., chronic inflammation increasing R_stress). TBI, for instance, involves both impaired ATP production due to mitochondrial damage and ischemia, and a surge in demand due to excitotoxicity and stress responses. The resulting decline in M=E/R drives secondary injury. Consequently, a state of energy insufficiency (low M) can arise from a primary defect in supply (low E), an excessive increase in demand (high R), or a combination of both. This understanding informs therapeutic strategies, which can aim to either bolster E, reduce pathological R, or both, to restore M towards sufficiency. Furthermore, because E and R fluctuate over time depending on physiological state and pathological activity, the disclosure encompasses methods for monitoring M or its components dynamically, providing insights into disease progression or response to therapy, rather than relying solely on static, single-timepoint assessments. Measures like the PCr/ATP ratio obtained via 31P-MRS provide an in vivo index reflecting the balance between ATP supply (E) and demand (R).

### Substrate Relativity and Compensatory Fuel Utilization

A cornerstone of the UTBS framework is the principle of “Substrate Relativity,” which posits that cells possess inherent mechanisms to adapt their fuel utilization patterns in response to limitations in primary substrate pathways. When the metabolism of a preferred fuel (e.g., glucose) is compromised, leading to a potential drop in E and M, cells attempt to compensate by increasing the uptake and oxidation of alternative, available fuels. The relative contribution of these secondary fuels to overall energy production increases as the primary pathway falters. This dynamic compensation is crucial for maintaining bioenergetic homeostasis.

Ketone Bodies (β-hydroxybutyrate (BHB) and Acetoacetate): Ketones serve as a paradigmatic example of compensatory fuel. Produced primarily by the liver during periods of low carbohydrate availability (fasting, ketogenic diet) or impaired glucose utilization (e.g., insulin resistance), they circulate in the blood and can be readily taken up by extrahepatic tissues, including the brain and heart. Transport across cell membranes and the blood-brain barrier is mediated by Monocarboxylate Transporters (MCTs), particularly MCT1 and MCT2. Inside the cell, ketones are converted back to acetyl-CoA via enzymes like β-hydroxybutyrate dehydrogenase 1 (BDH1) and succinyl-CoA: 3-ketoacid CoA transferase (SCOT), entering the TCA cycle for ATP production. This pathway becomes particularly important in conditions like AD, where brain glucose metabolism is often impaired (glucose hypometabolism). Evidence from PET studies using tracers like 11C-acetoacetate confirms that the AD brain retains the ability to take up and utilize ketones, potentially bypassing the defects in glucose handling and providing a vital alternative energy source. Similarly, in many forms of heart failure where fatty acid oxidation may be impaired or inefficient, the failing heart demonstrates increased uptake and oxidation of ketone bodies, suggesting a compensatory shift. Therapeutic interventions aimed at increasing ketone availability, such as administration of exogenous ketone esters (e.g., D-β-hydroxybutyrate-(R)-1,3-butanediol), ketone salts, MCTs (which are metabolized to ketones), or adherence to ketogenic diets, leverage this principle to directly increase E_ketone and thereby improve M. Clinical trials exploring ketone esters in AD aim to assess cognitive benefits, while trials in HF have shown improvements in cardiac output and filling pressures. It is noteworthy that while therapeutically administered ketones appear beneficial by providing a controlled boost to E, high levels of endogenously produced ketones observed in severe disease states (e.g., advanced HF) are often associated with poor prognosis. This apparent paradox can be understood within the UTBS framework: therapeutic ketosis aims to proactively increase M, whereas high endogenous ketosis in severe disease may reflect a state of profound metabolic derangement (critically low M) where primary fuel pathways have failed, compensatory mechanisms are overwhelmed, and the high ketone levels signify uncontrolled catabolism rather than effective energy rescue. The present disclosure focuses on the controlled, therapeutic use of ketones and related interventions to improve M. Furthermore, the benefits of ketones may extend beyond simple energy provision. BHB, for example, has been shown to possess signaling properties, including inhibition of histone deacetylases (HDACs), potentially leading to favorable epigenetic modifications, as well as exhibiting anti-inflammatory (e.g., inhibiting NLRP3 inflammasome) and antioxidant effects. These pleiotropic actions could contribute significantly to the overall therapeutic efficacy of ketogenic interventions.

Lactate: Traditionally viewed primarily as a byproduct of anaerobic glycolysis, lactate is now increasingly recognized as an important energy substrate and metabolic shuttle. Produced by various cells (including muscle and glial cells like astrocytes), lactate can be transported via MCTs to other cells and tissues (e.g., neurons, cardiomyocytes, muscle fibers) where it is converted back to pyruvate and readily oxidized in the TCA cycle, particularly during periods of high energy demand or metabolic stress. The brain, for instance, can utilize lactate derived from astrocytes as a significant fuel source, a process potentially impaired in AD. Lactate production can increase even under aerobic conditions. Therapeutic administration of lactate or lactate precursors is being explored, particularly in acute energy crises such as TBI, where it might help bridge the energy gap. Elevated lactate levels can also serve as a prognostic marker in conditions like HF and cardiogenic shock, reflecting impaired tissue perfusion or metabolic stress. Like ketones, lactate also exhibits signaling functions, potentially influencing gene expression and cellular functions.

Amino Acids: While primarily known as building blocks for proteins, amino acids also contribute significantly to energy metabolism, especially under certain conditions. Branched-chain amino acids (BCAAs: leucine, isoleucine, valine) and glutamine are particularly relevant. Their carbon skeletons can be catabolized to enter the TCA cycle directly or serve as anaplerotic precursors (replenishing TCA cycle intermediates), which is crucial for maintaining oxidative capacity. This role becomes particularly important during states of prolonged fasting, stress, or significant muscle wasting (cachexia). Leucine also acts as a potent signaling molecule. Therapeutic strategies involving supplementation with specific amino acids, such as BCAAs or glutamine, are employed in clinical nutrition to support muscle mass and provide energy substrates, particularly in cachectic patients. However, the metabolic roles of amino acids can be complex; for instance, chronically elevated BCAA levels have been associated with insulin resistance.

Understanding substrate relativity and the roles of these compensatory fuels is essential for designing effective “Energy Rescue” therapies tailored to specific metabolic deficits identified through diagnostic methods described herein.

### Partition Factors and Metabolic Flexibility

The allocation of available fuel substrates towards different metabolic fates (e.g., immediate oxidation for energy, storage as glycogen or fat, or use in biosynthetic pathways) is a critical regulatory node within cellular energy management. UTBS describes this allocation process using the concept of dynamic “Partition Factors” (PFs). For each substrate i, PFs determine the fraction directed towards specific pathways (e.g., PF glucose ox for glucose oxidation, PF_FA_storage for fatty acid esterification). These PFs are not static but are continuously modulated by a complex interplay of signals, ensuring that fuel utilization is matched to the cell's needs and the body's overall physiological state.

### Key Factors Influencing PFs Include:

Hormonal Signals: Hormones like insulin and glucagon exert powerful control over fuel partitioning. Insulin typically promotes glucose uptake and utilization (increasing U_glucose and PF_glucose_ox) while suppressing lipolysis and fatty acid release (decreasing U_FA and PF_FA_ox), favoring energy storage in the fed state. Glucagon and catecholamines generally have opposing effects, promoting glucose production and fatty acid mobilization/oxidation during fasting or stress.

Substrate Availability: The concentrations of substrates themselves influence their fate. High availability of one fuel can inhibit the utilization of another, a classic example being the Randle cycle, where high levels of fatty acids or ketones can suppress glucose oxidation. Transporter and enzyme kinetics (e.g., saturation) also play a role.

Cellular Energy Status (M): The cell constantly senses its own energy state, often through ratios like ATP/ADP, AMP levels (activating AMPK), or the NADH/NAD+redox state. Low energy status (low M) typically activates pathways that increase ATP production (e.g., increasing oxidative PFs for available fuels) and suppress energy-consuming processes.

Genetic and Epigenetic Factors: An individual's genetic background influences baseline expression levels of transporters and enzymes, setting inherent biases in fuel preference and partitioning. Epigenetic modifications can further modulate these patterns in response to environmental cues.

“Metabolic Flexibility” is defined within UTBS as the capacity of a system (cell, tissue, or whole organism) to dynamically and appropriately adjust its PFs in response to changing conditions, thereby maintaining bioenergetic sufficiency (M). A metabolically healthy individual readily switches from primarily oxidizing glucose after a meal to oxidizing fatty acids during fasting or exercise. “Metabolic Inflexibility,” conversely, represents a failure of this adaptive capacity, characterized by rigid or inappropriately set PFs. A common example is found in insulin resistance and T2D, where tissues like muscle fail to adequately switch from fatty acid to glucose oxidation in the postprandial state, contributing to hyperglycemia and lipotoxicity. This inflexibility is often an early sign of declining metabolic health and impending energy insufficiency. Measures like the change in Respiratory Quotient (RQ) after a glucose challenge can quantify metabolic flexibility.

Interventions such as specific dietary patterns (e.g., ketogenic diets, caloric restriction) and regular physical exercise are known to improve metabolic flexibility, likely by influencing the signaling pathways that regulate PFs. Therefore, therapeutic strategies within the scope of this disclosure may include not only providing alternative fuels but also interventions aimed at restoring or enhancing metabolic flexibility itself, potentially by targeting the regulatory networks controlling PFs. Enhancing the system's intrinsic ability to adapt its fuel usage represents a distinct therapeutic goal complementary to direct fuel supplementation.

### Cellular and Organ Hierarchy and Vulnerability

UTBS recognizes that different tissues and organs within the body exhibit distinct metabolic characteristics, creating a functional hierarchy based on energy needs and fuel preferences. Organs with exceptionally high and constant energy demands, such as the brain and the heart, typically have priority access to circulating fuels but may also possess specific vulnerabilities due to their specialized metabolic profiles. The brain, for example, relies heavily on glucose under normal physiological conditions and is acutely sensitive to disruptions in glucose supply or utilization. The heart, needing to sustain continuous contractile activity, has a very high mitochondrial density and relies significantly on the high ATP yield from fatty acid oxidation (FAO) under normal conditions, making it vulnerable to defects in this pathway or shifts in substrate preference during failure.

UTBS posits that systemic metabolic stress-such as that induced by chronic insulin resistance, systemic inflammation, prolonged nutrient deprivation, or aging—can lead to a generalized decrease in the Metabolic Sufficiency Ratio (M) across multiple tissues simultaneously. However, the clinical manifestation of disease often appears first in the organ or tissue that represents the “weakest link” in the metabolic chain. This weakest link is the tissue whose M value drops below its specific critical threshold (M_crit) most readily under the given stress. This vulnerability is determined by a combination of factors: a high intrinsic energy requirement (high R_basal or R_activity), limited capacity to utilize compensatory fuels, specific dependence on a pathway that is impaired, or a lower maximal energy generation capacity (E_max_capacity) relative to its needs.

This concept of hierarchical vulnerability helps explain why a systemic metabolic problem, like metabolic syndrome (MetS), significantly increases the risk for a diverse range of seemingly organ-specific diseases, including AD, HF, T2D complications, and kidney disease. The underlying systemic metabolic dysfunction (e.g., insulin resistance, chronic low-grade inflammation) lowers M across the board, but the brain, heart, or kidneys may be the first to cross their respective M_crit thresholds and exhibit clinical signs of failure. The tissue-specific M_crit can be conceptually defined based on the minimum energy rate required for essential function (R_min_functional) and the maximum possible energy generation rate (E_max capacity), as M_crit,j=R_min functional,j/E_max_capacity,j.

This hierarchical perspective has significant implications for diagnosis and prevention. Assessing systemic metabolic health markers (e.g., insulin sensitivity, inflammatory markers like CRP, IL-6, integrated scores like MVX, measures of metabolic flexibility) can provide insights into the overall metabolic stress level. Combining this systemic assessment with knowledge of tissue-specific vulnerabilities allows for risk stratification and potentially targeted preventative strategies aimed at protecting the most susceptible organs before irreversible damage occurs.

### Bioenergetic Laws and Entropy Accumulation

UTBS incorporates fundamental principles of thermodynamics to provide a deeper understanding of the consequences of sustained bioenergetic insufficiency.

First Law of Thermodynamics (Conservation of Energy): Energy is neither created nor destroyed, only transformed. In biological systems, the chemical energy stored in fuel substrates is converted into ATP and heat. The efficiency of this conversion (represented by η_i in the definition of E) is crucial for maintaining M. Different substrates yield different amounts of ATP per mole oxidized or per unit of oxygen consumed (P/O ratio). For instance, ketone bodies have been suggested to be potentially more oxygen-efficient fuels than fatty acids, meaning they might yield more ATP per molecule of oxygen consumed. This could offer a metabolic advantage in situations where oxygen supply is limited or energy demand is extremely high. Maximizing the efficiency of ATP production from available fuels is a key strategy for maintaining a high M.

Second Law of Thermodynamics (Entropy): Living systems are highly complex, low-entropy structures that require a continuous input of free energy (derived from maintaining a high M) to maintain their organization and function, counteracting the universal tendency towards increasing disorder (entropy). All irreversible processes, including metabolic reactions, generate entropy (o). UTBS proposes a direct link between bioenergetic sufficiency and the rate of entropy generation within a cell or tissue (o). Specifically, it postulates that σ is inversely related to M: when M is high (sufficiency), the system has adequate energy for efficient operation, maintenance, and repair, thereby minimizing the rate of disorder accumulation. Conversely, as M declines below critical levels (M<1, insufficiency), cellular processes become less efficient, stress responses are activated (which are themselves entropy-generating), repair mechanisms falter, and damage accumulates, leading to an accelerated rate of entropy production. Mathematically, this relationship might be modeled, for instance, as σ_j=k/M_j+σ baseline, where k is a constant reflecting the inefficiency and stress associated with low M. The total accumulated entropy (representing cumulative damage, disorder, and loss of function) in a tissue j over time t can be viewed as the integral of the entropy generation rate: Entropy_j(t)=[[0 to t]σ_j(t) dr. This formulation provides a theoretical basis for understanding aging and the progression of chronic diseases as consequences of sustained or worsening bioenergetic deficits driving the system towards a higher entropy state. Chronic metabolic insufficiency, therefore, is not just a state of impaired function but a driver of accumulating disorder and eventual system failure. Loss of proteostasis, a hallmark of aging and AD, may be linked to this entropy accumulation, and interventions like ketone bodies might influence protein clearance mechanisms.

From this thermodynamic perspective, therapeutic interventions aimed at improving and maintaining M are fundamentally strategies to slow the rate of entropy accumulation, preserve cellular order, and thereby delay the onset or progression of age-related dysfunction and chronic disease. This potentially elevates the significance of targeting M=E/R beyond treating specific disease symptoms to addressing more fundamental aspects of healthspan and the aging process itself. Claims related to slowing the rate of biological aging or reducing the accumulation of entropy-related damage through M-modulating therapies may be contemplated, provided sufficient supporting evidence linking the intervention to both M improvement and established biomarkers of aging or cellular disorder can be demonstrated.

### Mathematical Modeling of UTBS

To operationalize the UTBS framework for predictive and quantitative applications, a mathematical formulation is proposed. The core equations include:

Metabolic Sufficiency Ratio: M=E/R

Effective Energy Generation: E=Σi (PF i*U_i*η_i)

Total Energy Requirement: R=R_basal+R_stress+R_activity+R_repair

Illustrative models can be developed for the key components:

Partition Factors (PFs): Modeled as functions dependent on regulatory inputs ([Insulin], [Glucagon]), energy state indicators (ATP/ADP, AMPK, NADH/NAD⁺), and substrate concentrations, incorporating known regulatory motifs (e.g., Randle cycle).

Fuel Competition: Simulated using systems of ordinary differential equations (ODEs) describing intracellular substrate pools and intermediates (e.g., Acetyl-CoA), incorporating influx (U_i), efflux, enzyme kinetics, and regulation by PFs.

Ketone Substitution: Explicitly modeled by incorporating E_ketone=PF_ketone_ox*U_ketone*n_ketone, including transporter (MCT) and enzyme (SCOT) saturation kinetics.

Hierarchy Thresholds: Tissue-specific M_crit,j defined or estimated based on tissue energy requirements (R_min_functional) and capacities (E_max_capacity).

Entropy Accumulation: Dynamics linked to M over time: Entropy j(t)=[[0 to t](k/M_j (T)+σ_baseline) dr.

Such a system of equations constitutes a dynamic mathematical model of UTBS that can be implemented computationally. This allows for: Simulating the time course of M in different tissues. Predicting dysfunction when M falls below M_crit. Performing in silico experiments to evaluate therapeutic interventions by simulating their effects on model parameters (U_i, PFs, η_i, R) and predicting impact on M and entropy. Guiding personalized medicine by incorporating individual-specific parameters (genetics, inflammation, substrate levels) to predict M status and response to interventions.

### Methods for Diagnosing Energy Insufficiency and Determining Metabolic Stage

A key aspect of the present disclosure involves methods for diagnosing or assessing the state of bioenergetic sufficiency (M=E/R status) in a subject or biological sample, and using this assessment to determine a metabolic stage relevant for prognosis and therapy selection. Given that M is a ratio of complex physiological rates (E and R), its direct measurement is challenging. Therefore, practical diagnostic approaches rely on measuring multiple parameters that reflect the components or consequences of the M=E/R balance, often integrating information from various modalities.

Metabolic Imaging: Non-invasive imaging techniques provide powerful tools to assess substrate uptake and energy status in specific tissues: Positron Emission Tomography (PET): ¹⁸F-FDG-PET is widely used to measure regional glucose uptake (related to U_glucose), revealing characteristic patterns of hypometabolism in conditions like AD (often in temporoparietal cortex, posterior cingulate cortex (PCC), precuneus). PET quantification typically involves calculating Standardized Uptake Value Ratios (SUVR) relative to a reference region like cerebellar gray matter, often requiring spatial normalization, potentially aided by MRI or deep learning techniques. Novel PET tracers are being developed or used to measure the uptake of other substrates, such as ketones (e.g., 11C-acetoacetate), allowing assessment of compensatory fuel utilization. Synthesis protocols for tracers like 11C-acetoacetate have been automated for potential clinical use, and dual-tracer protocols (e.g., 11C—AcAc and ¹⁸F-FDG) enable direct comparison of fuel utilization. Tracers targeting mitochondrial complexes, neuroinflammation (e.g., TSPO ligands), or specific enzymatic pathways could offer further information related to E, η_i, and R_stress. Magnetic Resonance Spectroscopy (MRS): MRS allows non-invasive quantification of various metabolites within a defined tissue volume. 31P-MRS can directly measure levels of high-energy phosphates like ATP and PCr, as well as inorganic phosphate (Pi). The PCr/ATP ratio is considered a sensitive indicator of the cellular energy reserve or buffering capacity, reflecting the balance between ATP supply (E) and demand (R).A decrease in PCr/ATP often signals impending or existing energy insufficiency, observed consistently in HF (correlating with severity and prognosis) but with more variable findings reported in AD. Absolute quantification of ATP and PCr is also possible but technically challenging. 1H-MRS can detect metabolites such as lactate (indicating glycolytic activity or stress), N-acetylaspartate (NAA, a marker of neuronal integrity), choline compounds, and potentially ketones. ¹³C-MRS, especially with labeled substrates, allows dynamic tracking of metabolic fluxes. MRS protocols often involve specialized hardware (coils), localization techniques (e.g., ISIS, CSI), and quantification methods.

Blood/Plasma Biomarkers: Analysis of blood or plasma provides systemic information about substrate availability, hormonal regulation, and markers of metabolic stress: Standard Laboratory Tests: Measurements of glucose, insulin, HbA1c, lipid profiles, lactate, and ketone bodies (BHB) provide fundamental information. Advanced Metabolomics: Techniques like NMR or MS quantify numerous metabolites, providing detailed metabolic snapshots and potentially identifying patterns or integrated scores (e.g., Metabolic Vulnerability Index, MVX) reflecting systemic metabolic stress relevant to M=E/R balance. Specific amino acid profiles (e.g., BCAAs) can also be informative. Inflammatory Markers: Elevated levels of markers like C-reactive protein (CRP), Interleukin-6 (IL-6), and Tumor Necrosis Factor-alpha (TNF-α) indicate systemic inflammation (contributing to R_stress) and are associated with increased risk or severity of conditions like AD and HF.

Biochemical Assays on Samples: Direct measurement of key metabolites in accessible samples (blood, plasma, tissue biopsies) using specific assays provides quantitative data. ATP Measurement: Commercially available kits often utilize the ATP-dependent luciferase reaction, where light emission is proportional to ATP concentration. General protocol involves lysing cells/tissue to release ATP, adding a reagent containing luciferase and luciferin, and measuring luminescence using a luminometer. These kits are sensitive (pM to UM range) and suitable for various sample types. Care must be taken to avoid ATP degradation and contamination. BHB Measurement: Enzymatic assay kits are widely available (colorimetric or fluorometric/luminescent). The principle involves the enzyme β-hydroxybutyrate dehydrogenase oxidizing BHB, coupled to the reduction of NAD⁺ to NADH. NADH is then detected directly or via a coupled reaction producing a measurable signal (color, fluorescence, or luminescence). Kits are suitable for plasma, serum, urine, and cell/tissue lysates, typically requiring a plate reader. Detection ranges vary but can be sensitive enough for physiological and ketotic levels (e.g., ~10 μM to 1 mM or lower). Sample preparation may involve deproteinization. Lactate Measurement: Enzymatic assay kits (colorimetric or fluorometric) are common. Assays typically use lactate dehydrogenase (producing NADH) or lactate oxidase (producing H2O2), coupled to a probe generating a measurable signal. Suitable for various biological fluids and lysates. Requires a plate reader. Detection ranges typically cover physiological and pathological lactate levels (e.g., ~0.02 mM to 10-20 mM). Sample preparation may involve deproteinization or spin filtering.

Functional Tests: Dynamic tests assess the system's ability to adapt to metabolic challenges: Respiratory Quotient (RQ): Measured via indirect calorimetry (CO2 produced/02 consumed). Assessing the change in RQ after a glucose load or during a clamp provides an index of metabolic flexibility. Impaired switching indicates inflexibility.

A comprehensive assessment of M=E/R status likely requires integrating information from multiple modalities. For example, combining PET data on substrate uptake with MRS data on energy state (PCr/ATP) and plasma biomarkers reflecting systemic milieu (MVX, ketones, inflammation) can provide a more robust picture than any single measure alone. This necessity for integration arises partly because individual biomarkers can be ambiguous; for instance, high circulating BHB can reflect beneficial therapeutic ketosis (increased E) or severe underlying metabolic stress (low M, compensatory failure) Similarly, lactate can be a fuel substrate or a marker of tissue distress, and even PCr/ATP findings can vary between studies or disease stages. The UTBS framework, particularly through its staging system, provides the context needed to interpret these multi-modal data points collectively. The use of machine learning or artificial intelligence approaches may be particularly valuable for identifying complex patterns within high-dimensional datasets (e.g., metabolomics, multi-modal imaging) that correlate with M=E/R status or predict clinical outcomes.

### Unified Metabolic Staging System (UTBS Stages):

Based on the assessment of M=E/R status using the modalities described above, UTBS enables the development of a universal metabolic staging system applicable across different diseases characterized by underlying energetic failure. This system aims to provide a clinically meaningful classification of severity, grounded in the fundamental bioenergetic principles of UTBS. An illustrative staging system is presented in Table 2.

TABLE 2
UTBS Universal Metabolic Staging System (Illustrative)

| | M = E/R | Key Biomarker Profile Examples | Clinical Correlates | |
|---|---|---|---|---|
| | Range | (Imaging, MRS, Blood, | (Examples: AD/HF/ | |
| Stage | (Conceptual) | Flexibility) | T2D/Systemic) | Therapeutic Goal |
| 0 | M >> 1.0 | Normal PET (e.g., FDG SUVR > | Asymptomatic; | Primordial |
| | (Robust | 1.3); Normal MRS (e.g., PCr/ATP > | Metabolically | Prevention; |
| | Buffer) | 1.8); High metabolic flexibility | Healthy; Optimal | Maintain |
| | | (RQ shift > 0.1); Low inflammatory | resilience; Normal | Flexibility. |
| | | markers (CRP < 1, IL-6 < 2 pg/mL); | glucose tolerance, | |
| | | Low MVX. | | |
| 1 | M > 1.0 | Subtle PET changes possible; | At Risk; Early MetS; | Lifestyle |
| | (Reduced | Normal/Slightly ↓ MRS (e.g., | Pre-diabetes; HF | Intervention |
| | Buffer) | PCr/ATP 1.6-1.8); Reduced insulin | Stage A/B; Potential | (Diet/Exercise); |
| | | sensitivity; Mild metabolic | subtle cognitive | Restore |
| | | inflexibility (↓RQ shift); | changes?; | Flexibility; |
| | | Slightly ↑ Inflamm. | Asymptomatic but | Prevent |
| | | markers/MVX. | reduced resilience. | Progression. |
| 2 | M ≈ 1.0 | Clear PET deficit (e.g., FDG | Mild Symptoms; Mild | Energy Rescue |
| | (Compensated) | SUVR < 1.1); ↓MRS (e.g., | Cognitive Impairment | (e.g., Ketones); |
| | | PCr/ATP 1.2-1.6); ↑Compensatory | (MCI); NYHA Class | Enhance |
| | | fuel use (Ketones ↑, Lactate ↑); | II HF; Early T2D | Compensation; |
| | | ↑MVX; ↑Inflamm. markers | complications; | Address Primary |
| | | | Manifest inflexibility. | Deficit; Improve |
| | | | | Flexibility. |
| 3 | M < 1.0 | Severe PET/MRS abnormalities | Moderate-Severe | Aggressive |
| | (Decompensated) | (e.g., FDG SUVR << 1.0, PCr/ATP < | Symptoms; Mild- | Energy Rescue; |
| | | 1.2); High compensatory fuels | Moderate AD | Support Organ |
| | | (stress marker); High | Dementia; NYHA | Function; Manage |
| | | inflammatory/entropy markers; | Class III HF; | Symptoms; Slow |
| | | High MVX. | Advanced T2D | Entropy |
| | | | complications; | Accumulation. |
| | | | Functional | |
| | | | limitations. | |
| 4 | M << 1.0 | Profound metabolic collapse in | Severe Disability; | Palliative Care; |
| | (Critical | biomarkers; Multi-organ | Severe AD Dementia; | Maximize Quality |
| | Failure) | involvement possible; Very high | NYHA Class IV HF; | of Life. |
| | | MVX. | Refractory Cachexia; | |
| | | | End-stage disease. | |

This staging system provides a framework for classifying patients based on their underlying bioenergetic status, linking measurable biological parameters to clinical severity and guiding the intensity and type of therapeutic intervention. Table 3 summarizes the key diagnostic modalities relevant to assessing M=E/R status.

TABLE 3
Diagnostic Modalities for Assessing M = E/R Status

| | Parameters | Relevance to | Examples/Key |
|---|---|---|---|
| Modality | Measured | M = E/R | Technologies |
| Metabolic | | | |
| Imaging | | | |
| PET | Substrate Uptake (Glucose, | Reflects U_i component | FDG-PET, 11C- |
| | Ketones, FAs, AAs), | of E; R_stress | Acetoacetate PET, Fatty |
| | Inflammation (TSPO) | | Acid Tracers, TSPO |
| | | | Ligands |
| MRS | High-Energy Phosphates (ATP, | Reflects balance of E vs | 31P-MRS (PCr/ATP), 1H- |
| | PCr, Pi); Metabolites (Lactate, | R (PCr/ATP); Fuel | MRS (Lactate, NAA), |
| | NAA, Ketones); Flux | pools; Pathway activity | 13C-MRS (Flux), |
| | | (η_i) | Hyperpolarized 13C-MRS |
| Blood/Plasma | | | |
| Biomarkers | | | |
| Standard Labs | Glucose, Insulin, Lipids, Lactate, | Reflects systemic U_i, | Clinical chemistry assays, |
| | Ketones (BHB), Hormones, | hormonal milieu (PF | Immunoassays (CRP, IL- |
| | Inflamm. Markers | related), R_stress | 6) |
| Metabolomics | Comprehensive profile of small | Snapshot of fuels, | NMR Spectroscopy, Mass |
| | molecules | intermediates, stress | Spectrometry (LC-MS, |
| | | markers | GC-MS) |
| Integrated | Composite scores from | Proxy for overall | Metabolic Vulnerability |
| Scores | biomarker panels (e.g., | metabolic | Index (MVX) |
| | metabolomics, proteomics) | stress/vulnerability, | |
| | | reflecting low M | |
| Biochemical | Specific metabolites (ATP, BHB, | Direct measure of key | Luciferase ATP assay, |
| Assays | Lactate) | E/R related components | Enzymatic BHB assay, |
| | | | Enzymatic Lactate assay |
| Functional | | | |
| Tests | | | |
| Metabolic | Ability to switch fuel oxidation | Reflects adaptive | Indirect Calorimetry (RQ |
| Flexibility | in response to challenge | capacity of PF | changes), |
| | | regulation | Hyperinsulinemic- |
| | | | euglycemic clamp with |
| | | | tracers |

### Therapeutic Methods, Compositions, and Kits for Treating Energy Insufficiency

Based on the UTBS framework, the present disclosure provides therapeutic methods, compositions, and kits aimed at treating or preventing conditions associated with energy insufficiency by modulating the M=E/R ratio. These “Energy Rescue” strategies target either increasing the effective energy generation (E) or decreasing the energy requirement (R), or both. The specific approach can be personalized based on the diagnosed primary metabolic deficit (e.g., impaired glucose vs. fatty acid metabolism identified via PET or MRS) and the patient's determined UTBS metabolic stage (from Table 2). This diagnostic-therapeutic linkage, where assessment modalities like PET or MRS directly inform the selection of interventions like ketone therapy, represents a rational and potentially more effective approach compared to empirical treatment.

Methods to Increase Effective Energy Generation (E): These strategies aim to boost the numerator of the M=E/R ratio. Ketogenic Interventions: Providing ketone bodies or their precursors bypasses potential blocks in glucose metabolism (e.g., insulin resistance, GLUT deficiency, PDH issues) or can supplement energy supply when other pathways (like FAO) are impaired or insufficient. This directly increases the E_ketone term in the energy generation equation. Agents: Exogenous ketone esters (e.g., D-β-hydroxybutyrate-(R)-1,3-butanediol), ketone salts, medium-chain triglycerides (MCTs, hepatically converted to ketones). Diets: Ketogenic diets (very low carbohydrate, high fat) induce endogenous ketone production. Rationale: Provide readily usable alternative fuel, potentially more oxygen-efficient, bypass defects (e.g., glucose hypometabolism in AD, Complex I in PD). Additional benefits may arise from BHB signaling (HDAC inhibition, anti-inflammatory/antioxidant effects). Evidence: Promising results in preclinical models and clinical studies for AD (potential cognitive benefits), PD, and HF (hemodynamic improvements like increased cardiac output, reduced filling pressures). Lactate Supplementation: Providing lactate or its precursors can supply fuel, particularly in acute high-demand states. Rationale: Lactate is readily oxidized via pyruvate. May be beneficial in TBI or severe stress. Specific Amino Acid Supplementation: Certain amino acids can serve as fuel sources or support mitochondrial function via anaplerosis. Agents: BCAAs (leucine, isoleucine, valine), glutamine. Rationale: Support TCA cycle function, provide energy substrates, support muscle protein synthesis (relevant in cachexia). Improving Energy Conversion Efficiency (η_i): Targeting mitochondrial function to enhance ATP yield from available fuels. Agents: Mitochondrial cofactors (e.g., Coenzyme Q10), NAD⁺ precursors (e.g., nicotinamide riboside, nicotinamide mononucleotide), agents improving OXPHOS coupling or reducing ROS production. Rationale: Increase ATP output per unit of substrate/oxygen consumed. Enhancing Primary Fuel Utilization: Improving the function of pathways for glucose or fatty acid oxidation when they are impaired but potentially salvageable. Agents: Insulin sensitizers (e.g., metformin, thiazolidinediones (TZDs)) improve glucose uptake and partitioning (PF glucose) in insulin-resistant states. Potential agents enhancing FAO activity or efficiency (e.g., PPAR agonists), used cautiously depending on context (e.g., HF complexity).

Methods to Decrease Energy Requirement (R): These strategies aim to lower the denominator of the M=E/R ratio, particularly pathological increases in demand. Anti-inflammatory Agents: Reducing chronic inflammation (e.g., lowering IL-6, TNF-α) lowers the associated energy cost (R_stress). Antioxidants: Mitigating oxidative stress reduces the ATP required for repairing molecular damage (R_repair). Workload Reduction: Pharmacological agents that reduce the workload of specific organs, such as beta-blockers in HF, decrease R_activity, contributing to improved energy balance alongside their hemodynamic effects.

### Mechanistic Rationale for Treating Orphan Diseases

The therapeutic rationale for using exogenous ketones in orphan diseases stems from their ability to rescue cellular ATP production, improve the NAD⁺/NADH redox balance, and reduce toxic metabolite buildup under conditions where primary energy pathways are impaired. Key mechanistic points include:

Alternative Fuel for ATP Generation: Ketone bodies (D-βHB and AcAc) are water-soluble energy carriers that can be oxidized by mitochondria in heart, muscle, and brain to generate ATP. In situations of glucose unavailability or mitochondrial blocks, ketones bypass metabolic bottlenecks. For example, in GLUT1 deficiency, the brain cannot import enough glucose, but ketones cross the blood-brain barrier via monocarboxylate transporters and enter the Krebs cycle as acetyl-CoA. In fatty acid oxidation disorders, long-chain fats cannot be fully broken down, but ketones provide a downstream fuel that does not rely on the defective β-oxidation enzymes. By supplying acetyl-CoA to the TCA cycle, ketones help maintain ATP production in tissues that would otherwise be energy-starved. Indeed, studies in an AD (Alzheimer's) patient population showed that raising blood ketones can compensate for part of the brain's glucose deficit and improve cerebral energy metabolism.

Improved NAD⁺/NADH Ratio (Redox Shift): Oxidation of BHB to acetoacetate within cells consumes NAD⁺ and produces NADH (via the enzyme β-hydroxybutyrate dehydrogenase). At first glance, this might seem to worsen the cytosolic NAD⁺/NADH ratio; however, when comparing the net NAD⁺ usage of producing ATP from different substrates, ketones are more “NAD-sparing” than glucose. Notably, to yield two acetyl-CoA from glucose, glycolysis and pyruvate dehydrogenase consume 4 NAD⁺, whereas to yield two acetyl-CoA from BHB/AcAc requires only 1 NAD⁺. In other words, ketone metabolism can produce ATP with less NAD⁺ turnover, thereby potentially increasing the steady-state NAD⁺/NADH ratio in cells. A higher NAD⁺/NADH ratio is associated with improved metabolic health and activation of pro-longevity signaling pathways (e.g., sirtuins). Consistent with this, brain imaging studies in healthy adults demonstrated that induction of nutritional ketosis raised the brain NAD⁺/NADH ratio. In this context, many mitochondrial disorders have an over-reduced NADH-rich state due to blockages in electron transport; providing ketones may help alleviate this by altering substrate utilization and promoting more efficient NADH use in the remaining functional respiratory chain.

Reduction of Lactate and Other Toxic Metabolites: In conditions like PDH deficiency or mitochondrial complex defects, cells often resort to anaerobic glycolysis, leading to accumulation of lactate (lactic acidosis) and other partially oxidized metabolites. By giving ketones, we shift cellular metabolism away from glycolysis. Ketones do not produce lactate when used as fuel; instead, they enter oxidative pathways more directly. Clinical reports have shown that ketogenic therapy can reduce blood lactate in PDH deficiency. In vitro data with PDH-deficient fibroblasts showed that media lactate levels dropped by >50% when BHB (2 mM) was provided as an alternate substrate (prophetic example based on known KD effect). Similarly, in long-chain FAOD, patients accumulate acyl-carnitines and triglycerides in tissues; ketone provision spares the need to oxidize fatty acids, thereby preventing the formation of excess acyl-carnitines. Lowering of such toxic metabolites can directly translate to clinical benefit (e.g., less cardiac damage from acyl-carnitine accumulation in the heart, less rhabdomyolysis in muscle).

Neurotransmitter and Signaling Effects: Beyond pure energetics, ketone bodies have ancillary effects that can be beneficial. BHB is an inhibitor of class I histone deacetylases (HDACs), which can increase expression of oxidative stress resistance genes and brain-derived neurotrophic factor (BDNF). BHB and AcAc also moderate neuronal excitability by hyperpolarizing neurons (through ATP-sensitive potassium channels, adenosine modulation, etc.), explaining the anti-seizure efficacy observed in ketosis. In some embodiments, the inclusion of certain moieties (like the sugar or amino acid components) provides additional neuroprotective signaling—for instance, trehalose (in SNV-T1) inducing autophagy could help clear protein aggregates in disorders like ALS or Huntington's, complementing BHB's fuel provision. Similarly, ketones have been reported to increase levels of cerebral adenosine, a neuromodulator with anticonvulsant properties.

Anaplerosis and Buffering: The design of some compounds purposefully addresses secondary issues in energy disorders. α-Ketoglutarate provided in SNV—K2 helps replenish TCA cycle intermediates (important in conditions where intermediates are depleted or in high demand). Creatine in SNV—C1 buffers ATP by regenerating it from ADP in situ, potentially smoothing out energy supply during fluctuations. Carnitine in carnitine-BHB esters could support mitochondrial transport of metabolites. These synergistic mechanisms mean the compounds do more than raise ketones; they tackle multiple angles of the metabolic deficit.

The potential exists for synergistic benefits by combining therapies that target different aspects of the M=E/R balance. For example, combining an agent that increases E (like a ketone ester) with an agent that decreases R (like an anti-inflammatory) could logically produce a greater improvement in M than either agent alone, particularly in patients exhibiting deficits in both energy supply and excessive demand.

Compositions: The disclosure includes compositions formulated for administration to subjects, comprising one or more of the therapeutic agents described above. These can be pharmaceutical compositions prepared according to standard practices, containing agents like ketone esters, ketone salts, lactate precursors, specific amino acids, mitochondrial modulators, or insulin sensitizers, along with pharmaceutically acceptable carriers or excipients. They can also be nutritional compositions or medical foods, such as formulations containing MCT oil or specific blends of amino acids designed to support energy metabolism.

Kits: The disclosure also provides kits for facilitating the diagnosis, monitoring, or treatment of energy insufficiency based on UTBS. A diagnostic kit might contain reagents for measuring specific biomarkers related to M=E/R (e.g., assay components for ATP, BHB, lactate, inflammatory markers, or antibodies for transporter proteins; or standards and precursors for PET tracer synthesis) and instructions for interpreting the results within the UTBS staging framework (Table 2). A therapeutic kit might contain a supply of an M=E/R modulating agent (e.g., a course of ketone ester supplements) along with instructions for administration, potentially tailored to a specific UTBS stage or condition. Combination kits containing both diagnostic components and therapeutic agents are also envisaged.

Synergy with Standard of Care (SoC): It is important to emphasize that UTBS-based metabolic interventions are generally intended to be used as adjuncts to, rather than replacements for, existing standard-of-care therapies for the specific underlying conditions. The goal is to achieve synergistic benefits by addressing the fundamental energy deficit alongside other pathological aspects. AD: Ketone therapy could complement cholinesterase inhibitors or potential disease-modifying therapies targeting amyloid or tau. HF: Metabolic modulators like ketone esters could be added to guideline-directed medical therapies (GDMT) including ACE inhibitors, ARBs, ARNis, beta-blockers, MRAs, and SGLT2 inhibitors. Notably, some benefits of SGLT2 inhibitors may themselves be mediated through metabolic shifts consistent with UTBS, such as inducing mild ketosis and improving cardiac energetics. PD: Ketogenic approaches could be used alongside levodopa or other dopaminergic therapies. Cachexia: Targeted nutritional support focusing on energy provision (potentially including ketones) and specific nutrients like BCAAs should be integrated with treatment of the primary cause and potentially anti-inflammatory drugs.

Table 4 summarizes the diverse therapeutic strategies encompassed by the disclosure.

TABLE 4
Therapeutic Strategies Targeting M = E/R

| | Primary Target/ | Examples of Agents/ | Target Conditions |
|---|---|---|---|
| Strategy Category | Mechanism | Interventions | (Examples) |
| Increase E | | | |
| (Energy Supply) | | | |
| Provide Alternative | Increase U_i for | Ketone Esters (e.g., D-BHB- | AD, PD, HF, TBI |
| Fuel | compensatory fuels | (R)-1,3-butanediol), Ketone | |
| | (Ketones, Lactate) | Salts, MCT Oil, Ketogenic Diet; | |
| | | Lactate Precursors | |
| Provide | Increase U_i for | BCAA Supplementation, | Cachexia, Stress |
| Anaplerotic/Energy | specific AAs, | Glutamine Supplementation | States |
| AAs | support TCA cycle | | |
| Increase E | | | |
| (Efficiency) | | | |
| Enhance | Improve η_i (ATP | NAD⁺ Precursors (NR, NMN), | Aging, |
| Mitochondrial | yield per | Coenzyme Q10, Mitochondrial | Neurodegeneration, |
| Function | substrate/O2) | Antioxidants, OXPHOS | HF |
| | | Coupling Modulators | |
| Increase E | | | |
| (Primary Fuel Use) | | | |
| Improve Glucose | Increase | Insulin Sensitizers (Metformin, | T2D, MetS, Insulin |
| Utilization | U_glucose, | TZDs) | Resistance |
| | improve | | |
| | PF_glucose_ox | | |
| Enhance FAO | Increase PF_FA_ox | PPAR Agonists (potential use, | Certain Metabolic |
| (where appropriate) | or efficiency | context-dependent) | Disorders |
| Improve Adaptive | | | |
| Capacity | | | |
| Enhance Metabolic | Improve dynamic | Lifestyle (Exercise, Diet), | MetS, T2D, Aging |
| Flexibility | regulation of PFs | Potential Pharmacological | |
| | | Modulators of PF regulators | |
| | | (e.g., AMPK activators) | |
| Decrease R | | | |
| (Energy Demand) | | | |
| Reduce | Decrease R_stress, | Anti-inflammatory Agents, | Chronic Inflammatory |
| Pathological Stress | R_repair | Antioxidants | Diseases, AD, HF |
| Reduce Organ | Decrease R_activity | Beta-blockers (in HF), | HF, potentially |
| Workload | | potentially agents reducing | Epilepsy/TBI |
| | | neuronal hyperexcitability | |

### Ketosis as a Mechanistic State: Equivalence of Means of Induction

A central mechanistic principle underlying the present disclosure is that the therapeutic efficacy of the ketotic state is determined by the circulating ketone body exposure . . . specifically, the concentration and duration of elevation of β-hydroxybutyrate (BHB) and acetoacetate (AcAc)—and the degree of engagement of downstream target mechanisms, independent of the particular means by which the ketotic state is achieved. This principle, referred to herein as the “Ketosis=Ketosis” equivalence principle, is supported by extensive convergent evidence across the scientific and medical literature.

Dietary ketosis has served as the reference standard for therapeutic ketone body elevation for nearly a century. The ketogenic diet induces sustained elevation of circulating BHB, typically reaching 2-7 mM under strict dietary adherence, through hepatic ketone production consequent upon severe carbohydrate restriction. At these concentrations, multiple downstream target mechanisms are simultaneously engaged: histone β-hydroxybutyrylation (Kbhb) occurs at measurable occupancy rates depending on tissue and histone isoform; NLRP3 inflammasome suppression is robust and dose-dependent; HCAR2/GPR109A receptor signaling is engaged with measurable immune and metabolic effects; circadian clock components BMAL1 and PER2 are reset; fibroblast growth factor 21 (FGF21) is induced; and class I histone deacetylases are inhibited through direct BHB binding. Importantly, these mechanisms are driven by circulating BHB elevation and operate independently of weight loss, energy deficit, or any particular baseline metabolic phenotype.

Ketone monoester compounds rapidly elevate circulating BHB to high levels —reaching 5-10 mM within 20-30 minutes of oral dosing-before clearing with a half-life of approximately 20-30 minutes. Despite the transient nature of this elevation, ketone monoesters engage the same downstream target mechanisms as dietary ketosis: histone acetylation and Kbhb are induced, NLRP3-mediated inflammatory markers are suppressed, and circulating cytokines shift in the same direction as observed with dietary ketosis. Studies in athletic populations have demonstrated that ketone monoester administration improved time-to-exhaustion performance through mechanisms consistent with metabolic state-dependent enhancement of mitochondrial efficiency and fuel utilization kinetics, not energy provision alone. Additional research has documented that even transient BHB elevation produces detectable effects on circulating metabolites, immune markers, and exercise physiology. The mechanism of action is identical to dietary ketosis; the kinetics differ, but the target-engagement mechanisms are the same.

Ketone salts induce a more modest and sustained elevation of circulating BHB compared to monoesters—typically 0.5-2 mM over several hours- and have been shown in multiple studies to engage target mechanisms including NLRP3 suppression, immune-cell metabolic reprogramming, and circulating cytokine shifts, despite the lower absolute BHB concentration. This observation supports the Ketosis=Ketosis principle: target engagement is a function of circulating BHB concentration integrated over time, not a function of peak concentration or the particular kinetic profile of the agent. SGLT2 inhibitors, which induce a mild ketosis through reduced glucose reabsorption, have been shown in multiple clinical trials to produce immune and metabolic benefits consistent with target mechanism engagement-including reduced heart failure hospitalizations and improved glycemic control-effects increasingly understood to be mediated by ketosis-driven mechanisms rather than glucose lowering alone. Medium-chain triglycerides (MCT) similarly engage measurable immune and metabolic shifts at circulating BHB levels of 0.5-1.5 mM. The convergence across multiple different ketogenic agents-dietary, monoester, salt, SGLT2 inhibitor, MCT, and 1,3-butanediol—is that target-engagement mechanisms are achieved at BHB concentrations as low as 0.5 mM and become progressively stronger at higher concentrations, independent of the agent used.

The most direct test of this equivalence principle comes from studies examining direct intravenous infusion of BHB or acetoacetate. When BHB is infused directly to achieve a circulating concentration of 2-5 mM, the same target-engagement mechanisms are observed as with dietary ketosis or pharmacological agents: NLRP3 inflammasome suppression, histone acetylation and Kbhb induction, circulating cytokine shifts, and T-cell metabolic reprogramming. The fact that direct infusion of BHB produces the identical target-engagement phenotype as dietary ketosis, ketone monoester, or any other ketogenic agent demonstrates that the mechanism of action is determined by circulating BHB concentration and duration, not by the particular biochemical route or kinetic profile of delivery. This equivalence principle forms the foundation for the broad, means-agnostic scope of the present disclosure and enables the claiming of methods encompassing any ketogenic agent without limitation to a particular chemical entity or modality. For purposes of the present disclosure, it is explicitly affirmed that any means of achieving the required circulating ketone body exposure engages the same downstream mechanisms-Kbhb deposition, NLRP3 inflammasome suppression, HCAR2/GPR109A signaling, NAD⁺/NADH-driven redox rebalancing, mitochondrial bioenergetic optimization, and circadian rhythm synchronization —and produces substantively equivalent therapeutic effects across MERIT-responsive conditions.

### Practical Clinical Feasibility and the Limitations of Competing Modalities

While the Ketosis=Ketosis principle establishes the mechanistic equivalence of all ketogenic agents, clinical reality imposes significant constraints on what can be deployed as a primary mode of therapy in real patient populations. The present disclosure documents the disqualifying limitations of each competing modality and establishes that a pharmacological, oral, titratable, sustained, well-tolerated, ambulatory, and diet-independent means of achieving therapeutic ketosis-exemplified by ketone-carrier conjugate compositions or functionally equivalent platforms-represents the only practical route for delivering reliable therapeutic ketosis in mainstream clinical practice.

The ketogenic diet remains the most physiologically robust means of achieving therapeutic ketosis but is burdened by a constellation of well-documented clinical limitations. Non-compliance is the primary barrier: long-term adherence to a strict ketogenic diet requires sustained carbohydrate restriction below 5-10% of caloric intake, which is exceedingly difficult for most patients, with dropout rates of 30-50% within 6-12 weeks and near-zero long-term adherence beyond 12 months in the absence of intensive dietetic support. The diet cannot be titrated-either a patient achieves the carbohydrate threshold necessary to maintain ketosis, or they do not, and any single dietary deviation can suppress ketosis for 12-24 hours. In hospitalized patients, patients with acute illness, or those requiring parenteral nutrition, dietary ketosis is simply not feasible. The dietary ceiling—the maximum population penetration achievable through dietary ketosis alone—is estimated at less than 5-10% of the populations that might benefit from therapeutic ketosis.

Fasting and fasting-mimicking diets can induce robust ketosis but are unsuitable as primary therapy. Hunger, fatigue, cognitive dysfunction, and irritability are nearly universal during fasting periods and limit adherence. Fasting is contraindicated in malnutrition-prone populations, patients with diabetes on glucose-lowering medications, patients in critical illness, and those requiring ongoing oral medications. Fasting cannot be sustained indefinitely, limiting the duration of achievable ketosis, and it cannot be deployed in hospitalized patients or those requiring concurrent pharmacological intervention.

Ketone monoester compounds represent a major advance in the ability to achieve rapid, robust ketosis without dietary intervention, but carry disqualifying limitations. Gastrointestinal intolerance is the primary barrier, with 20-40% of subjects experiencing nausea, vomiting, diarrhea, or abdominal discomfort at doses sufficient to achieve therapeutic BHB levels. Poor palatability-commonly described by subjects as reminiscent of industrial solvents-leads to patient refusal and inability to complete closing in a meaningful percentage of subjects. The rapid clearance (half-life approximately 20-30 minutes) necessitates multiple daily dosing, and the extraordinary cost per dose renders ketone monoesters inaccessible to the vast majority of patients. Clinical development programs for ketone monoesters have been discontinued or deprioritized at multiple major pharmaceutical companies due to these tolerability and deployment challenges.

Ketone salts are constrained by a fundamental pharmacological ceiling: the cation dose ceiling. To deliver clinically meaningful BHB concentrations, one must administer quantities of sodium, potassium, or calcium that exceed safe daily intake thresholds, inducing hypernatremia, hyperkalemia, or hypercalcemia concerns, respectively. This ceiling is immutable and cannot be overcome without shifting to a fundamentally different chemical architecture, limiting ketone salts to doses that induce only mild, transient ketosis and rendering them unsuitable for the chronic, sustained, treat-to-target ketosis necessary for most therapeutic applications.

1,3-Butanediol, a metabolic precursor to acetoacetate, induces robust ketosis but carries a well-documented toxicity profile that disqualifies it from therapeutic use at ketogenic doses. At doses sufficient to achieve therapeutic ketosis, 1,3-butanediol produces intoxication-like side effects-including dizziness, altered mental status, ataxia, slurred speech, and sedation —through its direct interaction with alcohol dehydrogenase and aldehyde dehydrogenase, the same enzymatic pathway that metabolizes ethanol. Critically, the most widely studied exogenous ketone monoester —the (R)-3-hydroxybutyl (R)-3-hydroxybutyrate ester developed by Veech and colleagues-metabolizes through 1,3-butanediol as an obligate intermediate, meaning that every dose exposes the subject to the full 1,3-butanediol toxicity burden. This toxicological liability is inherent to the chemical structure and cannot be engineered away while retaining the 1,3-butanediol backbone. By contrast, ketone-carrier conjugate compositions that employ polyol backbones-such as trehalose, myo-inositol, D-ribose, creatine, glycine, carnitine, or taurine-release BHB and/or AcAc upon hydrolysis without generating any 1,3-butanediol intermediate, representing a structurally distinct and safety-advantaged architecture.

Medium-chain triglycerides induce a mild ketosis and are generally well-tolerated, but the degree of ketosis achieved—typically 0.5-1.5 mM BHB—is usually insufficient to reliably engage target mechanisms when sustained engagement is required, and higher doses induce gastrointestinal side effects. SGLT2 inhibitors induce only a mild ketosis as a secondary pharmacological effect, carry a documented risk of euglycemic diabetic ketoacidosis in certain populations, and are primarily optimized for glucose control rather than for target-mechanism engagement. Direct intravenous or intra-arterial infusion of BHB or acetoacetate can achieve precise kinetic control but is wholly impractical outside of intensive care unit settings, requiring ongoing vascular access, continuous clinical monitoring, and non-ambulatory conditions incompatible with chronic outpatient use. This inventory reveals a critical gap: every existing means of inducing therapeutic ketosis carries one or more disqualifying clinical limitations that prevent it from serving as the primary mode of delivery in mainstream clinical practice. A pharmacological, oral, titratable, sustained, well-tolerated, diet-independent, ambulatory means of achieving and maintaining therapeutic ketosis therefore represents the only practical route for mainstream clinical deployment.

### MERIT Pillars and Their Mechanisms

A foundational advance of the present disclosure is the recognition that ketone bodies-β-hydroxybutyrate (BHB) and acetoacetate (AcAc)—exert therapeutic effects through five orthogonal mechanistic pillars, collectively termed the MERIT pillars: Metabolic, Epigenetic, Redox, Immune, and Signaling. Understanding the mechanisms underlying each pillar is essential for designing and interpreting therapeutic and diagnostic applications within the scope of this disclosure.

The Epigenetic Pillar: Histone β-Hydroxybutyrylation and HDAC Inhibition. The epigenetic pillar encompasses the direct chemical and biochemical effects of BHB and its metabolites on chromatin structure, histone modification, and gene expression. The central mechanism is histone β-hydroxybutyrylation (Kbhb), a histone post-translational modification catalyzed by acyl-CoA synthetase short-chain family member 2 (ACSS2) when intracellular butyryl-CoA is elevated in response to circulating BHB elevation. Kbhb-marked histones preferentially engage bromodomain proteins that recruit transcriptional machinery enriched in metabolic adaptation genes, stress-response genes, and immune-tolerance genes, representing a metabolic-state-dependent reprogramming of transcription toward catabolism stress resistance, and immune quiescence. Independently, BHB has been demonstrated to be a direct inhibitor of class I histone deacetylases (HDAC1, 2, 3, 8) at physiologically relevant concentrations of 0.5-5 mM, through non-competitive inhibition of the catalytic zinc center in the HDAC active site—a mechanism distinct from classical synthetic HDAC inhibitors. The combined effects of Kbhb deposition and HDAC inhibition constitute a powerful epigenetic remodeling of the genome in response to ketosis, producing global shifts in histone acetylation patterns and altered expression of genes involved in cell-cycle control, apoptosis, differentiation, and metabolic adaptation. Critically, these epigenetic effects occur in response to circulating BHB elevation alone, independent of energy status, glucose metabolism, or any particular baseline metabolic phenotype. The epigenetic pillar is particularly relevant to oncological and neuropsychiatric MERIT-responsive conditions, where epigenetic dysregulation is a core pathogenic mechanism. In oncology, HDAC inhibitors are approved therapeutics for certain hematologic malignancies, and broader HDAC inhibition may enhance immunotherapy efficacy. In psychiatry, epigenetic dysregulation is a core mechanism in depression and anxiety, and the Kbhb and HDAC-inhibitory effects of ketosis provide an endogenous, metabolic means of achieving epigenetic remodeling equivalent to small-molecule HDAC inhibitors, without their associated toxicity profile.

The Immune Pillar: NLRP3 Inflammasome Suppression and T-Cell Metabolic Reprogramming. The immune pillar encompasses the effects of ketosis on innate immune activation, T-cell and B-cell metabolism, barrier function, and the cytokine milieu. The central mechanism is BHB-mediated suppression of the NLRP3 inflammasome —the canonical innate immune activation platform responsible for the maturation and secretion of pro-inflammatory cytokines IL-18 and IL-18. BHB directly suppresses NLRP3 inflammasome activation in macrophages and monocytes by preventing the association of NLRP3 with the adaptor protein ASC and pro-caspase-1, thereby blocking formation of the active inflammasome complex and downstream caspase-1 activation. This suppression occurs at BHB concentrations as low as 0.5-1 mM and is dose-dependent, with maximal suppression at 2-4 mM. The NLRP3 inflammasome is a central hub in innate immune activation and is abnormally activated in multiple MERIT-responsive conditions, including autoinflammatory syndromes, systemic lupus erythematosus, rheumatoid arthritis, inflammatory bowel disease, certain infections, and sepsis. Beyond NLRP3 suppression, ketosis also shifts circulating T cells-particularly the CD8+ cytotoxic subset responsible for anti-tumor immunity-toward an oxidative-phosphorylation-dominant metabolic state characterized by elevated fatty acid oxidation, reduced glycolysis, and elevated NAD⁺/NADH ratio, a phenotype associated with long-lived memory T cells and enhanced effector function in response to checkpoint inhibitor stimulation. Preclinical research has demonstrated that combining ketogenic diet with anti-PD-1 monoclonal antibody produced a 70% complete response rate and durable tumor cures in immunologically cold syngeneic tumors, compared to approximately 10% with anti-PD-1 alone, with mechanisms involving both T-cell metabolic reprogramming and NLRP3-mediated remodeling of the tumor microenvironment. Ketosis additionally enhances intestinal barrier function through promotion of tight-junction protein expression (zonula occludens-1, claudins, occludin) and intestinal stem cell renewal, mechanisms driven by histone acetylation and NLRP3 suppression independently of any dietary exclusion. Barrier function is relevant to infectious disease, autoimmune disease, and inflammatory disease generally, where barrier failure is a critical step in disease pathogenesis.

The Redox Pillar: NAD⁺/NADH Ratio, Mitochondrial Function, and Oxidative Stress Buffering. The redox pillar encompasses the effects of ketosis on the NAD+/NADH ratio, mitochondrial biogenesis and function, reactive oxygen species (ROS) production, and the activation of endogenous antioxidant systems. The β-hydroxybutyrate dehydrogenase complex converts BHB to acetoacetate with simultaneous generation of NADH, leading to a shift in whole-cell NAD+/NADH toward the oxidized (NAD+) state—a change with profound effects on cellular metabolism and signaling. An elevated NAD+/NADH ratio enhances the activity of sirtuins (NAD+-dependent deacetylases that promote mitochondrial biogenesis, autophagy, and stress resistance), enhances fatty acid oxidation enzyme activity, and suppresses glycolytic flux, producing a net shift toward a catabolic, stress-resistant phenotype. Additionally, the oxidation of ketone bodies through the TCA cycle and electron transport chain produces ATP with greater efficiency per oxygen consumed compared to glucose oxidation, leading to more efficient mitochondrial energy production and reduced ROS generation from the electron transport chain. The transcription factor Nrf2, a master regulator of the antioxidant response, is activated by multiple ketosis-driven mechanisms and drives expression of antioxidant response elements controlling antioxidant enzyme expression, including catalase, superoxide dismutase, and glutathione peroxidase. The net result is a coordinated enhancement of redox buffering capacity and reduction in baseline oxidative stress in response to ketosis. This redox pillar is particularly relevant to neurodegenerative diseases characterized by impaired mitochondrial function and elevated oxidative stress, to aging, and to metabolic diseases including diabetes, where impaired mitochondrial function and oxidative stress are core pathogenic features.

The Signaling Pillar: HCAR2/GPR109A, FGF21, and Circadian Regulation. The signaling pillar encompasses the effects of ketone bodies as endogenous ligands for cell-surface receptors and as triggers for the release of circulating hormonal signals that communicate metabolic state throughout the organism. BHB is an endogenous ligand for HCAR2 (hydroxycarboxylic acid receptor 2, also known as GPR109A), a G-protein coupled receptor expressed on immune cells, adipocytes, and other cell types. BHB binds to HCAR2 at physiologically relevant concentrations of 0.1-1 mM, meaning that circulating BHB elevation during ketosis engages HCAR2 signaling, driving a shift toward a more tolerogenic, less inflammatory immune phenotype in macrophages and dendritic cells, and promoting thermogenesis and metabolic flexibility in adipocytes. FGF21 is a circulating hormone produced by the liver and other tissues in response to metabolic stress, fasting, and ketosis; fasting and ketogenic diet have been shown to induce robust FGF21 expression and secretion, driving enhanced fatty acid oxidation, mitochondrial biogenesis, glucose uptake in certain contexts, insulin sensitivity, and suppression of inflammation across target tissues including adipose tissue, liver, skeletal muscle, and immune cells. BHB has further been demonstrated to directly activate circadian clock components BMAL1 and PER2, leading to resynchronization of the circadian rhythm—a mechanism of particular relevance to MERIT-responsive conditions in which circadian dysregulation is a core feature, including depression, bipolar disorder, Parkinson's disease, cancer, and metabolic disease. Together, HCAR2 signaling, FGF21 induction, and circadian resynchronization constitute a paracrine and endocrine communication system through which circulating ketone bodies modulate immune activation, metabolic function, and stress resistance throughout the organism, independent of dietary composition, glucose metabolism, or any particular baseline metabolic phenotype.

A unique pharmacological property of ketosis, and one that distinguishes it from all existing single-target therapeutic agents, is its capacity to engage all five MERIT pillars simultaneously through five distinct molecular mechanisms at overlapping, physiologically achievable BHB concentration thresholds. At circulating BHB concentrations of 0.5-1 mM, NLRP3 inflammasome suppression begins; at 0.5-5 mM, HDAC inhibition and Kbhb deposition occur; at 0.1-1 mM, HCAR2/GPR109A signaling is engaged; at 1-3 mM, the NAD+/NADH ratio is elevated and redox buffering is enhanced; and at physiologically relevant BHB levels, circadian clock components are activated. When circulating BHB is elevated into a therapeutic range of approximately 1.5-3 mM, all five MERIT pillars are engaged concurrently, creating a multi-pillar pharmacological synergy that cannot be achieved by any combination of single-target drugs. An HDAC inhibitor will improve epigenetic dysregulation but will not suppress inflammasome activation or enhance NAD+/NADH ratios. An anti-IL-1B monoclonal antibody will suppress inflammation but will not reset circadian rhythm or enhance redox buffering. The multi-pillar simultaneity achieved by pharmacologically induced ketosis, and the resulting therapeutic breadth across diseases as divergent as oncology, autoimmunity, neurodegeneration, and infectious disease, is fundamentally impossible to replicate through any pharmacological combination of single-target agents.

### State-Gated Drug Efficacy: Metabolic State as a Therapeutic Multiplier

A foundational observation from recent clinical and preclinical research is that the efficacy of many therapeutic agents is profoundly gated by the metabolic state of the subject receiving the agent. This state-gating represents a fundamental departure from the classical pharmacology model-in which drug efficacy is determined solely by target engagement and dose-toward a state-gated pharmacology model, in which drug efficacy is determined by target engagement, dose, and the metabolic state of the tissue or cell populations expressing the target. Understanding this principle is essential to the diagnostic and therapeutic methods of the present disclosure, which encompasses both the use of pharmacologically induced ketosis as a direct monotherapy across multiple disease categories, and its use as a metabolic state-gate that dramatically enhances the efficacy of co-administered agents.

It is critical to recognize that pharmacologically induced ketosis provides direct therapeutic benefit as a monotherapy across multiple disease categories, independent of co-administered agents. The MERIT pillars are engaged directly by elevated circulating BHB and produce therapeutic effects in isolation: NLRP3-driven autoinflammatory diseases respond to ketosis monotherapy without requiring additional pharmacological agents; neurodegenerative diseases show disease-modifying benefit from monotherapy ketosis due to enhanced fuel substrate delivery and metabolic stress resilience; and psychiatric disorders show efficacy improvements when ketosis is used as a standalone intervention targeting circadian and epigenetic MERIT mechanisms. The power of ketosis as a monotherapy is distinct from, and independent of, its role as an efficacy-enhancing co-therapy with other drugs.

Ketogenic Diet and Anti-PD-1 Synergy: Preclinical Evidence. Preclinical research conducted in mice bearing two immunologically cold (non-inflamed, non-T-cell-infiltrated) syngeneic tumors —a colon cancer model and a renal cancer model —has provided the most direct demonstration of state-gated drug efficacy. In the control arm, in which tumor-bearing mice were fed standard chow with ad libitum access, anti-PD-1 monoclonal antibody at standard therapeutic doses produced a complete response rate of 10-15% with near-zero durable cures. In the experimental arm, the identical anti-PD-1 monoclonal antibody was administered to mice on a ketogenic diet achieving 3-5 mM circulating BHB. Under these conditions, the same anti-PD-1 monoclonal antibody at identical doses produced a 70% complete response rate and durable tumor cures in more than 60% of responding animals. When the ketogenic diet was administered as a monotherapy without anti-PD-1, there was modest tumor growth inhibition but no complete responses. The combination of ketogenic diet plus anti-PD-1 therefore represented a true synergy, not merely an additive effect. The mechanisms underlying this synergy were shown to involve three convergent processes: first, NLRP3-dependent remodeling of the tumor microenvironment, wherein the ketogenic diet suppressed NLRP3 inflammasome activation in tumor-associated myeloid cells, shifting the immune infiltrate from a pro-tumor, IL-18-producing, immunosuppressive phenotype toward a more activated, cytotoxic, Th1-enriched phenotype; second, a ketosis-induced shift of infiltrating T cells toward an oxidative-phosphorylation-dominant metabolic state, which enhanced their proliferation and effector function in response to PD-1 blockade; and third, histone acetylation-driven epigenetic remodeling of T cells and myeloid cells, enhancing the expression of anti-tumor genes and suppressing the expression of immunosuppressive genes. This body of evidence constitutes the most direct preclinical proof of state-gated drug efficacy, demonstrating that a 7-fold improvement in complete response rate can be achieved by pre-treating subjects with a metabolic intervention prior to drug administration, and these findings have been validated in independent laboratories and are consistent with multiple parallel observations in the broader literature.

Metabolic State-Dependent Drug Toxicity and Rescue. A striking example of how metabolic state can pharmacologically rescue drugs that would otherwise be too toxic to use comes from preclinical research examining a potent PI3Kα inhibitor-targeting the phosphoinositide 3-kinase alpha subunit, a therapeutic target in oncology. In the control arm on standard chow, the PI3Kα inhibitor induced severe, dose-limiting insulin feedback hyperactivation, leading to elevated circulating insulin, hyperglycemia, impaired glucose tolerance, and weight gain. This toxicity was of sufficient severity that meaningful therapeutic doses could not be achieved. However, when the identical PI3Kα inhibitor was administered to mice on a ketogenic diet, the insulin feedback hyperactivation was dramatically suppressed, glucose homeostasis was maintained, and the drug achieved excellent tumor-suppressive efficacy. The mechanism involves the suppression of baseline insulin secretion consequent upon the reduced glycemic excursions characteristic of ketogenic diet; when the PI3Kα inhibitor is administered in this context, the expected insulin feedback hyperactivation is attenuated because baseline insulin secretion is already low. This finding implies that many drugs that have been deemed too toxic or unsuitable for clinical development in unselected populations may in fact become viable therapeutic agents when administered to subjects in whom the ketotic state has been pharmacologically induced.

The clinical epidemiological evidence for state-gated efficacy is convergent and substantial. A retrospective analysis of more than one thousand melanoma patients treated with anti-PD-1 or anti-PD-L1 monoclonal antibodies examined the association between body mass index (BMI)—a proxy for metabolic state- and treatment response. The striking finding was an inverted U-shaped relationship: patients in the overweight BMI range (25-30 kg/m²) showed superior response rates of approximately 50%, compared to approximately 30% in normal-weight patients and approximately 20% in obese patients. This pattern has a clear mechanistic explanation within the MERIT framework. Patients with normal BMI are in excellent metabolic health with suppressed inflammatory tone and suppressed FGF21 signaling, resulting in suboptimal T-cell metabolic activation and reduced immunotherapy response. Patients with obese BMI have severe metabolic dysregulation-elevated insulin, insulin resistance, elevated inflammatory markers, and elevated NLRP3 inflammasome activation—that impairs T-cell function and reduces immunotherapy response. Patients with a BMI in the overweight range occupy a metabolic zone in which elevated but not yet severely dysregulated metabolic stress responses, including FGF21 induction and NAD+-dependent stress resistance activation, are sufficient to enhance T-cell metabolic fitness without the immunosuppressive consequences of frank obesity. The implication is that inducing a therapeutic ketotic state in normal-BMI or obese-BMI patients could restore immunotherapy efficacy by recreating the metabolic milieu of the overweight-BMI responders.

A large analysis of patients with non-small-cell lung cancer treated with an anti-PD-L1 monoclonal antibody further confirmed this relationship. Patients with metabolic syndrome-defined as the combination of elevated fasting glucose, elevated triglycerides, elevated blood pressure, and reduced HDL cholesterol-showed markedly reduced response rates of approximately 10%, compared to approximately 50% in metabolically healthy patients. This approximately 5-fold difference in immunotherapy response based on metabolic health status was maintained after adjustment for age, sex, smoking history, and tumor PD-L1 expression. Metabolic syndrome is characterized by dysregulated glucose homeostasis, elevated insulin, elevated inflammatory tone, elevated NLRP3 inflammasome activation, and impaired T-cell metabolic flexibility; the markedly reduced response to immunotherapy in this population is therefore mechanistically explained by these metabolic state-dependent defects in T-cell function and tumor microenvironment inflammatory state. A separate prospective study of patients with non-small-cell lung cancer receiving an anti-PD-1 monoclonal antibody assessed baseline metabolic phenotype using a composite score integrating BMI, waist circumference, blood glucose, triglycerides, and inflammatory markers. Metabolically healthy patients achieved a 50% response rate, whereas metabolically dysregulated patients achieved only 15%—a 3.3-fold difference that was independent of tumor PD-L1 expression, confirming that the effect is driven by host metabolic state rather than tumor characteristics. Finally, a systematic review and meta-analysis of thirteen independent studies encompassing over 7,000 patients treated with immune checkpoint inhibitors across multiple tumor types-including melanoma, non-small-cell lung cancer, renal cell carcinoma, urothelial cancer, and gastric cancer-demonstrated a consistent negative association between metabolic dysregulation and immunotherapy response. The pooled analysis showed that metabolically dysregulated patients were approximately 35% less likely to respond to immunotherapy than metabolically healthy patients, with even greater effect sizes in certain tumor types, and with metabolic state estimated to account for up to 30% of the variance in immunotherapy response across these populations. The convergent interpretation of these findings is unambiguous: the metabolic state of the host is a major determinant of drug efficacy, and subjects in whom the ketotic state has been induced show superior drug response compared to metabolically dysregulated cohorts.

Chronotherapeutic Timing of the Ketotic Window with Co-Administered Drug Pharmacokinetics. The therapeutic benefit of state-gated drug efficacy may be further optimized through temporal alignment of the pharmacokinetic window of the ketotic state the period during which circulating BHB remains above the target-engagement threshold for MERIT-pillar mechanisms—with the pharmacokinetic window of the co-administered drug —the period during which the drug remains above its EC50 or Cmax at the target tissue. This chronotherapeutic approach leverages the principle that maximal target engagement occurs when both the ketone-mediated MERIT-pillar signaling and the drug-target interaction are active simultaneously. The method involves calculating the Tmax and half-life of both the ketogenic agent and the co-administered drug using pharmacokinetic modeling informed by single-dose pharmacokinetic studies in the target population, then administering the ketogenic agent at a time calculated to produce peak BHB exposure coincident with peak drug concentration or to maintain supra-threshold BHB throughout the period of maximal drug exposure. For example, if a checkpoint inhibitor achieves maximal tissue-level target engagement 24-48 hours after intravenous administration, and if the ketogenic agent achieves sustained BHB elevation for 6-8 hours with a Tmax of 1.5-2 hours, the closing schedule would be designed to maintain continuous supra-threshold BHB throughout the critical window of checkpoint inhibitor-target interaction. This chronotherapeutic approach is further supported by the documented circadian coupling of BHB metabolism with BMAL1 and PER2 and by the emerging evidence for circadian variation in immune checkpoint receptor expression, T-cell metabolic fitness, and tumor microenvironment inflammatory state. The timing relationship can be optimized using pharmacokinetic/pharmacodynamic modeling informed by the ketogenic agent's Tmax and duration of BHB elevation, the co-administered drug's Tmax and tissue residence time, and the known circadian sensitivity of target pathway components. This chronotherapeutic optimization represents a precision approach to state-gated drug efficacy, wherein the magnitude of the therapeutic synergy is enhanced by temporal alignment of the two pharmacological interventions. The principle is particularly relevant to compounds with short Tmax and limited duration of action —such as ketone monoesters with a Tmax of 20-30 minutes and a half-life of 20-30 minutes- and is expected to be even more relevant to optimized ketone-carrier conjugates with a Tmax of 1-2 hours and a duration of action of 6-8 hours.

Systematic Method of Drug Development: Failed-Trial Resurrection as a Pipeline Strategy. The state-gated efficacy principle enables a systematic method of identifying drug compounds that failed clinical trial endpoints in unselected, metabolically heterogeneous patient populations and prospectively re-evaluating those compounds in populations in whom the ketotic state has been pharmacologically induced and maintained throughout the trial. This represents a fundamentally novel approach to drug development—not merely a treatment method for an individual patient, but a generalizable pipeline-generation strategy for rescuing compounds and programs that would otherwise be deemed clinically unsuccessful and abandoned. The operational methodology involves four sequential steps. First, a drug compound is identified that failed to meet a primary or secondary endpoint in a Phase II or Phase III clinical trial in an unselected patient population. Second, a retrospective metabolic analysis of the failed trial's patient population is conducted, including assessment of metabolic heterogeneity using fasting glucose, hemoglobin Alc, insulin levels, insulin resistance markers such as HOMA-IR, inflammatory biomarkers including CRP, IL-6, and TNF-alpha, lipid profiles, and body mass index distribution; the trial population is then stratified by baseline metabolic phenotype and a post-hoc efficacy analysis is performed in metabolically healthier versus metabolically dysregulated subgroups. Third, a prospectively randomized re-evaluation trial is designed in which the identical drug compound is administered to all enrolled subjects after pharmacological induction of a sustained ketotic state via ketone-carrier conjugate or other ketogenic agent, dosed to maintain circulating BHB at a predefined target concentration. Fourth, the trial assesses whether the drug now meets the primary efficacy endpoint in the metabolically controlled population. This pipeline-resurrection strategy is enabled by the convergent clinical and preclinical evidence that metabolic state gates drug efficacy across disease categories, spanning immunotherapy, metabolic disease, and oncology beyond immunotherapy. Every major pharmaceutical company maintains a portfolio of drug programs that failed to meet efficacy endpoints in Phase II or Phase III trials—a portfolio typically valued conservatively at zero, representing sunk research and development costs. The owner of an effective MERIT platform and the pharmacological means to reliably induce and maintain therapeutic ketosis possesses a systematic competitive advantage in identifying, acquiring, and rescuing these failed programs. Rather than developing entirely new drugs, this approach mines the accumulated failures of the pharmaceutical industry for compounds whose efficacy was obscured by metabolic state heterogeneity in the original trial populations, generating a second pipeline of drug candidates without requiring de novo drug discovery, preclinical development, or early-phase human trials, and operating on disclosed compounds that have already survived the most expensive phase of drug development.

The mechanisms underlying state-gated drug efficacy are multifaceted. The T-cell metabolic gate operates through the shift induced by ketosis from a glycolytic, mTORC1-dependent T-cell phenotype toward an oxidative-phosphorylation-dominant, mTORC2-dependent phenotype equipped with enhanced proliferative and effector capacity in response to checkpoint inhibitor stimulation. The tumor microenvironment inflammatory gate operates through BHB-mediated NLRP3 suppression in tumor-associated myeloid cells, shifting the immune infiltrate from a pro-tumor, immunosuppressive phenotype toward a cytotoxic, Th1-enriched phenotype permissive for T-cell infiltration and anti-tumor immunity. Secondary pharmacokinetic contributions —such as altered Fc-receptor expression and therapeutic antibody internalization kinetics in different metabolic states—may contribute to the overall effect, though the primary mechanisms are pharmacodynamic rather than pharmacokinetic. Taken together, these mechanisms establish that pharmacological induction of a ketotic state prior to or concurrent with drug administration can convert non-responders to responders, increase the magnitude of response in partial responders, or restore efficacy to drugs that had previously failed in unselected populations.

### MERIT-Responsive Conditions: Mechanistic Basis and Therapeutic Rationale

A MERIT-responsive condition is defined operationally as a disease, disorder, or pathological state characterized by dysregulation of one or more of the five MERIT pillars —Metabolic, Epigenetic, Redox, Immune, and Signaling- and therapeutically responsive to the pharmacological induction and maintenance of a ketotic state. The common mechanistic thread across all MERIT-responsive conditions is not a shared etiology or organ system, but rather shared dysregulation of one or more MERIT pathways and shared responsiveness to the restoration of MERIT homeostasis through ketosis induction. The following categories enumerate the major MERIT-responsive conditions and describe the relevant pillar dysregulations underlying each.

Oncological Conditions: Melanoma, non-small-cell lung cancer, renal cell carcinoma, colorectal cancer, gastric cancer, head and neck squamous cell carcinoma, and other solid malignancies are responsive to checkpoint inhibitor immunotherapy in a subset of patients, with response rates gated by the metabolic state of T cells and the tumor microenvironment, as demonstrated by the convergent clinical evidence reviewed above. Pharmacological induction of a ketotic state prior to or concurrent with checkpoint inhibitor administration enhances response rates by recreating the metabolic milieu favorable for T-cell activation and anti-tumor immunity, through simultaneous engagement of the immune pillar (NLRP3 suppression, T-cell metabolic reprogramming) and the epigenetic pillar (Kbhb-driven upregulation of anti-tumor and immune-tolerance genes). Hematologic malignancies and other oncological conditions may additionally be responsive through direct epigenetic effects on cancer cell apoptosis, redox-mediated enhancement of chemotherapy efficacy, or other MERIT mechanisms. Metabolic reprogramming is a hallmark of cancer, and the ability to therapeutically modulate cancer cell metabolism and the tumor microenvironment through ketosis induction represents a broadly applicable strategy in oncology.

Autoimmune and Inflammatory Diseases: Systemic lupus erythematosus, rheumatoid arthritis, inflammatory bowel disease, ankylosing spondylitis, and related conditions are characterized by aberrant NLRP3 inflammasome activation, dysregulated T-cell and B-cell metabolism, impaired barrier function, and elevated systemic inflammation. NLRP3 inflammasome suppression induced by ketosis directly addresses the core pathogenic mechanism in NLRP3-dependent autoimmune conditions. Ketosis-induced enhancement of barrier function through intestinal epithelial tight-junction protein expression and stem cell renewal addresses a key mechanism of autoimmune pathogenesis, while the epigenetic remodeling induced by ketosis promotes the differentiation of T-regulatory cells and suppresses pro-inflammatory gene expression in T cells and myeloid cells. Metabolic state-dependent disease susceptibility has been clearly documented in autoimmune disease, with obesity and metabolic syndrome significantly increasing disease severity and reducing response to immunosuppressive therapy.

Infectious Diseases and Sepsis: Bacterial sepsis, fungal sepsis, certain viral infections, and post-infectious syndromes are characterized by dysregulated innate immune activation, elevated NLRP3 inflammasome activity, excessive cytokine production, impaired T-cell function, and organ injury secondary to excessive inflammation. Ketosis-induced NLRP3 inflammasome suppression directly addresses the core pathogenic mechanism driving tissue injury in sepsis. The NAD+/NADH shift and mitochondrial bioenergetic enhancement induced by ketosis promote immune cell function and metabolic resilience in the setting of critical illness, while the redox-enhancing effects of ketosis directly address oxidative stress-driven organ injury.

Neurodegenerative Diseases: Parkinson's disease, Alzheimer's disease, amyotrophic lateral sclerosis, progressive supranuclear palsy, and other neurodegenerative diseases are characterized by mitochondrial dysfunction, elevated oxidative stress, microglial activation and pro-inflammatory signaling, neuroinflammation, and impaired clearance of misfolded proteins. Ketosis directly addresses multiple pathogenic mechanisms simultaneously: enhanced mitochondrial bioenergetics via elevated NAD+/NADH and ketone-supported oxidative phosphorylation improves neuronal and glial energy production and reduces ROS generation; NLRP3 inflammasome suppression dampens microglial activation and neuroinflammation; histone acetylation-driven upregulation of autophagy-related genes promotes clearance of misfolded proteins; and circadian rhythm resetting normalizes sleep-wake cycles and glial circadian function. The neuroprotective effects of ketogenic diet in multiple neurodegenerative conditions are well-documented and attributable to these convergent MERIT pillar mechanisms.

Neuropsychiatric Conditions: Major depressive disorder, anxiety disorders, bipolar disorder, post-traumatic stress disorder, and related neuropsychiatric conditions are characterized by impaired histone acetylation, elevated HDAC expression, dysregulated circadian rhythm, impaired stress-response hormonal signaling, and altered neurotrophic signaling. Ketosis-induced histone acetylation and HDAC inhibition directly address the epigenetic dysregulation underlying these conditions, including histone acetylation-driven upregulation of brain-derived neurotrophic factor (BDNF), a critical mediator of neuroplasticity and depression remission. The circadian rhythm resetting induced by ketosis normalizes disrupted sleep-wake cycles, and the NAD+-dependent enhancement of stress-resistance pathways enhances the organism's capacity to handle physiological and psychological stress. The anti-inflammatory effects of ketosis and the HCAR2-signaling-induced shift toward tolerogenic immune responses address the emerging evidence for neuroinflammation as a pathogenic mechanism in depression and anxiety.

Cardiovascular Conditions: Heart failure with preserved ejection fraction, atrial fibrillation, post-myocardial infarction remodeling, atherosclerosis, and related cardiovascular conditions are characterized by impaired myocardial and endothelial metabolism, elevated oxidative stress, dysregulated autophagy, and inflammatory infiltration of myocardium and vasculature. Ketosis enhances myocardial and endothelial mitochondrial function through the NAD+/NADH shift and mitochondrial biogenesis, reduces myocardial and endothelial oxidative stress through NAD+-dependent Nrf2 activation, enhances autophagy through NAD+-dependent sirtuin-mediated autophagy upregulation, and suppresses cardiac and vascular inflammation through NLRP3 suppression and FGF21-mediated anti-inflammatory signaling. Clinical trials of SGLT2 inhibitors in heart failure have shown remarkable benefits in hospitalization reduction and survival improvement, benefits increasingly understood to be mediated by ketosis-driven mechanisms rather than glucose lowering alone.

Renal and Urological Conditions: Chronic kidney disease, lupus nephritis, IgA nephropathy, and related renal conditions are characterized by glomerular inflammation, tubular dysfunction, impaired mitochondrial function in renal tubular cells and podocytes, elevated oxidative stress, and progressive renal fibrosis. Ketosis enhances renal tubular cell mitochondrial function, reduces glomerular and tubular oxidative stress, suppresses NLRP3-driven glomerular inflammation, and promotes autophagy-mediated clearance of damaged mitochondria and proteins. Clinical observations in patients receiving SGLT2 inhibitors show protection against renal progression independent of glucose control, consistent with ketosis-mediated MERIT pillar engagement.

Metabolic and Endocrine Disorders: Type 2 diabetes mellitus, obesity, and metabolic syndrome are characterized by insulin resistance, dysregulated glucose homeostasis, ectopic fat deposition, elevated inflammatory tone, and impaired metabolic flexibility. Pharmacological induction of ketosis directly addresses these mechanisms: the metabolic shift toward fatty acid oxidation enhances metabolic flexibility, reduces hepatic glucose production, improves insulin sensitivity through NAD+-dependent improvements in insulin signaling, suppresses circulating inflammation through NLRP3 suppression and FGF21-mediated anti-inflammatory signaling, and promotes favorable changes in body composition through enhanced energetic efficiency and FGF21-mediated effects on appetite.

Aging-Related Conditions and Senescence: Biological aging, frailty, age-related cognitive decline, and sarcopenia are characterized by mitochondrial dysfunction, elevated ROS production, impaired autophagy, dysregulated immune signaling (inflammaging), and epigenetic dysregulation. Ketosis addresses all these mechanisms simultaneously: enhanced mitochondrial function through the NAD+/NADH shift and mitochondrial biogenesis; enhanced ROS scavenging and antioxidant enzyme expression; enhanced autophagy through NAD+-dependent sirtuin upregulation; suppression of inflammaging through NLRP3 suppression and FGF21-mediated anti-inflammatory signaling; epigenetic remodeling through histone acetylation upregulation and Kbhb deposition; and NAD+-dependent activation of stress-resistance pathways. Longevity studies in model organisms have demonstrated lifespan extension with caloric restriction and intermittent fasting-both of which induce ketosis —with the NAD+/NADH shift and sirtuin activation identified as critical mediators of longevity.

Cancer Cachexia and Wasting Syndromes: Cancer cachexia, cachexia of critical illness, and age-related sarcopenia are characterized by impaired protein synthesis, enhanced protein degradation, mitochondrial dysfunction, elevated oxidative stress, systemic inflammation, and impaired metabolic flexibility. Ketosis enhances muscle mitochondrial function, suppresses inflammatory mediators driving muscle wasting through NLRP3 suppression and FGF21-mediated anti-inflammatory signaling, promotes autophagy-mediated clearance of damaged organelles, and enhances muscle metabolic flexibility. Preliminary studies suggest that ketosis combined with physical activity enhances muscle protein synthesis and attenuates wasting compared to either intervention alone.

### Biomarker-Guided Methods: Companion Diagnostics and Treat-to-Target Titration

The pharmacological induction of ketosis can be guided and optimized through the measurement of MERIT biomarkers in peripheral blood, saliva, urine, or tissue biopsies. Biomarker-guided approaches serve multiple purposes within the scope of this disclosure: patient selection and enrichment, dose titration to achieve predefined target biomarker readouts, response prediction, and mechanism-of-action validation. The rationale, methodology, and clinical utility of these biomarker-guided methods are described in the present section.

Biomarker-Guided Patient Selection. Subjects likely to benefit from ketogenic agent treatment can be identified through baseline measurement of MERIT biomarkers, with enrichment for subjects showing evidence of MERIT dysregulation-including low NAD+/NADH ratio, elevated NLRP3 inflammasome activation, reduced histone acetylation, and elevated oxidative stress markers—at baseline. Such biomarker-guided enrichment can increase the response rate to treatment and reduce the number of subjects needed to treat to achieve clinical benefit. In an oncology context, for example, subjects with baseline evidence of elevated NLRP3 inflammasome activation in tumor-associated myeloid cells or in circulating monocytes would be enriched for response to a ketogenic agent plus immunotherapy combination, enabling smaller and faster clinical trials. Subjects with low baseline NLRP3 inflammasome activation and high NAD+/NADH ratio may be classified as metabolically healthy and enrolled in standard control arms. MERIT biomarkers used for patient selection include, without limitation, circulating BHB concentration, histone β-hydroxybutyrylation in peripheral blood cells measured by mass spectrometry, NLRP3 inflammasome activation status in monocytes or other leukocyte subsets measured by caspase-1 activity or ASC speck formation assays, NAD+/NADH ratio in accessible tissues, histone deacetylase activity, circulating cytokine levels, T-cell metabolic phenotype, and composite MERIT biomarker indices integrating multiple readouts into a single score (e.g., the MVX composite index).

Biomarker-Guided Dose Titration. Rather than employing a fixed-dose approach to ketogenic agent administration, doses can be titrated individually to achieve predefined target biomarker readouts—an approach analogous to therapeutic drug monitoring in immunosuppressive therapy and target-engagement-guided closing of kinase inhibitors. Circulating BHB target titration involves escalating doses until circulating BHB reaches the indication-specific target range (e.g., 1-2 mM for most MERIT-responsive conditions). Histone β-hydroxybutyrylation target titration involves assaying Kbhb occupancy in peripheral blood lymphocytes or monocytes and escalating doses until Kbhb enrichment reaches the predefined threshold (e.g., ≥50% of total histones). NLRP3 inflammasome activity target titration involves collecting circulating monocytes, stimulating them ex vivo, assaying caspase-1 activity or pro-IL-1β cleavage, and escalating doses until NLRP3 inflammasome activity is suppressed by the predefined threshold (e.g., >60% relative to baseline). NAD+/NADH ratio target titration involves measuring the ratio in circulating lymphocytes or platelets and escalating doses until the predefined elevation is achieved (e.g., ≥1.5-fold relative to baseline). A composite MERIT Biomarker Index (MVX) integrating multiple biomarkers from different pillars—including Kbhb enrichment, NLRP3 suppression, NAD+/NADH elevation, histone acetylation enrichment, ROS markers, and circulating cytokine levels —can provide a comprehensive MERIT activation score and serve as the treat-to-target endpoint, with doses titrated until a predefined MVX threshold is achieved. Treat-to-target closing reduces the number of subjects requiring high doses by individualizing dose to the degree of biomarker change needed to achieve therapeutic benefit and enables accommodation of pharmacogenetic variability.

Biomarker Response Prediction and Adaptive Trial Design. Baseline and on-treatment MERIT biomarkers can predict which subjects will achieve clinical benefit and which will not, enabling adaptive trial designs in which subjects showing inadequate biomarker response are re-randomized or de-escalated from trial arms. In a randomized controlled trial of ketogenic agent plus immunotherapy versus immunotherapy alone, subjects randomized to the combination arm can be assessed for biomarker response-such as Kbhb enrichment reaching ≥50% and NLRP3 suppression reaching ≥60%—at an early interim timepoint, such as week four of treatment. Subjects showing inadequate biomarker response are re-randomized to higher-dose ketogenic agent or alternative ketogenic agent formulation, while subjects showing adequate biomarker response continue on the same dose. This adaptive design enriches the trial population for true biomarker responders and improves the signal-to-noise ratio in detecting clinical efficacy. The present disclosure encompasses the use of MERIT biomarker assays, diagnostic panels, and companion diagnostics as part of such adaptive trial designs, as well as their use in routine clinical practice for patient selection, risk stratification, treatment response prediction, and dose titration guidance.

The disclosure further encompasses systems designed to implement the diagnostic and therapeutic methods based on the UTBS framework. These systems typically integrate measurement capabilities with data processing and decision support functionalities, embodying the core logic of UTBS by synthesizing diverse data inputs within the M=E/R framework to generate actionable outputs.

### Acetoacetate Quantification and BHB:AcAc Ratio Optimization

Acetoacetate (AcAc) is a primary active ketone body with distinct and complementary mechanisms of action compared to β-hydroxybutyrate (BHB). While BHB serves as both a fuel substrate and a signaling molecule-engaged in histone β-hydroxybutyrylation (Kbhb), epigenetic remodeling, and HCAR2 activation-AcAc functions as a substrate for lysine acetoacetylation (Kacac), a histone post-translational modification distinct from Kbhb that engages a complementary transcriptional program. Additionally, AcAc is the immediate precursor to acetone, which is volatile and readily crosses the blood-brain barrier, providing rapid reactive oxygen species scavenging activity independent of BHB metabolism. AcAc also inhibits the vesicular glutamate transporter (VGLUT), a mechanism wholly independent of BHB and particularly relevant to epilepsy and psychiatric disorders where pathologically elevated glutamatergic tone is a core feature of disease.

The optimal therapeutic ketotic state may therefore require optimization not merely of total circulating ketone body concentration, but specifically of the ratio of BHB to AcAc. This ratio varies with the MERIT-responsive condition being treated and the specific MERIT pillar or pillars intended to be engaged. A subject with an oncological condition being treated for NLRP3 inflammasome suppression and metabolic remodeling of tumor-infiltrating lymphocytes may benefit from a different BHB:AcAc ratio than a subject with epilepsy or a neuropsychiatric disorder where VGLUT inhibition and acetone-mediated neuroprotection are the primary therapeutic targets. The ability to measure and rationally optimize this ratio—at the level of both the formulation and the individual subject-represents a meaningful advance in the rational design of ketosis-based therapeutics.

Acetoacetate can be quantified by liquid chromatography-tandem mass spectrometry (LC-MS/MS), which represents the gold-standard analytical approach, or by enzymatic assay using ketoacetyl-CoA transferase or similar enzyme-based detection platforms. Breath acetone concentration serves as a practical non-invasive surrogate for circulating AcAc and can be measured by portable ketone breath analyzers in real time, enabling non-invasive feedback in outpatient settings. Breath acetone correlates with circulating AcAc and offers the advantage of non-invasive, rapid measurement suitable for repeated daily monitoring without the burden of capillary or venous blood sampling.

A key advantage of the ketone-carrier conjugate architecture disclosed herein is that when the acyl component of the conjugate is composed of acetoacetate, AcAc is delivered directly into systemic circulation upon hydrolysis, circumventing reliance on hepatic interconversion from BHB and enabling BHB:AcAc ratio optimization at the formulation level itself. This represents a structural and mechanistic advantage unavailable with traditional exogenous ketone salts or monoester compositions, which deliver predominantly BHB or are metabolized predominantly to BHB after oral administration and therefore cannot independently control the ratio of circulating AcAc to BHB without additional formulation engineering. By designing conjugate compositions with defined proportions of BHB and AcAc acyl components, a clinician or formulation scientist can achieve indication-specific BHB:AcAc ratios independently of individual subject metabolism, enabling precise ratio targeting without dietary co-intervention.

Clinical development of ketogenic agent therapeutics must prioritize quantification of circulating AcAc and systematic BHB:AcAc ratio optimization in order to unlock the full therapeutic potential of the acetoacetate-specific MERIT mechanisms described herein. The initial MERIT biomarker studies have predominantly measured circulating BHB as the primary pharmacokinetic readout. Future clinical development should expand to include simultaneous AcAc quantification, calculation of the BHB:AcAc molar ratio, and investigation of whether indication-specific ratio optimization produces superior MERIT target engagement compared to BHB-dominant formulations. This represents a priority area for follow-up clinical investigation across the MERIT-responsive indication categories.

### Glucose-Ketone Index (GKI) as a Clinical Biomarker for MERIT-Responsive Conditions

The Glucose-Ketone Index (GKI) is a composite clinical biomarker that integrates both the glucose and ketone dimensions of metabolic state into a single actionable numerical value, enabling simplified treat-to-target closing in ambulatory clinical settings. GKI is calculated according to the formula: GKI=[glucose (mg/dL)] divided by ([β-hydroxybutyrate (mmol/L)] multiplied by 18.01), where the denominator accounts for the molecular weight conversion between BHB expressed in mmol/L and an equivalent glucose-scale denominator. A lower GKI value indicates a deeper shift toward ketosis and glucose metabolic subordination; a higher GKI value indicates glucose reliance and an insufficient ketotic state. GKI thereby integrates two independent dimensions of metabolic state-glucose level and ketone level-into a single scalar value that reflects the overall direction and depth of the metabolic shift toward ketosis.

GKI has been validated in the ketogenic diet literature as a predictor of therapeutic response across multiple indications. A GKI below 1.0 has been associated with enhanced seizure control in drug-resistant epilepsy. GKI-guided dietary ketosis optimization has been reported to improve metabolic markers in cardiovascular disease populations. In oncological contexts, GKI thresholds have been associated with tumor growth inhibition in glioblastoma models and subcutaneous xenografts, with a GKI at or below 1.0 associated with maximal metabolic stress to malignant cells. These findings establish GKI as a mechanistically grounded and clinically validated biomarker suitable for guiding pharmacological ketosis induction across the full range of MERIT-responsive conditions.

Indication-specific GKI targets can be defined based on the mechanistic requirements of each MERIT-responsive condition and the depth of ketotic state required to engage downstream MERIT pillars at the relevant threshold concentrations. For oncological and psychiatric indications, where maximal NLRP3 inflammasome suppression and epigenetic remodeling via Kbhb deposition are mechanistically critical, a target GKI at or below 1.0-corresponding to deep ketosis —is supported by the available mechanistic and clinical literature. For sepsis and infectious disease, where moderate inflammasome suppression and immune metabolic rebalancing are the primary goals, a GKI at or below 1.2 may be sufficient to engage the requisite degree of NLRP3 suppression. For neurodegenerative diseases, where neuroprotection through enhanced mitochondrial bioenergetics and ketone-supported oxidative phosphorylation are the primary therapeutic goals, a GKI at or below 2.0 may be adequate to provide meaningful MERIT pillar engagement. For metabolic and cardiovascular indications, where metabolic normalization without the necessity of extreme ketosis is sought, a GKI at or below 3.0 provides a practical and achievable target for ambulatory management. These thresholds represent rational starting points for clinical protocol development derived from the mechanistic evidence base; individual subjects may require optimization within and around these ranges depending on baseline metabolic phenotype, body composition, and co-administered medications.

GKI can be measured at the point of care using dual-analyte meters capable of measuring both blood glucose and BHB from a single capillary blood sample obtained by fingerstick, eliminating the logistical burden of separate glucose and BHB measurements and enabling rapid bedside calculation of the index without laboratory infrastructure. Real-time GKI computation can be integrated with continuous glucose monitors (CGMs) and continuous or intermittent ketone monitors to enable automated GKI calculation and dose-adjustment recommendations delivered via mobile application or cloud-connected platform. This technological integration transforms GKI from a research-grade biomarker into a practical clinical tool suitable for outpatient management of patients receiving pharmacological ketosis induction, enabling clinicians to monitor metabolic state in real time and adjust ketogenic agent dose based on objective numerical feedback.

GKI-guided closing represents the practical clinical implementation of the treat-to-target ketosis principle in ambulatory settings. Rather than prescribing fixed doses of ketogenic agent without individual optimization, a GKI-guided approach enables clinicians to measure individual metabolic response, calculate the current metabolic state of the subject, and adjust the dose of the ketogenic agent to achieve the indication-specific target threshold. This approach is directly analogous to hemoglobin Alc-guided insulin closing in diabetes management or INR-guided warfarin closing in anticoagulation therapy—it embeds pharmacodynamic feedback into the routine therapeutic workflow without requiring specialized laboratory infrastructure or research-grade biomarker platforms. When combined with the broader MERIT biomarker panel —including histone Kbhb enrichment, NLRP3 inflammasome activity, and NAD+/NADH ratio-GKI provides a simplified yet mechanistically grounded first-line approach to rational ketosis optimization that is accessible to clinicians operating in standard outpatient settings. GKI monitoring is therefore recommended as the minimum standard of metabolic monitoring in all clinical studies employing pharmacological ketosis induction within the scope of this disclosure, with expansion to the full MERIT biomarker panel in studies where mechanistic validation and target-engagement confirmation are primary objectives.

### Systems for Diagnosis and Treatment

The disclosure further encompasses systems designed to implement the diagnostic and therapeutic methods based on the UTBS framework. These systems typically integrate measurement capabilities with data processing and decision support functionalities, embodying the core logic of UTBS by synthesizing diverse data inputs within the M=E/R framework to generate actionable outputs.

A representative system might include:

Data Acquisition Module(s): One or more devices for measuring parameters related to M=E/R. This could involve a PET scanner configured for specific metabolic tracers (e.g., FDG, ketone tracers), an MRS system capable of acquiring 31P or 1H/¹³C spectra from target tissues, laboratory instruments for analyzing blood/plasma biomarkers using specific assay kits (e.g., automated analyzers for ATP, BHB, lactate, inflammatory markers), or devices for functional testing (e.g., indirect calorimeter for RQ).

Processor Module: A computer or embedded processor operably connected to the data acquisition modules.

Software/Algorithms: The software is configured to: Receive and process raw data from measurement devices. Calculate or estimate M=E/R or related indices (e.g., PCr/ATP ratio, substrate uptake SUVR®, flexibility index, MVX score). Compare calculated values or biomarker profiles to predefined thresholds or patterns corresponding to the UTBS metabolic stages (Table 2) to determine the subject's stage. Optionally, implement mathematical models of UTBS to simulate metabolic dynamics, predict response to potential therapies, or personalize treatment recommendations. Potentially incorporate machine learning or artificial intelligence algorithms for enhanced pattern recognition from complex, multi-modal data (integrating imaging, metabolomics, clinical data) to improve diagnostic accuracy, prognostic prediction, or therapy selection.

Output Module: A display or interface presenting the diagnostic results (e.g., estimated M, UTBS stage), prognostic information, or therapeutic recommendations to a clinician or user. In some embodiments, the system might be integrated with a therapeutic delivery device (e.g., an infusion pump) to enable closed-loop control or automated adjustment of therapy based on real-time metabolic monitoring.

Such integrated systems offer the potential for more objective, quantitative, and personalized management of conditions characterized by bioenergetic insufficiency, moving beyond subjective symptom assessment towards therapies guided by the underlying metabolic state as defined by UTBS. The novelty lies not just in the hardware components but in the processor executing UTBS-specific algorithms to integrate multi-modal data in a physiologically meaningful way.

### Exemplary Applications in Specific Pathologies

The power of the UTBS framework lies in its applicability across a wide range of chronic diseases previously viewed as distinct entities. This section illustrates how the diagnostic and therapeutic principles described herein can be applied to specific conditions frequently associated with metabolic dysfunction. The ability to use the same core framework (M=E/R assessment, staging, energy rescue) across diverse pathologies highlights the fundamental and unifying nature of UTBS, distinguishing it from purely disease-specific paradigms and supporting its potential as a broad therapeutic platform.

Neurodegenerative Diseases (Alzheimer's Disease, Parkinson's Disease): UTBS Framing: Chronic brain energy insufficiency (low M). AD: Impaired cerebral glucose utilization (low E_glucose, ↓FDG-PET) often precedes symptoms. PD: Mitochondrial dysfunction (Complex I) leads to deficient ATP production (low E/η_i).Diagnosis/Staging: FDG-PET identifies hypometabolism. Ketone PET (11C-AcAc) assesses compensatory capacity MRS measures neuronal health (NAA) and energy state (PCr/ATP-though findings vary, lactate). Plasma biomarkers (MVX, inflammation) contribute to UTBS staging (Table 2). Treatment: Energy rescue via alternative fuels. Ketones (esters 16, MCTs, diet) bypass glucose deficit in AD and potentially Complex I in PD. Potential cognitive/motor benefits shown. Adjunctive to standard therapies.

### Cardiovascular Disease (Heart Failure):

UTBS Framing: Myocardial energy starvation (low M=E/R). E often impaired (↓FAO, inefficient glucose use). Compensatory ketone utilization upregulated. Low PCr/ATP ratio is a key indicator. Diagnosis/Staging: 31P-MRS assesses cardiac PCr/ATP ratio. PET assesses substrate uptake (glucose, FAs, ketones). Plasma biomarkers (NT-proBNP, MVX, lactate, ketones) inform staging (Table 2), complementing NYHA/CKM.Treatment: Ketone supplementation (e.g., esters) provides efficient fuel, improving cardiac output, reducing filling pressures. Adjunctive to GDMT, potential synergy with SGLT2i. Careful consideration of the “ketone paradox” (high endogenous ketones associated with poor prognosis) is warranted when interpreting baseline levels versus therapeutic intervention.

### Metabolic Syndrome and Type 2 Diabetes (T2D):

UTBS Framing: Systemic metabolic inflexibility (impaired PFs) driven by insulin resistance. Leads to low M in vulnerable tissues over time. Diagnosis/Staging: Standard measures (glucose, insulin, HbA1c, lipids), flexibility tests (RQ shift). UTBS staging reflects progression (Stage 1: inflexibility/risk; Stage 2-3: complications). Treatment: Lifestyle interventions, insulin sensitizers (Metformin, TZDs). Ketones (e.g., via SGLT2i or supplementation) may play role in specific contexts (e.g., T2D+HFpEF).

### Traumatic Brain Injury (TBI):

UTBS Framing: Acute, severe brain energy crisis (precipitous ↓M due to JE [ischemia, mito damage]+↑R [excitotoxicity, inflammation]). Diagnosis/Staging: Acute MRS monitoring (lactate, potentially PCr/ATP). Treatment: Acute energy rescue with alternative fuels (lactate, ketones) to bridge energy gap.

### Cachexia:

UTBS Framing: Profound negative energy balance (low M, esp. muscle) due to inflammation, anorexia, hypermetabolism. Diagnosis/Staging: Body composition, function, inflammation markers. UTBS stage reflects severity. Treatment: Multi-modal: treat primary cause, nutritional support targeting E (protein, AAs, potentially ketones), anti-inflammatories.

### Aging:

UTBS Framing: Gradual decline in M (lefficiency, ↓flexibility, ↑mito dysfunction), leading to Tentropy accumulation and susceptibility to chronic disease. Diagnosis/Staging: Flexibility tests, mito markers, aging biomarkers (e.g., related to proteostasis). UTBS stage reflects bioenergetic resilience. Treatment/Prevention: Maintain M via lifestyle, mitochondrial support (e.g., NAD⁺ precursors), intermittent ketosis. Goal: slow entropy accumulation, extend healthspan.

### Orphan Diseases

Describe herein are several “basket” categories, each comprising multiple specific orphan disease indications that could be treated under the inventive concept. This basket trial strategy reflects the platform nature of ketone therapy. Epilepsy & Neurological Disorders (IGNITE Basket): This group includes rare epilepsy syndromes and metabolic epilepsies where providing an alternative fuel can quell neuronal hyperexcitability. Examples:

GLUT1 Deficiency Syndrome (Glut1-DS): As discussed, these patients benefit from any source of ketones. Exogenous ketone therapy can serve as primary or adjunct treatment to reduce seizures and improve cognitive development, especially in patients who cannot maintain a strict ketogenic diet.

Pyruvate Dehydrogenase Complex Deficiency (PDHc-D): Patients typically present with lactic acidosis and seizures. Ketone therapy bypasses the PDH block by feeding acetyl-CoA via ketolysis, thereby both generating ATP and consuming NADH (reducing lactic acid formation). Significant improvements are anticipated in neurodevelopmental outcomes by early ketone intervention (supported by historical KD use improving survival).

Developmental and Epileptic Encephalopathies (DEE) like Dravet Syndrome, Lennox-Gastaut Syndrome (LGS), Doose Syndrome, SLC13A5 Deficiency: These are severe childhood epilepsies often refractory to meds. Many DEE patients show metabolic inflexibility or mitochondrial issues. Ketogenic diet has shown efficacy in a portion of these patients, implying a metabolic mechanism. Exogenous ketones could provide seizure control without the dietary burden. SLC13A5, for instance, is a citrate transporter defect causing seizures; ketones might help by substituting for the energy shortfall and potentially modulating citrate levels.

Other neurometabolic conditions: e.g., Leigh syndrome (subacute necrotizing encephalopathy) and certain mitochondrial DNA disorders that present with seizures and regression could also be included here if seizures are prominent. (They also fit the mito basket below, showing overlap.)

Mitochondrial Encephalomyopathies (EMBER Basket): These are rare genetic disorders directly affecting mitochondrial function (electron transport chain, mtDNA mutations, etc.). They often involve the brain and muscle with symptoms like encephalopathy, strokes, myopathy, ataxia.

Leigh Syndrome (various nuclear or mtDNA causes, e.g., NDUFS4 mutations): A prototypical mitochondrial disease where brain regions suffer energy failure. Ketone therapy could prolong survival or improve neurologic function. A prophetic Example 6 below outlines treating Ndufs4−/−mice with ketone ester to extend lifespan.

MELAS (Mitochondrial Encephalomyopathy Lactic Acidosis and Stroke-like episodes): Caused by mtDNA mutations (e.g., m.3243A>G in tRNA\hat{ }Leu). Patients have brain energy deficits (seen on FDG-PET sometimes). Ketones might reduce stroke-like episodes by fueling neurons during crises. Also, by improving NAD⁺ levels, ketones might counteract some of the redox imbalance of MELAS.

MERRF (Myoclonic Epilepsy with Ragged Red Fibers): Another mtDNA disorder; features myoclonus, seizures, muscle problems. Could benefit similarly to MELAS, especially for seizure control and muscle fatigue.

Friedreich's Ataxia: Although nuclear-encoded (frataxin gene), it leads to mitochondrial iron handling issues and energy deficit in nervous system and heart. Ketone supplementation might improve neuronal survival and delay progression of ataxia (some preclinical support exists for ketosis improving motor function in models).

Other related: Juvenile ALS with FUS mutation (a very rare early-onset ALS) might be included here or neurodegenerative basket due to overlapping metabolic impairment.

The unifying element in this basket is mitochondrial ATP synthesis is impaired. By providing acetyl-CoA and boosting NAD⁺/NADH, ketones help remaining mitochondria work more efficiently. Many of these conditions currently have no disease-modifying treatment, so ketone therapy could be a breakthrough.

Fatty Acid Oxidation Disorders & Acute Metabolic Crises (AVALANCHE Basket): This category covers inborn errors of metabolism where the primary defect is in fat utilization, resulting in hypoketotic states and life-threatening crises during fasting or illness. It also includes acute scenarios like neonatal hypoglycemia that behave similarly.

VLCAD Deficiency (Very Long Chain Acyl-CoA Dehydrogenase Deficiency): Patients cannot burn long-chain fats, causing energy failure especially during fasting. They typically must avoid fasting and often use cornstarch or MCT oil. Exogenous ketones would provide immediate alternate fuel and have already shown dramatic effects: case reports note improved exercise tolerance and normalized cardiac function on supplemental BHB. Example 3 demonstrates that in a VLCAD−/−mouse, ketone ester administration during fasting prevented the usual lethal outcome.

LCHAD Deficiency/TFP Deficiency: Similar to VLCAD but at a different enzyme; causes cardiomyopathy, liver issues. Triheptanoin is sometimes used here. Ketone esters can similarly be applied. One advantage of BHB over triheptanoin is that BHB directly fuels the brain and heart without needing mitochondrial β-oxidation at all.

MCAD Deficiency: Medium-chain defect; these patients do make some ketones but could still benefit from extra ketones in prolonged fasting or illness.

MADD (Multiple Acyl-CoA Dehydrogenase Deficiency, a.k.a. GA-II): This is an ETF/ETF-DH defect affecting multiple fatty acid steps. Patients often respond to high-dose riboflavin and low-fat diet, but still have crises. Notably, a recent international survey reported that use of D,L-3-hydroxybutyrate (racemic BHB) in MADD patients was safe and improved their exercise tolerance and reduced hospitalizations. Ketone therapy is extremely well-suited for MADD since it bypasses the entire upstream beta-oxidation chain. Compounds described herein (particularly SNV—C1 or SNV-G3) could essentially replace the defective energy pathway by providing a continuous alternative fuel.

Organic Acidemias (e.g., HMG-COA lyase deficiency, which ironically cannot produce ketones, or propionic acidemia, etc.): While primarily amino acid disorders, they result in secondary energy deficits and often benefit from some degree of ketosis. HMG-COA lyase deficiency patients can't make ketones and suffer from hypoketotic hypoglycemia-giving ketones could be directly life-saving as a bridge in acute episodes.

Other acute crises: This basket also conceptually covers acute neonatal hypoglycemia, hyperinsulinism episodes, or even glycogen storage disease type III or 0 (where ketones can help when glucose runs out). Ketone esters can serve as a “bridge” energy source until definitive management (like glucose infusion) is in place.

For FAODs especially, ketone therapy is differentiated by being ketogenic-diet-like without the fat load. Ketogenic diet is actually contraindicated in FAOD patients (because high fat can precipitate metabolic crises since they can't break it down). Thus, exogenous ketones offer a way to get the benefits of ketosis without the burden of fat metabolism.

Cardiomyopathy & Muscle Energy Disorders (TITAN Basket): This group comprises rare genetic diseases where the heart and/or skeletal muscle suffer from energy production problems, leading to cardiomyopathy or muscle weakness. Many overlap with mitochondrial or FAOD, but some are distinct.

Barth Syndrome: An X-linked disorder affecting cardiolipin synthesis in mitochondria, causing severe cardiomyopathy and muscle weakness. The heart failure in Barth is partly due to inefficient energy metabolism (cardiolipin is needed for optimal ETC function). Ketone therapy could bypass some of the inefficient steps and fuel the heart; ketones are known to be a preferred fuel in failing hearts, potentially improving cardiac output.

Duchenne Muscular Dystrophy (DMD)—associated Cardiomyopathy: While DMD is a structural muscle disease (dystrophin mutation), as patients age they develop dilated cardiomyopathy. Managing heart failure in DMD is challenging. Ketone supplementation (like D-BHB) has been shown to improve cardiac efficiency in heart failure models by shifting the heart's fuel preference. Subset of DMD patients could be targeted with heart failure as an orphan population where ketone ester (perhaps SNV—C1 with creatine for muscle) might enhance cardiac ATP and delay progression. (Note: DMD prevalence is high, but “DMD cardiomyopathy” as a subset could be orphan.)

SMA Type II (Spinal Muscular Atrophy): A motor neuron disease (developmental) which also leads to muscle atrophy. Recent data suggests metabolic deficiencies in muscle contribute. There is anecdotal interest in ketogenic therapy for SMA (to boost muscle endurance and reduce oxidative stress). A ketone ester with an autophagy inducer (like trehalose) could hypothetically help clear accumulations in motor neurons and also energize muscle fibers.

LMNA-related Dilated Cardiomyopathy: Mutations in lamin A/C cause a form of muscular dystrophy and cardiomyopathy often qualifying as orphan. Mice with LMNA mutation have improved survival on ketogenic diet in some studies (though data is sparse).

The disclosure's approach could provide those benefits more practically. Ketones might help stabilize the energetics of heart muscle which in laminopathies is failing due to chronic stress.

Others: Friedreich's Ataxia could also fit here as it involves hypertrophic cardiomyopathy in addition to neurodegeneration. Mitochondrial myopathies (like TK2, POLG, etc.) affecting muscle endurance would also benefit from ketone fuels and creatine (hence SNV—C1 being targeted for muscle/heart).

Across these cardiac/muscle conditions, one consistent observation is that the heart often shifts to ketone usage in heart failure as a compensatory mechanism. Providing exogenous ketones may augment that mechanism and improve function. For muscle, ketones can reduce lactate and provide a more efficient ATP per oxygen substrate, possibly improving exercise tolerance. In FAODs, as noted, there are documented cases where cardiomyopathy reversed when patients were on supplemental ketosis-one child's ejection fraction normalized after months on oral BHB, according to case reports. This basket therefore might show some of the most dramatic improvements with therapy (e.g., measured by echo cardiography or 6-minute walk tests).

Neurodegenerative Diseases: While not all neurodegenerative diseases are orphan, certain subsets or early-onset forms are, and the mechanistic overlap with energy metabolism is strong, conditions like:

ALS (Amyotrophic Lateral Sclerosis): Sporadic ALS is not orphan (though rare), but juvenile ALS or familial forms are individually very rare. There is evidence neurons in ALS have impaired mitochondria and that ketosis might be protective. Provided herein is a prophetic example for SOD1-G93A ALS mice wherein ketone ester might preserve grip strength and prolong survival. Additionally, ketones might upregulate neurotrophic factors and reduce excitotoxicity, beneficial in ALS.

Alzheimer's Disease (Early-onset familial AD could be considered orphan): The brain of AD has glucose hypo-metabolism; ketones can compensate for this as shown in studies. While AD itself is common, certain genetic early AD (e.g., Down syndrome Alzheimer's or PSEN1 mutations) are rare. Regardless, the concept is that providing ketones improves cognitive function and possibly slows neurodegeneration by multiple pathways (energy, less amyloid deposition through BHB signaling, etc.).

Parkinson's Disease (certain atypical forms like PARK7 or PINK1 mutations are rare): PD brains also show mitochondrial deficits. A project (like the one with USF mentioned in the SRA) is investigating ketones in PD and AD. The disclosure could foresee formulations like SNV—K2 (with α-KG for PD perhaps, since PD may benefit from anaplerosis and antioxidant effect) for these indications.

Huntington's Disease (juvenile form is rare): Some studies have looked at ketogenic interventions; trehalose-BHB could help by clearing aggregates (trehalose) and fueling neurons.

Others: Progressive Supranuclear Palsy, certain spinocerebellar ataxias, etc., where metabolism might be part of the pathology.

In some embodiments, the present disclosure provides a method for treating or preventing a condition associated with energy insufficiency in a subject. The method comprises administering to the subject a therapeutically effective amount of an agent or intervention selected or adjusted based on the assessed state of bioenergetic sufficiency or insufficiency, wherein the agent or intervention increases a Metabolic Sufficiency Ratio (M=E/R) in a target tissue of the subject.

In some embodiments, the condition associated with energy insufficiency is selected from the group consisting of Alzheimer's disease, Parkinson's disease, heart failure, type 2 diabetes, metabolic syndrome, traumatic brain injury, cachexia, age-related functional decline, and the orphan diseases GLUT1 deficiency syndrome, Pyruvate Dehydrogenase Complex Deficiency (PDHc-D), SLC13A5 epileptic encephalopathy, PCDH19-Related Epilepsy, Dravet syndrome, Lennox-Gastaut syndrome, infantile spasms, CDKL5 Deficiency Disorder, Doose syndrome (myoclonic-astatic epilepsy), Mitochondrial Complex I Deficiencies, Leigh syndrome, MELAS, MERRF, Friedreich's ataxia, juvenile ALS, multiple acyl-CoA dehydrogenase deficiency (MADD), Very Long-Chain Acyl-CoA Dehydrogenase Deficiency (VLCAD), Long-Chain 3-Hydroxyacyl-CoA Dehydrogenase Deficiency/Trifunctional Protein Deficiency (LCHAD/TFP), medium-chain acyl-CoA dehydrogenase deficiency (MCAD deficiency), Carnitine Palmitoyltransferase II Deficiency (CPT-II), HMG-CoA Lyase Deficiency, Barth syndrome, LMNA-related dilated cardiomyopathy, Duchenne muscular dystrophy-associated cardiomyopathy, spinal muscular atrophy type II, mtDNA Depletion Syndromes, early-onset familial Alzheimer's or Parkinson's disease, Febrile Infection-Related Epilepsy Syndrome (FIRES), Glioblastoma Multiforme (GBM), Diffuse Intrinsic Pontine Glioma (DIPG), Aicardi-Goutières Syndrome (AGS), SCN8A-Related Epilepsy (SCN8A), Tuberous Sclerosis Complex (TSC), Rasmussen Encephalitis (RE), IDH-mutant High-Grade Glioma (HGG), Atypical Teratoid Rhabdoid Tumors (ATRT), Rett Syndrome (RTT), Angelman Syndrome (AS), Lafora Disease (LD), Frontotemporal Dementia (FTD), Acute Necrotizing Encephalopathy (ANEC), Autoimmune Limbic Encephalitis (ALE), Focal Cortical Dysplasia (FCD), Hypothalamic Hamartoma (HH), Neuronal Ceroid Lipofuscinoses (NCLs), Unverricht-Lundborg Disease (EPM1), Sturge-Weber Syndrome (SWS), Hemimegalencephaly (HME), and Krabbe Disease (KD).

In certain embodiments, the condition associated with energy insufficiency is selected from the group consisting of Alzheimer's disease, Parkinson's disease, Huntington's disease, heart failure, stroke, spinal cord injury, type 2 diabetes, obesity, non-alcoholic fatty liver disease (NAFLD), non-alcoholic steatohepatitis (NASH), polycystic ovary syndrome (PCOS), epilepsy, traumatic brain injury, amyotrophic lateral sclerosis, cancer, cancer cachexia, peripheral vascular disease, mitochondrial disorders, chronic fatigue syndrome, metabolic syndrome, bipolar disorder, major depressive disorder, anxiety disorders, schizophrenia, autism spectrum disorder, attention deficit hyperactivity disorder (ADHD), and any condition in which the subject's tissue exhibits a quantifiable reduction in glucose utilization.

In certain embodiments, the condition associated with energy insufficiency is Alzheimer's disease. In certain embodiments, the condition associated with energy insufficiency is Parkinson's disease. In certain embodiments, the condition associated with energy insufficiency is Huntington's disease. In certain embodiments, the condition associated with energy insufficiency is heart failure. In certain embodiments, the condition associated with energy insufficiency is stroke. In certain embodiments, the condition associated with energy insufficiency is spinal cord injury. In certain embodiments, the condition associated with energy insufficiency is type 2 diabetes. In certain embodiments, the condition associated with energy insufficiency is obesity. In certain embodiments, the condition associated with energy insufficiency is non-alcoholic fatty liver disease (NAFLD). In certain embodiments, the condition associated with energy insufficiency is non-alcoholic steatohepatitis (NASH). In certain embodiments, the condition associated with energy insufficiency is polycystic ovary syndrome (PCOS). In certain embodiments, the condition associated with energy insufficiency is epilepsy. In certain embodiments, the condition associated with energy insufficiency is traumatic brain injury. In certain embodiments, the condition associated with energy insufficiency is amyotrophic lateral sclerosis. In certain embodiments, the condition associated with energy insufficiency is cancer. In certain embodiments, the condition associated with energy insufficiency is cancer cachexia. In certain embodiments, the condition associated with energy insufficiency is peripheral vascular disease. In certain embodiments, the condition associated with energy insufficiency is a mitochondrial disorder. In certain embodiments, the condition associated with energy insufficiency is chronic fatigue syndrome. In certain embodiments, the condition associated with energy insufficiency is metabolic syndrome. In certain embodiments, the condition associated with energy insufficiency is bipolar disorder. In certain embodiments, the condition associated with energy insufficiency is major depressive disorder. In certain embodiments, the condition associated with energy insufficiency is an anxiety disorder. In certain embodiments, the condition associated with energy insufficiency is schizophrenia. In certain embodiments, the condition associated with energy insufficiency is autism spectrum disorder. In certain embodiments, the condition associated with energy insufficiency is attention deficit hyperactivity disorder (ADHD). In certain embodiments, the condition associated with energy insufficiency is any condition in which the subject's tissue exhibits a quantifiable reduction in glucose utilization.

In some embodiments, the disclosure provides compositions for use in treating or preventing a condition associated with energy insufficiency. The composition may comprise a therapeutically effective amount of an agent that increases a Metabolic Sufficiency Ratio (M=E/R) in a target tissue of a subject, wherein the agent is selected from the group consisting of a ketone body, a ketone ester, a ketone salt, a medium-chain triglyceride (MCT), a lactate precursor, one or more specific amino acids, a mitochondrial modulator, an insulin sensitizer, an anti-inflammatory agent, an antioxidant, and combinations thereof.

In some embodiments, the condition treated or prevented by the compositions described herein is selected from the group consisting of Alzheimer's disease, Parkinson's disease, heart failure, type 2 diabetes, metabolic syndrome, traumatic brain injury, cachexia, age-related functional decline, and the orphan diseases GLUT1 deficiency syndrome, Pyruvate Dehydrogenase Complex Deficiency (PDHc-D), SLC13A5 epileptic encephalopathy, PCDH19-Related Epilepsy, Dravet syndrome, Lennox-Gastaut syndrome, infantile spasms, CDKL5 Deficiency Disorder, Doose syndrome (myoclonic-astatic epilepsy), Mitochondrial Complex I Deficiencies, Leigh syndrome, MELAS, MERRF, Friedreich's ataxia, juvenile ALS, multiple acyl-CoA dehydrogenase deficiency (MADD), Very Long-Chain Acyl-CoA Dehydrogenase Deficiency (VLCAD), Long-Chain 3-Hydroxyacyl-CoA Dehydrogenase Deficiency/Trifunctional Protein Deficiency (LCHAD/TFP), medium-chain acyl-CoA dehydrogenase deficiency (MCAD deficiency), Carnitine Palmitoyltransferase II Deficiency (CPT-II), HMG-COA Lyase Deficiency, Barth syndrome, LMNA-related dilated cardiomyopathy, Duchenne muscular dystrophy-associated cardiomyopathy, spinal muscular atrophy type II, mtDNA Depletion Syndromes, early-onset familial Alzheimer's or Parkinson's disease, Febrile Infection-Related Epilepsy Syndrome (FIRES), Glioblastoma Multiforme (GBM), Diffuse Intrinsic Pontine Glioma (DIPG), Aicardi-Goutières Syndrome (AGS), SCN8A-Related Epilepsy (SCN8A), Tuberous Sclerosis Complex (TSC), Rasmussen Encephalitis (RE), IDH-mutant High-Grade Glioma (HGG), Atypical Teratoid Rhabdoid Tumors (ATRT), Rett Syndrome (RTT), Angelman Syndrome (AS), Lafora Disease (LD), Frontotemporal Dementia (FTD), Acute Necrotizing Encephalopathy (ANEC), Autoimmune Limbic Encephalitis (ALE), Focal Cortical Dysplasia (FCD), Hypothalamic Hamartoma (HH), Neuronal Ceroid Lipofuscinoses (NCLs), Unverricht-Lundborg Disease (EPM1), Sturge-Weber Syndrome (SWS), Hemimegalencephaly (HME), and Krabbe Disease (KD).

In certain embodiments, the condition associated with energy insufficiency is characterized by one or more symptoms selected from the group consisting of cardiomyopathy, seizures, hypoketotic hypoglycemia, lactic acidosis, ataxia, cognitive decline, cognitive impairment, developmental delay, exercise intolerance, failure to thrive, hypoglycemia, intracranial pressure, lethargy, metabolic crises, multiple seizure types, muscle weakness, myoclonic-astatic seizures in childhood, neurodegeneration, neurodevelopmental delay, neuropathy, recurrent non-ketotic hypoglycemia, recurrent rhabdomyolysis, refractory epilepsy, refractory seizures, super-refractory status epilepticus, and tumor progression.

In some aspects, the compositions described herein may be administered to subjects exhibiting one or more of the foregoing symptoms, alone or in combination with a confirmed diagnosis of one or more of the energy-insufficient diseases described above.

Major mechanistic “axes” that MERIT therapy impacts and by therapeutic areas or indication clusters are described herein. It should be understood that these categories overlap and interrelate—e.g., in a given disease like Alzheimer's, multiple axes (redox, epigenetic, immunometabolic) are all relevant and MERIT addresses them together. The sub sections serve to explain each facet of the therapy and provide ample support for the breadth of claims.

### Mechanistic Axes of MERIT Therapy

Each of the following sub-sections details a specific biochemical or cellular consequence of therapeutic ketosis, which also represents an independent axis of patentability and therapeutic effect. Each axis can be considered a distinct rationale for using MERIT in various diseases, and in many cases. In each mechanism described, “ketone exposure” refers primarily to exposure to BHB and AcAc at levels achieved in therapeutic titratable ketosis (generally 1-10 mM range for BHB and proportional AcAc unless specified otherwise), whether via intravenous infusion, oral dosing, etc., and “controlled” exposure means modulating the dose to achieve desired effects on that mechanism.

### Redox Rebalancing and NAD⁺/NADH Modulation (The NADH Drain via AcAc)

One of the foundational pillars of MERIT is correcting redox dysregulation, specifically by reducing excess NADH and boosting NAD⁺ availability in cells. Many diseases exhibit a pathological NADH/NAD⁺ ratio: for instance, ischemic tissues accumulate NADH due to lack of oxygen as an electron acceptor; cancer cells (Warburg effect) often have high cytosolic NADH (evidenced by high lactate from pyruvate); diabetic hearts and livers can have altered NADH/NAD⁺ from substrate overload; neurodegenerative diseases show drops in NAD⁺ levels in neurons (impairing sirtuins and DNA repair enzymes). This sub-section explains how MERIT uses ketone biology to fix these issues.

Mechanistic Explanation: D-β-hydroxybutyrate dehydrogenase (BDH1) catalyzes a reversible reaction: Acetoacetate+NADH+H+⇄β-hydroxybutyrate+NAD+

In tissues, the forward direction (AcAc to BHB) consumes NADH, whereas the reverse (BHB to AcAc) consumes NAD⁺. In a high-NADH environment, Le Chatelier's principle drives AcAc→BHB, regenerating NAD⁺. In normal physiology, the liver exports BHB with a certain BHB:AcAc ratio reflecting its mitochondrial redox state; peripheral tissues take up BHB and often convert a portion to AcAc for oxidation in the TCA cycle, producing NADH in that step. In MERIT, exogenous AcAc or molecules are provided that yield AcAc intracellularly. This could be done via infusing a stabilized acetoacetate solution or an AcAc ester that releases AcAc, or via a ketogenic precursor like a triacylglycerol that yields AcAc preferentially.

When exogenous AcAc is provided, cells (especially mitochondria with active BDH1) will use up NADH to turn some of that AcAc into BHB. Essentially, AcAc acts as an electron acceptor akin to how pyruvate can accept electrons to form lactate. The outcome is an increase in NAD⁺/NADH ratio. This has multiple beneficial consequences: Restored Mitochondrial Function: Many dehydrogenase enzymes in the TCA cycle and electron transport chain require NAD⁺. If NAD⁺ is in short supply (NADH buildup), these pathways slow, and cell resorts to less efficient means (glycolysis). By increasing NAD⁺, high-flux oxidative metabolism is re-enabled, improving ATP production. For example, in ischemic muscle or brain, providing AcAc could help use up accumulating NADH, slightly alleviating the block in metabolism even before oxygen is fully back (we see parallels in reperfusion where ketones help recovery). Activation of NAD+-dependent signaling: Sirtuins (class III HDACs), PARP (DNA repair, though excess PARP can deplete NAD⁺ in damage), and other NAD⁺-requiring enzymes (CD38, etc.) play roles in stress resistance, inflammation regulation, and genomic stability. By raising NAD⁺, MERIT can activate sirtuin pathways. SIRT1 and SIRT3, for instance, promote mitochondrial biogenesis and antioxidant defense (via deacetylating and activating PGC-1α, etc.). SIRT2/6 influence inflammation and metabolism. This ties into epigenetic and immune axes too, but the trigger is NAD⁺ availability. —Lactate/Pyruvate (L/P) normalization: The lactate/pyruvate ratio in blood or tissues is another reflection of cytosolic redox (via LDH equilibrium and NADH). In cancer or during intense exercise or in sepsis, L/P skyrockets. By draining NADH, lactate production is expected to drop (pyruvate less forced to accept electrons), thus L/P falls. In MERIT, one could monitor L/P as a marker and titrate ketone infusion until L/P drops below a threshold (say from 40:1 towards a normal ~10:1). This is a direct measurable outcome of redox improvement.

Key Biomarkers such as NAD⁺ and NADH levels (in tissue biopsies or blood if feasible), the NAD⁺/NADH ratio (though measuring that ratio in vivo is tricky, are inferred from proxies like BHB:AcAc or L: P). BHB:AcAc ratio is a convenient systemic proxy as mentioned. If maintaining a lower BHB:AcAc ratio in blood (say 1:1 instead of 4:1), suggests peripheral tissues are more oxidized. Another related marker is the β-hydroxybutyrate dehydrogenase equilibrium constant—but practically, BHB:AcAc is measured. Also, ratio of free cytosolic [NAD⁺]/[NADH] can be inferred by the lactate: pyruvate ratio (the LDH equilibrium: lactate/pyruvate=NADH/NAD⁺*(K_eq)). In Example experiments, it will be shown how lactate levels change with ketone closing. Also, blood ketone profiles themselves (BHB vs AcAc fraction) are markers—e.g., if BHB monoester is given vs an AcAc ester, the resulting ratio differs and which correlates with outcome.

How Ketones Modulate Them: Summarizing: BHB itself tends to increase NADH when it's oxidized to AcAc (costing NAD⁺), whereas AcAc tends to increase NAD+when reduced to BHB (costing NADH). Thus, a MERIT formulation biased towards AcAc (like an acetoacetate diester or salt) will have a stronger NAD⁺-regenerating effect. However, even BHB administration can indirectly raise NAD⁺ if it spares NAD⁺ consumption in other pathways or if it gets excreted partly. But indeed the more straightforward is via AcAc. Previously it has been noted that giving pure acetoacetate could cause too much load on heart if sole substrate-hence, combination of BHB and AcAc in balanced ratio will be administered to avoid any single-metabolite issues (the heart prefers a mix of substrates). The composition might be tuned: e.g., 60% BHB, 40% AcAc by ketone molarity, to gently drain NADH without causing e.g. hypoglycemia.

Indication Clusters Where Redox Axis is Central: Many. Examples include:

Ischemia/Reperfusion Injury: Heart attack (myocardial infarction) or stroke-during ischemia, NADH accumulates, contributing to damage; ketosis before or during reperfusion might reduce injury (some studies have shown ketone bodies protect tissues from radiation and possibly ischemia).

Heart Failure & Cardiomyopathy: Failing hearts often have a redox imbalance and turn to ketones as fuel. MERIT could help by providing ketones that simultaneously fuel and improve redox. Ketones also induce expression of myocardial genes (e.g. BHB via HDAC inhibition upregulates antioxidant enzymes). But on pure redox, studies show improved cardiac efficiency on ketones, partially because of NADH/NAD shifts.

Cancer cachexia and metabolism: Many cancers are in a reductive state (due to hypoxia and high glycolysis). For instance, an aggressive tumor has areas of NADH build-up. By refeeding NAD⁺ (systemically increasing it), you might slow cancer cell growth or sensitize them to treatments (some proposals involve giving NAD⁺ precursors; here we do via ketones).

Neurodegenerative diseases: Brain aging is associated with a decline in NAD (there's popular interest in NAD⁺ boosters like NR or NMN for cognition). Ketosis might serve a similar role via SIRT3 and others. Also, in Alzheimer's, glucose utilization is impaired; ketones provide alternate fuel but also alter NAD⁺ levels in neurons, possibly preserving synaptic function. In Parkinson's, mitochondrial complex I issues cause NADH buildup. Redox correction could help.

Mitochondrial diseases: Primary mitochondrial disorders (e.g., MELAS, Leigh syndrome) have defective oxidative phosphorylation and often abnormal NAD⁺/NADH. Ketone therapy could bypass some blocks and also increase NAD⁺ to maximize residual enzyme activity.

Metabolic syndrome/NAFLD/MASH: Overnutrition leads to a reduced cytosolic state in liver (e.g., high NADH from alcohol or fructose metabolism). By modifying this, steatosis could be reduced. Indeed, acetoacetate infusion is known to improve redox; there were some old attempts to use sodium acetoacetate infusion in alcoholic acidosis.

Psychiatric (schizophrenia, bipolar): There is evidence of redox dysregulation in schizophrenia (GSH depletion, NADPH issues) and bipolar (mitochondrial slight inefficiencies). Some case reports of ketogenic diet helping bipolar or schizophrenia exist. Treatment described herein might be more via neurotransmitters and neuronal energy, but redox and NAD⁺ might also play a role (e.g., NAD⁺ is needed for dopamine beta-hydroxylase, etc.).

TTK Targeting of Redox Axis: In practice, MERIT therapy would include explicit goals like: maintain patient's blood BHB:AcAc ratio below 3:1 for at least 6 hours/day, or achieve an increase in whole-blood NAD⁺ by 50% from baseline. Starting with continuous ketone infusion and adjusting the blend of BHB vs AcAc. For example, if BHB:AcAc in blood goes too high (meaning patient's getting too reduced), additional AcAc may be needed or reduction in BHB closing rate. In a clinic setting, measure BHB and AcAc from fingerstick or sensor, and the doctor adjusts a “redox dial”.

To illustrate: Example Prophetic Scenario: A patient with heart failure with reduced ejection fraction (HFrEF) has high filling pressures partly due to energetic deficits. Admitted for a MERIT trial: infuse a ketone solution (80% Na-BHB+20% Na—AcAc by moles) intravenously. Monitor his hemodynamics and blood gas. Over an hour, BHB rises to 3 mM, AcAc to 1 mM (BHB:AcAc=3:1). Mmeasure blood NAD⁺ (could be done via a lab assay) and find it increased from 50 to 70 μM. The patient's lactate dropped from 2.5 to 1.5 mM. He subjectively feels more energetic. Over weeks on oral ketone supplements designed to release some acetoacetate, his exercise tolerance improves. This scenario ties mechanistic markers to clinical outcome.

### Bioenergetic Enhancement and Mitochondrial Efficiency (Reversing Warburg Effect)

Closely related to redox, but deserving its own emphasis, is the ability of ketone therapy to enhance cellular energy production and mitochondrial function. Ketone bodies are efficient fuels-oxidation of BHB yields more ATP per oxygen than glucose (BHB is more reduced, giving more NADH per carbon). Acetoacetate can enter the TCA cycle as acetoacetyl-CoA then acetyl-CoA, bypassing some need for glycolysis. In conditions where energy production is compromised or where cells rely on inefficient pathways (e.g., cancer cells heavy on glycolysis), providing ketones can boost ATP output and potentially shift metabolism.

Mechanistic Explanation: When a cell takes up BHB, it's converted to AcAc (yielding NADH as noted, which is then used in ETC) and then AcAc-COA (via succinyl-CoA: 3-oxoacid CoA transferase, SCOT, an enzyme notably absent in liver but present in muscle, brain, etc.), and then to acetyl-CoA (via ketothiolase). This acetyl-CoA enters the TCA cycle. One important point: ketone oxidation produces less lactic acid than glucose oxidation. So in a hypoxia-tolerant or Warburg cell, forcing them to use ketones can reduce acid buildup and be more O₂ efficient.

Warburg Reversal: Cancer cells often ferment glucose to lactate even in oxygen (Warburg effect). There is evidence that in presence of ketones, some cancer cells will consume ketones and perhaps reduce their glycolysis. By providing an alternate fuel that feeds into mitochondria, MERIT might push cancer cells towards oxidative metabolism, which paradoxically could slow proliferation (Warburg effect is thought to aid biosynthesis for growth—forcing oxidative metabolism might divert them away from proliferation or expose them to ROS). Also, some normal tissues (like brain in Alzheimer's) have a Warburg-like glucose hypometabolism which ketones can bypass. So “Warburg reversal” can mean in cancer: prompting tumor cells to oxidize more (making them vulnerable to ROS or to mitochondrial apoptosis), and in degenerative disease: giving neurons an oxidative fuel to compensate for their inability to use glucose properly.

Key Biomarkers: Cellular or tissue oxygen consumption rate (OCR) vs extracellular acidification (ECAR) can measure how metabolic flux changes. In patients, one might measure VO₂ max improvement or muscle lactate production during exercise with vs without ketones. On a molecular level, PGC-1α activation (master regulator of mitochondria) could be a biomarker-known that ketogenic diet can increase PGC-1α in some tissues. Ketone therapy also often raises blood β-hydroxybutyrate dehydrogenase levels or changes in respiratory quotient (RQ)—RQ closer to 0.7 indicates more fat/ketone usage vs 1.0 for pure carbs. So an indicator is a drop in RQ when on therapy, meaning the body shifted to fat/ketone oxidation.

### How Ketones Modulate Bioenergetics:

ATP Production: Studies show the heart uses ketones efficiently. In heart failure patients, infusing ketones acutely increased cardiac output. Mechanistically, BHB yields more free energy change on oxidation than pyruvate. If oxygen is limited, using a fuel that gives more ATP per O₂ is beneficial (this is one rationale NASA and others looked at ketone esters for extreme environments).

Mitochondrial Biogenesis: There's some evidence that ketosis (especially chronic) triggers creation of more mitochondria, possibly via SIRT1/AMPK/PGC-1a pathway. BHB's HDAC inhibition could increase PGC-1α expression (as HDACs often repress metabolic genes). So MERIT might over weeks lead to the cells actually increasing their mitochondrial mass and enzyme capacity, a longer-term benefit.

Autophagy & Mitophagy: Not exactly energy production, but intimately tied-fasting ketosis induces autophagy, clearing dysfunctional mitochondria, which then improves overall efficiency. BHB has been seen to induce autophagy in certain contexts (perhaps via mTOR inhibition due to low insulin, or via direct signaling).

Neuroprotective Energy: In brain, energy deficit is tied to neuronal death. Ketones can provide fuel to neurons and glia, improving their viability under stress. There's a classic example: in hypoglycemia or certain toxin models, ketones prevent ATP drop.

Decrease Reliance on Glucose/Insulin: This matters in insulin resistant states. If muscles and other tissues use ketones, they draw less on glucose, which could improve blood sugar control and reduce insulin demand (hence seeing some patents about ketones lowering blood glucose). That effect is metabolic but also energetic-they provide alternative energy so that cells don't need to force glucose uptake via high insulin.

### Indications for Bioenergetic Axis:

Neurodegenerative (Alzheimer's, Parkinson's, Huntington's): these diseases show reduced brain metabolism (FDG-PET scans in AD show low uptake). Ketones can penetrate and provide immediate fuel, improving cognition and potentially slowing degeneration. There have been trials of ketone esters for AD (ACCs). Additionally, increasing ATP in neurons might stabilize synapses and reduce toxic pathways (e.g., amyloid production might be modulated by metabolism).

Epilepsy and other Channelopathies: The ketogenic diet's original use is epilepsy-mechanistically partly because ketones provide a stable fuel and perhaps help maintain ion gradients (neurons with more ATP can keep K⁺/Na⁺ balance, reducing hyperexcitability). Also BHB opens K_ATP channels in neurons (hyperpolarizing them). Those aspects could also go here or Neurotransmission axis below.

Muscle diseases and exercise intolerance: Ketones could improve muscle energetics in McArdle's disease (glycogen storage disease V where they can't use glycogen well, ketones give alternate fuel), or in mitochondrial myopathies (some ability to bypass complex I by providing succinyl-CoA. Also exercise performance in healthy—that was actually a major focus of some prior art: the TdeltaS patent for muscle power and performance on ketones.

Heart failure and cardiometabolic: as mentioned, failing hearts appear to upregulate ketone usage as an adaptive fuel. Providing extra ketones might help the heart contract better (some studies saw improved EF on ketone infusion).

Cancer: from a different angle, the bioenergetic effect in cancer might not be beneficial to the cancer, but conceptually using ketones might starve the tumor of glucose by improving systemic glycemic control (the Natural Alternatives style idea: ketones lowering blood glucose which could slow tumor growth that needs glucose). Also, some evidence indicates some tumor cells cannot use ketones effectively (especially if key enzymes are downregulated), so giving ketones might selectively fuel normal cells and not tumor cells.

TTK for Bioenergetics: Here, the treat-to-target might involve achieving a certain GKI (Glucose Ketone Index)—which is the ratio of blood glucose to ketone in same units. A low GKI means high ketosis relative to glucose-considered therapeutic for brain cancer and such (GKI<1 or 2). Aim for GKI~<3 for example in some neuro cases. That index ensures both sides (glucose down, ketone up). Or simpler, target a BHB level above 2 mM for X hours. The result hoped would be improvements in physical measures: e.g., a target 6-minute walk test distance improvement (for heart failure), or a target cognitive score.

Example Prophetic Data: In a model of Alzheimer's disease (transgenic mice or even a patient), after 8 weeks of daily ketone ester closing achieving BHB ~2 mM for 8 h/day, measure mitochondrial enzyme activities in muscle or brain and see increases (citrate synthase, complex I, etc.), increased ATP levels in brain tissue, and better memory in a maze. Or in a patient with CHF, a 2-week infusion of ketones increases their ejection fraction by a few percentage points and lowers NT-proBNP(a heart failure marker)—reflecting improved energy availability.

### Immunometabolic Modulation-NLRP3 Inflammasome, Inflammation and T-Cell Metabolism

This axis covers how ketosis affects the immune system's behavior by altering immune cell metabolism and signaling. Ketone bodies have direct and indirect anti-inflammatory effects, and can improve the function of immune cells (especially in chronic disease contexts) by reprogramming their metabolism.

NLRP3 Inflammasome Inhibition: A seminal discovery was that β-hydroxybutyrate can inhibit the NLRP3 inflammasome, which is a protein complex in innate immune cells (like macrophages) that, when triggered (by pathogens, crystals, etc.), activates caspase-1 and leads to IL-1B and IL-18 release (key inflammatory cytokines). NLRP3 is a common denominator in many inflammatory diseases: gout (triggered by urate crystals via NLRP3), Alzheimer's (amyloid and tau can activate NLRP3 in microglia), atherosclerosis (cholesterol crystals in plaques), type 2 diabetes (islet amyloid and metabolic factors), non-alcoholic steatohepatitis (danger signals from fat, etc.), and even general aging (NLRP3 gets activated by various age related mitochondrial dysfunction).

The mechanism by which BHB inhibits NLRP3 isn't fully a receptor-ligand classical one; research indicates that it might involve reducing K+efflux from the cell (since high extracellular K+or blocked K+efflux prevents NLRP3 assembly) or inhibiting NEK7 binding (NEK7 is a protein required for NLRP3 activation). Also, it may need a mildly acidic pH to be effective (one study said at physiological pH BHB doesn't inhibit, but in slight acidosis it does)—relevant in that during fasting, blood pH might drop a bit, or in local microenvironments.

Ketone Receptor HCA2 (HCAR2): BHB is an agonist of hydroxycarboxylic acid receptor 2 (HCA2, also known as GPR109A or Niacin receptor) on certain immune cells (monocytes, macrophages, certain glia). Activation of HCA2 by BHB can have anti-inflammatory outcomes, similar to how high-dose niacin's flushing effect is mediated by HCA2 on dermal immune cells releasing prostaglandin. In the context of inflammation, HCA2 activation by BHB on macrophages was shown to reduce neuroinflammation in a model of MS-like disease (the receptor triggers anti-inflammatory pathways). So BHB acts like an endogenous nicotinic acid on immune cells.

T-Cell Exhaustion and Metabolic Fitness: T lymphocytes, especially CD8+ cytotoxic T cells, when fighting chronic infections or cancer, often enter an “exhausted” state characterized by metabolic insufficiency (they upregulate inhibitory receptors like PD-1 and can't proliferate or kill well). Part of exhaustion is that these cells cannot sustain glycolysis and have mitochondrial dysfunction. There's emerging evidence that forcing T cells to use oxidative metabolism (or providing fuels for it) can rejuvenate them. In tumors, it's been observed that tumor cells consume glucose leaving only ketone bodies and fatty acids for T cells; T cells that can use those (via high mitochondrial capacity) survive better. Ketone bodies could support tumor infiltrating T cells by giving them a fuel that tumor cells might not steal as readily, and by boosting their NAD⁺ (which is needed for T cell function and for enzymes like SIRT1 that help form memory T cells). Additionally, a study found that ketone supplementation improved some aspects of T cell responses in a chronic infection model.

Cytokine profile changes: Ketosis often leads to lower levels of pro-inflammatory cytokines (IL-1β, TNFα) and possibly higher levels of anti-inflammatory ones (IL-10, adiponectin from adipose etc.). Partly through NLRP3, partly via less glucose (excess glucose metabolism in immune cells tends to promote inflammatory M1 macrophage polarization; ketones promote an M2 phenotype, more tissue-repair oriented).

Key Biomarkers:—IL-1B and IL-18 levels: since those are direct outputs of NLRP3. We can measure them in blood or from stimulated immune cells (like an ex vivo LPS+ATP stimulation of patient's PBMCs to see how much IL-1B they produce; after ketone therapy that should drop). —Inflammasome activation markers: like cleaved caspase-1 presence, ASC speck formation (in research context). —C-reactive protein (CRP) and other systemic inflammation markers might decrease with MERIT. —T cell phenotyping: e.g., proportion of PD-1+TIM3+exhausted T cells in a tumor biopsy, or the expression of CD62L, CD127 (markers of memory vs exhaustion)—these might improve (more memory markers, less exhaustion markers) under ketone therapy. —Spare Respiratory Capacity (SRC) of immune cells: in lab, one can test T cells' mitochondrial function by oxygen consumption assays; ketone-treated T cells often have higher SRC, meaning they can produce more energy under stress. —NLRP3-related gene expression: perhaps lower NLRP3 itself or IL1B gene expression after chronic ketone treatment due to epigenetic changes.

Indication Clusters: —Autoimmune and Autoinflammatory Diseases: e.g., Rheumatoid arthritis, Lupus, Multiple Sclerosis, Psoriasis —these involve both innate and adaptive immunity. Ketones might calm the innate side (inflammasomes, macrophages) and also affect T cells. There are anecdotes and small trials of ketogenic diets or exogenous ketones helping arthritis pain or MS. Particularly, a disease like Gout is a prime NLRP3-driven disease: urate crystals trigger inflammasome in joints; BHB (which is produced by fasting- and interestingly, fasting was a traditional gout remedy) might help quell flares. Indeed, our spec already mentions an embodiment of 10g D-βHB reducing gout flares. —Neuroinflammatory conditions: Alzheimer's disease has neuroinflammation component (NLRP3 in microglia responding to amyloid). Position MERIT as both metabolic support for neurons and anti inflammatory in the brain. Likewise, post-stroke inflammation or TBI (traumatic brain injury)—ketones are being researched as neuroprotective (partly by reducing neuroinflammation and excitotoxicity). —Sepsis or Cytokine Storm: possibly outside the usual, but interestingly ketones in severe inflammation might modulate the hyperinflammation. —Cancer (immunotherapy context): Very relevant now because checkpoint inhibitors (like anti-PD-1) have revolutionized cancer treatment, but many patients don't respond (often due to T cell exhaustion or immunosuppressive metabolism in tumor microenvironment). If ketone therapy can improve T cell metabolism and reduce tumor inflammation, it might synergize. —COVID-19 or other cytokine-mediated illnesses: This is speculative, but COVID had NLRP3 involvement (SARS-COV-2 proteins can activate NLRP3).

TTK Implementation for Immune Axis: measure IL-1B levels weekly or CRP, and increase ketone dose until those markers drop by, say, 30%. Or using continuous ketone monitor and known pharmacokinetics: e.g., in gout prophylaxis, instruct patient to drink ketone supplement at first tingle of gout flare and aim to quickly raise BHB to >3 mM for a few hours-maybe aborts the flare by turning off NLRP3 activation.

Or using combination: e.g., in MS, combine ketones with standard disease modifying drug, track NLRP3 gene expression in blood as a PD marker.

Example: A patient with Adult-Onset Still's Disease (an IL-1 driven auto-inflammatory condition) is on an IL-1 blocker with partial effect. Start on MERIT: thrice daily oral ketone ester drinks targeting BHB ~2.5 mM. After a month, their IL-18 level (which was sky high) drops 50%, ferritin and CRP drop, and clinically they have fewer fever spikes. This might allow lowering steroid dose.

Another: a cancer patient on pembrolizumab (PD-1 antibody) has not responded after 12 weeks. Add a ketone infusion during each treatment cycle and daily ketone drinks. This could reduce tumor lactate (thus less immunosuppression) and provide T cells with fuel. After 8 weeks of combination, their CT scan shows tumor shrinkage whereas prior it was growing-anecdotal evidence of synergy. Measure T cells from blood: they show increased mitochondrial content and less expression of exhaustion markers.

### Epigenetic Reprogramming-Histone Deacetylase Inhibition and Histone Acylation (Kbhb, Kacac)

This axis addresses how ketone bodies alter the epigenetic landscape by multiple mechanisms: direct inhibition of histone deacetylases (HDACs), serving as substrates for histone modifications (like β hydroxybutyrylation and acetoacetylation of lysines), and altering levels of acetyl-CoA which is the substrate for histone acetyltransferases (HATs).

HDAC Inhibition by β-hydroxybutyrate: The Veillonella and Newman/Verdin research found that β hydroxybutyrate is a class I and IIa HDAC inhibitor at physiological concentrations achieved by fasting (e.g., 1-5 mM). Specifically, it was shown to inhibit HDAC1, HDAC3, and HDAC4/5 (IIa) somewhat, increasing global histone acetylation (H3K9ac, H3K14ac) in certain tissues, upregulating genes like FOXO3, Mt2, and oxidative stress resistance genes. The Ki for HDAC inhibition is in low mM, so you do need significant ketosis. The effect likely contributes to healthspan benefits of fasting and the anti-oxidant enzyme upregulation.

Our MERIT therapy exploits this by achieving those BHB levels chronically or intermittently to modulate gene expression in target tissues. For instance, in cognitive disorders, HDAC2 is often elevated and represses synaptic plasticity genes; BHB can inhibit HDAC2 (HDAC1/2 are class I) thereby lifting repression on genes like BDNF, GluN2B, etc., improving synaptic plasticity and memory. Unlike broad-spectrum synthetic HDAC inhibitors (which have side effects), BHB is a naturally tuned mild HDAC inhibitor —a plus for safety.

Histone β-hydroxybutyrylation (Kbhb): this modification was found enriched in liver of fasting mice, notably on H3 lysine 9 (H3K9bhb) at promoters of genes upregulated in fasting. It's proposed that when BHB (or rather its CoA) accumulates, it non-enzymatically or enzymatically can attach to histones. There's interplay: some HATs might use β-hydroxybutyryl-CoA similarly to acetyl-CoA (p300 can add crotonyl, so maybe can add BHB). The significance is that Kbhb is a mark of active chromatin and may potentiate transcription of metabolism-linked genes. If we keep a patient in ketosis for extended periods, induce Kbhb in various tissues, potentially “reprogramming” cells to a fasting-like gene expression pattern conducive to health (like upregulating antioxidant enzymes, downregulating inflammation genes, etc.). Measure Kbhb levels by mass spec in accessible cells as a PD biomarker.

Histone Acetoacetylation (Kacac): lysine acetoacetylation can occur when acetoacetyl-CoA is available. It's less studied, but likely it also marks active genes (as most acylations do aside from maybe succinylation can be different). Acetoacetate therapy could directly increase acetoacetyl COA (some will come from ketone metabolism or from partial oxidation of ketones). Perhaps one scenario: if acetoacetate levels are high, some fraction enters an alternate pathway generating acetoacetyl-CoA in the nucleus. The effect on gene expression could be overlapping with acetylation or unique.

Acetyl-CoA Increase: Ketone metabolism spares or generates acetyl-CoA in certain contexts. In a low-carb state, acetyl-CoA from fat is largely turned into ketones in liver, but in peripheral tissues when they oxidize BHB, they produce acetyl-CoA. If a cell is in energy surplus, some acetyl-CoA can go to the nucleus to acetylate histones (coupling metabolism to epigenetics). So giving ketones might raise acetyl-CoA levels in some cells relative to glucose feeding (especially if PDH was inhibited by some reason and now acetoacetate bypasses that). Additionally, BHB might inhibit class I HDACs, which raises histone acetylation by lowering removal, while at same time providing more acetyl-CoA (the substrate)—a one-two effect to hyperacetylate histones.

Gene Reactivation in Specific Indications: —Neurological/psychiatric: In depression or bipolar, some genes (like BDNF) might be repressed; HDAC inhibitors have antidepressant effects in models. BHB from ketogenic diet showed antidepressant effects in some rodent tests. MERIT can help treatment-resistant depression by epigenetically upregulating neuroplasticity genes. For neurodevelopmental disorders like Rett (MECP2 mutation leads to widespread repression)—ketones might help because there was an animal study showing a KD improved symptoms. Possibly by increasing BDNF via HDAC inhibition. —Cancer: Epigenetics in cancer-some tumor suppressor genes are silenced by deacetylation or methylation. Potentially ketones unsilence them; there was a study that ketone supplementation changed histone modifications in a way that inhibited cancer growth (some noted changes in β-hydroxybutyrylation in tumors). —Aging: As cells age, histones globally change (generally loss of acetylation at some sites, etc.). Ketosis epigenetically might promote a youthful pattern. Indeed, BHB's HDAC inhibition has been postulated as a mechanism for lifespan extension by fasting. —Metabolic memory in diabetes: Epigenetics is implicated in the “memory” of high glucose damage. Possibly ketones could reset some of that by modulating histone marks.

Key Biomarkers: —Global histone acetylation (H3K9ac, H3K14ac) measured via Western blot or mass spec in PBMCs or target tissues. —Levels of H3K9β-hydroxybutyrylation (H3K9bhb)—new assays can detect this. Perform a before/after MERIT therapy biopsy, expect increase in Kbhb. —HDAC activity assays: e.g., isolate nuclear extracts from patient's cells, measure HDAC enzyme activity with and without therapy; should be reduced during therapy (because BHB is present in cells). —Gene expression of specific HDAC-regulated genes: For example, FOXO3 or Mt2 or catalase-known to increase with BHB. —HDAC protein levels: Over long term, ketosis might downregulate some HDACs at transcriptional level.

Indications heavily featuring Epigenetic Axis: —Cognitive impairment & neurodegeneration: Alzheimer's, MCI-HDAC2 is a target. MERIT could yield cognitive improvement in part by epigenetic means. —Trauma and ischemia recovery: Epigenetic reprogramming to a cell-protective state can help recovery. E.g., after stroke, enhancing histone acetylation aids neuroplasticity for rehab. —Rare genetic disorders of chromatin: e.g., Kabuki syndrome (caused by mutations in a H3K4 demethylase, leads to immunodeficiency and developmental delay; HDAC inhibitors have been proposed to compensate for certain epigenetic imbalance; ketones could be a milder alternative). Rett syndrome (MECP2 mutation, global transcription issues; ketogenic diet had some positive data in mice). FOXG1 syndrome (some overlap with Rett, KD also proposed). Angelman syndrome (UBE3A mutation-one study found ketone diet improved motor function in model). —Psychiatric (mood disorders, schizophrenia): There is evidence of altered histone acetylation in these conditions (from stress etc.). Some HDAC inhibitors produce antidepressant-like effects. A ketone therapy might serve similar function without side effects. —Inflammation and immune memory: Epigenetic training of immune cells (e.g., suppressing inflammatory gene promoters via increased acetylation of anti-inflammatory genes, etc.). Possibly relevant to chronic inflammatory states.

TTK for Epigenetic Targets: For example, maintain BHB>1.5 mM for at least 4 hours/day on 5 days a week because transient ketosis might not fully set epigenetic marks; may need sustained or frequent closing for the marks to accumulate. Alternatively, one could plan cyclical treatments (like a few days on, few days off) to mimic fasting cycles that cause beneficial epigenetic changes. Monitor surrogate marker: e.g., measure HDAC activity in PBMC after a month; if still high, increase dose or frequency.

Example: A patient with mild cognitive impairment (prodromal AD) is put on daily ketone drink aiming for ~3 mM BHB each afternoon. After 6 months, cognitive tests stabilize or improve. Take PBMCs and find higher H3 acetylation and Kbhb than baseline, and increased expression of BDNF and other neuroprotective genes in those cells, which likely reflect similar changes in the brain.

Another: a child with Rett syndrome (due to MeCP2 deficiency, which normally represses genes, so in Rett, gene expression is off kilter). After 3 months on ketosis therapy, some motor and communication improvements are noted. Epigenetic analysis might show increased acetylation at certain promoters that could compensate for MeCP2 loss. Traditional HDAC inhibitors (like valproate, SAHA) could increase histone acetylation but don't fix metabolism or redox and have more side effects. MERIT is multi-faceted.

Neurotransmission and Neuroexcitation Stabilization (GABA/Glutamate, K ATP Channels. Neuronal Firing)

This axis focuses on how ketosis affects the nervous system's excitability and

neurotransmitter balance, which is crucial in conditions like epilepsy, migraine, pain, and psychiatric disorders.

GABA-Glutamate Balance: Ketogenic therapy is known to increase the ratio of GABA to glutamate in the brain. Possible mechanisms: —Ketone metabolism in neurons or astrocytes may increase glutamate conversion to GABA (some evidence in KD that glutamic acid decarboxylase (GAD) activity goes up, or less glutamate is released). —BHB might act on some receptors directly (some say BHB could mimic GABA at GABA_B. —Additionally, ketosis reduces glycolysis, which might reduce glutamate generation from glucose (since some goes to glutamate through TCA). The net effect is often anti-convulsant and mood stabilizing-GABA is inhibitory, glutamate excitatory; raising GABA relative to glutamate calms neural circuits.

ATP-sensitive potassium (K_ATP) channels: These channels in neurons open when ATP is low, hyperpolarizing the cell and preventing firing (a protective thing during metabolic stress). In ketogenic diet, particularly in seizures, it's found that low glucose and higher adenosine (another thing ketosis elevates) can activate K_ATP channels, which contribute to seizure protection. One study showed that blocking K_ATP channels diminished the anti-seizure effect of a ketone diet in mice, implying K_ATP is involved.

Adenosine Increase: Ketosis (esp. dec glucose) leads to increased adenosine in brain extracellular space. Adenosine is an inhibitory neuromodulator (it accumulates in tired brain, makes you sleepy, and has anticonvulsant properties). That is partly why ketogenic states can be sedating for some initially.

Brain-Derived Neurotrophic Factor (BDNF) Upregulation: This overlaps epigenetic axis, but relevant: KD increases BDNF in brain regions, which helps dampen hyperexcitability and promotes neuron health (BDNF can have mixed roles but generally, in epilepsy, increased BDNF can suppress seizures up to a point or help rewire circuits adaptively). BDNF increases in rat hippocampus on ketone monoester vs control, so that's a known effect.

Membrane Hyperpolarization via ketones: Some evidence suggests ketone metabolism produces more ATP in mitochondria, which might paradoxically lead to more efficient pumping of ions (like better Na/K ATPase function). Or ketones might alter ionic gradients by consuming protons differently. Many patients and animals on ketones have more stable EEGs, less random firing.

Pain Pathways: Similar logic-migraines and chronic pain involve neuronal hyperexcitability and inflammation. Ketones might calm neurons and also reduce neuroinflammation (NLRP3 in trigeminal system for migraine). Small trials using ketone supplements for migraine are underway.

Psychiatric and Cognitive Effects: On mood: many anecdotal reports that ketosis has mood-stabilizing effect in bipolar (possibly due to the same mechanisms as anti-seizure, since many bipolar drugs are anti seizure meds). On anxiety: ketone supplements shown to reduce anxiety behaviors in rats. Mechanism might be via GABA increase or maybe through acetoacetate acting on vagal tone.

Key Biomarkers: —GABA and glutamate levels in cerebrospinal fluid or microdialysis-tough in humans, but could do in animals or measure in blood (there is peripheral GABA pool but not sure if indicative). —EEG changes—e.g., reduction in epileptiform discharges or increased seizure threshold. That's a clinical outcome but also measurable in trials. —K_ATP channel subunit expression or polymorphisms—not usually measured clinically, but one could conceive measuring an increase in Kir6.2 or SUR1 subunits trafficking in neurons after ketosis (maybe too granular). —BDNF levels—can measure in CSF or indirectly in serum (though serum BDNF mostly from platelets, still could reflect brain trends). —Neurotransmitter turnover—e.g., ketones might reduce lactate in brain, which can modulate excitability too.

Indications: —Epilepsy: the ketogenic diet is an established epilepsy treatment, and exogenous ketones are being investigated to mimic it. MERIT would allow a more controlled “medical ketosis” for epilepsy without the need for a restrictive diet, and possibly allow dynamic titration (e.g., at times of day when seizures are more likely or around known triggers, boost ketone level). —Migraine: Migraines share mechanisms with epilepsy (cortical spreading depression is like a slow seizure). There are case reports that KD reduces migraine frequency. Also, some migraines are associated with mitochondrial issues (like familial hemiplegic migraine). —Autism Spectrum Disorder (ASD): Some ASD children benefit from ketogenic diets (observationally, improved cognitive and social function). Proposed reasons include modulation of GABA/glutamate (ASD often has E/I imbalance in brain), and less inflammation. So MERIT might be useful in certain ASD phenotypes. —Bipolar Disorder: Historically, some bipolar patients improved on KD (there's sparse data but some clinicians have noted mood stabilization). Mechanistically, similar to anti-epileptic drug effect since many bipolar meds are anti convulsants. —Schizophrenia: A few case studies of symptom reduction on keto diet. Possibly related to improved bioenergetics in brain plus offsetting some NMDA receptor hypofunction by altering glutamate cycling. —Chronic pain/fibromyalgia: If central sensitization is lowered by ketones that increase GABA or adenosine. Some anecdotal improvements reported in fibro with keto diet. —Neurodegeneration tremors and spasticity: e.g., PD patients sometimes have less tremor on keto (maybe due to overall calmer networks).

TTK in practice for this axis: One might have a patient wear a seizure-detecting watch which triggers a ketone infusion when abnormal activity is sensed—a sort of on-demand titration (this is speculative but future tech). Or instruct patient to take extra ketone dose at prodrome of migraine aura to abort an attack (like how people take glucose at aura sometimes).

Aim for a minimum level of BHB (say ≥2 mM) for anti-seizure effect (some correlation exists that higher ketones correlate with better seizure control). Possibly use AcAc containing formulas because some evidence suggests AcAc itself might directly reduce seizure (there was one study showing AcAc injection stopped seizures in animals more effectively than BHB, IIRC).

Example: A child with refractory epilepsy on MERIT titration: They wear a CGM-like continuous BHB monitor that alarms if BHB<1 mM (meaning risk of losing ketosis). The parent adjusts feeding of ketone drink to keep above that. Over 3 months, seizures reduce by 80%. Cognitive alertness improved because unlike high dose sedative drugs, ketones control seizures without sedation (maybe even improves cognition due to better fuel). Or an adult with severe anxiety on exogenous ketones finds lower anxiety scores, presumably because of increased GABA in brain (as indicated by MR spectroscopy perhaps-one could measure GABA with special MRI sequences in research).

### Metabolic Rewiring-Insulin Sensitivity, Fat Oxidation (FAO), De Novo Lipogenesis (DNL) Suppression, Resting Energy Expenditure

Ketones also broadly affect metabolism outside of immediate energy. They can improve insulin sensitivity, shift the body to fat-burning mode, reduce fat storage (DNL), and even raise basal metabolic rate (some studies found ketogenic diets have a slight metabolic advantage in energy expenditure).

Insulin Sensitivity: By providing energy from fat/ketones, blood glucose levels drop and insulin levels drop, which often leads to improved insulin sensitivity in muscle and liver. Also, some ketone molecules (like BHB) might directly bind to GPCRs on adipose (HCA2) causing release of adiponectin, an insulin-sensitizing hormone. Adiponectin increase is indeed seen in ketogenic diets. Adiponectin improves muscle's response to insulin and encourages fat oxidation.

Additionally, ketosis reduces visceral fat (over time) which is a major cause of insulin resistance via inflammatory cytokines.

FAO (Fatty Acid Oxidation): Ketosis is basically a state of high FAO-you're burning fat at a high rate (which produces the ketones in diet or uses them in exogenous supply scenario). In muscle and liver, ketones (particularly if accompanied by low insulin) activate PPARα and related pathways, boosting expression of FAO enzymes (like CPT1, long-chain acyl-CoA dehydrogenase, etc.). Over weeks, MERIT could raise a patient's capacity to oxidize fatty acids, reducing ectopic fat buildup.

DNL Suppression: DNL (de novo lipogenesis, conversion of carbs to fat in liver) is active in overfeeding states and contributes to fatty liver. Ketogenic conditions naturally suppress DNL because insulin is low and substrates (malonyl-CoA) are diverted to oxidation not lipogenesis. In fact, BHB itself might inhibit lipogenesis by activating AMP-kinase (not confirmed but likely because energy charge changes). Regardless, clinically ketogenic diets reduce liver fat (there's evidence they rapidly cut liver fat in NAFLD). Exogenous approach can do similarly even if patient isn't strictly dieting, by providing an alternate fuel that keeps insulin low and competes with carbs.

Resting Energy Expenditure (REE): people on ketosis have slightly higher REE, possibly due to inefficiencies of gluconeogenesis or due to mitochondrial uncoupling from increased FAO (some speculation BHB might cause mild uncoupling via UCPs or via increased futile cycles). Also, exogenous ketones themselves have a caloric value but not all of it may be efficiently used (some lost as heat or excreted as acetone). MERIT therapy could raise REE, it helps weight loss or weight maintenance.

Appetite Suppression: Ketosis is known to reduce hunger-BHB may act on the brain (perhaps the hypothalamus) to signal satiety, beneficial for metabolic syndrome patients to spontaneously eat less. Ghrelin (hunger hormone) tends to be lower on keto diets after initial adaptation.

Brown Fat Activation: Some evidence that ketone bodies or the hormonal milieu they cause (low insulin, high adiponectin) can activate brown adipose tissue (BAT) or beige fat-increasing thermogenesis.

Key Biomarkers: —HOMA-IR (Homeostatic Model Assessment of Insulin Resistance)—calculated from fasting insulin & glucose. Should improve (lower) under ketone therapy due to lower insulin. —Adiponectin levels-expected to rise. —Fasting insulin and fasting glucose-likely drop with chronic exogenous ketone usage (like Natural Alternatives patent found about beta-alanine improving insulin sensitivity, here ketones do similar). —Liver fat content—can measure by MRI or enzymes (ALT). Over time should drop. —Triglycerides —typically drop on keto, except perhaps transient rises early on. —Respiratory Exchange Ratio (RER)—lower RER means more fat burning vs carb. On ketones, RER might drop to ~0.7-0.8 from near 0.9 if someone was high carb. Could measure in metabolic chamber. —Weight and body composition-if relevant, patients might lose fat mass while preserving lean mass (as often reported in keto diets). —Leptin-often drops with weight loss, but ketosis sometimes spares lean mass and causes less drop in metabolic rate.

Indications: —Obesity and Overweight: Exogenous ketones can be part of obesity therapy, though caution needed because they have calories. But as an appetite suppressant and metabolic booster, perhaps given before meals to reduce appetite or as meal replacement, e.g., administer to maintain BHB ~2 mM until patient reaches certain weight goal). —Type 2 Diabetes and Insulin Resistance: There's the risk that giving ketones to a diabetic could cause high ketones on top of high glucose (ketoacidosis concern), but if done carefully and if they also moderate carb, it might improve glycemic control. Indeed, some T2D patients on keto diet can reduce meds drastically. Exogenous ketones specifically have been shown to lower blood glucose acutely-possibly by triggering insulin (contrary to typical thinking, a rise in BHB can cause a small insulin release which then lowers glucose, or by replacing some substrate usage). —NAFLD/NASH (here called MASH-metabolic dysfunction associated steatotic liver disease): weight loss via ketosis rapidly cuts liver fat. A ketone therapy to treat NASH, aiming to reduce liver fat and inflammation by metabolic means. —PCOS (Polycystic ovary syndrome): Insulin resistance and hyperinsulinemia are central in PCOS, and ketogenic diets have shown to improve hormonal profiles in PCOS. MERIT could be used in PCOS women to improve fertility and reduce weight, etc. —Rare metabolic disorders: glycogen storage diseases (where providing ketones bypasses need for glucose), or congenital hyperinsulinism (where ketones can keep brain fuel while controlling glucose infusion), etc. Also, Pyruvate dehydrogenase deficiency—these kids are actually treated with ketogenic diet because they can't use carbs well. Exogenous ketones would be literally life-saving to provide alternate fuel. —Athletic performance/Endurance: more fat burn, preserving glycogen, etc. For example, method of improving muscle performance in a subject (like the TdeltaS patent does) e.g., keep ketones 1-5 mM during event, etc.

TTK Implementation: Might involve patient self-monitoring ketones and glucose for a diabetic, maintain GKI below 4 for most of day. Or maintain morning fasting BHB ~1 mM (with an overnight slow release ketone maybe)—which leads to lower fasting glucose via Randle effect or others.

Can combine ketones with a GLP-1 agonist might yield even better weight loss (GLP-1 reduces appetite from another angle, ketones provide alternative energy and also reduce appetite).

Example: An obese, insulin-resistant patient is started on daily ketone esters before breakfast and dinner. They are told to still eat a healthy diet but not mandatory to be keto. Over 12 weeks, their HOMA-IR improves from 4 to 2, A1c from 6.0% to 5.5%, weight down 5%. MRI shows 30% reduction in liver fat. They report less hunger and spontaneously smaller portions. Blood tests show increased adiponectin and reduced triglycerides. This without major side effects aside maybe some GI upset initially. This illustrates MERIT as metabolic therapy akin to a medication.

Cardiovascular and Endothelial Function (eNOS Recoupling, ROS Reduction, Microcirculation)

Ketones have direct effects that could benefit the cardiovascular system: —Endothelial Nitric Oxide Synthase (eNOS) recoupling: In diseases like atherosclerosis or diabetes, eNOS can become “uncoupled”, meaning instead of producing NO (vasodilator), it produces superoxide (harmful ROS), often due to lack of its cofactor BH4 or due to oxidative stress. Some studies suggest ketogenic diet enhances NO bioavailability. Mechanisms: increased NAD⁺ might activate SIRT1 which deacetylates and activates eNOS. Also, less ROS from mitochondria means less quenching of NO. —Improved Endothelial Function: There are reports that ketone bodies (esp. BHB via HCA2 receptor on endothelial cells or immune cells) can reduce vascular inflammation and improve endothelial function tests (like flow-mediated dilation). —Arrhythmia threshold changes: Some research indicates hearts on ketones are more electrically stable (due to ATP-sensitive K+channels in heart or changed ion channel expression). Possibly beneficial for arrhythmias. —Blood pressure: Weight loss and improved insulin sensitivity from ketosis often lower blood pressure. Also, direct vasodilation from more NO. So hypertensives may benefit. —Microcirculation: Ketones may help microcirculation in part by RBC effects-interestingly, ketone bodies might improve RBC deformability or reduce blood viscosity. Also, by providing more energy, microvascular cells (which often suffer first in metabolic syndrome) can function better to deliver oxygen.

ROS and Oxidative Stress in Vessels: BHB-induced HDAC inhibition upregulates antioxidants like manganese superoxide dismutase (MnSOD) in endothelial cells, reducing oxidative stress, which protects NO. Also, as earlier, NAD⁺ increase can feed into poly(ADP-ribose) polymerase (PARP) which if overactivated was causing damage, but careful because too much PARP is harmful. Actually, NAD⁺ increase more helps sirtuins which protect against atherosclerosis by deacetylating NF-κB and reducing inflammation in vessels.

Key Biomarkers: —Flow Mediated Dilation (FMD): a functional measure of endothelial health. Could improve with ketone therapy. —NO metabolites (nitrite, cGMP): possibly up if eNOS better coupled. —BP readings: Lower systolic/diastolic BP after therapy, if hypertensive baseline. —VCAM-1/ICAM-1(adhesion molecules): should reduce if endothelial activation is lowered. —Arterial stiffness (Pulse Wave Velocity): might improve with metabolic improvements and less glycation. —Coronary blood flow or exercise tolerance: in heart disease, if heart uses ketones, it may function better under exercise. —BNP/NT-proBNP: heart failure marker-if improves, indicates better cardiac function. —Troponin/leak in chronic heart stress might reduce if hearts under less metabolic stress.

Indications: —Heart Failure: As discussed, energy for heart plus maybe anti-remodeling due to less ROS. —Peripheral Arterial Disease: Perhaps help by improving muscle metabolism and endothelial function in limbs (improved walking distance). —Hypertension: Indirect via weight loss and direct via vascular effects. —Diabetic Cardiomyopathy: Diabetes cause stiff heart even without coronary disease; ketones might alleviate via metabolic improvement. —Atherosclerosis prevention: If ketones reduce inflammation (NLRP3 in plaques) and improve HDL (often keto raises HDL), that could slow plaque progression. BHB's effect on monocytes also lowers foam cell formation. Use MERIT as an add-on to statin therapy for high-risk patients to address the metabolic component of their disease. —Stroke recovery: Overlaps with neuro, but vascular event. Some evidence that ketones can improve outcomes if given post-stroke by providing fuel and reducing ROS. —Migraine (vascular aspect): Migraine was partly covered in neuro, but also it's a vascular phenomenon. If endothelial function improves, migraine frequency may drop.

TTK Application: One might aim for chronic moderate ketosis in these patients, not extremely high, because they are often older or comorbid so we might be conservative (like target BHB ~1-2 mM stable). They could wear a ketone patch monitor to ensure overnight levels (when a lot of cardiovascular events happen e.g. morning surge) are maintained.

Example: A patient with type 2 diabetes and known coronary artery disease takes daily ketone supplements. After 6 months, their BP drops from 140/90 to 125/80, their HDL increased, triglycerides halved, CRP went from 4 to 1 mg/L. Endothelial function test (FMD) improved from 5% to 8%. Clinically, they have fewer angina episodes. This kind of multi-faceted improvement is expected from combining weight loss and direct ketone effects.

### Autophagy and Proteostasis (Cellular Cleanup and Protein Homeostasis)

Autophagy is a cellular self-cleaning process that removes damaged organelles and misfolded proteins. Fasting and ketosis strongly induce autophagy, which is beneficial in many contexts: neurodegenerative diseases (clear protein aggregates), aging (remove senescent organelles), cancer (short-term autophagy can promote survival but also can kill cancer cells under certain stress), etc.

Mechanism: Low insulin and low nutrients (especially low glucose, low amino acids) trigger autophagy via inhibiting mTOR and activating AMPK. Exogenous ketone therapy, if done with carbohydrate moderation, can simulate this nutrient state even if some calories are given. BHB itself may have signaling roles: some data suggests BHB can directly inhibit class I HDACs, which leads to activation of genes like those in FoxO family that induce autophagy-related genes. Also, BHB might bind to certain GPCRs or even histone sites that upregulate autophagy factors.

One interesting angle: BHB was reported to inhibit the inflammasome partly by inducing autophagy (some studies tied those together, autophagy can degrade pro-IL1β or the inflammasome complex). Also, in liver, ketone bodies reduce steatosis partly via autophagy of lipid droplets (so-called lipophagy).

Mitophagy (Selective autophagy of mitochondria): Ketosis might enhance mitophagy, thus cleaning up dysfunctional mitochondria and stimulating renewal (ties to PGC-1a induction for new mitochondria). This is great for diseases like Parkinson's (where mitophagy is impaired due to PINK1/Parkin issues)—maybe ketosis can bypass some of that by ramping up general autophagy.

Proteostasis: In aging and diseases like Alzheimer's, misfolded proteins accumulate. Autophagy plus chaperone-mediated autophagy (CMA) are ways to clear them. Ketogenic diet has been shown in some models to reduce accumulation of Aβ plaques and phospho-tau (in part possibly via autophagy). There's even evidence that ketone bodies can act as chaperones or stabilizers for some proteins (less documented, but e.g., acetoacetate was reported to protect against certain amyloid formation in an in vitro study).

Key Biomarkers: —LC3-II levels (autophagosome marker) in cells or PBMC-increased under autophagy. —p62/SQSTM1 (gets degraded by autophagy)—decreased if autophagy is active. —Ultrastructural evidence-more autophagic vacuoles on electron microscopy (not clinical but research). —Ketone therapy improving clearance of aggregate-prone proteins—e.g., measure α-synuclein or Tau in model systems with and without ketones, see reduction. —mTOR pathway markers—e.g., phospho-S6 ribosomal protein down=more autophagy. Possibly measure in PBMC or muscle biopsy.

Indications: —Neurodegenerative diseases (AD, PD, Huntington's): Use MERIT to promote clearance of toxic protein aggregates (amyloid, tau, Lewy bodies). The evidence: In animal models, keto or fasting improved clearance somewhat. —Liver disease (NAFLD/NASH): Autophagy in liver can clear lipid droplets and reduce fat (and indeed, fasting is a way to reduce liver fat partly via lipophagy). In metabolic axis but here mechanistically, autophagy is it. —Aging/longevity: Inducing autophagy is thought to extend lifespan (as seen with intermittent fasting, TOR inhibitors). MERIT could be a targeted therapy to mimic fasting in people who cannot fast easily. —Cancer: Autophagy has a dual role. Some cancer cells use autophagy to survive stress (so inhibiting autophagy can kill them), but other times, promoting autophagy triggers cell death if it goes uncontrolled.

TTK for Autophagy: To maximize autophagy, one might do intermittent fasting combined with exogenous ketones—e.g., skip breakfast but drink ketones to maintain energy and still have low insulin environment. Actually, giving exogenous ketones does raise insulin a bit (some evidence of small insulin response), but if properly formulated (especially pure BHB vs BHB salt), the insulin response is minimal, so autophagy can still proceed. Perhaps an intermittent closing schedule: e.g., 5 days a week at moderate ketosis, 2 days very high ketosis+calorie restriction to super-induce autophagy (kind of a “fasting mimicking” extreme).

Example: A patient with early Parkinson's disease uses a MERIT regimen: every evening, they fast and take a ketone ester, reaching BHB ~3-4 mM overnight. This is hypothesized to clear misfolded α-synuclein from neurons during sleep (a time when brain glymphatic clearance and autophagy are active). Over a year, their progression is slower than typical PD patients. Another example: an older adult uses a weekly 24 h ketone assisted fast to engage autophagy for anti-aging-after these fasts, blood tests show increased ketone, lowered insulin, elevated LC3-II in circulating white cells temporarily, indicating autophagy surge. It's a lifestyle treatment but also could be considered medical for age-related issues (like reducing risk of cancers, etc.).

### Unique Ketone Biomarkers-BHB/AcAc Ratio, “M-Score” Composite

This axis is focused on diagnostic and monitoring aspects.

BHB:AcAc or AKBR (Arterial Ketone Body Ratio): normally ~1:1 after overnight fast, rising to maybe 3:1 after 2-day fast (meaning more BHB), in diabetic ketoacidosis might be 6:1 or higher. This ratio is a direct readout of hepatic mitochondrial NADH/NAD⁺. In shock or liver failure, BHB:AcAc skyrockets because liver is super reduced (and indeed, measuring AKBR was studied as a prognostic in ICU-low AKBR (meaning high BHB:AcAc) correlated with mortality). For MERIT, use this ratio as a systemic marker to ensure delivering enough AcAc relative to BHB if our goal is to oxidize NADH. Or conversely, if want more BHB relative (some scenarios might be if AcAc was causing some side effect use BHB). Consider BHB:AcAc as the “dial” for redox tuning. In therapy, define an optimal ratio for a given patient—e.g., perhaps in Alzheimer's, delivering more BHB is fine (so ratio high is okay, as BHB is HDAC inhibitor), while in heart failure, want lower ratio (to maximize NAD⁺ gen). The ability to measure and adjust this ratio is something unique.

M-Score Composite: an index combining multiple axes. For instance, an M Score formula could be:

NAD+w2×+GSSG

M-Score=w1×+NADH

GSHw3×(H3acetylation %)−w4×(IL-6level)−w5×(TBARS oxidative

Normalized to some scale. A high score means good metabolic health, a low means dysregulation.

Use such a score to decide who needs therapy (target those with low score) and to track therapy (aim to raise M-Score by X points).

HCAR2 Activation assay: The receptor HCA2 (Niacin receptor) when activated by BHB leads to intracellular signaling (Gi pathway, reducing CAMP in certain cells). Measure an increase in prostaglandin D2 as an index (since niacin flush is mediated by PGD2 release from macrophages via HCA2).

3-Hydroxybutyrylation levels: covered, but listing as a biomarker too.

“Ketone challenge test”: as a diagnostic, give a small ketone dose and see how certain biomarkers respond—e.g., how much does NAD⁺ rise or how quickly does BHB get cleared (reflecting metabolic flexibility). For instance, two individuals get a ketone drink, one's BHB peaks at 1 mM and stays 3 hours, another's goes to 3 mM and stays 5 hours-differences might reflect insulin sensitivity (because insulin causes tissues to uptake ketones faster) and baseline metabolism. Diagnostic test of metabolic health based on response to a ketone bolus. If abnormal, that identifies a patient for MERIT. Integration into Therapy: schedule periodic lab panels to compute M-Score, and adjust closing or add combination therapies if the score isn't improving enough. For example, if after 2 months, NAD⁺ increased but IL-1β didn't drop, maybe add an anti-inflam drug or up ketone dose.

Example: A trial might be run where patients with metabolic syndrome are given MERIT therapy. They all have baseline M-Scores <=50(arbitrary scale). After 6 months, 80% of them have M-Score >70, correlating with improvements in their clinical metrics. Versus control group with minimal change.

### MERIT Axes Integration and Bioenergetic State Control

The present disclosure further defines the MERIT axes-Metabolic, Epigenetic, Redox, and Immune—as quantifiable therapeutic domains under the Unified Theory of Bioenergetic Sufficiency (UTBS). Each axis is associated with one or more measurable biomarkers that collectively determine the subject's M-Score (metabolic sufficiency index). Representative biomarkers include:
Metabolic: blood BHB 1-5 mM, AcAc 0.5-3 mM, glucose/ketone index≤3;
Redox: NAD⁺/NADH ratio ≥1.0, BHB:AcAc≤3:1, lactate/pyruvate≤20;
Epigenetic: H3K9ac ↑≥25%, H3K9bhb ↑≥20%;
Immune: IL-1β↓≥30%, CRP ↓≥40%, T-cell NAD⁺↑≥15%.

Therapy is titrated to achieve improvements across at least two axes, signifying restoration of systemic bioenergetic balance.

### Combination-Therapy Matrix

In some embodiments, MERIT therapy is administered in combination with established therapeutic agents according to the Combination-Therapy Matrix Table 5 below, which correlates disease categories, partner drug classes, and target biomarker endpoints.

TABLE 5
Combination-Therapy Matrix

| | | | Example | |
|---|---|---|---|---|
| | | Mechanistic | Target | |
| | Partner Therapeutic | Rationale under | BHB | Representative |
| Disease Cluster | Class | MERIT | (mM) | Outcome |
| Oncology | Immune- | Redox | 2-4 | Enhanced response |
| | checkpoint | normalization + T- | | rate ≥ 25% |
| | inhibitors; | cell re-energization | | |
| | metformin | | | |
| Neurodegenerative | Anti-amyloid or | Epigenetic | 1.5-3 | Improved cognitive |
| | dopaminergic | reprogramming + | | or motor scores |
| | agents | metabolic rescue | | |
| Cardiometabolic | SGLT2 or GLP-1 | Synergistic fuel | 1-2.5 | ↓ NT-proBNP ≥ |
| | agonists | utilization and | | 30% |
| | | oxidative stress | | |
| | | reduction | | |
| Autoimmune/ | Anti-cytokine | Inflammasome | 2-3 | ↓ CRP ≥ 50% |
| Inflammatory | biologics | suppression + | | |
| | | HDAC-modulated | | |
| | | tolerance | | |
| Psychiatric | Antidepressant or | Neuro-energetic | 1-2 | Improved symptom |
| | mood stabilizer | and GABAergic | | scales |
| | | stabilization | | |

Each matrix intersection defines a treat-to-target regimen comprising (i) a programmed therapeutic ketonemia window; (ii) co-administration schedule relative to the partner therapy; and (iii) axis-specific biomarker endpoints used to assess synergy. The matrix thereby operationalizes MERIT within UTBS, providing a unified framework for adaptive combination therapy across disease classes.

### Indication-Focused Implementations of MERIT

In this section, focus is on how MERIT/TTK can be applied to specific disease domains, highlighting the particular axes that are most relevant for each domain, and the clinical endpoints or biomarkers in those diseases that are targeted to improve.

Each sub-section below corresponds to a major “cluster” of indications: Oncology, Serious Mental Illness (depression, bipolar, etc.), Autism & Rare Neurodevelopmental, Neurodegenerative (AD/PD), Obesity/Metabolic/NASH, Epilepsy, Rare genetic/epigenetic disorders, Autoimmune/Inflammatory, Cardiovascular/Ischemic, and Mitochondrial disorders.

For each cluster, described are the relevant pathophysiology as MRED (which aspects of metabolic, redox, epigenetic, immune dysfunction are present). —How MERIT/TTK addresses each axis in that disease (practically and mechanistically). —Key biomarkers and endpoints to measure success (both clinical outcomes and mechanistic markers).

### Oncology

Pathology as MRED: Cancer is prototypical of metabolic and epigenetic dysregulation: —Tumors often exhibit the Warburg effect (a metabolic dysregulation where even in oxygen, they ferment glucose, indicating mitochondrial dysfunction or reprogramming). —Many cancers have regions of hypoxia, leading to reductive stress (NADH buildup) and use of alternate pathways (like reductive glutamine metabolism). —Cancer cells generate high ROS but also adapt to buffer it; they often have redox imbalance and depend on glutathione/NADPH to survive; tipping redox can kill them. —On the epigenetic side, cancers frequently have global DNA/histone hypomethylation and focal hypermethylation, and altered acetylation patterns. They often silence tumor suppressors via HDAC upregulation. Metabolites like 2-hydroxyglutarate in IDH mutant tumors cause epigenetic changes. So epigenetic dysregulation is critical in oncogenesis. —Immune dysfunction: advanced cancers create an immunosuppressive microenvironment (T-cell exhaustion, M2 macrophages, high IL-1β or TGF-β). There's evidence that some of this is driven by metabolic competition (cancer cells hog glucose, starving T-cells), and by certain oncogenic signals (e.g., inflammasome can be active in tumor-associated macrophages promoting inflammation that helps the tumor). Given all that, many axes of MERIT apply: —Metabolic: attempt to reverse Warburg by providing ketones (an oxidative substrate) possibly forcing tumor cells to oxidize or at least depriving them of some glucose (if patient is on lower carb plus exogenous ketones, blood glucose may drop and insulin is low, which can slow tumor growth; also certain tumors cannot use ketones well if they downregulated SCOT enzyme). —Redox: Using AcAc to drain NADH might push cancer cells (often highly reduced cytosol) into a state that triggers cell death. Additionally, elevated NAD⁺ could activate sirtuins that in some contexts act as tumor suppressors (Sirtuin 1 deacetylates p53 though, which is complex; but Sirt3 is a mitochondrial tumor suppressor). —Epigenetic: HDAC inhibition by BHB could re-express tumor suppressor genes or pro differentiation genes. For example, some trials use HDAC inhibitors for certain cancers; BHB might be a milder way to do similar. Kbhb on histones might also oppose cancer proliferation by activating anti proliferative genes. —Immune: If MERIT can reduce lactate (which tumors produce to suppress immunity) and provide fuel to T-cells, it can enhance immunosurveillance. Also, by lowering systemic insulin and IGF, it reduces growth signals to tumor. There have been small studies of ketogenic diets in glioblastoma, etc., showing some slowed growth in combination with chemo. The aim is for pharmacological ketosis that is consistent and combinable with therapy.

MERIT/TTK Implementation in Oncology: —Likely as adjunct to standard therapies, not usually sole treatment except possibly in low-grade tumors or as maintenance. For immunotherapy (checkpoint inhibitors: anti-PD1, CTLA4, etc.), hypothesize that keeping patients in mild ketosis during treatment improves response rates. Monitor increase in tumor infiltrating lymphocytes (TILs) with therapy. —For chemo or radiation, ketosis might selectively protect normal cells (e.g., normal cells adapt to ketones, while cancer cells that rely on glucose get hit—this concept is known as differential stress resistance). —pair with metformin (metformin lowers glucose and has anti-cancer metabolic effects; ketones+metformin might be synergistic to stress cancer metabolism).

Biomarkers/Endpoints in Cancer: —Obvious clinical ones: tumor size (imaging), progression-free survival, etc. —Mechanistic ones: measure FDG-PET uptake changes (if MERIT is working, tumor FDG uptake might drop after some weeks on therapy, indicating less glucose avid). —Circulating tumor DNA—not directly metabolic but if therapy is working, ctDNA drops. —For immune-responsive tumors, measure PD-L1 expression or T-cell infiltration in serial biopsies. —Side effect wise: ensure MERIT doesn't cause weight loss beyond desired in cancer patients.

Example Indications within Oncology: —Glioblastoma multiforme (GBM): The brain tumor often doesn't use ketones well because it's so glycolytic. Case reports of KD in GBM show some slowed growth. Exogenous ketones could reach the tumor through blood-brain barrier (they cross easily) and possibly starve it by competing with glucose plus providing fuel to surrounding normal brain to better withstand radiation. Use MERIT with standard temozolomide chemo and radiation for GBM. —Lung Cancer or Melanoma on immunotherapy: Combine MERIT to overcome resistance. —Breast Cancer: Possibly hormone-related metabolism aspects; some breast cancers might adapt to ketones though so need to target.

### Psychiatric Disorders (Serious Mental Illness-Depression, Bipolar, Schizophrenia)

Pathology as MRED: Serious mental illnesses (SMI) like major depressive disorder (especially treatment resistant depression, TRD), bipolar disorder, and schizophrenia have emerging evidence of metabolic and redox anomalies: —Mitochondrial dysfunction and low energy metabolism in the brain (e.g., lower pH and higher lactate observed in some MRS studies of bipolar and schizophrenia brains, suggesting glycolytic shift). —Inflammation and oxidative stress are often present (e.g., elevated cytokines in some depression, increased markers of oxidative damage in schizophrenia). —Some patients have insulin resistance even without obesity-interestingly, insulin resistance correlates with poorer response to antidepressants, etc. —Epigenetic factors: chronic stress changes histone acetylation patterns in the brain (ex: more HDAC activity has been linked to depression-like states in animals; HDAC inhibitors showed antidepressant effects in preclinical models). —In schizophrenia, an epigenetic phenomenon is the altered methylation of GABA and glutamate-related genes; while that's not directly ketone-targeted, the metabolic context could influence it.

MERIT Mechanisms for Psych: —Mood Stabilization via Neurotransmitter Balance: Ketosis increases GABA relative to glutamate, which can reduce anxiety and stabilize mood swings (bipolar mania is glutamate-high perhaps, and KD might calm that; indeed some bipolar patients off-label tried KD with reported benefit). —Antidepressant via Neurotrophic and Epigenetic Effects: BHB's HDAC inhibition can increase BDNF and other factors important for mood. Also, ketosis may normalize HPA-axis by affecting hypothalamic energy sensing. —Antipsychotic Adjunct via Metabolic: Many antipsychotics cause weight gain and metabolic syndrome. MERIT could counteract that by improving metabolism (which is helpful since a big issue in schizophrenia treatment is medication-induced diabetes; using ketones might mitigate weight gain and provide energy to the brain that might improve negative symptoms or cognitive symptoms). —Cognitive clarity: Many on ketosis report improved focus (some call it “keto clarity”). Possibly due to steady fuel supply and less brain insulin resistance. This could help cognitive symptoms of depression or schizophrenia. —Anxiolytic: As mentioned, research by Ari and D'Agostino showed reduced anxiety behaviors with ketone supplements. Mechanism likely GABA or maybe ketones modulating certain brain circuits. —Redox in psych: Schizophrenia's one hypothesis is glutathione (an antioxidant) deficit in parvalbumin interneurons leads to oxidative damage and dysfunction (hence disrupted oscillations). Raising NAD⁺ and improving redox by ketones might boost glutathione or at least reduce oxidative burden.

MERIT Implementation in Psych: —a drink or pills taken daily as adjunct to existing meds. For TRD, maybe as an augmentation to antidepressant or instead of ECT in some cases. —For bipolar, could be maintenance therapy to reduce frequency of mood episodes, given along with or in place of lithium or valproate. A benefit: exogenous ketones might avoid side effects like lithium toxicity or valproate weight gain. —For schizophrenia, possibly as add-on to clozapine or others to improve outcomes and metabolic health. —It's critical to manage the metabolic side effects of psychiatric meds, so MERIT might find a niche simply for preventing antipsychotic-induced weight gain and diabetes (which is very common).

Biomarkers/Endpoints: —Clinical scales (MADRS or HAM-D for depression, YMRS for mania, PANSS for schizophrenia). Biomarkers: maybe inflammatory markers (CRP, IL-6 often elevated in depression; see if they drop). —Imaging: ketone therapy changes brain metabolism on FDG-PET (could normalize certain hypometabolic areas). —Cognitive tests: ketogenic interventions often improve cognitive speed, which could be measured. —In anxiety, behavioral metrics like time in open arm of maze (in preclinical) or self-reported anxiety scales in humans.

Example Indications: —Treatment-Resistant Depression (TRD): trial giving TRD patients a ketone ester supplement in addition to their antidepressant; measure depression scores. There's case anecdote of a woman with chronic depression improved on keto diet after nothing else worked. —Bipolar Disorder: Possibly target the depressive phase or rapid cycling. There's evidence that metabolic dysfunction (like diabetes) worsens bipolar; addressing metabolism could stabilize mood. —Schizophrenia Cognitive Impairment: Hard to treat with standard meds; maybe metabolic therapy yields slight improvements in cognition and negative symptoms (due to better brain energy usage).

### Autism Spectrum & Rare Neurodevelopmental Disorders

Pathology as MRED: Many neurodevelopmental disorders (NDDs) have underlying metabolic or epigenetic components: —Autism Spectrum Disorder (ASD): Subsets have mitochondrial dysfunction (some ASD kids have lower ETC activity or carnitine deficiency), also often have increased oxidative stress markers. Some have hyperactive mTOR signaling (like TSC tuberous sclerosis or PTEN mutations causing autism, related to metabolism). Also, GABA/glutamate imbalance is implicated in ASD-many ASD have low GABA levels relative to glutamate in certain brain regions (excitatory/inhibitory imbalance). Inflammation (neuroinflammation) and gut metabolic issues are also noted in ASD. Epigenetically, environmental factors and mutations in chromatin regulators (e.g., CHD8, MeCP2 duplication, etc.) can cause autism or similar features. —Rett Syndrome: Caused by MECP2 mutation (epigenetic reader protein). The result is improper gene expression, including of metabolic genes. Rett girls often have low muscle tone, some autonomic dysregulation. KD has shown improvements in animal models likely due to overall neuroprotection, possibly epigenetic compensation (MECP2 normally represses some genes-its absence might cause chaos, but maybe global HDAC inhibition by BHB helps normalize some open chromatin issues). —Kabuki Syndrome: Mutations in KMT2D or KDM6A (histone modulator enzymes). Leads to intellectual disability and immune issues. Because it's directly an epigenetic mutation, using a metabolite that affects histones (BHB) might help adjust the epigenetic landscape. For instance, Kabuki had success in mouse model by giving HDAC inhibitor AR-42 (which improved memory). BHB could act similarly but gentler. —Angelman Syndrome: UBE3A gene lost; some evidence ketogenic diet reduced seizures and improved EEG in Angelman kids. Possibly because it addresses neuroexcitation or some metabolic synergy. —Other Rare Metabolic Brain Disorders: PDH deficiency (already mentioned, these kids can be put on KD for life, but exogenous ketones would be a straightforward therapy here, because they simply need alternative fuel); Glut1 deficiency (lack the transporter for glucose into brain-currently treated with KD since brain must use ketones; exogenous ketones are perfect for them as a therapy-indeed, triheptanoin and others are being used. —Lysosomal storage disorders or peroxisomal disorders that have neuro effects (like ALD): maybe not direct for ketones, but ketones might improve general neuronal resilience.

MERIT in ASD & NDD: —Seizure control: Many NDDs (Rett, Angelman, tuberous sclerosis, etc.) have epilepsy; ketosis known to help seizures. —Behavioral improvement: Some small studies of KD in autism reported improved social interaction and communication. Possibly by better energy use, calmer brain, less oxidative stress. —Cognition and motor: In Rett, anecdotal improvement in motor skills on KD. Possibly due to increased BDNF and synaptic proteins (since BHB increases those). —Immune modulation: Some autism cases involve neuroinflammation; NLRP3 might be part (there's speculation maternal inflammation could predispose, etc.). If ketones reduce inflammation, could help symptoms. —Social behavior modulators: There's research showing certain metabolic drugs (like pioglitazone, a diabetes drug) helped irritability in autism. If metabolic improvement helps, ketones might similarly via decreasing inflammation.

Biomarkers/Endpoints: —Behavioral scales (ADOS for autism severity, etc.), but those are subjective somewhat. —Possibly EEG improvements (some non-seizure ASD kids still have abnormal EEG; if ketosis normalizes some patterns that might correlate with better function). —Mitochondrial biomarkers if applicable (like lactate: pyruvate ratio, some ASD have elevated lactate). —For specific disorders: e.g., in Kabuki model, memory test improved; in Rett, breathing irregularities (Rett has episodes of hyperventilation and apnea which might improve because KD stabilizes autonomic function). —Angelman's ataxia might improve (like gait measurement).

Examples: —A case: 10-year-old boy with autism and uncontrolled epilepsy-put on exogenous ketone regimen reaching ~4 mM BHB, seizures reduced, plus his social eye contact improved and he started speaking single words where previously non-verbal. Attribute to combined anti-seizure and perhaps direct neuronal modulation by ketones. —Or a Kabuki syndrome teenager: after MERIT therapy, their recurrent infections decreased (because ketones improved immune function, maybe epigenetically corrected some immune cell gene issues), and cognitive focus improved. Hard to measure but maybe teacher reports better attention.

### Alzheimer's, Parkinson's & Neurodegeneration

Pathology as MRED: —Alzheimer's Disease (AD): Marked by brain glucose hypometabolism (even in preclinical AD, PET scans show reduced glucose uptake in parietal cortex). Also by accumulation of misfolded proteins (amyloid, tau)—proteostasis failure, oxidative stress from plaques and dysfunctional mitochondria, insulin resistance in brain (some call AD type-3 diabetes). Also significant neuroinflammation (activated microglia, NLRP3 has been implicated as responding to amyloid and driving pathology). Epigenetic changes in AD: some key synaptic and memory gene promoters become hypoacetylated (HDAC2 upregulation observed in AD models). So AD hits all axes: metabolic deficit, redox stress, epigenetic silencing, immune activation. —Parkinson's Disease (PD): Primary issue is loss of dopaminergic neurons partly due to mitochondrial dysfunction (complex I deficiency in substantia nigra), oxidative stress (dopamine metabolism generates ROS, plus environmental toxins cause oxidative damage), and accumulation of misfolded protein (α-synuclein). There's also a central and peripheral insulin resistance link with PD risk (diabetics have higher PD risk, and trials show GLP-1 agonist improved PD symptoms). Neuroinflammation in PD (activated microglia releasing IL-1β that can kill neurons). Epigenetics: less studied but some PD genes are epigenetically regulated. —Others: ALS (amyotrophic lateral sclerosis)—extremely energy-demanding motor neurons, some evidence KD extended survival in mouse model. Mechanism: maybe giving fuel to neurons, reducing glutamate toxicity, and possibly improving motor neuron metabolism which is impaired by TDP-43 pathology. Huntington's disease-deficits in energy metabolism too (weight loss is common early, brain can't use glucose normally), some positive signs of KD in models and small trials. Multiple system atrophy and other atypical Parkinsonism could also benefit similarly to PD logic. —Overall: these diseases share neurodegeneration where cells are failing in part due to energy deficits, oxidative damage, and toxic proteins.

MERIT in AD/PD: —Fueling neurons: Provide ketones to bypass brain's inability to effectively use glucose (in AD, neurons might have insulin resistance or transport issues; but they can uptake and use ketones via monocarboxylate transporters which are often upregulated in AD brain ironically). Trials of medium-chain triglycerides (MCT) in mild AD showed modest cognitive improvement (this is essentially raising ketones a bit). Our approach can raise them higher and more consistently. —Reducing pathology load: There's evidence ketosis reduces amyloid burden in mice (possibly via autophagy or decreased amyloid production due to lower insulin signaling). In PD models, ketones protected neurons from toxins like MPTP. Mechanism: improved mitochondrial function plus maybe a direct effect on neurotransmission (some PD patients report improved tremor or less off-time on KD). —Improving cognitive function via epigenetics: BHB's HDAC inhibition might reopen expression of memory-related genes in hippocampus. In AD, reactivating these could help memory independent of amyloid changes. —Reducing neuroinflammation: as mentioned, NLRP3 in microglia contributes to AD progression-BHB can quell that, potentially slowing disease. —Mobility and energy: In PD, providing ketones may improve motor function by fueling surviving neurons and muscles (PD often causes muscle weakness also partly central partly peripheral). —Safety neuroprotective in general: If a cell is starved or stressed, ketones provide a stable energy supply and cause less oxidative stress per ATP than glucose (since less ROS by-product in some comparisons). Also, NAD⁺ increase by AcAc could activate Sirtuins (SIRT3 and SIRT1) which have been shown to protect neurons (SIRT3 protects against neuronal stress by preserving mitochondria, SIRT1 deacetylates transcription factors to promote survival).

Endpoints: —AD: cognitive tests (ADAS-Cog), and potentially biomarker like FDG-PET (should improve uptake or at least slow decline if using combined with some metabolism from ketones). CSF biomarkers (amyloid, tau) might or might not change-if MERIT slows disease, tau accumulation might slow. Neuroimaging of brain volume-maybe slower atrophy. —PD: motor scores (UPDRS), maybe reduction in daily levodopa needed or improved “on” time. Possibly ketone therapy could reduce dyskinesias (since some evidence PD dyskinesias relate to fluctuations in energy/metabolism-speculative). —Biological: MRI spectroscopy could measure brain ketone usage or NAD levels (there's 31P MRS for NAD). —“Fluorinated ketone PET tracer”: maybe in future to see ketone uptake, but something like that could show that brain is indeed taking up the exogenous ketones.

Example for AD: A 70-year-old with mild AD uses daily a ketone ester drink to keep fasting BHB ~2 mM. After 6 months, his memory tests decline less than expected. FDG-PET shows typical reduced glucose usage but interestingly his cognitive function is stable, possibly because brain is now using ketones (one might try doing a PET scan with a ketone tracer if that existed, or infer via cognitive effect). Also, some functional connectivity MRI might improve because energy supply is better.

Example for PD: A patient with moderate PD tries a daily ketosis regimen. He notes improved energy and slight improvement in tremor and stiffness, perhaps because of altered neurotransmitter environment (some speculation that ketones can increase brain acetylcholine which might help balance dopamine, or just improved ATP in neurons reduces their firing irregularities).

### Obesity, Metabolic Syndrome & Fatty Liver

Pathology as MRED: —Obesity and Metabolic Syndrome: Characterized by insulin resistance (metabolic dysregulation), often chronic inflammation in adipose (NLRP3 likely active in fat macrophages), oxidative stress from overnutrition, and sometimes epigenetic changes from long-term high-fat/high-sugar diet (some evidence of epigenetic differences in muscle of diabetics). Adipocytes in obesity release lots of fatty acids-high NADH in liver from those can cause problems (so reductive stress in liver). —Type 2 Diabetes (T2D): Beta cell dysfunction partly from glucolipotoxicity which involves redox and ER stress; insulin resistance with redox/inflammatory components. Epigenetics in diabetes: hyperglycemia can cause persistent epigenetic changes (“metabolic memory”). —NAFLD/NASH (Metabolic Associated Fatty Liver Disease): Excess fat in liver caused by insulin resistance leads to fat accumulation (metabolic issue), oxidative stress (fat oxidation producing ROS or by overloaded ETC, plus NADH excess), inflammation (NASH has high IL-1β, likely NLRP3 in Kupffer cells), and even fibrogenic signals (some triggered by oxidative stress). Fatty liver also involves DNL which is epigenetically regulated by carbohydrate response element binding protein (ChREBP) and others-high insulin causes certain histone acetylation that leads to lipogenic gene transcription. So MASH is very much metabolic and redox problem with an inflammatory fibrotic outcome. —Polycystic Ovary Syndrome (PCOS): Considered a female metabolic syndrome-high insulin, often overweight, leading to ovarian dysfunction. Insulin and maybe epigenetic changes cause high androgen. PCOS patients benefit from keto diets in studies (improved hormones, weight). —Rare obesity or lipodystrophy: Some genetic conditions where metabolism issues cause extreme NAFLD or diabetes; ketosis might help as a workaround or to reduce fat burden.

MERIT in Obesity/Metabolic: —Weight loss and body composition: Ketones suppress appetite, increase fat burn, and preserve lean mass (due to protein sparing effect of ketosis and possibly mild elevations in growth hormone during fasting states). —Use TTK to ensure safe levels (so they don't go into ketoacidosis even if they have mild insulin deficiency). —Combine with calorie restriction or just use as adjuvant (like a person diets but also takes ketones to feel less hungry and get into ketosis faster). —Insulin Sensitivity improvement: Already covered how NAD⁺ and less ectopic fat help that. —Liver improvement: Provide energy to body so that liver doesn't have to engage in gluconeogenesis as much (maybe reducing stress), plus direct use of fat for fuel empties liver fat stores. Autophagy (lipophagy) in liver is induced, clearing fat and possibly preventing progression to NASH. —Reduce inflammation: Adipose tissue gets less inflamed when insulin goes down and fat is being burned (fewer macrophages crowding, NLRP3 less triggered once fat cells shrink). —Some interesting data: ketone bodies directly inhibit inflammasome in adipose tissue; in a study, obese mice had less IL-1β after BHB. —Epigenetics of metabolic memory: If someone had years of high sugar, their blood vessels remain at risk even after control (metabolic memory of damage). Perhaps intense ketosis resets some epigenetic marks of that memory (like via HDAC inhibition might remove repressive marks at protective genes). —There's also the concept of brown fat activation-Niacin (HCA2 activation) triggers a flush and maybe slight browning of fat; BHB hitting the same receptor could potentially do something similar, which can increase energy expenditure. —PCOS: by lowering insulin and weight, restore ovulation. Maybe by specific signals too (adiponectin up might improve ovarian function as adiponectin receptors in ovary influence steroidogenesis).

Practical Implementation: —Possibly a medically-supervised weight loss program: instead of fully fasting the patient (which is effective but tough), give them exogenous ketones to mimic fasting metabolism, thereby losing weight with less hunger. Could also incorporate intermittent full fast days with heavy ketone support to maintain energy (fast-mimicking approach). —For NAFLD, maybe not even requiring weight loss; some lean NAFLD patients might just need metabolic tweak-ketones could reduce liver fat via metabolism directly. —Combine with standard care: e.g., with GLP-1 agonists for obesity (complement each other: GLP-1 reduces appetite strongly and improves glycemic control, while ketones provide alternate fuel and possibly avoid lean mass loss). —In T2D, maybe try to replace some glucose lowering meds or at least reduce them (if patient runs on some ketones, their glucose may spontaneously drop).

Biomarkers/Endpoints: —Weight, BMI. —Waist circumference, which reflects visceral fat-likely to drop with ketosis as visceral fat is quickly used. —HOMA-IR, fasting insulin/glucose. —Liver enzymes (ALT/AST), liver MRI-PDFF (proton density fat fraction to quantify fat in liver). —Liver stiffness by Fibroscan for NASH (should reduce if inflammation & fibrosis reduce). —Lipid profile: typically, on keto, triglycerides drop, HDL rises, LDL sometimes rises in some but often particle size shifts to less atherogenic. Monitor and ensure any LDL rise is mitigated (maybe by adding fiber or medication). —PCOS: track menstrual regularity return, androgen levels (testosterone down, SHBG up if insulin lowers). —In diabetes: A1c reduction and time—in range for glucose if they use CGM.

Example: A 45-year-old with BMI 35, T2D, NAFLD: put on MERIT therapy with daily ketone esters and some diet guidance. In 6 months: lost 10% of body weight, Alc from 8% to 6.5% (with reduction of some meds), liver fat fraction on MRI from 20% to 5% (essentially resolved NAFLD), ALT normalized. Their M-score soared from low to high, reflecting improved metabolism. They also subjectively feel energetic and not hungry often. This would be a showcase of multi-axis success: metabolic fix, redox (perhaps measure that lactate is lower in exercise now), epigenetic (maybe measure that some genes like PPARGC1A (PGC1a) have more acetylation meaning more active, etc.), immune (CRP down from 5 to <1 mg/L).

### Epilepsy

Pathology as MRED: Epilepsy, especially refractory epilepsy-Many childhood epilepsies are due to metabolic enzyme defects (GLUT1 deficiency as classic for KD; PDH deficiency causing seizures; mitochondrial disorders). —Even in idiopathic epilepsy, the seizure focus can have abnormal energy use (some PET scans show either hyper or hypo metabolism in foci). —Redox: seizures cause a surge of ROS from intense firing; recurrent seizures can cause oxidative damage that then lowers seizure threshold further. Some epileptic syndromes have antioxidant issues. —Inflammation: A lot of epilepsy conditions have neuroinflammation (post-status epilepticus leads to IL-1β release which then makes brain more excitable; some patients benefit from anti-IL-1 or ketosis which lowers IL-1). —Epigenetic: Chronic seizures can lead to changes in gene expression (e.g., increased HDAC expression, changes in neuron receptors). HDAC inhibitors have shown anticonvulsant effects in labs (valproate, a common epilepsy drug, is actually an HDAC inhibitor too, though weak).

MERIT for Epilepsy: Ketones provide a stable fuel so that even if a seizure causes a local energy crisis (neurons fire so much they exhaust local glucose and oxygen, leading to depolarization and more seizures), ketones being available can mitigate that energy crisis, raising the threshold for seizures. —GABA increase via ketosis directly calms neuronal firing. —BHB's effect on K_ATP channels hyperpolarizes neurons making it harder to fire. —NAD⁺ increase might activate sirtuin 1 which suppresses transcription of some proconvulsant genes, or Sirtuin 3 which protects neurons from stress. —Many epilepsies have an inflammation component (like febrile infection-related epilepsy syndrome-IL-1 antagonists help some cases; ketosis likely reduces that inflammation). —Also, ketones raise the seizure threshold by altering ion gradients—for example, less lactic acidosis during activity means less acid effect on neurons (acidosis in brain can either stop or trigger seizures depending on context, complex but global effect). —There's also evidence that ketosis upregulates neuropeptides like adenosine (a potent endogenous anticonvulsant; KD increases adenosine levels by reducing its uptake). —For certain genetic epilepsies: e.g., Dravet syndrome (SCN1A mutation) which is severe-KD is an option in those kids. Possibly exogenous ketones could complement new drugs like fenfluramine for Dravet.

TTK specifically: perfrom closed-loop: e.g., a device that detects a seizure onset and delivers a bolus of ketones nasogastrically or injection to abort it (fasting spontaneously stops some seizures, so an acute ketone spike might). Maintain continuous moderate ketosis. Or in cycles (some kids do intermittent fasting to control seizures). Measure blood BHB to ensure it's in therapeutic range—in KD therapy they measure it anyway as a compliance check, refine that (maybe target 4 mM for severe epilepsies vs 2 mM for milder etc)

Biomarkers/Endpoints: —Seizure frequency. —EEG improvements (reduced epileptiform discharges). —Developmental progress in children (stop seizures, often kids start developing better). —Possibly IL-6 or IL-1 in CSF lowering if we measured that—not routine though. —Ketone levels correlate somewhat with seizure control: find threshold like keep BHB>2 mM 90% of time to ensure minimal seizures—an actionable metric.

Example: A 3-year-old with Lennox-Gastaut syndrome (multiple seizure types daily, cognitively impaired). They start a MERIT regimen via a gastrostomy tube delivering a balanced BHB/AcAc ester continuously (like a slow feed) maintaining BHB ~3-5 mM. Seizures drop from 50 a day to 5 a day. EEG shows fewer spikes. Over 6 months, the child starts to speak a few words and cognitive gains are noted (because less time seizing). This is essentially replicating KD effect but with a stable, titratable approach (which could be easier than a diet).

### Rare Genetic/Epigenetic Syndromes (Rett, Kabuki, Angelman, Mitochondrial, etc.)

Rett (MECP2): Already covered in neurodevelopment, but specifically, a hallmark is regression at 1-2 years old, loss of speech, seizures, breathing issues. KD in a small trial in Rett girls improved motor skills and alertness, and reduced breathing irregularities. Likely via hyperexcitation reduction and maybe increasing BDNF (MeCP2 deficiency leads to low BDNF, but KD can raise BDNF). —Kabuki (KMT2D/KDM6A): Intellectual disability plus immune deficiency. Possibly MERIT could rescue some memory deficits (like HDAC inhibitor did in mice). Also, these patients often have growth hormone issues (just anecdote, but metabolism is weird; ketones might improve energy use). —Angelman (UBE3A): They have severe developmental delays, ataxia, seizures, happy demeanor ironically. A case series on KD showed reduced seizures and modest cognitive gains. Perhaps due to increased inhibition (lack of UBE3A may affect GABA receptors—there's known GABAergic deficits in Angelman, and KD increases GABA which might partly compensate). —Glut1 Deficiency: Already recognized-they absolutely need ketosis to feed their brain. MERIT is basically a treatment to provide what their body can't (glucose can't cross properly, so give ketones which do cross via MCT1). —Pyruvate Dehydrogenase Deficiency: Another one where brain can't use carbs well, ketosis provides alternate route. Known indication for KD. —Mitochondrial disorders: e.g., MELAS (mito encephalopathy lactic acidosis stroke-like episodes), MERIT could help by providing an easier fuel (BHB oxidation uses less complex I relative to NADH because some goes through succinyl-CoA route etc-maybe helpful if complex I is partially defective, plus the NAD⁺ boosting from AcAc might help). —Leigh syndrome (many causes): KD sometimes tried in these severe neuro disorders with variable results. Possibly helps some by bypassing blocks. —Glycogen Storage Disease (like type III, V): They can't mobilize glucose properly, so ketones can serve as alternate fuel. Some GSD patients are put on cornstarch therapy or MCT; we can use BHB esters for more direct effect.

For each, mechanistically:—In metabolic enzyme deficiencies, ketones circumvent the block (like PDH, Glut1). —In epigenetic, ketones modulate gene expression to offset the defect in epigenetic machinery (like Kabuki's haploinsufficiency of H3K4 methylation might be partly overcome by more histone acetylation to keep chromatin open, as one example). —In mitochondrial DNA mutations, providing alternative fuels plus NAD⁺ maybe helps maximal function of remaining ETC capacity.

Biomarkers: disease-specific (e.g., in MELAS measure lactate levels and frequency of stroke-like episodes; in Glut1 measure neurodevelopment and seizure frequency; in PDH deficiency measure lactate and neurodevelopment). For epigenetic ones, maybe measure gene expression changes—e.g., in Kabuki model, see if synaptic genes upregulate on ketones.

Example: A child with Glut1 deficiency who had daily seizures and developmental stagnation goes on exogenous ketone supplementation (drinks every 4 hours). Seizures stop (as expected, because brain gets fuel), and they start developing cognitive and motor skills better. Or an adult with MELAS-before, had frequent stroke-like episodes (metabolic strokes); after MERIT therapy with daily ketone infusion or high dose drinks, lactate levels are down and they haven't had a stroke episode in a year, quality of life improved (some anecdotal support: there was a case of a MELAS patient on KD with improved exercise tolerance).

### Autoimmune & Inflammatory Diseases

Pathology as MRED: —Autoimmune diseases (like rheumatoid arthritis, lupus, multiple sclerosis, type 1 diabetes, psoriasis, inflammatory bowel disease) involve an overactive immune system often fueled by metabolic shifts. For example, activated immune cells rely on glycolysis (Warburg-like metabolism to rapidly proliferate and produce cytokines). Chronic inflammation shows characteristics of metabolic dysregulation: e.g., rheumatoid patients often have altered muscle metabolism (cachexia), high lactate in joints, and overall increased resting energy expenditure but fatigue. —Redox: In inflammation, ROS are produced and cause tissue damage (e.g., in lupus, low NAD⁺ in T cells lead to aberrant T cell death or something; in RA, lots of oxidative damage in joints). —Epigenetic: Autoimmune diseases show changes in DNA methylation and histone marks in immune cells (e.g., FOXP3 gene in Tregs can be modulated by acetylation affecting regulatory T cell function; lupus T cells have issues with DNMT1 levels leading to overexpression of certain genes). —Inflammasome (NLRP3) is known to be central in gout, also implicated in complications of lupus and in atherosclerosis of RA etc. —Also many autoimmune patients have metabolic syndrome from chronic steroid use or disease—an additional target.

MERIT effects: —Suppressing innate inflammation: As heavily discussed, BHB inhibiting NLRP3 and reducing IL-1, etc. So conditions like gout, adult Still's disease (IL-1 driven), cryopyrinopathies could be directly helped. —Modulating adaptive immunity: Possibly by boosting NAD⁺ in Tregs or altering T cell metabolism so that more regulatory and less effector responses. Some studies in MS mice showed KD increased frequency of regulatory T cells which help quell autoimmunity. Mechanism: probably through metabolic reprogramming of T cells to favor oxidative metabolism (reg T cells use lipids and OXPHOS more, whereas effector T cells use glycolysis). —In MS, a pilot study of KD had improvements in fatigue and some immune markers. Also, ketones might help remyelination by providing energy to oligodendrocytes. —RA: weight loss from ketosis would help RA by reducing load on joints and lowering systemic inflammation (adipose releases inflammatory adipokines). —Pain and inflammation reduction: Ketones might act as analgesic indirectly by reducing inflammation and possibly direct effect on pain pathways (BHB activating HCA2 on macrophages triggers release of prostaglandins that can cause flush but also might modulate pain). Niacin (which uses same receptor as BHB) has been used in osteoarthritis trial to see if it helps cartilage. BHB might similarly affect chondrocytes (just speculation). —Epigenetic tolerance induction: HDAC inhibition by BHB could promote expression of PD-L1 or other checkpoints on cells to reduce autoimmunity (some HDAC inhibitors show anti-inflammatory effect by increasing CTLA4 or PD-L1 on T cells).

Endpoints: —Disease-specific: e.g., MS relapse rate or MRI lesion load (if MERIT reduces new lesions). —RA: ESR/CRP, swollen joint counts improvement. —Gout: frequency of flares (like how often need colchicine). —Biomarkers: IL-1 or IL-6 levels go down, maybe autoantibody titers might drop if inflammation overall down (like rheumatoid factor or ANA). —For MS, number of Tregs via flow cytometry might increase on therapy.

Example: A patient with RA and metabolic syndrome tries MERIT in addition to their usual medication, after 3 months they find CRP halved, they lost weight, joint pain improved more than with meds alone, and they could reduce dose of steroids. Or a teenager with MS does KD or exogenous ketones and feels less fatigue, possibly fewer relapses (some anecdotal accounts exist).

### Cardiovascular, Ischemic & Mitochondrial Disorders

This includes: —Atherosclerotic Cardiovascular Disease (ASCVD): Covered partly in metabolic for risk factors and in immuno for inflammation. Specifically, NLRP3 in plaques, etc. MERIT might stabilize plaques by reducing macrophage inflammation and increasing efferocytosis (phagocytosis of dead cells) due to autophagy. The combination with statins or PCSK9 would be typical. —Myocardial Ischemia (Angina, MI): If a heart has low blood flow, ketones being efficient fuel could help it produce more ATP per oxygen (some studies: infusion of BHB in pigs during ischemia improved cardiac output). So in stable angina, raising ketones might reduce chest pain episodes by fueling heart better. In acute MI, maybe an infusion of ketones during reperfusion reduces injury (less reperfusion arrhythmia, smaller infarct size due to NAD⁺ replenishment). —Heart Failure: Failing hearts reprogram to use more ketones; exogenous ketones improve hemodynamics in studies. Could implement as chronic therapy. —Peripheral ischemia: e.g., critical limb ischemia-maybe ketones help tissues survive low blood flow by providing alternate fuel and less ROS (some evidence hyperketonemia protects muscles from ischemic damage). —Shock (septic or other): In shock states, the body's metabolism is severely deranged (high lactate, very low AKBR ratio meaning high BHB:AcAc in poor perfusion). Perhaps giving ketones can sustain vital organs and reduce organ failure. —Mitochondrial genetic disorders: e.g., MELAS, etc. Also include: —Barth syndrome (mitochondrial cardiolipin issue)—maybe ketones help heart function there. —Leigh's disease (mentioned)—Friedreich's ataxia—a mitochondrial disease with spinocerebellar degeneration; some evidence high-dose B3 (niacin) helps by raising frataxin and NAD, similarly BHB could help by NAD and fuel. —Aging in general (as a risk): Many age-related issues like sarcopenia (muscle loss) are partly due to mitochondrial decline; ketosis in older adults might preserve muscle via better energy and plus direct anti-catabolic effect (ketones reduce protein breakdown).

In summary for these disorders: MERIT can help by: —Providing superior metabolic support under stress (ischemia, heart failure). —Reducing oxidative damage and inflammation in vessels (so slower progression of atherosclerosis, less reperfusion injury). —Improving exercise tolerance by fueling muscles with ketones and raising VO₂max possibly (like in peripheral artery disease, maybe walk longer). —For genetic mitochondrial, providing fuel that bypasses their block or stimulates residual function.

Endpoints: —Heart failure: ejection fraction, 6-min walk distance, BNP reduction. —Angina: frequency of chest pain episodes, exercise treadmill time to angina. —MI: infarct size measured by enzymes or imaging. —PAD: claudication distance improvement. —Vascular: perhaps carotid IMT (intima-media thickness) or plaque volume by ultrasound if doing long term. —Mito diseases: specific scales (like Newcastle Mitochondrial Disease Scale, etc., improvement).

Example: A 60-year-old with HFrEF (EF 30%) is put on ketone ester thrice a day. After 3 months, EF improved to 35%, VO2max increased slightly, and quality of life scores improved. At cellular level, his muscle biopsy shows increased expression of oxidative enzymes and less lipid accumulation in muscle. Or a patient given ketone infusion during a cardiac bypass surgery had lower markers of cell injury (troponin, etc.) post-op, implying tissue protection.

The methods, compositions, and uses described herein do not require that the subject exhibit glucose hypometabolism, reduced cerebral metabolic rate of glucose, elevated blood glucose, glucose intolerance, insulin resistance, or any other particular glucose metabolic phenotype. A subject maintaining normal glucose homeostasis, normal fasting glucose, normal insulin sensitivity, and optimal glucose metabolic rate will still achieve therapeutic benefit from the pharmacologically induced ketotic state through engagement of MERIT mechanisms that operate independently of glucose metabolic state. Critically, the MERIT pillars-histone β-hydroxybutyrylation, NLRP3 inflammasome suppression, HCAR2/GPR109A signaling, NAD⁺/NADH rebalancing, and circadian rhythm resynchronization—are each engaged by circulating BHB elevation alone and do not require any impairment of glucose uptake, glucose oxidation, or cerebral glucose metabolism. A neurologically and metabolically healthy subject with a normal brain metabolic rate of glucose will still achieve meaningful Kbhb deposition in peripheral blood lymphocytes, NLRP3 inflammasome suppression in monocytes, and NAD⁺/NADH elevation in lymphocytes upon pharmacological elevation of circulating BHB to therapeutic levels. Nothing in the present specification should be construed as limiting the invention to subjects characterized by glucose hypometabolism or reduced glucose oxidation.

The methods, compositions, and uses described herein are independent of dietary restriction and may be practiced in subjects on entirely unrestricted diets. A subject maintaining ad libitum intake of carbohydrates, proteins, and fats will still achieve a therapeutic ketotic state through pharmacological ketogenic agent administration and will still experience engagement of downstream MERIT mechanisms at the concentrations described herein. Nothing in the present specification should be construed as requiring carbohydrate restriction, caloric restriction, or any modification of baseline diet as a condition of achieving the therapeutic ketotic state. The capacity to achieve therapeutic ketosis without dietary restriction is a critical distinguishing feature of pharmacological ketosis induction and is not limited to any particular ketogenic agent chemistry or formulation. The independence from dietary restriction is a necessary implication of the Ketosis=Ketosis equivalence principle: if the therapeutic mechanisms are determined by circulating BHB concentration and target-engagement duration-—not by the dietary route of induction —then they must be achievable through any means that elevates circulating BHB, including means that do not require any dietary modification whatsoever.

Nothing in the present specification is to be construed as limiting the invention to subjects having an energy deficit, reduced whole-body energy expenditure, negative energy balance, reduced baseline metabolic rate, or any particular baseline metabolic phenotype except where expressly so limited in a specific claim. The therapeutic effects of ketosis are independent of whether the subject is in energy deficit or energy balance; they are determined by circulating ketone body elevation and target engagement of downstream MERIT pillars. A subject in energy balance-consuming adequate calories to maintain weight and metabolic rate—will still achieve therapeutic benefit from pharmacologically induced ketosis through MERIT pillar engagement. The present disclosure explicitly encompasses subjects who are overweight, of normal weight, underweight, insulin sensitive, insulin resistant, diabetic, non-diabetic, metabolically healthy, or metabolically dysregulated; the only prerequisite for MERIT pillar engagement is the pharmacological elevation of circulating BHB above the relevant target-engagement threshold for the indication in question.

The means of ketosis induction is not limiting. Any pharmacological, dietary, behavioral, or compositional means that achieves the recited ketotic state —circulating BHB at or above 0.5 mM with sufficient target-engagement duration-falls within the scope of the methods and compositions described herein unless expressly excluded in a specific claim. The enumeration of ketogenic agents within the operative definitions of the present disclosure-including dietary restriction, fasting, ketone monoester, ketone salt, 1,3-butanediol, medium-chain triglyceride, ketone-carrier conjugate, direct ketone infusion, SGLT2 inhibitor, NAD⁺ precursor, PPAR agonist, and mitochondrial biogenesis enhancer—is not exhaustive; any means of elevating circulating ketone bodies to therapeutic levels is encompassed. This broad means-agnostic scope reflects the Ketosis=Ketosis principle: the therapeutic efficacy of the ketotic state is determined by circulating BHB concentration and target-engagement duration, not by the particular means of induction. A competitor that develops a novel ketogenic agent not yet described in the literature-whether a new chemical entity, a new dietary formulation, or a new device-based means of inducing ketosis-would nonetheless fall within the scope of the methods claims if it achieves the recited circulating BHB level for the recited duration and thereby engages the recited MERIT mechanisms.

In another aspect, provided herein is a method of treating a metabolic-epigenetic-redox-immune (MERIT) responsive condition in a subject, comprising:
administering to the subject a therapeutically effective amount of a ketogenic agent selected from the group consisting of: (a) dietary restriction in the form of a ketogenic diet; (b) fasting selected from intermittent fasting and prolonged fasting; (c) an exogenous ketone monoester comprising (R)-3-hydroxybutyryl-(R)-3-hydroxybutyrate or an analog thereof; (d) an exogenous ketone salt selected from sodium β-hydroxybutyrate, potassium β-hydroxybutyrate, calcium β-hydroxybutyrate, sodium acetoacetate, potassium acetoacetate, and combinations thereof; (e)_{1,3}-butanediol or a derivative thereof; (f) a medium-chain triglyceride selected from C6, C8, C10, and C12 triglycerides; (g) a ketone-carrier conjugate comprising a polyol linked via a linker to one or more ketone bodies; (h) an SGLT2 inhibitor selected from empagliflozin, dapagliflozin, canagliflozin, ertugliflozin, and combinations thereof; (i) any combination of (a)-(h);
in an amount sufficient to induce and maintain in the subject a circulating β-hydroxybutyrate concentration of at least 0.5 millimolar (mM) for a duration of at least 7 days, thereby engaging metabolic state remodeling, epigenetic chromatin deacetylation, redox rebalancing, and immune tolerance;
wherein the method does not require that the subject exhibit glucose hypometabolism prior to, during, or after said administration, and is independent of dietary macronutrient restriction beyond that inherent to the ketogenic agent itself.

In some embodiments, the ketogenic agent is a dietary ketogenic diet comprising a macronutrient ratio of carbohydrate: protein: fat of approximately 5:20:75 by caloric weight.

In some embodiments, the ketogenic agent is fasting, and the subject undergoes intermittent fasting comprising 16-hour or 24-hour fasting windows separated by 8-hour or 12-hour feeding windows, maintained for a duration of at least 14 days.

In some embodiments, the ketogenic agent is an exogenous ketone monoester administered orally at a dose of 0.5-2.0 grams per dose, once or twice daily.

In some embodiments, the ketogenic agent is an exogenous ketone salt administered orally at a dose of 1-5 grams per day, divided into one or more doses.

In some embodiments, the ketogenic agent is 1,3-butanediol administered orally at a dose of 0.25-1.0 gram per kilogram of body weight per day.

In some embodiments, the ketogenic agent is a medium-chain triglyceride, specifically C8 (octanoic acid triglyceride), administered at a dose of 5-30 milliliters per day.

In some embodiments, the ketogenic agent is an SGLT2 inhibitor, and the subject is administered empagliflozin 10-25 mg daily or dapagliflozin 5-10 mg daily.

In some embodiments, the ketogenic agent is a combination of dietary ketogenic restriction and intermittent fasting.

In some embodiments, the ketogenic agent is a therapeutically effective amount of a compound of Formula (A):

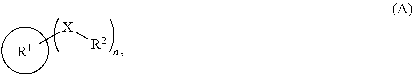

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH—or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

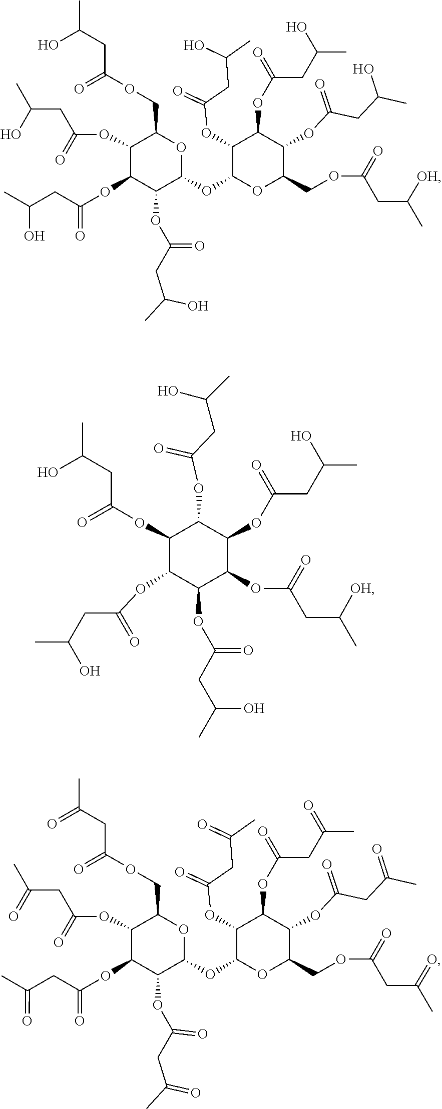

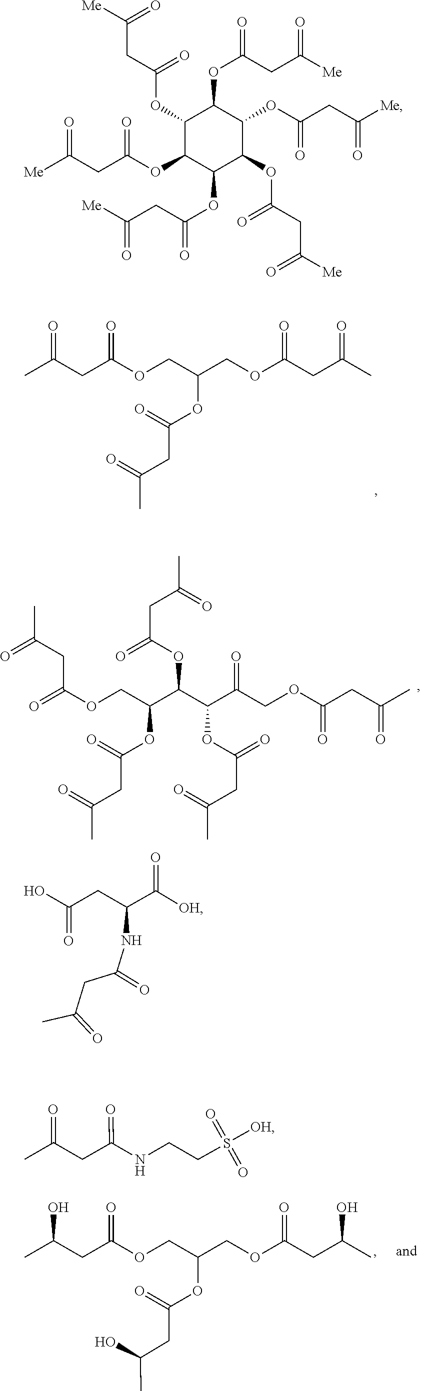

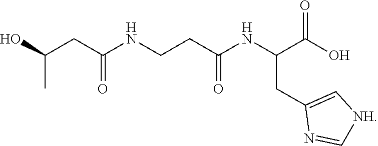 [SMILES: C[C@@H](O)CC(=O)NCCC(=O)NC(Cc1c[nH]cn1)C(=O)O]

In some embodiments, the circulating β-hydroxybutyrate concentration is maintained at 1.0-3.0 mM.

In some embodiments, the circulating β-hydroxybutyrate concentration is maintained at 1.5-5.0 mM.

In some embodiments, the duration of maintenance of said circulating β-hydroxybutyrate concentration is at least 14 days, 28 days, 90 days, or longer.

In some embodiments, the method further comprises measuring baseline and on-treatment MERIT biomarkers selected from circulating β-hydroxybutyrate, urinary ketones, adipose tissue gene expression profile, peripheral blood mononuclear cell (PBMC) NAD⁺/NADH ratio, NLRP3 inflammasome activity, HCAR2 signaling activity, plasma histone deacetylase (HDAC) activity, and circulating IL-1β and IL-18 levels.

In some embodiments, the subject is administered said ketogenic agent in an ambulatory setting, without hospitalization or intensive monitoring.

In some embodiments, the subject is not required to exhibit abnormal fasting glucose, glucose intolerance, or impaired glucose oxidation capacity prior to, during, or after administration.

In some embodiments, the therapeutic benefit is mediated, in part, by suppression of NLRP3 inflammasome activation, as evidenced by reduced circulating IL-1β or IL-18 levels during or within 7 days of reaching target BHB concentration.

In some embodiments, the therapeutic benefit is mediated, in part, by elevation of the NAD⁺/NADH ratio in metabolically active cell populations, resulting in increased SIRT family deacetylase activity and enhanced cellular stress resistance.

In some embodiments, the method comprises administering the ketogenic agent to a subject in an outpatient clinic or home setting, with periodic monitoring of circulating BHB concentration at days 3, 7, 14, and 28, and dose adjustment to maintain target BHB threshold.

In another aspect, provided herein is a method of increasing the therapeutic efficacy of a second pharmaceutical agent in a subject having a MERIT-responsive condition, comprising:
inducing in the subject a ketotic metabolic state by administering a ketogenic agent provided herein, in an amount sufficient to achieve and maintain a circulating β-hydroxybutyrate concentration of at least 0.5 mM for a duration of at least 7 days;
subsequently administering to the subject said second pharmaceutical agent in a dose and schedule that would be therapeutically ineffective or sub-efficacious in the absence of the ketotic state;
wherein the second pharmaceutical agent exhibits greater therapeutic efficacy, as measured by tumor response rate, disease remission rate, symptom reduction, or biomarker improvement, when administered during or immediately following the ketotic state compared to historical baseline or control populations receiving said second agent without concomitant ketotic state induction;
wherein the method does not require that the subject exhibit glucose hypometabolism, and is independent of dietary restriction of the subject.

In some embodiments, the second pharmaceutical agent is an immune checkpoint inhibitor selected from anti-PD-1 (nivolumab, pembrolizumab), anti-PD-L1 (durvalumab, atezolizumab), anti-CTLA-4 (ipilimumab, tremelimumab), and combinations thereof.

In some embodiments, the second pharmaceutical agent is a tyrosine kinase inhibitor or serine/threonine kinase inhibitor selected from sunitinib, sorafenib, erlotinib, gefitinib, and PI3K pathway inhibitors (alpelisib, idelalisib).

In some embodiments, the second pharmaceutical agent is a chemotherapeutic agent selected from taxanes, platinum compounds, antimetabolites, and targeted biologics.

In some embodiments, the second pharmaceutical agent is a psychiatric or neurologic agent selected from selective serotonin reuptake inhibitors (SSRIs), serotonin-norepinephrine reuptake inhibitors (SNRIs), mood stabilizers, and antiepileptic agents.

In some embodiments, the subject previously failed to respond to said second agent when administered as monotherapy in an unselected population, and the ketotic state induction results in conversion of said subject from non-responder to responder phenotype.

In some embodiments, the second pharmaceutical agent is administered during the maintenance phase of the ketotic state, defined as the period during which circulating β-hydroxybutyrate is maintained at or above 0.5 mM.

In some embodiments, the second pharmaceutical agent is administered within 0-14 days prior to initiation of ketotic state induction, allowing pre-treatment sensitization.

In some embodiments, the subject exhibits a baseline MERIT biomarker signature consistent with metabolic dysfunction, epigenetic heterochromatin deposition, or immune exhaustion, and the ketotic state induction reverses said signature as measured by on-treatment biomarker readout.

In some embodiments, the therapeutic efficacy improvement is quantified as: (a) absolute response rate improvement of at least 15%, (b) progression-free survival improvement of at least 3 months, or (c) clinical symptom improvement by at least 50% relative to historical monotherapy controls.

In some embodiments, the ketogenic agent is administered concurrently with the second pharmaceutical agent for the entire duration of second agent treatment.

In some embodiments, the method comprises withdrawing the ketogenic agent after the subject achieves sustained response (complete response, partial response, or stable disease lasting ≥12 weeks) and monitoring for loss of response; if loss of response occurs within 8 weeks, re-initiating ketotic state induction.

In some embodiments, the second pharmaceutical agent is an antimicrobial (antibiotic or antiviral) agent, and the ketotic state enhances immune-mediated clearance of chronic viral or bacterial infection, as evidenced by viral or bacterial load reduction or serologic conversion.

In some embodiments, the second pharmaceutical agent is an immunotherapy agent, and the ketotic state induction precedes said agent administration by 3-14 days to allow epigenetic and immune remodeling.

In some embodiments, the method comprises restoring therapeutic efficacy to said second pharmaceutical agent in a subject in whom said agent previously failed to meet an efficacy endpoint in a clinical trial conducted in an unselected population, by inducing a concurrent ketotic state.

In some embodiments, the circulating β-hydroxybutyrate concentration is maintained at 1.0-3.0 mM during second agent administration.

In some embodiments, the subject is assessed for baseline MERIT biomarker dysregulation prior to induction, and the ketotic state is initiated only if the subject meets criteria for MERIT-responsive phenotype.

In some embodiments, the method is practiced in an ambulatory or clinic setting, without requirement for hospitalization, and comprises home-based monitoring of ketotic state achievement via urinary ketone strips or point-of-care serum BHB measurement.

In another aspect, provided herein is a method of treating an immune-inflammatory condition characterized by dysregulation of NLRP3 inflammasome-mediated inflammation, in a subject not required to exhibit glucose hypometabolism, comprising:
administering a ketogenic agent provided herein in an amount sufficient to induce and maintain a circulating β-hydroxybutyrate concentration of at least 0.5 mM for at least 7 days;
wherein the ketotic state suppresses NLRP3 inflammasome activation, as evidenced by reduced circulating IL-1β, IL-18, or IL-6 levels;
wherein the method does not require that the subject exhibit glucose hypometabolism, and is independent of dietary macronutrient restriction beyond that inherent to the ketogenic agent itself.

In some embodiments, the immune-inflammatory condition is selected from rheumatoid arthritis, systemic lupus erythematosus, Sjögren's syndrome, and scleroderma.

In some embodiments, the immune-inflammatory condition is inflammatory bowel disease, comprising Crohn's disease or ulcerative colitis, and the therapeutic endpoint is reduction in fecal calprotectin, endoscopic disease activity, or clinical symptom score (Crohn's Disease Activity Index, Mayo Score).

In some embodiments, the method further comprises co-administering a standard-of-care immunosuppressive or biologic agent, such as a TNF-α inhibitor, IL-6 receptor antagonist, or JAK inhibitor, wherein the ketotic state potentiates the efficacy of said biologic.

In some embodiments, the method comprises measuring baseline and on-treatment inflammasome biomarkers (circulating IL-1β, IL-18, caspase-1 activity) and continuing ketotic state induction if biomarkers remain elevated, or discontinuing if biomarkers normalize.

In some embodiments, the subject achieves clinical remission (defined as absence of symptoms, return to normal function, or resolution of objective inflammation markers) and the ketotic state is maintained chronically to prevent relapse.

In another aspect, provided herein is a method of treating a dermatologic condition characterized by inflammatory infiltrate and dysregulated cutaneous immunity, in a subject not required to exhibit glucose hypometabolism, comprising:
administering a ketogenic agent provided herein in an amount sufficient to achieve and maintain a circulating β-hydroxybutyrate concentration of at least 1.0 mM for at least 14 days;
wherein the ketotic state remodels cutaneous immune cell function and reduces inflammatory cytokine production;
measuring clinical endpoints including lesion erythema, induration, extent of affected body surface area, and pruritus intensity;
wherein the method does not require that the subject exhibit glucose hypometabolism, and is independent of dietary restriction of the subject.

In some embodiments, the dermatologic condition is atopic dermatitis, and the therapeutic endpoint is improvement in Eczema Area and Severity Index (EASI) score by at least 50% or achievement of clear/almost clear status on Investigator's Global Assessment (IGA).

In some embodiments, the dermatologic condition is psoriasis, and the therapeutic endpoint is improvement in Psoriasis Area and Severity Index (PASI) score by at least 75% (PASI-75 response).

In some embodiments, the method further comprises biopsying affected skin at baseline and week 4 of ketotic state induction and analyzing for reduction in inflammatory immune infiltrate (T cell, B cell, macrophage, and dendritic cell density) by immunohistochemistry or flow cytometry.

In some embodiments, the dermatologic condition is selected from psoriatic arthritis, lichen planus, and alopecia areata.

In another aspect, provided herein is a method of treating a psychiatric or neurologic condition mediated at least in part by reduced nicotinamide adenine dinucleotide (NAD⁺) signaling, histone deacetylase (HDAC) dysregulation, or metabolic insufficiency in the central nervous system, in a subject not required to exhibit glucose hypometabolism, comprising:
administering a ketogenic agent provided herein in an amount sufficient to induce and maintain a circulating β-hydroxybutyrate concentration of at least 1.0 mM for at least 7 days;
wherein the circulating and central nervous system BHB acts as a substrate for NAD+-dependent signaling, inhibits HDAC activity via histone acetylation, and enhances mitochondrial function in neuronal and glial cells;
measuring clinical endpoints including symptom severity score, functional outcome measure, and mood or cognition assessment;
wherein the method does not require that the subject exhibit glucose hypometabolism, and is independent of dietary restriction of the subject.

In some embodiments, the psychiatric condition is major depressive disorder, and the subject is treatment-resistant (failure of at least two first-line serotonergic antidepressants), and the therapeutic endpoint is improvement in Montgomery-Åsberg Depression Rating Scale (MADRS) score by at least 50%.

In some embodiments, the psychiatric condition is bipolar disorder, depressive or manic episode, and the ketotic state induction is combined with mood-stabilizing pharmacotherapy (lithium, valproate, lamotrigine).

In some embodiments, the neurologic condition is Alzheimer's disease or mild cognitive impairment, and the therapeutic endpoint is stabilization or improvement in Mini-Cog score, Montreal Cognitive Assessment (MoCA), or functional independence measure.

In some embodiments, the neurologic condition is Parkinson's disease, epilepsy, or amyotrophic lateral sclerosis, and the therapeutic endpoint is improvement in motor function score, seizure frequency reduction, or slowing of motor decline.

In another aspect, provided herein is a method of treating a bacterial or viral infection, including chronic infection or sepsis, in a subject not required to exhibit glucose hypometabolism, comprising:
administering a ketogenic agent provided herein in an amount sufficient to induce and maintain a circulating β-hydroxybutyrate concentration of at least 0.5 mM for a duration dependent on infection type and severity;
wherein the ketotic state suppresses NLRP3 inflammasome-mediated pathologic inflammation while preserving or enhancing adaptive immune response (T cell and B cell function), and
co-administering an antimicrobial agent (antibiotic or antiviral) specific to the pathogen;
measuring endpoints including pathogen load (viral titer, bacterial colony—forming units), inflammatory biomarkers (IL-1β, TNF-α, IL-6), organ function (creatinine, transaminases, coagulation parameters), and clinical outcome (survival, ventilator-free days, ICU-free days);
wherein the method does not require that the subject exhibit glucose hypometabolism, and is independent of dietary restriction of the subject.

In some embodiments, the infection is gram-negative bacterial sepsis, and the ketotic state induction is initiated within 24-48 hours of diagnosis, concurrently with broad-spectrum antibiotics pending culture results.

In some embodiments, the infection is a chronic viral infection selected from cytomegalovirus (CMV), Epstein-Barr virus (EBV), human immunodeficiency virus (HIV), or hepatitis C virus (HCV), and the ketotic state enhances viral-specific T cell function, as measured by CD8+ T cell cytotoxic response or reduction in viral load.

In some embodiments, the infection is coronavirus infection (SARS-COV-2 or other), and the therapeutic endpoint is improved oxygenation, reduced progression to mechanical ventilation, and shortened hospitalization duration compared to matched controls receiving antimicrobial therapy without ketotic state induction.

In another aspect, provided herein is a method of treating an oncologic indication via immune checkpoint inhibitor therapy combined with metabolic state manipulation, in a subject not required to exhibit glucose hypometabolism, comprising:
inducing a ketotic metabolic state by administering a ketogenic agent provided herein in an amount sufficient to achieve and maintain a circulating β-hydroxybutyrate concentration of at least 1.0 mM;
maintaining said ketotic state for a period of 7-14 days prior to the first dose of immune checkpoint inhibitor therapy;
subsequently administering an immune checkpoint inhibitor selected from anti-PD-1, anti-PD-L1, anti-CTLA-4, or combinations thereof;
maintaining concurrent ketotic state induction throughout the duration of checkpoint inhibitor therapy;
measuring clinical endpoints including objective response rate, duration of response, progression-free survival, and overall survival;
wherein the ketotic state remodels tumor-associated immune dysfunction, reduces tumor-infiltrating regulatory T cells and myeloid-derived suppressor cells, enhances tumor-infiltrating effector T cell function, and increases tumor immunogenicity;
wherein the method does not require that the subject exhibit glucose hypometabolism, and is independent of dietary restriction of the subject.

In some embodiments, the oncologic indication is melanoma, and the therapeutic endpoint is objective response rate improvement from historical 30-40% monotherapy baseline to >60% with combined ketotic state induction and checkpoint inhibitor.

In some embodiments, the oncologic indication is non-small-cell lung cancer (NSCLC), renal cell carcinoma (RCC), hepatocellular carcinoma (HCC), or colorectal cancer (CRC), and the therapeutic endpoint is checkpoint inhibitor efficacy restoration in previously refractory disease populations.

In some embodiments, the subject undergoes baseline tumor biopsy and transcriptomic profiling to assess tumor-infiltrating lymphocyte (TIL) density, PD-L1 expression, myeloid cell infiltration, and metabolic gene expression signature; ketotic state induction is continued if baseline profiling indicates cold tumor phenotype (low TIL, high myeloid suppression); and on-treatment biopsy at week 4 is obtained to document immune remodeling.

In another aspect, provided herein is a method of selecting and monitoring subjects for ketosis-based therapeutic intervention, comprising:
measuring in a biological sample obtained from the subject at least one MERIT biomarker selected from the group consisting of: (i) circulating β-hydroxybutyrate concentration; (ii) urinary ketone concentration; (iii) adipose tissue transcriptomic signature indicating dysregulated energy metabolism; (iv) peripheral blood mononuclear cell (PBMC) NAD⁺/NADH ratio; (v) NLRP3 inflammasome activity, as measured by circulating IL-1β, IL-18, or caspase-1 activity; (vi) HCAR2 signaling activity in immune cell populations; (vii) histone deacetylase (HDAC) activity; (viii) a composite metabolic vulnerability index derived from a multiplex biomarker panel;
comparing the measured biomarker value(s) to an established threshold or reference range;
selecting for ketosis-based therapeutic intervention any subject whose biomarker value(s) indicate dysregulation of metabolic state, epigenetic tone, redox balance, or immune function;
wherein the method enables identification of MERIT-responsive subjects independent of traditional disease classification;
wherein the method does not require that the subject exhibit glucose hypometabolism; and wherein the method is independent of dietary restriction of the subject.

In some embodiments, the sample is venous blood or capillary blood, and circulating β-hydroxybutyrate is measured by liquid chromatography-mass spectrometry (LC-MS), gas chromatography-mass spectrometry (GC-MS), or colorimetric enzyme assay.

In some embodiments, the PBMC NAD⁺/NADH ratio is measured by high-performance liquid chromatography (HPLC) or mass spectrometry, and a ratio of <2.0 indicates dysregulation warranting ketosis-based intervention.

In some embodiments, NLRP3 inflammasome activity is measured by quantifying circulating IL-1β (>50 μg/mL) or IL-18 (>500 μg/mL) using high-sensitivity immunoassay.

In some embodiments, the composite metabolic vulnerability index is derived from a machine-learning algorithm incorporating >5 biomarkers, weighted by their association with MERIT-responsive disease phenotype, and a calculated index>0.6 (on a scale of 0-1) indicates ketosis-based intervention candidacy.

In another aspect, provided herein is a method of monitoring therapeutic response to a ketogenic agent in a subject with a MERIT-responsive condition, comprising:
measuring baseline target-engagement biomarkers in said subject selected from the group consisting of NLRP3 inflammasome activity, HCAR2 signaling activity, PBMC NAD⁺/NADH ratio, HDAC activity, circulating metabolic substrate ratios, and epigenetic chromatin histone acetylation state;
initiating ketogenic agent administration in an amount sufficient to induce and maintain a circulating β-hydroxybutyrate concentration of at least 0.5 mM;
measuring on-treatment target-engagement biomarkers at defined timepoints (days 3, 7, 14, 28);
continuing ketogenic agent administration if target-engagement biomarkers demonstrate the intended mechanistic effect;
adjusting ketogenic agent dose or switching to an alternative ketogenic agent provided herein if target-engagement biomarkers fail to demonstrate expected engagement;
measuring clinical outcome biomarkers (disease-specific endpoints) at days 7, 14, 28, and 90 to assess therapeutic efficacy;
wherein target-engagement biomarker-guided adjustment optimizes therapeutic response independent of empirical dose escalation alone;
wherein the method does not require that the subject exhibit glucose hypometabolism, and is independent of dietary restriction of the subject.

In some embodiments, the target-engagement biomarker is NLRP3 inflammasome activity (circulating IL-1β or IL-18), and a reduction of ≥30% from baseline indicates adequate MERIT target engagement.

In some embodiments, the target-engagement biomarker is PBMC NAD⁺/NADH ratio, and an elevation to ≥2.5 indicates restoration of NAD+-dependent signaling.

In some embodiments, the clinical outcome biomarker is disease-specific (e.g., EASI for atopic dermatitis, PASI for psoriasis, MADRS for depression, tumor response rate for oncology), and a predefined improvement threshold triggers assessment of biomarker-guided dose optimization.

In some embodiments, the method further comprises measuring circulating β-hydroxybutyrate at each timepoint to confirm adequate ketotic state induction (≥0.5 mM), and escalating ketogenic agent dose if BHB remains below threshold.

In some embodiments, the method comprises performing optional adipose tissue or tumor biopsy and transcriptomic analysis at baseline and week 4, to directly assess epigenetic and metabolic gene expression changes in response to ketotic state induction.

In another aspect, provided herein is a kit for MERIT-responsive subject selection and therapeutic monitoring, comprising:
(a) a ketogenic agent provided herein;
(b) one or more MERIT biomarker assay(s) provided herein in a form suitable for point-of-care or laboratory measurement;
(c) a reference table or nomogram relating biomarker values to MERIT-responsive phenotype and ketosis-based intervention eligibility;
(d) written instructions for subject selection, ketogenic agent closing, on-treatment biomarker monitoring at defined intervals (days 3, 7, 14, 28, 90), and dose adjustment criteria;
(e) optionally: a portable meter or assay cartridge for measuring circulating β-hydroxybutyrate in a clinic or home setting;
wherein the kit is configured for use with subjects who do not exhibit glucose hypometabolism, and is independent of dietary restriction of the subject.

In some embodiments, the ketogenic agent is an oral exogenous ketone salt or monoester, pre—formulated in unit-dose packets, oral liquid, suspension, or tablets.

In some embodiments, the MERIT biomarker assay comprises a multiplexed immunoassay panel for simultaneous measurement of circulating IL-1β, IL-18, TNF-α, and IL-6.

In some embodiments, the written instructions comprise a treat-to-target algorithm specifying circulating β-hydroxybutyrate target of 1.0-3.0 mM, measurement frequency, and decision rules for dose escalation, maintenance, or discontinuation based on biomarker readout and clinical response.

In another aspect, provided herein is a pharmaceutical composition for enhancing therapeutic efficacy of a second pharmaceutical agent via concomitant metabolic state modulation, comprising:
(a) a ketogenic agent selected from the group consisting of: (i) an exogenous ketone monoester comprising (R)-3-hydroxybutyryl-(R)-3-hydroxybutyrate; (ii) an exogenous ketone salt selected from sodium, potassium, or calcium β-hydroxybutyrate; (iii) a ketone-carrier conjugate comprising a polyol linked to one or more ketone bodies; (iv) 1,3-butanediol or a derivative thereof; (v) a medium-chain triglyceride; (vi) an SGLT2 inhibitor selected from empagliflozin, dapagliflozin, canagliflozin, or ertugliflozin;
(b) a second pharmaceutical agent selected from the group consisting of: (i) an immune checkpoint inhibitor (anti-PD-1, anti-PD-L1, anti-CTLA-4); (ii) a kinase inhibitor (PI3K, MEK, JAK, or tyrosine kinase inhibitor); (iii) a chemotherapeutic agent; (iv) a psychiatric or neurologic agent (SSRI, SNRI, mood stabilizer, antiepileptic); (v) an antimicrobial agent (antibiotic or antiviral); (vi) a biologic immune-modulating agent; (vii) optionally, a pharmaceutically acceptable excipient, carrier, or vehicle;
formulated for co-administration in amounts sufficient to (1) induce and maintain a circulating-hydroxybutyrate concentration of at least 0.5 mM, and (2) achieve therapeutic concentrations of the second agent at the site of action;
wherein the composition is formulated to provide synergistic or super-additive therapeutic benefit via state-dependent enhancement of second agent efficacy;
wherein the composition does not require that the subject exhibit glucose hypometabolism, and is independent of dietary restriction of the subject.

In some embodiments, the ketogenic agent is an exogenous ketone monoester and the second pharmaceutical agent is an immune checkpoint inhibitor, co—formulated in a unit-dose oral preparation.

In some embodiments, the ketogenic agent is a ketone-carrier conjugate and the second pharmaceutical agent is a PI3K inhibitor or JAK inhibitor, co—formulated as a fixed-dose combination oral liquid or suspension.

In some embodiments, the ketogenic agent and second pharmaceutical agent are formulated in separate, temporally staggered unit-dose forms, with instructions to administer the ketogenic agent 7-14 days prior to the second agent, to allow for metabolic priming.

In some embodiments, the composition further comprises a MERIT biomarker assay kit or device to enable patient selection or on-treatment therapeutic monitoring.

In another aspect, provided herein is a kit for treating a MERIT-responsive condition via combined ketosis induction and therapeutic intervention, comprising:
(a) a ketogenic agent in a form suitable for oral or intravenous administration, in a unit-dose or multi-dose presentation;
(b) a second pharmaceutical agent provided herein in a therapeutic unit-dose;
(c) a MERIT biomarker assay or point-of-care measurement device provided herein;
(d) written instructions comprising: (i) subject selection criteria based on MERIT biomarker profile; (ii) ketogenic agent closing, administration route, and frequency; (iii) timing of second agent administration relative to ketogenic agent initiation; (iv) on-treatment biomarker monitoring schedule and decision rules; (v) clinical outcome assessment timepoints and target endpoints; (vi) safety monitoring parameters and dose adjustment algorithms;
(e) optionally: a patient adherence tracking tool, such as a mobile application or paper diary;
wherein the kit is configured for use with subjects who do not exhibit glucose hypometabolism, and is independent of dietary restriction of the subject.

In some embodiments, the written instructions specify that the ketogenic agent shall be administered to achieve and maintain a circulating β-hydroxybutyrate concentration of 1.0-3.0 mM for a minimum of 7 days prior to initiation of the second agent.

In some embodiments, the ketogenic agent comprises a combination of an exogenous ketone salt and an SGLT2 inhibitor, formulated for concurrent or sequential administration.

In some embodiments, the MERIT biomarker assay comprises a multiplexed panel enabling measurement of at least three biomarkers selected from circulating BHB, NLRP3 inflammasome activity, PBMC NAD⁺/NADH ratio, and HCAR2 signaling activity.

In another aspect, provided herein is a therapeutic combination product comprising a ketogenic agent and a diagnostic biomarker measurement component, integrated for use in identifying and treating MERIT-responsive subjects, comprising:
(a) a pharmaceutical composition containing a ketogenic agent provided herein;
(b) an integrated diagnostic device or assay capable of measuring a MERIT biomarker provided herein in a real-time or near-real-time fashion (results available within 5-60 minutes);
(c) a computational algorithm or display interface that correlates the measured biomarker value(s) to subject MERIT-responsiveness and provides a recommendation for (i) ketogenic agent initiation, (ii) dose, (iii) duration, or (iv) discontinuation;
(d) written instructions for use, including subject selection, biomarker measurement procedure, interpretation, and ketogenic agent administration guidance;
wherein the combination product enables bedside or clinic-based MERIT phenotyping and real-time therapeutic guidance;
wherein the composition does not require that the subject exhibit glucose hypometabolism, and is independent of dietary restriction of the subject.

In some embodiments, the diagnostic device is a point-of-care meter measuring circulating β-hydroxybutyrate from a finger-stick blood sample or breath acetone.

In some embodiments, the computational algorithm incorporates machine-learning classification trained on MERIT-responsive and MERIT-non-responsive subject populations, and generates a MERIT-responsiveness probability score (0-100%) based on measured biomarker value(s).

In some embodiments, the ketogenic agent is pre-packaged in a single-unit dose form with instructions specifying administration only if the biomarker measurement indicates MERIT-responsive status (e.g., circulating BHB<0.5 mM at baseline, NLRP3 activity >threshold, NAD⁺/NADH ratio <2.0).

In another aspect, provided herein is a method of manufacturing a fixed-dose combination pharmaceutical composition comprising a ketogenic agent and a second therapeutic agent, comprising:
mixing a therapeutically effective amount of a ketogenic agent provided herein with a therapeutically effective amount of a second agent provided herein;
incorporating the mixture into a pharmaceutically acceptable carrier, excipient, or vehicle; forming unit-dose forms (oral liquids, suspensions, tablets, capsules, sachets, or vials);
quantitatively assaying the final composition to verify target β-hydroxybutyrate equivalent concentration (molar basis) and second agent concentration;
packaging the unit-dose forms with labeling and instructions specifying use for MERIT-responsive condition treatment;
wherein the fixed-dose combination is manufactured to ensure stoichiometric equivalence across unit doses, enabling predictable bioavailability and pharmacodynamic target engagement;
wherein the method does not require that the subject exhibit glucose hypometabolism, and is independent of dietary restriction of the subject.

In some embodiments, the ketogenic agent is an exogenous ketone salt and the second agent is an SGLT2 inhibitor, and the unit-dose form is an oral liquid or suspension comprising 2 grams of ketone salt and 10 mg of SGLT2 inhibitor.

In some embodiments, the assay comprises high-performance liquid chromatography (HPLC) or mass spectrometry to quantify ketone body concentration and second agent concentration independently, ensuring compositional consistency within +10% of target values.

In another aspect, provided herein is a pharmaceutical composition in a form suitable for oral administration to a human subject, comprising:
(a) a ketogenic agent selected from the group consisting of an exogenous ketone monoester, an exogenous ketone salt, a ketone-carrier conjugate, 1,3-butanediol, or a medium-chain triglyceride;
(b) a taste-masking agent selected from the group consisting of microcrystalline cellulose, silica, citric acid, or a proprietary taste-masking polymer;
(c) a bioavailability enhancer selected from the group consisting of permeation enhancers, surfactants, or lipid-based formulation excipients;
(d) optionally, a coloring agent approved for food or pharmaceutical use;
(e) optionally, a sweetening agent;
formulated as an oral liquid, suspension, powder sachet, tablet, or capsule;
wherein the composition is formulated to optimize palatability and oral bioavailability of the ketogenic agent, enabling consistent achievement of circulating β-hydroxybutyrate concentrations of at least 0.5 mM within 60-120 minutes of administration.

In another aspect, provided herein is a method of treating a MERIT-responsive condition in a subject, comprising: administering a ketogenic agent in an amount sufficient to achieve a target ratio of circulating β-hydroxybutyrate to acetoacetate (BHB:AcAc) in the range of about 1:1 to about 5:1; monitoring the BHB:AcAc ratio by measuring both circulating BHB and circulating AcAc; and adjusting the dose or composition of the ketogenic agent to maintain the target BHB:AcAc ratio for a duration sufficient to engage at least one of histone β-hydroxybutyrylation, NLRP3 inflammasome suppression, or HCAR2 signaling; wherein the method does not require that the subject exhibit glucose hypometabolism; and wherein the method is independent of dietary restriction of the subject.

In some embodiments, AcAc is measured by LC-MS/MS or enzymatic assay in a biological sample from the subject.

In some embodiments, the target BHB:AcAc ratio is optimized for the specific MERIT-responsive condition.

In some embodiments, the ketogenic agent is a ketone-carrier conjugate wherein R² comprises acetoacetate, such that AcAc is delivered directly rather than relying solely on hepatic interconversion from BHB.

In some embodiments, the method further comprises measuring total ketone body exposure (BHB+AcAc+acetone) and adjusting the dose to maintain a total ketone body concentration of at least 0.8 mM.

In some embodiments, the method further comprises measuring breath acetone concentration as a non-invasive surrogate for circulating acetoacetate.

In some embodiments, the BHB:AcAc ratio is used to stratify the subject for selection of a specific ketogenic agent from the group consisting of a BHB-dominant composition, an AcAc-dominant composition, and a dual-ketone composition.

In some embodiments, the MERIT-responsive condition is selected from the group consisting of epilepsy, psychiatric disorder, neurodegenerative disorder, and oncology, and the target BHB:AcAc ratio is indication-specific.

In some embodiments, the AcAc-dominant composition achieves lysine acetoacetylation (Kacac) at histone sites distinct from β-hydroxybutyrylation (Kbhb), thereby engaging an epigenetic program complementary to but distinct from BHB-mediated Kbhb.

In some embodiments, the method further comprises measuring site-specific histone modifications selected from the group consisting of K18ac, K27ac, K27bhb, and K36bhb to verify target engagement of the optimized BHB:AcAc ratio.

In another aspect, provided herein is a method of treating a MERIT-responsive condition in a subject, comprising: determining a circadian phase of the subject by measuring at least one circadian biomarker selected from the group consisting of cortisol awakening response, core body temperature nadir timing, melatonin onset, and clock gene expression level of at least one of BMAL1, PER2, CRY1, and CLOCK; administering a ketogenic agent at a time of day selected to align peak circulating β-hydroxybutyrate concentration with a target circadian phase of the subject; wherein the target circadian phase is selected to maximize engagement of at least one MERIT pillar mechanism; wherein the method does not require that the subject exhibit glucose hypometabolism; and wherein the method is independent of dietary restriction of the subject.

In some embodiments, the target circadian phase is the morning cortisol peak phase and the MERIT pillar mechanism is BMAL1-mediated circadian metabolic synchronization.

In some embodiments, the target circadian phase is the evening inflammatory peak phase and the MERIT pillar mechanism is NLRP3 inflammasome suppression.

In some embodiments, the method further comprises co-administering a second therapeutic agent, and the ketogenic agent is administered at a time selected to align peak BHB concentration with peak plasma concentration (Cmax) of the second therapeutic agent.

In some embodiments, the second therapeutic agent is an immune checkpoint inhibitor and the aligned pharmacokinetic windows maximize T-cell metabolic fitness during peak drug-target interaction.

In some embodiments, the circadian biomarker is measured using a wearable device that monitors at least one of skin temperature, heart rate variability, and actigraphy to infer circadian phase.

In some embodiments, the method is repeated daily with adaptive timing adjustment based on serial circadian biomarker measurements.

In some embodiments, the subject has a circadian rhythm disorder and the method further comprises using the ketogenic agent to entrain the circadian clock via BHB-mediated activation of BMAL1 and PER2.

In some embodiments, the ketogenic agent is administered in a modified-release formulation designed to produce a BHB exposure profile that matches a predetermined circadian target curve.

In some embodiments, the MERIT-responsive condition is selected from the group consisting of a psychiatric disorder characterized by circadian disruption, a neurodegenerative disorder characterized by sleep-wake cycle dysregulation, and an oncology condition in which immune checkpoint receptor expression varies with circadian phase.

In another aspect, provided herein is a method of monitoring and optimizing therapeutic ketosis in a subject being treated for a MERIT-responsive condition, comprising: measuring circulating glucose concentration and circulating β-hydroxybutyrate concentration in the subject; calculating a Glucose-Ketone Index (GKI) according to the formula: GKI=[glucose (mg/dL)]/([BHB (mmol/L)]×18.01); comparing the calculated GKI to a target GKI threshold; and adjusting the dose of a ketogenic agent administered to the subject to achieve or maintain a GKI at or below the target GKI threshold; wherein the method does not require that the subject exhibit glucose hypometabolism; and wherein the method is independent of dietary restriction of the subject.

In some embodiments, the target GKI threshold is indication-specific and selected from: GKI≤1.0 for an oncology indication, GKI≤1.0 for a psychiatric indication, GKI≤1.2 for a sepsis or infectious disease indication, GKI≤2.0 for a neurodegenerative indication, and GKI≤3.0 for a metabolic or cardiovascular indication.

In some embodiments, glucose and BHB are measured using a point-of-care dual-analyte meter capable of measuring both glucose and BHB from a single capillary blood sample.

In some embodiments, the GKI is calculated in real-time by a software algorithm integrated with a continuous glucose monitor and a continuous or intermittent ketone monitor.

In some embodiments, the method further comprises measuring at least one MERIT biomarker selected from the group consisting of Kbhb, NLRP3 activity, and NAD+/NADH ratio, and using the combination of GKI and the at least one MERIT biomarker to guide dose adjustment.

In some embodiments, the subject is being co-administered a second therapeutic agent and the GKI target threshold is adjusted based on the pharmacodynamic requirements of the second agent.

In another aspect, provided herein is a kit comprising: a dual-analyte point-of-care meter for measuring glucose and β-hydroxybutyrate; instructions for calculating a Glucose-Ketone Index (GKI); and a reference card listing indication-specific target GKI thresholds for MERIT-responsive conditions; wherein use of the kit does not require that the subject exhibit glucose hypometabolism; and wherein use of the kit is independent of dietary restriction of the subject.

In another aspect, provided herein is a method of selecting a subject for ketosis-based therapy for a MERIT-responsive condition, comprising: measuring a baseline GKI in the subject; determining that the baseline GKI is above a threshold indicative of metabolic dysregulation for the subject's MERIT-responsive condition; and administering a ketogenic agent to the subject to reduce the GKI to at or below the indication-specific target threshold; wherein the method does not require that the subject exhibit glucose hypometabolism; and wherein the method is independent of dietary restriction of the subject.

In another aspect, provided herein is a method of treating a MERIT-responsive condition in a subject comprising: (a) determining whether the subject's condition is characterized as fuel-driven (where primary mechanism is energy substrate delivery to dysfunctional mitochondria) or signal-driven (where primary mechanism is activation of specific signaling pathways at defined concentration thresholds); (b) if fuel-driven, selecting a closing regimen of a ketogenic agent comprising high single-dose or infrequent (once to twice daily) administration to maximize total circulating β-hydroxybutyrate area-under-the-curve (AUC); (c) if signal-driven, selecting a closing regimen of a ketogenic agent comprising lower, frequent (three to four times daily) administration to maintain circulating β-hydroxybutyrate within a target concentration window (0.5-2.0 mM) for ≥2.6 hours daily; (d) administering the selected closing regimen; and (e) monitoring for therapeutic response; wherein the method does not require that the subject exhibit glucose hypometabolism; and wherein the method is independent of dietary restriction of the subject.

In some embodiments, the fuel-driven condition is selected from acute myocardial infarction with cardiogenic shock, severe traumatic brain injury, acute ischemic stroke, acute hepatic failure, acute mitochondrial depletion syndrome, or severe sepsis-associated mitochondrial dysfunction, and the ketogenic agent is dosed to achieve Cmax ≥1.0 mM and AUC ≥8 mM hours (measured as total BHB exposure above 0.5 mM baseline over a 24-hour period).

In some embodiments, the signal-driven condition is selected from NLRP3 inflammasome-driven autoinflammatory disease, autoimmune inflammatory disease, oncology requiring immune metabolic remodeling, or neuropsychiatric disease where epigenetic remodeling is primary, and the ketogenic agent is dosed to maintain circulating β-hydroxybutyrate 0.5-2.0 mM for ≥2.6 hours daily while remaining <2.4 mM.

In some embodiments, the method further comprises calculating a Glucose-Ketone Index (GKI) and adjusting dose or frequency to maintain an indication-specific target GKI.

In some embodiments, the determination of fuel-driven vs. signal-driven is made by assessing the predominant MERIT pillar(s) implicated in the condition: Metabolic pillar predominance indicates fuel-driven; Epigenetic, Immune, Redox, or Signaling pillar predominance indicates signal-driven.

In another aspect, provided herein is a method of optimizing pharmacologically-induced ketosis to simultaneously engage multiple beneficial mechanisms while avoiding off-target mechanisms, comprising: (a) identifying a subject with a MERIT-responsive condition where multiple beneficial mechanisms require engagement; (b) selecting a ketogenic agent and dose to maintain circulating β-hydroxybutyrate in a therapeutic optimization corridor of 0.5-2.0 mM for maximal duration per day, wherein the corridor simultaneously engages NLRP3 inflammasome suppression, HCAR2 signaling, and Kbhb histone modification while remaining below the class I HDAC engagement threshold of approximately 2.4 mM; (c) administering the selected ketogenic agent; and (d) measuring circulating BHB and at least one mechanism-specific biomarker to confirm multi-mechanism engagement; wherein the method does not require that the subject exhibit glucose hypometabolism; and wherein the method is independent of dietary restriction of the subject.

In some embodiments, the multi-mechanism engagement is confirmed by measuring at least two of: hi stone Kbhb enrichment, NLRP3 inflammasome activity in circulating monocytes, HCAR2-mediated signaling, and NAD+/NADH ratio.

In some embodiments, the method further comprises constructing a pharmacokinetic model describing BHB concentration as a function of dose, formulation, and time, and calculating integrated cumulative target engagement across NLRP3, HCAR2, and Kbhb mechanisms to identify the regimen maximizing integrated engagement within the 0.5-2.0 mM corridor.

In some embodiments, the ketogenic agent achieves pharmacokinetic parameters substantially equivalent to Study 25-101 at 5 g/kg: Cmax approximately 1.542 mM, time above 0.5 mM approximately 7.6 hours, time above 1.0 mM approximately 2.6 hours, and time above 2.4 mM approximately 0 hours.

In another aspect, provided herein is a method of treating a MERIT-responsive condition in a subject comprising: (a) classifying the subject's condition into one of the following categories: (i) sepsis or systemic infection; (ii) autoimmune or inflammatory disease; (iii) oncology with immune-exclusion phenotype; (iv) neurodegenerative disease; (v) psychiatric or mood disorder; (vi) cardiac disease; (vii) dermatologic disease; or (viii) kidney disease; (b) selecting an indication-specific target circulating β-hydroxybutyrate range based on the condition category: 0.5-1.0 mM for sepsis; 1.0-2.0 mM for autoimmune disease; 1.5-3.0 mM for oncology; 0.5-1.5 mM for neurodegeneration; 0.5-1.5 mM for psychiatry; 0.5-1.0 mM for cardiac disease; 0.5-1.5 mM for dermatology; or 0.5-1.0 mM for kidney disease; (c) administering a ketogenic agent in an amount to achieve the indication-specific target; and (d) measuring circulating BHB at intervals and adjusting dose to maintain the target range; wherein the method does not require that the subject exhibit glucose hypometabolism; and wherein the method is independent of dietary restriction of the subject.

In some embodiments, for sepsis (category i), the target range of 0.5-1.0 mM is selected to achieve rapid NLRP3 inflammasome suppression in innate immune cells.

In some embodiments, for autoimmune disease (category ii), the target range of 1.0-2.0 mM is selected to achieve sustained NLRP3 suppression combined with Kbhb-mediated regulatory T cell differentiation.

In some embodiments, for oncology (category iii), the target range of 1.5-3.0 mM is selected to achieve deep Kbhb deposition in tumor-infiltrating T cells for immune-metabolic remodeling of the tumor microenvironment.

In some embodiments, for neurodegenerative disease (category iv), the target range of 0.5-1.5 mM is selected to provide combined fuel-substrate replacement and HCAR2-mediated neuroinflammation suppression.

In some embodiments, for psychiatric disorder (category v), the target range of 0.5-1.5 mM is selected to achieve Kbhb-mediated epigenetic modulation in mood-circuit neurons combined with HCAR2 signaling in microglia.

In some embodiments, for cardiac disease (category vi), the target range of 0.5-1.0 mM is selected to provide myocardial energy substrate replacement and NAD+/NADH redox rebalancing.

In some embodiments, for dermatologic disease (category vii), the target range of 0.5-1.5 mM is selected to achieve NLRP3 suppression in skin-resident immune cells combined with restoration of epidermal barrier function.

In some embodiments, for kidney disease (category viii), the target range of 0.5-1.0 mM is selected to achieve renal mitochondrial protection and NLRP3 suppression in glomerular and tubular immune cells.

In another aspect, provided herein is a method of treating an autoinflammatory disease in a subject, comprising: (a) identifying a subject with an autoinflammatory condition selected from familial Mediterranean fever, cryopyrin-associated periodic syndromes (FCAS, Muckle-Wells syndrome, NOMID), gout, or other monogenic autoinflammatory syndromes where NLRP3 inflammasome hyperactivation is the validated pathophysiologic mechanism; (b) administering a ketogenic agent in an amount sufficient to induce and maintain circulating β-hydroxybutyrate in the range 1.0-2.0 mM for at least 2.6 hours per day, while remaining below 2.4 mM; (c) measuring circulating BHB and NLRP3 inflammasome activation at intervals; and (d) adjusting dose to maintain the specified BHB time-window; wherein the method does not require that the subject exhibit glucose hypometabolism; and wherein the method is independent of dietary restriction of the subject.

In some embodiments, a ketogenic agent achieving the pharmacokinetic profile of Study 25-101 at 5 g/kg (Cmax 1.542 mM, time above 1.0 mM approximately 2.6 hours, maximum below 2.4 mM) directly produces the NLRP3 time-window.

In some embodiments, NLRP3 inflammasome activation is measured by phospho-caspase-1 intracellular staining in circulating monocytes by flow cytometry and by circulating IL-1β and IL-18 levels.

In some embodiments, the autoinflammatory disease is a monogenic NLRP3 gain-of-function condition responsive to IL-1β antagonism.

### Enumerated Embodiments

Embodiment 1. A method for assessing a state of bioenergetic sufficiency or insufficiency in a biological system, the method comprising: a. measuring, using one or more measurement modalities, a plurality of parameters reflecting components of an effective energy generation rate (E) and a total energy requirement rate (R) in the biological system; and b. determining, using a processor configured with an algorithm based on the Unified Theory of Bioenergetic Sufficiency (UTBS), the state of bioenergetic sufficiency or insufficiency based on an integrated relationship between said measured parameters, wherein the relationship reflects a Metabolic Sufficiency Ratio (M) defined as M =E/R or a derivative thereof.

Embodiment 2. The method of embodiment 1, wherein the biological system is selected from the group consisting of a cell, a tissue, an organ, and a mammalian subject.

Embodiment 3. The method of embodiment 1, wherein the plurality of parameters reflecting E comprise at least one parameter selected from the group consisting of: a level, concentration, uptake rate, or metabolic flux of an energy substrate selected from glucose, a fatty acid, a ketone body, lactate 8, and an amino acid; a level or activity of a substrate transporter or metabolic enzyme; an ATP level; a phosphocreatine (PCr) level; an ATP/ADP ratio; a PCr/ATP ratio; and an oxygen consumption rate.

The method of embodiment 3, wherein the substrate transporter is a Monocarboxylate Transporter (MCT) or a Glucose Transporter (GLUT).

Embodiment 4. The method of embodiment 1, wherein the plurality of parameters reflecting R comprise at least one parameter selected from the group consisting of: a marker of basal metabolic rate; a marker of cellular stress; a marker of inflammation; a marker of oxidative damage; a marker of tissue-specific activity; and a marker of cellular repair processes.

Embodiment 5. The method of embodiment 1, wherein the one or more measurement modalities are selected from the group consisting of positron emission tomography (PET)₈, magnetic resonance spectroscopy (MRS), metabolomic analysis, indirect calorimetry, and biochemical assay of a biological sample.

Embodiment 6. The method of embodiment 6, wherein the PET utilizes a tracer selected from the group consisting of a glucose analog (e.g., ¹⁸F-FDG), a ketone body analog (e.g., 11C-acetoacetate), a fatty acid analog, and a neuroinflammation marker (e.g., TSPO ligand).

Embodiment 7. The method of embodiment 6, wherein the MRS is 31P-MRS measuring ATP or PCr levels or PCr/ATP ratio, or 1H-MRS measuring lactate or ketone body levels, or ¹³C-MRS measuring metabolic flux.

Embodiment 8. The method of embodiment 6, wherein the biochemical assay comprises a luciferase-based ATP assay, an enzymatic β-hydroxybutyrate (BHB) assay, or an enzymatic lactate assay.

Embodiment 9. The method of embodiment 6, wherein the metabolomic analysis provides an integrated biomarker score reflecting systemic metabolic stress, optionally the Metabolic Vulnerability Index (MVX).

Embodiment 10. The method of embodiment 1, further comprising measuring an index of metabolic flexibility, optionally using indirect calorimetry to measure a change in Respiratory Quotient (RQ) in response to a metabolic challenge.

Embodiment 11. The method of embodiment 1, wherein determining the state comprises comparing the relationship reflecting M to a predetermined, tissue-specific critical threshold value (M_crit).

Embodiment 12. The method of embodiment 1, further comprising assigning a metabolic stage to the biological system based on the determined state of bioenergetic sufficiency or insufficiency, wherein the metabolic stage corresponds to a predefined UTBS classification system reflecting severity (e.g., Stages 0-4).

Embodiment 13. A method for treating or preventing a condition associated with energy insufficiency in a subject diagnosed using the method of embodiment 1, the method comprising administering to the subject a therapeutically effective amount of an agent or intervention selected or adjusted based on the assessed state of bioenergetic sufficiency or insufficiency, wherein the agent or intervention increases a Metabolic Sufficiency Ratio (M=E/R) in a target tissue of the subject.

Embodiment 14. The method of embodiment 14, wherein the agent or intervention increases M by increasing the effective energy generation rate (E).

Embodiment 15. The method of embodiment 15, wherein the agent comprises an alternative energy substrate or a precursor thereof, selected from the group consisting of a ketone body, a ketone ester, a ketone salt, a medium-chain triglyceride (MCT), a lactate precursor, and one or more specific amino acids.

Embodiment 16. The method of embodiment 16, wherein the ketone ester is D-β-hydroxybutyrate-(R)-1,3-butanediol.

Embodiment 17. The method of embodiment 15, wherein the agent enhances mitochondrial function or efficiency, optionally comprising an NAD⁺ precursor or Coenzyme Q10.

Embodiment 18. The method of embodiment 15, wherein the agent enhances the utilization of a primary fuel substrate, optionally an insulin sensitizer for enhancing glucose utilization.

Embodiment 19. The method of embodiment 14, wherein the agent or intervention increases M by decreasing the total energy requirement rate (R).

Embodiment 20. The method of embodiment 20, wherein the agent reduces inflammation, oxidative stress, or tissue workload.

Embodiment 21. The method of embodiment 14, wherein the agent or intervention enhances metabolic flexibility.

Embodiment 22. The method of embodiment 14, wherein the condition associated with energy insufficiency is selected from the group consisting of Alzheimer's disease, Parkinson's disease, heart failure, type 2 diabetes, metabolic syndrome, traumatic brain injury cachexia, age-related functional decline, and the orphan diseases GLUT1 deficiency syndrome, Pyruvate Dehydrogenase Complex Deficiency (PDHc-D), SLC13A5 epileptic encephalopathy, PCDH19-Related Epilepsy, Dravet syndrome, Lennox-Gastaut syndrome, infantile spasms, CDKL5 Deficiency Disorder, Doose syndrome (myoclonic-astatic epilepsy), Mitochondrial Complex I Deficiencies, Leigh syndrome, MELAS, MERRF, Friedreich's ataxia, juvenile ALS, multiple acyl-CoA dehydrogenase deficiency (MADD), Very Long-Chain Acyl-CoA Dehydrogenase Deficiency (VLCAD), Long-Chain 3-Hydroxyacyl-CoA Dehydrogenase Deficiency/Trifunctional Protein Deficiency (LCHAD/TFP), medium-chain acyl-CoA dehydrogenase deficiency (MCAD deficiency), Carnitine Palmitoyltransferase II Deficiency (CPT-II), HMG-COA Lyase Deficiency, Barth syndrome, LMNA-related dilated cardiomyopathy, Duchenne muscular dystrophy-associated cardiomyopathy, spinal muscular atrophy type II, mtDNA Depletion Syndromes, early-onset familial Alzheimer's or Parkinson's disease, Febrile Infection-Related Epilepsy Syndrome (FIRES), Glioblastoma Multiforme (GBM), Diffuse Intrinsic Pontine Glioma (DIPG), Aicardi-Goutières Syndrome (AGS), SCN8A-Related Epilepsy (SCN8A), Tuberous Sclerosis Complex (TSC), Rasmussen Encephalitis (RE), IDH-mutant High-Grade Glioma (HGG), Atypical Teratoid Rhabdoid Tumors (ATRT), Rett Syndrome (RTT), Angelman Syndrome (AS), Lafora Disease (LD), Frontotemporal Dementia (FTD), Acute Necrotizing Encephalopathy (ANEC), Autoimmune Limbic Encephalitis (ALE), Focal Cortical Dysplasia (FCD), Hypothalamic Hamartoma (HH), Neuronal Ceroid Lipofuscinoses (NCLs), Unverricht-Lundborg Disease (EPM1), Sturge-Weber Syndrome (SWS), Hemimegalencephaly (HME), and Krabbe Disease (KD).

Embodiment 23. The method of embodiment 14, wherein the condition associated with energy insufficiency is selected from the group consisting of Alzheimer's disease, Parkinson's disease, heart failure, type 2 diabetes, metabolic syndrome, traumatic brain injury, cachexia, and age-related functional decline.

Embodiment 24. The method of embodiment 14, wherein the condition associated with energy insufficiency is selected from the group consisting of the orphan diseases GLUT1 deficiency syndrome, Pyruvate Dehydrogenase Complex Deficiency (PDHc-D), SLC13A5 epileptic encephalopathy, PCDH19-Related Epilepsy, Dravet syndrome, Lennox-Gastaut syndrome, infantile spasms, CDKL5 Deficiency Disorder, Doose syndrome (myoclonic-astatic epilepsy), Mitochondrial Complex I Deficiencies, Leigh syndrome, MELAS, MERRE, Friedreich's ataxia, juvenile ALS, multiple acyl-CoA dehydrogenase deficiency (MADD), Very Long-Chain Acyl-CoA Dehydrogenase Deficiency (VLCAD), Long-Chain 3-Hydroxyacyl-CoA Dehydrogenase Deficiency/Trifunctional Protein Deficiency (LCHAD/TFP), medium-chain acyl-CoA dehydrogenase deficiency (MCAD deficiency), Carnitine Palmitoyltransferase II Deficiency (CPT-II), HMG-COA Lyase Deficiency, Barth syndrome, LMNA-related dilated cardiomyopathy, Duchenne muscular dystrophy-associated cardiomyopathy, spinal muscular atrophy type II, mtDNA Depletion Syndromes, early-onset familial Alzheimer's or Parkinson's disease, Febrile Infection-Related Epilepsy Syndrome (FIRES), Glioblastoma Multiforme (GBM), Diffuse Intrinsic Pontine Glioma (DIPG), Aicardi-Goutières Syndrome (AGS), SCN8A-Related Epilepsy (SCN8A), Tuberous Sclerosis Complex (TSC), Rasmussen Encephalitis (RE), IDH-mutant High-Grade Glioma (HGG), Atypical Teratoid Rhabdoid Tumors (ATRT), Rett Syndrome (RTT), Angelman Syndrome (AS), Lafora Disease (LD), Frontotemporal Dementia (FTD), Acute Necrotizing Encephalopathy (ANEC), Autoimmune Limbic Encephalitis (ALE), Focal Cortical Dysplasia (FCD), Hypothalamic Hamartoma (HH), Neuronal Ceroid Lipofuscinoses (NCLs), Unverricht-Lundborg Disease (EPM1), Sturge-Weber Syndrome (SWS), Hemimegalencephaly (HME), and Krabbe Disease (KD).

Embodiment 25. The method of embodiment 14, wherein the condition associated with energy insufficiency is selected from the group consisting of Dravet Syndrome, GLUT1 deficiency syndrome, Friedreich's ataxia, Leigh Syndrome, Pyruvate Dehydrogenase Complex Deficiency (PDHc-D), Carnitine Palmitoyltransferase II Deficiency (CPT-II), Long-Chain 3-Hydroxyacyl-CoA Dehydrogenase Deficiency/Trifunctional Protein Deficiency (LCHAD/TFP), Very Long-Chain Acyl-CoA Dehydrogenase Deficiency (VLCAD), Febrile Infection-Related Epilepsy Syndrome (FIRES), and HMG-COA Lyase Deficiency.

Embodiment 26. The method of embodiment 14, wherein the energy insufficiency comprises glucose hypometabolism identified by PET.

Embodiment 27. The method of embodiment 14, wherein selecting or adjusting the agent or intervention is further based on a metabolic stage determined according to the method of embodiment 13.

Embodiment 28. A composition for use in treating or preventing a condition associated with energy insufficiency according to the method of embodiment 14, the composition comprising a therapeutically effective amount of an agent that increases a Metabolic Sufficiency Ratio (M=E/R) in a target tissue of a subject, wherein the agent is selected from the group consisting of a ketone body, a ketone ester, a ketone salt, a medium-chain triglyceride (MCT), a lactate precursor, one or more specific amino acids, a mitochondrial modulator, an insulin sensitizer, an anti-inflammatory agent, an antioxidant, and combinations thereof.

Embodiment 29. The composition for use of embodiment 29, wherein the condition is selected from the group consisting of Alzheimer's disease, Parkinson's disease, heart failure, type 2 diabetes, metabolic syndrome, traumatic brain injury, cachexia, age-related functional decline, and the orphan diseases GLUT1 deficiency syndrome, Pyruvate Dehydrogenase Complex Deficiency (PDHc-D), SLC13A5 epileptic encephalopathy, PCDH19-Related Epilepsy, Dravet syndrome, Lennox-Gastaut syndrome, infantile spasms, CDKL5 Deficiency Disorder, Doose syndrome (myoclonic-astatic epilepsy), Mitochondrial Complex I Deficiencies, Leigh syndrome, MELAS, MERRF, Friedreich's ataxia, juvenile ALS, multiple acyl-CoA dehydrogenase deficiency (MADD), Very Long-Chain Acyl-CoA Dehydrogenase Deficiency (VLCAD), Long-Chain 3-Hydroxyacyl-CoA Dehydrogenase Deficiency/Trifunctional Protein Deficiency (LCHAD/TFP), medium-chain acyl-CoA dehydrogenase deficiency (MCAD deficiency), Carnitine Palmitoyltransferase II Deficiency (CPT-II), HMG-COA Lyase Deficiency, Barth syndrome, LMNA-related dilated cardiomyopathy, Duchenne muscular dystrophy-associated cardiomyopathy, spinal muscular atrophy type II, mtDNA Depletion Syndromes, early-onset familial Alzheimer's or Parkinson's disease, Febrile Infection-Related Epilepsy Syndrome (FIRES), Glioblastoma Multiforme (GBM), Diffuse Intrinsic Pontine Glioma (DIPG), Aicardi-Goutières Syndrome (AGS), SCN8A-Related Epilepsy (SCN8A), Tuberous Sclerosis Complex (TSC), Rasmussen Encephalitis (RE), IDH-mutant High-Grade Glioma (HGG), Atypical Teratoid Rhabdoid Tumors (ATRT), Rett Syndrome (RTT), Angelman Syndrome (AS), Lafora Disease (LD), Frontotemporal Dementia (FTD), Acute Necrotizing Encephalopathy (ANEC), Autoimmune Limbic Encephalitis (ALE), Focal Cortical Dysplasia (FCD), Hypothalamic Hamartoma (HH), Neuronal Ceroid Lipofuscinoses (NCLs), Unverricht-Lundborg Disease (EPM1), Sturge-Weber Syndrome (SWS), Hemimegalencephaly (HME), and Krabbe Disease (KD).

Embodiment 30. The composition for use of embodiment 29, wherein the condition is selected from the group consisting of Alzheimer's disease, Parkinson's disease, heart failure, type 2 diabetes, metabolic syndrome, traumatic brain injury 8, cachexia, and age-related functional decline.

Embodiment 31. The composition for use of embodiment 29, wherein the condition is selected from the group consisting of the orphan diseases GLUT1 deficiency syndrome, Pyruvate Dehydrogenase Complex Deficiency (PDHc-D), SLC13A5 epileptic encephalopathy, PCDH19-Related Epilepsy, Dravet syndrome, Lennox-Gastaut syndrome, infantile spasms, CDKL5 Deficiency Disorder, Doose syndrome (myoclonic-astatic epilepsy), Mitochondrial Complex I Deficiencies, Leigh syndrome, MELAS, MERRF, Friedreich's ataxia, juvenile ALS, multiple acyl-CoA dehydrogenase deficiency (MADD), Very Long-Chain Acyl-CoA Dehydrogenase Deficiency (VLCAD), Long-Chain 3-Hydroxyacyl-CoA Dehydrogenase Deficiency/Trifunctional Protein Deficiency (LCHAD/TFP), medium-chain acyl-CoA dehydrogenase deficiency (MCAD deficiency), Carnitine Palmitoyltransferase II Deficiency (CPT-II), HMG-COA Lyase Deficiency, Barth syndrome, LMNA-related dilated cardiomyopathy, Duchenne muscular dystrophy-associated cardiomyopathy, spinal muscular atrophy type II, mtDNA Depletion Syndromes, early-onset familial Alzheimer's or Parkinson's disease, Febrile Infection-Related Epilepsy Syndrome (FIRES), Glioblastoma Multiforme (GBM), Diffuse Intrinsic Pontine Glioma (DIPG), Aicardi-Goutières Syndrome (AGS), SCN8A-Related Epilepsy (SCN8A), Tuberous Sclerosis Complex (TSC), Rasmussen Encephalitis (RE), IDH-mutant High-Grade Glioma (HGG), Atypical Teratoid Rhabdoid Tumors (ATRT), Rett Syndrome (RTT), Angelman Syndrome (AS), Lafora Disease (LD), Frontotemporal Dementia (FTD), Acute Necrotizing Encephalopathy (ANEC), Autoimmune Limbic Encephalitis (ALE), Focal Cortical Dysplasia (FCD), Hypothalamic Hamartoma (HH), Neuronal Ceroid Lipofuscinoses (NCLs), Unverricht-Lundborg Disease (EPM1), Sturge-Weber Syndrome (SWS), Hemimegalencephaly (HME), and Krabbe Disease (KD).

Embodiment 32. The composition for use of embodiment 29, wherein the condition is selected from the group consisting of Dravet Syndrome, GLUT1 deficiency syndrome, Friedreich's ataxia, Leigh Syndrome, Pyruvate Dehydrogenase Complex Deficiency (PDHc-D), Carnitine Palmitoyltransferase II Deficiency (CPT-II), Long-Chain 3-Hydroxyacyl-CoA Dehydrogenase Deficiency/Trifunctional Protein Deficiency (LCHAD/TFP), Very Long-Chain Acyl-CoA Dehydrogenase Deficiency (VLCAD), Febrile Infection-Related Epilepsy Syndrome (FIRES), and HMG-COA Lyase Deficiency.

Embodiment 33. The composition for use of embodiment 29, wherein the agent is a ketone ester, optionally D-β-hydroxybutyrate-(R)-1,3-butanediol.

Embodiment 34. A system for assessing a state of bioenergetic sufficiency or insufficiency in a biological system, the system comprising: a. at least one measurement device selected from the group consisting of a PET scanner, an MRS system, a biomarker analysis platform configured for assays, and a functional test device, the device(s) configured to acquire data corresponding to a plurality of parameters reflecting components of an effective energy generation rate (E) and a total energy requirement rate (R) in the biological system; and b. a processor operably connected to the at least one measurement device, the processor configured with instructions embodying an algorithm based on the Unified Theory of Bioenergetic Sufficiency (UTBS) to: i. receive the acquired data from the plurality of parameters; and ii. determine the state of bioenergetic sufficiency or insufficiency based on an integrated relationship between said parameters reflecting a Metabolic Sufficiency Ratio (M=E/R) or a derivative thereof.

Embodiment 35. The system of embodiment 35, wherein the processor is further configured to assign a metabolic stage based on the determined state according to a predefined UTBS classification system.

Embodiment 36. The system of embodiment 35, wherein the processor is further configured to output a recommendation for or control a therapeutic intervention aimed at increasing M=E/R, based on the determined state or assigned stage.

Embodiment 37. The system of embodiment 35, wherein the processor implements a mathematical model of UTBS simulating metabolic dynamics or a machine learning algorithm trained to predict M=E/R status or outcomes from the acquired data.

Embodiment 38 A kit for use in assessing or treating energy insufficiency according to the methods of embodiment 1 or embodiment 14, the kit comprising: a. one or more reagents for measuring, using biochemical assay, PET, or MRS, one or more parameters related to a Metabolic Sufficiency Ratio (M=E/R); or b. a supply of a therapeutic agent formulated to increase the Metabolic Sufficiency Ratio (M=E/R); and c. instructions for use correlating the measurement or administration to the Unified Theory of Bioenergetic Sufficiency (UTBS), optionally including reference to UTBS metabolic stages.

Embodiment 39. The kit of embodiment 39, wherein the one or more reagents comprise components for a luciferase-based ATP assay, an enzymatic BHB assay, an enzymatic lactate assay, or precursors for synthesis of a PET tracer.

Embodiment 40. The kit of embodiment 39, wherein the therapeutic agent is selected from the group consisting of a ketone body, a ketone ester, a ketone salt, a medium-chain triglyceride (MCT), a lactate precursor, one or more specific amino acids, and a mitochondrial modulator.

Embodiment 41. The method of embodiment 14, wherein the condition associated with energy insufficiency is characterized by one or more symptoms selected from the group consisting of cardiomyopathy, seizures, hypoketotic hypoglycemia, lactic acidosis, ataxia, cognitive decline, cognitive impairment, developmental delay, exercise intolerance, failure to thrive, hypoglycemia, intracranial pressure, lethargy, metabolic crises, multiple seizure types, muscle weakness, myoclonic-astatic seizures in childhood, neurodegeneration, neurodevelopmental delay, neuropathy, recurrent non-ketotic hypoglycemia, recurrent rhabdomyolysis, refractory epilepsy, refractory seizures, super-refractory status epilepticus, and tumor progression.

Embodiment 42. The composition for use of embodiment 29, wherein the condition is characterized by one or more symptoms selected from the group consisting of cardiomyopathy, seizures, hypoketotic hypoglycemia, lactic acidosis, ataxia, cognitive decline, cognitive impairment, developmental delay, exercise intolerance, failure to thrive, hypoglycemia, intracranial pressure, lethargy, metabolic crises, multiple seizure types, muscle weakness, myoclonic-astatic seizures in childhood, neurodegeneration, neurodevelopmental delay, neuropathy, recurrent non-ketotic hypoglycemia, recurrent rhabdomyolysis, refractory epilepsy, refractory seizures, super-refractory status epilepticus, and tumor progression.

Embodiment 43. A method of treating a disease characterized by metabolic, redox, or epigenetic dysregulation in a subject in need thereof, comprising administering to the subject a therapeutically effective amount of a ketone body or a pharmaceutically acceptable ketone precursor, thereby improving one or more metabolic, redox, or epigenetic parameters in the subject and treating the disease.

Embodiment 44 The method of embodiment 44, wherein the disease characterized by metabolic, redox, or epigenetic dysregulation is selected from the group consisting of: a neurodegenerative disease, a neurodevelopmental or psychiatric disorder, a metabolic or endocrine disease, an inflammatory or autoimmune disease, a cardiovascular or ischemic condition, a mitochondrial disorder, or a cancer.

Embodiment 45. The method of embodiment 44 or 452, wherein the disease is Alzheimer's disease, Parkinson's disease, amyotrophic lateral sclerosis (ALS), Huntington's disease, or another neurodegenerative disorder, and wherein administering the ketone body improves neuronal energy metabolism and reduces neurodegeneration in the subject.

Embodiment 46. The method of embodiment 44 or 45, wherein the disease is a psychiatric or neurodevelopmental condition selected from: major depressive disorder, bipolar disorder, schizophrenia, an autism spectrum disorder, Rett syndrome, or another disorder of neurodevelopment, and wherein administering the ketone body corrects an underlying metabolic or neurotransmitter imbalance.

Embodiment 47. The method of embodiment 44 or 45, wherein the disease is characterized by reductive stress-indicated by an elevated NADH/NAD⁺ ratio, elevated lactate/pyruvate ratio, or elevated β-hydroxybutyrate/acetoacetate ratio-and wherein the administering step increases the subject's NAD+/NADH ratio and alleviates said reductive stress.

Embodiment 48. The method of embodiment 48, wherein the disease characterized by reductive stress is selected from: ischemic heart disease, heart failure, stroke or brain ischemia, sepsis or shock, a mitochondrial disorder, cancer with Warburg-type metabolism, or a metabolic syndrome with insulin resistance.

Embodiment 49. The method of embodiment 44, wherein the disease involves epigenetic dysregulation, indicated by aberrant histone deacetylase activity or abnormal histone modification patterns, and wherein administering the ketone body increases histone acetylation or β-hydroxybutyrylation in affected cells of the subject.

Embodiment 50. The method of embodiment 50, wherein the disease involving epigenetic dysregulation is selected from: a cognitive disorder with HDAC overexpression, a neurodevelopmental syndrome caused by a mutation in a chromatin regulator, or a cancer with epigenetic silencing of tumor suppressor genes.

Embodiment 51. The method of embodiment 44, wherein the ketone body or precursor is administered in an amount sufficient to raise the subject's blood β-hydroxybutyrate level to between about 0.5 mM and about 8 mM.

Embodiment 52. The method of embodiment 44, wherein the ketone body or precursor comprises β-hydroxybutyrate, acetoacetate, or a combination thereof, optionally in a form selected from: a ketone salt, a ketone ester, a ketone amide, a ketoacid, or a plurality of these forms.

Embodiment 53. The method of embodiment 44, wherein the ketone body or precursor is administered orally, enterally, or parenterally as a composition that is substantially free of 1,3-butanediol, thereby avoiding alcohol related side effects while delivering therapeutic ketosis.

Embodiment 54. The method of embodiment 44, wherein administering the ketone body comprises providing a racemic or enantiomerically enriched D-β-hydroxybutyrate, and wherein in certain embodiments the composition yields at least 70% D-β-hydroxybutyrate in the bloodstream of the subject.

Embodiment 55. The method of embodiment 44, further comprising identifying the subject as having said disease characterized by metabolic, redox, or epigenetic dysregulation, by measuring one or more biomarkers indicative of metabolic, redox, or epigenetic dysfunction prior to treatment.

Embodiment 56. The method of embodiment 56, wherein the one or more biomarkers are selected from: blood or tissue NAD⁺ levels, NADH/NAD⁺ ratio, lactate level, β-hydroxybutyrate: acetoacetate ratio, reactive oxygen species level, an inflammatory cytokine level, or a histone acetylation level in a tissue sample.

Embodiment 57. The method of embodiment 44, wherein the subject's condition prior to treatment is characterized by an “M score” below a healthy threshold, the M-score being a composite index of metabolic, redox, and epigenetic biomarkers, and wherein administering the ketone body raises the subject's M-score.

Embodiment 58. The method of embodiment 44, wherein the disease is a cancer selected from solid tumors and hematologic malignancies, and metabolic, redox, or epigenetic dysregulation refers to the cancer cells' Warburg metabolism, elevated NADH, and aberrant gene expression, and wherein administering the ketone body is effective to alter the tumor metabolism or tumor microenvironment, thereby inhibiting cancer progression.

Embodiment 59. The method of embodiment 59, wherein the administering step further comprises co-administering an additional anti-cancer therapy, and the ketone body therapy improves the efficacy of the additional therapy by reprogramming tumor metabolism or enhancing immune response to the tumor.

Embodiment 60. The method of embodiment 44, wherein the disease is an autoimmune or autoinflammatory disease characterized by excessive immune activation and metabolic dysregulation of immune cells, and wherein administering the ketone body reduces inflammatory mediator levels and shifts immune cell metabolism from glycolytic to oxidative, thereby ameliorating the disease.

Embodiment 61. The method of embodiment 61, wherein the disease is selected from: rheumatoid arthritis, systemic lupus erythematosus, multiple sclerosis, psoriasis, type 1 diabetes (autoimmune diabetes), inflammatory bowel disease, or gout, and wherein ketone therapy results in a measurable reduction in IL-1β, IL-6, TNFα, or other inflammation markers in the subject.

Embodiment 62. The method of embodiment 44, wherein the disease is associated with mitochondrial dysfunction (including primary mitochondrial genetic disorders or age-related mitochondrial decline), and wherein ketone body administration bypasses one or more defective metabolic steps and improves ATP production in affected tissues.

Embodiment 63. The method of embodiment 63, wherein the disease is selected from: MELAS syndrome, Leigh syndrome, mitochondrial myopathy, Friedreich's ataxia, or another disorder of mitochondrial energy production, and wherein administering the ketone body improves exercise tolerance, reduces lactate accumulation, or improves neurological function in the subject.

Embodiment 64. A method of treating a patient in need of metabolic therapy comprising: (a) administering to the patient a ketone body or ketone precursor composition; (b) measuring in the patient at least one ketone-related value selected from blood β-hydroxybutyrate concentration, blood acetoacetate concentration, or a ratio thereof; (c) adjusting the dose or rate of administration of the ketone composition based on the measured value to achieve a predetermined target ketone level or ratio; and (d) maintaining the patient's ketone level or ratio within said target range for a therapeutically effective duration, thereby treating the patient's condition.

Embodiment 65. The method of embodiment 65, wherein the predetermined target ketone level comprises a blood β hydroxybutyrate concentration between about 1 mM and about 5 mM.

Embodiment 66. The method of embodiment 65, wherein the predetermined target includes a β hydroxybutyrate: acetoacetate ratio in blood of between about 0.8:1 and about 4:1.

Embodiment 67. The method of embodiment 65, wherein the ketone-related value is measured continuously or repeatedly (at intervals of minutes to hours) using a sensor device, and the adjusting of the dose is performed automatically by a feedback-controlled pump to maintain the target range (closed-loop ketosis control).

Embodiment 68. The method of embodiment 65, wherein the target range is defined not only by amplitude but also by time in-range, and the method comprises maintaining the patient's blood β-hydroxybutyrate within the target range for at least 50% of a 24-hour period, for a plurality of consecutive days.

Embodiment 69. The method of embodiment 65, further comprising measuring one or more secondary biomarkers indicative of therapeutic effect-selected from NAD⁺ level, NADH/NAD⁺ ratio, blood lactate, a plasma inflammatory cytokine level, or a tissue-specific molecular marker- and wherein the dose adjusting in step (c) is additionally based on achieving a target secondary biomarker value.

Embodiment 70. The method of embodiment 70, wherein the secondary biomarker is the patient's blood NAD⁺ concentration or NAD+/NADH ratio, and the method comprises adjusting the ketone closing until the patient's NAD⁺ level increases by at least a predetermined amount or the NAD+/NADH ratio reaches a target indicative of improved redox state.

Embodiment 71. The method of embodiment 70, wherein the secondary biomarker is an indicator of inflammation (IL-1β or CRP level) or an epigenetic mark (percentage of histone H3 acetylation in circulating mononuclear cells), and wherein the ketone dosage is adjusted upward if the inflammatory marker remains above a threshold or the epigenetic mark remains below a threshold, respectively.

Embodiment 72. The method of embodiment 65, wherein the patient is monitored with a dual sensor system measuring both blood glucose and blood ketones, and the target comprises maintaining a Glucose: Ketone Index (GKI) below a specified value, wherein adjusting the dose includes reducing glucose intake or increasing ketone delivery to achieve said GKI.

Embodiment 73. The method of embodiment 73, wherein the target GKI is less than or equal to 3.0, indicating a state of high therapeutic ketosis relative to glucose, suitable for treating conditions such as brain tumors, epilepsy, or Alzheimer's disease.

Embodiment 74. The method of embodiment 65, wherein the patient's condition being treated is epilepsy or a seizure disorder, and the target ketone level or ratio is chosen to correlate with seizure freedom (for example, β-hydroxybutyrate 4 mM+1 mM), and wherein step (d) comprises maintaining that ketone level for the majority of each day to prevent seizures.

Embodiment 75. The method of embodiment 65, wherein step (a) comprises initiating an infusion or ingestion of a ketone formulation, and step (c) comprises increasing the infusion rate or administering additional doses if the measured β-hydroxybutyrate is below target, or decreasing/skipping doses if above target, thereby titrating the therapy to the individual patient's pharmacokinetics.

Embodiment 76. The method of embodiment 65, wherein the predetermined target ketone range is personalized based on the patient's baseline metabolic state or genomic biomarkers, such that patients with a first phenotype are given a target range of X-Y mM β-hydroxybutyrate, while patients with a second phenotype are given a different target range.

Embodiment 77. The method of embodiment 65, wherein the ketone composition includes both β-hydroxybutyrate and acetoacetate sources, and the ratio of those sources is adjusted during treatment to achieve a target β-hydroxybutyrate: acetoacetate ratio in the patient's blood as part of the treat-to-target strategy.

Embodiment 78. The method of embodiment 65, wherein step (d) of maintaining the target range is carried out for a defined therapeutic period (e.g., at least 4 weeks of daily therapy), after which the patient's condition is reassessed, and further adjustments to the target or treatment schedule are made accordingly.

Embodiment 79. The method of embodiment 65, implemented using a computerized system comprising a ketone monitor and an automated closing pump, wherein the system is programmed with the target values and modulates closing in real time, thereby executing steps (b)-(d) without continuous manual intervention.

Embodiment 80. The method of embodiment 65, wherein the adjusting step (c) is performed at least in part by the patient or caregiver using guidance provided by a companion software or algorithm which, based on measured ketone values, recommends increasing, decreasing, or continuing the same dosage to reach the target.

Embodiment 81. The method of embodiment 65, wherein the condition being treated is characterized by daily or circadian fluctuations in symptom severity, and the treat-to-target method further comprises programming different target levels at different times of day to align with when therapeutic effect is most needed (for example, higher ketone levels overnight for a patient with early morning migraine or seizures).

Embodiment 82. The method of embodiment 65, wherein achieving the target ketone range produces a demonstrable improvement in a patient outcome measure (selected from seizure frequency, cognitive test score, blood pressure, pain score, or exercise endurance), confirming that the treat-to-target ketosis was therapeutically effective.

Embodiment 83. A method of treating a disease or condition associated with a high NADH/NAD⁺ ratio or reductive stress in a patient, comprising administering to the patient a therapeutically effective amount of acetoacetate or an acetoacetate-generating compound, thereby converting excess NADH to NAD⁺ in cells of the patient and alleviating the reductive stress.

Embodiment 84. The method of embodiment 84, wherein the disease associated with high NADH/NAD⁺ (reductive stress) is selected from: heart failure, myocardial ischemia, ischemic stroke, shock or critical illness, sepsis, a solid tumor with Warburg-type metabolism, fatty liver disease, or a mitochondrial disorder.

Embodiment 85. The method of embodiment 84, wherein the acetoacetate-generating compound is 1,3-butanediol diacetoacetate, an acetoacetate salt, an acetoacetyl-based ester, or any precursor that yields acetoacetate upon metabolism in the patient.

Embodiment 86. The method of embodiment 84, wherein administering the acetoacetate or precursor results in an increase in the patient's blood acetoacetate concentration to at least about 0.5 mM, and concurrently causes a decrease in blood lactate concentration or lactate: pyruvate ratio, indicating improved cytosolic redox balance.

Embodiment 87. The method of embodiment 84, wherein the patient's baseline arterial ketone body ratio (AKBR=[acetoacetate]/[β-hydroxybutyrate]) is abnormally low (indicative of a reduced state), and the method raises the AKBR to a normal range by increasing acetoacetate availability.

Embodiment 88. The method of embodiment 84, wherein the condition is acute ischemia (myocardial infarction or cerebral ischemia), and the acetoacetate or precursor is administered acutely (for example, via intravenous infusion during an infarct or stroke) in an amount sufficient to supply electron acceptors that oxidize NADH to NAD⁺, thereby limiting ischemic damage.

Embodiment 89. The method of embodiment 84, wherein the condition is chronic heart failure, and treatment with acetoacetate or its precursor over a period of at least 2 weeks leads to an increase in myocardial tissue NAD⁺ levels and improvement in cardiac output or ejection fraction relative to baseline.

Embodiment 90. The method of embodiment 84, wherein the acetoacetate or precursor is administered in combination with β-hydroxybutyrate or a β-hydroxybutyrate precursor in a formulation such that the molar ratio of delivered β-hydroxybutyrate equivalents to acetoacetate equivalents is less than 1:1, thereby biasing the patient's metabolism toward acetoacetate utilization and NADH consumption.

Embodiment 91. The method of embodiment 84, further comprising monitoring an indicator of redox state in the patient, selected from: blood β-hydroxybutyrate: acetoacetate ratio, blood lactate: pyruvate ratio, or tissue NAD⁺ levels, and adjusting the dosage of acetoacetate accordingly to reach a target indicator value.

Embodiment 92. The method of embodiment 84, wherein the disease or condition associated with reductive stress includes a cancer, and administering acetoacetate slows cancer cell proliferation or potentiates other cancer therapies by altering the redox environment of cancer cells (making them less able to deal with oxidative stress).

Embodiment 93. The method of embodiment 93, wherein the cancer is characterized by high intracellular NADH (for example, due to hypoxic regions of a tumor), and acetoacetate therapy selectively strains cancer cells' metabolism by forcing them to divert NADH to BHB production, thereby sensitizing them to cell death.

Embodiment 94. The method of embodiment 84, wherein the patient exhibits clinical signs of reductive stress such as lactic acidosis or pseudo-hypoxia, and within 1 hour of acetoacetate administration, an improvement in blood pH or reduction in lactate is observed.

Embodiment 95. The method of embodiment 84, wherein the acetoacetate is provided as a stabilized formulation, optionally including a buffer or in a controlled-release oral form, to ensure effective delivery to the bloodstream without rapid decomposition to acetone.

Embodiment 96. The method of embodiment 84, wherein treating reductive stress via acetoacetate further results in secondary benefits, including increased activity of NAD+-dependent enzymes (such as sirtuins) and improved mitochondrial respiration in the patient's cells.

Embodiment 97. The method of embodiment 84, wherein the patient's therapy is guided by a threshold: acetoacetate closing is initiated or increased when the patient's blood β-hydroxybutyrate: acetoacetate ratio exceeds a set value (e.g., >4:1), and continued until the ratio falls below a set value, indicating restoration of more oxidative conditions.

Embodiment 98. The method of embodiment 84, wherein the patient is concurrently administered a second agent that benefits from a higher NAD+/NADH ratio, such as a PARP inhibitor in cancer therapy or a sirtuin activating compound, such that acetoacetate-induced NAD⁺ elevation enhances the efficacy of said agent.

Embodiment 99. A method of modulating epigenetic marks in a subject in need of such modulation, comprising administering a ketone body in an amount sufficient to inhibit class I histone deacetylases (HDACs) and/or to increase levels of histone β-hydroxybutyrylation or acetoacetylation in cells of the subject, thereby altering gene expression in a therapeutically beneficial manner.

Embodiment 100. The method of embodiment 100, wherein the subject has a condition characterized by aberrant HDAC activity or histone hypoacetylation, and administering the ketone body increases histone acetylation at one or more gene loci associated with the condition.

Embodiment 101. The method of embodiment 100, wherein the ketone body administered is β-hydroxybutyrate (or a precursor thereof) at a blood concentration of at least about 2 mM, which concentration is effective to cause inhibition of HDAC1 and HDAC3 in vivo, leading to increased expression of genes normally suppressed by those HDACs.

Embodiment 102. The method of embodiment 100, wherein the condition is a neurological or cognitive disorder selected from: Alzheimer's disease, mild cognitive impairment, age-related cognitive decline, or a neurodevelopmental disorder such as Rett syndrome or autism, and wherein ketone-induced HDAC inhibition results in upregulation of synaptic plasticity genes and improvement in cognitive or developmental measures.

Embodiment 103. The method of embodiment 100, wherein the condition is a psychiatric disorder selected from: depression, bipolar disorder, post-traumatic stress disorder (PTSD), or schizophrenia, and wherein ketone administration leads to increased acetylation of histones at promoters of neurotrophic or plasticity related genes (including BDNF) in the brain, correlating with symptom improvement.

Embodiment 104. The method of embodiment 100, wherein the ketone body is administered chronically such that histone β hydroxybutyrylation (Kbhb) marks accumulate on histones of certain cell populations (for example, in liver or brain), mimicking a fasting-state epigenetic profile and thereby ameliorating a disease state.

Embodiment 105. The method of embodiment 100, wherein the subject's cells exhibit elevated levels of HDAC2 that contribute to pathology (for instance, HDAC2 in Alzheimer's-related memory impairment or in cognitive dysfunction), and β-hydroxybutyrate administration reduces HDAC2 activity or expression, thereby relieving HDAC2-mediated transcriptional repression.

Embodiment 106. The method of embodiment 100, wherein the subject suffers from a genetic syndrome involving a mutation in an epigenetic regulator (selected from: MECP2, KMT2D, KDM6A, UBE3A, or others), and ketone therapy partially compensates for the mutation by globally increasing histone acetylation and/or β hydroxybutyrylation, resulting in improved clinical features of the syndrome.

Embodiment 107. The method of embodiment 100, wherein the ketone body is acetoacetate (or an acetoacetate precursor) delivered in a manner to raise cellular acetoacetyl-CoA, and wherein this leads to an increase in histone acetoacetylation (Kacac) at lysines such as H3K27 or H3K9, thereby affecting gene expression.

Embodiment 108. The method of embodiment 100, wherein gene expression changes resulting from the ketone therapy are measured by an increase in mRNA or protein levels of one or more beneficial genes, examples of which include: FOXO3, catalase, mitochondrial enzymes (e.g., SOD2/MnSOD), neurotrophic factors (BDNF), or anti-inflammatory genes (e.g., IL-10), depending on tissue.

Embodiment 109. The method of embodiment 100, wherein the therapeutic benefit is evidenced by a reversal of an epigenetic signature of disease—for example, restoring normal levels of histone H3K9 acetylation in hippocampal neurons of an Alzheimer's patient (as may be measured indirectly via peripheral markers or animal models).

Embodiment 110. The method of embodiment 100, in which the ketone administration is combined with another epigenetic modulating therapy, such as a low dose of a synthetic HDAC inhibitor or a DNA methylation inhibitor, in order to synergistically correct epigenetic abnormalities in the subject.

Embodiment 111. The method of embodiment 100, wherein as a result of the ketone-induced epigenetic modulation, the subject exhibits a physiological improvement selected from: improved memory performance, increased muscle mitochondrial content (via PGC-1a activation), improved insulin sensitivity (via activation of metabolic genes in liver), or reduced inflammatory phenotype of immune cells.

Embodiment 112. The method of embodiment 100, wherein administering the ketone body yields detectable β-hydroxybutyryl histone marks in circulating immune cells of the subject (optionally measured via mass spectrometry of histone extracts), confirming epigenetic impact, and this correlates with clinical response.

Embodiment 113. The method of embodiment 100, wherein the ketone therapy is periodically cycled (e.g., 5 days on, 2 days off, or other regimen) to mimic intermittent fasting-induced epigenetic resets, thereby avoiding potential adaptation and maintaining responsiveness of gene expression.

Embodiment 114. The method of embodiment 100, wherein the subject is identified as likely to respond to epigenetic modulation by ketones by a companion diagnostic that shows low baseline histone acetylation or a specific epigenetic biomarker profile; those subjects are then treated with the ketone therapy with improved outcome relative to subjects without that profile.

Embodiment 115. A method of treating an NLRP3 inflammasome-associated disorder in a subject, comprising administering to the subject a therapeutically effective dose of a ketone body such that the subject's blood β-hydroxybutyrate level is elevated to at least about 1-2 mM for a sustained period, thereby suppressing NLRP3 inflammasome activation and reducing the release of IL-1β and IL-18 in the subject.

Embodiment 116. The method of embodiment 116, wherein the NLRP3 inflammasome-associated disorder is selected from: gout or urate crystal arthritis, familial cold autoinflammatory syndrome (FCAS) or other cryopyrinopathies, adult-onset Still's disease, systemic JIA, CAPS (Cryopyrin-Associated Periodic Syndromes), diabetic nephropathy (inflammasome-mediated), or other conditions wherein IL-1β plays a major pathogenic role.

Embodiment 117. The method of embodiment 116, wherein administering the ketone body results in a measurable decrease in an inflammation marker selected from: plasma IL-1β, IL-18, IL-6, C-reactive protein (CRP), or SAA (serum amyloid A) in the subject within a treatment period.

Embodiment 118. The method of embodiment 116, wherein the ketone body is β-hydroxybutyrate (or a precursor) that activates the hydroxycarboxylic acid receptor 2 (HCAR2/GPR109A) on immune cells, thereby mediating an anti-inflammatory effect which complements the direct inflammasome inhibition.

Embodiment 119. The method of embodiment 116, applied to treating gout, wherein the subject takes the ketone body supplement at the onset of a gout flare or as a prophylaxis daily, leading to reduced frequency or severity of gout flares compared to prior to treatment.

Embodiment 120. The method of embodiment 116, wherein the disorder is neuroinflammatory in nature (for example, Alzheimer's disease where NLRP3 in microglia contributes to pathology, or Multiple Sclerosis), and ketone administration lowers pro-inflammatory cytokines in the CNS milieu and improves clinical outcomes (such as cognitive scores or relapse rate).

Embodiment 121. The method of embodiment 116, wherein the subject is concurrently on an IL-1 blocking therapy (like anakinra or canakinumab) and ketone therapy is provided as an adjunct, potentially allowing a lower dose of the IL-1 blocker by synergistically reducing IL-1 production.

Embodiment 122. The method of embodiment 116, wherein the ketone therapy is initiated and adjusted to maintain an “anti inflammasome ketone range,” defined as a blood β-hydroxybutyrate level sufficient to inhibit NLRP3 activation ex vivo (for instance, ~3-5 mM based on in vitro studies), and wherein the subject's monocytes show reduced caspase-1 cleavage or ASC speck formation when tested.

Embodiment 123. The method of embodiment 116, aimed at treating an autoimmune condition (e.g., rheumatoid arthritis or systemic lupus erythematosus) wherein NLRP3 inflammasome contributes to disease flares, and ketone therapy leads to a longer remission period or decreased flare frequency.

Embodiment 124. The method of embodiment 116, wherein the ketone is delivered in a way to also provide an acidic microenvironment (for example, co—formulated with medium chain triglycerides that produce mild ketosis with slight metabolic acidosis), which enhances BHB's inflammasome-inhibiting effect (as BHB's effect on NLRP3 is potentiated by lower pH).

Embodiment 125. The method of embodiment 116, wherein beyond inflammasome markers, the ketone therapy also increases markers of T cell metabolic fitness, such as increased mitochondrial spare respiratory capacity in CD8+ T cells or increased frequency of memory T cells, thereby improving the subject's immune regulation.

Embodiment 126. The method of embodiment 116, wherein the disorder is cancer-related immune exhaustion (e.g., T cell exhaustion in tumor microenvironment associated with chronic antigen exposure), and ketone administration rejuvenates T cell function (through increased NAD⁺ and oxidative metabolism), indirectly inhibiting the chronic inflammasome-driven inflammation that impairs anti-tumor immunity.

Embodiment 127. The method of embodiment 116, wherein the effectiveness of inflammasome suppression is monitored by isolating the subject's peripheral blood monocytes at intervals and stimulating them ex vivo with LPS+ATP to activate NLRP3; the IL-1β release in the ketone-treated state is significantly lower than pre treatment, confirming target engagement.

Embodiment 128. A method of treating cancer in a subject, comprising administering to the subject (i) a ketone body or ketone precursor composition and (ii) at least one additional anti cancer therapy, wherein the ketone composition is dosed to induce a therapeutic level of ketosis in the subject and thereby modulate the subject's metabolism or immune response in a manner that enhances the efficacy of the additional anti-cancer therapy.

Embodiment 129. The method of embodiment 129, wherein the additional anti-cancer therapy is an immune checkpoint inhibitor selected from an anti-PD-1 antibody, an anti-PD-L1 antibody, or an anti-CTLA-4 antibody, and ketosis improves the metabolic fitness and functionality of T cells, resulting in an improved tumor response compared to the checkpoint inhibitor alone.

Embodiment 130. The method of embodiment 129, wherein the additional anti-cancer therapy is a chemotherapeutic agent or targeted therapy (selected from: platinum drugs, alkylating agents, antimetabolites, kinase inhibitors, hormone therapy, etc.), and the ketone therapy protects normal tissues or sensitizes cancer cells (for instance, by reducing cancer cell antioxidant capacity via NAD⁺ changes) leading to greater tumor kill at a given dose of chemotherapeutic.

Embodiment 131. The method of embodiment 129, wherein the additional anti-cancer therapy is radiation therapy, and ketone body administration is timed to coincide with radiation exposure, thereby potentially reducing radiation-induced normal tissue damage (through enhanced NAD⁺ for DNA repair in normals) and/or not protecting tumor (since tumor may not use ketones efficiently, or even increases ROS with ketones during radiation).

Embodiment 132. The method of embodiment 129, wherein the cancer is a brain tumor (such as glioblastoma), the additional therapy includes standard temozolomide chemotherapy and radiation, and the ketone composition (e.g., a ketone ester) is administered daily to maintain BHB levels of 2-3 mM, which has been shown to slow glioma growth and complement the cytotoxic therapy.

Embodiment 133. The method of embodiment 129, wherein the ketone composition is administered in combination with a PI3K/Akt/mTOR pathway inhibitor (or the ketone effect itself leads to mTOR inhibition due to low insulin), collectively providing a stronger anti-tumor effect by cutting off both growth signaling and providing a non-glucose fuel that tumor cells cannot effectively utilize.

Embodiment 134. A method of treating a metabolic disease (such as type 2 diabetes or obesity) in a subject, comprising administering a ketone body composition in combination with at least one existing metabolic therapy selected from: insulin or insulin secretagogues, biguanides (metformin), GLP-1 receptor agonists (or GLP-1/GIP co-agonists), SGLT2 inhibitors, DPP4 inhibitors, thiazolidinediones, or others, wherein the ketone therapy adds a complementary mechanism (improved energy utilization, appetite suppression, etc.) resulting in improved glycemic control or weight loss relative to the existing therapy alone.

Embodiment 135. The method of embodiment 135, wherein the subject is on a GLP-1 agonist for obesity or diabetes, and addition of ketone supplementation further suppresses appetite and increases resting energy expenditure, leading to greater weight reduction than GLP-1 agonist alone, and mitigates the loss of lean body mass.

Embodiment 136. The method of embodiment 135, wherein the subject is on metformin, and ketone therapy further lowers blood glucose by providing alternative fuel and possibly augmenting metformin's effect on hepatic gluconeogenesis (as both metformin and ketones reduce hepatic glucose output, by different mechanisms).

Embodiment 137. The method of embodiment 135, wherein the subject is on insulin for type 1 diabetes, and a carefully titrated low dose ketone infusion is used adjunctively as a brain energy supplement to reduce episodes of hypoglycemia unawareness or improve cognitive function, without causing diabetic ketoacidosis (the method includes monitoring and adjusting insulin accordingly).

Embodiment 138. A method of treating a neurological or psychiatric condition, comprising administering a ketone body composition together with one or more neuroactive drugs, wherein the ketone-induced metabolic state enhances the therapeutic effect or reduces side effects of the neuroactive drugs.

Embodiment 139. The method of embodiment 139, wherein the condition is epilepsy and the neuroactive drug is an anti epileptic drug (AED), and ketosis achieved by the ketone composition allows for a lower dose of the AED while maintaining seizure control, or provides additional seizure control in a refractory case (synergistic anticonvulsant effect).

Embodiment 140. The method of embodiment 139, wherein the condition is bipolar disorder and the subject is on a mood stabilizer (such as lithium or valproate), and the ketone therapy (which itself has mood stabilizing properties) allows for better mood stabilization or potentially a dose reduction of the mood stabilizer (for example, valproate dose can be lowered since BHB provides some overlapping HDAC inhibition and GABAergic effects).

Embodiment 141. The method of embodiment 139, wherein the condition is Alzheimer's disease and the subject is on a cholinesterase inhibitor or NMDA antagonist (standard AD drugs), and ketone therapy provides additional cognitive benefit by fueling neurons and reducing neuroinflammation, resulting in a significantly slower cognitive decline than the drug alone.

Embodiment 142. A method of modifying gene expression patterns for therapy, comprising administering a ketone body along with an epigenetic drug selected from: an HDAC inhibitor, a DNA methyltransferase inhibitor, a sirtuin activator, or a bromodomain inhibitor, such that the ketone's endogenous epigenetic effects complement the drug's mechanism.

Embodiment 143. The method of embodiment 143, wherein for example, a low-dose HDAC inhibitor (like valproate or SAHA) is combined with ketosis, potentially achieving a broader HDAC inhibition spectrum at lower drug concentrations, useful in diseases like cancer or certain neurodegenerative conditions.

Embodiment 144. A method of treating cardiovascular disease risk factors, comprising administering a ketone composition with one or more cardio-metabolic drugs such as statins, anti-hypertensives, or anti-inflammatory agents, wherein ketosis favorably alters lipid profiles, blood pressure, or vascular inflammation in synergy with said drugs.

Embodiment 145. The method of embodiment 145, wherein the subject is on a statin, and ketone therapy further raises HDL and lowers triglycerides, or reduces residual inflammatory risk (as indicated by high-sensitivity CRP) beyond what statin alone achieves.

Embodiment 146. A method of identifying a subject who will benefit from therapeutic ketosis (MERIT therapy), comprising: measuring in a subject a plurality of biomarkers reflecting metabolic, redox, and epigenetic status; calculating a composite “M-score” from said biomarkers; and selecting the subject for ketone-based therapy if the M-score is below a threshold indicative of metabolic/redox/epigenetic dysregulation, wherein subjects so selected are expected to have an improved therapeutic response to the ketone-based therapy.

Embodiment 147. The method of embodiment 147, wherein the plurality of biomarkers includes at least one metabolic biomarker (such as fasting insulin or HOMA-IR, or lactate level), at least one redox biomarker (such as blood GSH/GSSG ratio or NAD+level), and at least one epigenetic or inflammatory biomarker (such as histone acetylation level in PBMCs, or plasma IL-6), and the M-score is a weighted combination of these.

Embodiment 148. The method of embodiment 147, wherein the threshold is determined empirically such that patients below it have shown poor outcomes with standard care, and above it have better outcomes; thus a low M score identifies those likely to benefit from metabolic modulation via MERIT.

Embodiment 149. The method of embodiment 147, further comprising, after a period of ketone therapy, re-measuring the biomarkers and calculating an updated M-score, and if the M-score has improved to above a second threshold, adjusting the treatment (for example, tapering dose or focusing on maintenance).

Embodiment 150. A method of monitoring the efficacy of a ketone therapy in a subject, comprising periodically measuring at least one molecular marker of MERIT mechanism in the subject (such as NAD⁺ level, β hydroxybutyryl-histone levels, or inflammasome activity in blood cells) during therapy, and comparing it to baseline or target values, wherein an improvement in the marker toward the target confirms that the desired mechanistic effect of therapy is being achieved.

Embodiment 151. The method of embodiment 151, wherein the molecular marker monitored is the subject's blood β hydroxybutyrate: acetoacetate ratio and IL-1β level, and an observed decrease in ratio coupled with decrease in IL-1β during therapy indicates successful redox correction and inflammation reduction, suggesting the therapy is on target.

Embodiment 152. A diagnostic kit for evaluating candidacy for MERIT therapy, comprising reagents to measure a panel of biomarkers including at least NAD+/NADH or lactate/pyruvate (for redox), ketone body levels or insulin (for metabolic state), and histone acetylation or an inflammation marker (for epigenetic/immune state), and an instruction for computing an M-score or otherwise interpreting the results to identify if MERIT therapy is recommended.

Embodiment 153. A method of determining a personalized ketone target level for a subject, comprising: performing an ex vivo assay on the subject's cells (for example, exposing the subject's isolated PBMCs or serum to varying ketone concentrations) and measuring a mechanistic readout (like inflammasome activation or gene expression changes), and selecting a target ketone concentration that yields a desired 50% or greater improvement in the readout, wherein that concentration is then used as the therapeutic target in vivo for that subject.

Embodiment 154. A method of predicting therapeutic response to ketone therapy in a subject with a given disease, comprising identifying a metabolic or genetic signature in the subject (for instance, the presence of an APOE4 allele in Alzheimer's, or a specific metabolomic profile in a cancer patient), wherein if the signature is present, the subject is predicted to have an enhanced response to ketone therapy (or vice versa), thus informing clinical decision to employ MERIT.

Embodiment 155. A system for maintaining therapeutic ketosis in a subject, comprising: (a) a ketone level sensor configured to continuously or intermittently measure a ketone related value in the subject (such as blood β-hydroxybutyrate concentration); (b) a delivery device containing a ketone body formulation (such as a pump or infuser); and (c) a controller operatively connected to the sensor and delivery device, the controller programmed with a target ketone level or range and adapted to adjust the delivery device output to achieve and maintain the subject's ketone level within that target range.

Embodiment 156. The system of embodiment 156, wherein the ketone level sensor is a minimally invasive continuous ketone monitor (for example, a subcutaneous sensor measuring interstitial β-hydroxybutyrate) or a breath ketone analyzer, and the delivery device is an insulin pump adapted to deliver ketone solution instead (or a dual-chamber pump delivering both insulin and ketones in a diabetic scenario).

Embodiment 157. The system of embodiment 156, wherein the controller implements a feedback algorithm (for example, a PID controller or model-predictive control) that increases infusion rate when ketone level falls below setpoint and decreases or pauses when above setpoint, thus mimicking a closed-loop “artificial pancreas” but for ketone regulation.

Embodiment 158. The system of embodiment 156, further comprising a user interface that allows input of different target levels at different times (circadian profile) and displays real-time ketone values and infusion status to the user or clinician.

Embodiment 159. The system of embodiment 156, wherein the ketone formulation in the delivery device is a concentrated β hydroxybutyrate saline or other biocompatible solution, or a stable acetoacetate preparation, and the system is configured for either intravenous, subcutaneous, or intraperitoneal infusion as needed.

Embodiment 160. A portable device for therapeutic ketosis maintenance, comprising: a reservoir pre-loaded with a ketone ester liquid, a battery-operated pump, a sensor for BHB levels, and a microprocessor, integrated into a wearable unit, such that a patient can ambulate while the device monitors and auto-adjusts ketone delivery.

Embodiment 161. A kit for inducing and monitoring therapeutic ketosis in a patient, comprising: (i) a pharmaceutical composition comprising at least one ketone body or ketone precursor in unit dosage form; (ii) a ketone monitoring means (such as blood test strips and a meter, or a breath analyzer device); and (iii) instructions for use that guide the patient or caregiver to administer the composition to achieve a target ketone range and to measure ketone levels to adjust closing accordingly.

Embodiment 162. The kit of embodiment 162, wherein the ketone composition is provided in multiple forms (for example, a rapid-release form for quick elevation and a sustained-release form for maintenance), and the instructions detail how to use both (e.g., take rapid form to reach target within 30 minutes, then sustained form every 4 hours to maintain).

Embodiment 163. The kit of embodiment 162, wherein the instructions include a schedule for measuring additional biomarkers (like weekly morning fingerstick for blood NAD⁺ or periodic CRP measurement) to document improvement in mechanistic endpoints.

Embodiment 164. The kit of embodiment 162, specialized for epilepsy management, further including a chart or app for tracking seizures in relation to ketone readings, enabling correlation and optimization of the ketosis level for seizure control in that specific patient.

Embodiment 165. The kit of embodiment 162, specialized for weight loss/metabolic syndrome, further including dietary guidance (optionally moderate carb restriction advice to complement ketones) and an activity tracker, essentially packaging a full lifestyle program with the ketone pharmacotherapy.

Embodiment 166. A pharmaceutical composition comprising a β-hydroxybutyrate compound and an acetoacetate compound for use in treating [specific disease], wherein the treatment comprises achieving a target blood ketone level and maintaining it to modulate [metabolic/redox/epigenetic] parameters in the patient.

Embodiment 167. A composition according to embodiment 167 for use in treating a solid tumor, wherein the composition is to be administered in combination with an immune checkpoint inhibitor.

Embodiment 168. A composition comprising D-β-hydroxybutyrate or a precursor thereof, for use in increasing histone acetylation in neurons of a subject suffering from cognitive impairment, thereby improving cognitive function.

Embodiment 169. Use of a ketone body composition in the manufacture of a medicament for the treatment of an NLRP3 inflammasome-mediated inflammatory disease, wherein the medicament is adapted to raise blood β-hydroxybutyrate to at least 2 mM in the patient.

Embodiment 170. A pharmaceutical formulation comprising (a) β-hydroxybutyrate or a pharmaceutically acceptable salt thereof, and (b) acetoacetate or a stabilized acetoacetate derivative, in a ratio providing 1-10 parts β-hydroxybutyrate per 1 part acetoacetate on a molar basis, together with a pharmaceutically acceptable carrier, for use in treating a condition of reductive stress or metabolic dysregulation.

Embodiment 171. The formulation for use of embodiment 171, wherein the condition is heart failure, and the formulation is adapted for intravenous infusion to deliver both β-hydroxybutyrate and acetoacetate to the patient's bloodstream.

Embodiment 172. The formulation for use of embodiment 171, wherein the condition is epilepsy, and the formulation is adapted as an oral liquid or gel providing a high ratio of β-hydroxybutyrate for brain penetration and a sustained release effect.

Embodiment 173. A method of treating a metabolic-epigenetic-redox-immune (MERIT) responsive condition in a subject, comprising:
administering to the subject a therapeutically effective amount of a ketogenic agent selected from the group consisting of: (a) dietary restriction in the form of a ketogenic diet; (b) fasting selected from intermittent fasting and prolonged fasting; (c) an exogenous ketone monoester comprising (R)-3-hydroxybutyryl-(R)-3-hydroxybutyrate or an analog thereof; (d) an exogenous ketone salt selected from sodium β-hydroxybutyrate, potassium β-hydroxybutyrate, calcium β-hydroxybutyrate, sodium acetoacetate, potassium acetoacetate, and combinations thereof; (e)_{1,3}-butanediol or a derivative thereof; (f) a medium-chain triglyceride selected from C6, C8, C10, and C12 triglycerides; (g) a ketone-carrier conjugate comprising a polyol linked via a linker to one or more ketone bodies; (h) an SGLT2 inhibitor selected from empagliflozin, dapagliflozin, canagliflozin, ertugliflozin, and combinations thereof; (i) any combination of (a)-(h);
in an amount sufficient to induce and maintain in the subject a circulating β-hydroxybutyrate concentration of at least 0.5 millimolar (mM) for a duration of at least 7 days, thereby engaging metabolic state remodeling, epigenetic chromatin deacetylation, redox rebalancing, and immune tolerance;
wherein the method does not require that the subject exhibit glucose hypometabolism prior to, during, or after said administration, and is independent of dietary macronutrient restriction beyond that inherent to the ketogenic agent itself.

Embodiment 174. The method of embodiment 174, wherein the ketogenic agent is a dietary ketogenic diet comprising a macronutrient ratio of carbohydrate: protein: fat of approximately 5:20:75 by caloric weight.

Embodiment 175. The method of embodiment 174, wherein the ketogenic agent is fasting, and the subject undergoes intermittent fasting comprising 16-hour or 24-hour fasting windows separated by 8-hour or 12-hour feeding windows, maintained for a duration of at least 14 days.

Embodiment 176. The method of embodiment 174, wherein the ketogenic agent is an exogenous ketone monoester administered orally at a dose of 0.5-2.0 grams per dose, once or twice daily.

Embodiment 177. The method of embodiment 174, wherein the ketogenic agent is an exogenous ketone salt administered orally at a dose of 1-5 grams per day, divided into one or more doses.

Embodiment 178. The method of embodiment 174, wherein the ketogenic agent is 1,3-butanediol administered orally at a dose of 0.25-1.0 gram per kilogram of body weight per day.

Embodiment 179. The method of embodiment 174, wherein the ketogenic agent is a medium-chain triglyceride, specifically C8 (octanoic acid triglyceride), administered at a dose of 5-30 milliliters per day.

Embodiment 180. The method of embodiment 174, wherein the ketogenic agent is an SGLT2 inhibitor, and the subject is administered empagliflozin 10-25 mg daily or dapagliflozin 5-10 mg daily.

Embodiment 181. The method of embodiment 174, wherein the ketogenic agent is a combination of dietary ketogenic restriction and intermittent fasting.

Embodiment 182. The method of embodiment 174, wherein the circulating β-hydroxybutyrate concentration is maintained at 1.0-3.0 mM.

Embodiment 183. The method of embodiment 174, wherein the circulating β-hydroxybutyrate concentration is maintained at 1.5-5.0 mM.

Embodiment 184. The method of embodiment 174, wherein the duration of maintenance of said circulating β-hydroxybutyrate concentration is at least 14 days, 28 days, 90 days, or longer.

Embodiment 185. The method of embodiment 174, further comprising measuring baseline and on-treatment MERIT biomarkers selected from circulating β-hydroxybutyrate, urinary ketones, adipose tissue gene expression profile, peripheral blood mononuclear cell (PBMC) NAD+/NADH ratio, NLRP3 inflammasome activity, HCAR2 signaling activity, plasma histone deacetylase (HDAC) activity, and circulating IL-1β and IL-18 levels.

Embodiment 186. The method of embodiment 174, wherein the subject is administered said ketogenic agent in an ambulatory setting, without hospitalization or intensive monitoring.

Embodiment 187. The method of embodiment 174, wherein the subject is not required to exhibit abnormal fasting glucose, glucose intolerance, or impaired glucose oxidation capacity prior to, during, or after administration.

Embodiment 188. The method of embodiment 174, wherein the therapeutic benefit is mediated, in part, by suppression of NLRP3 inflammasome activation, as evidenced by reduced circulating IL-1β or IL-18 levels during or within 7 days of reaching target BHB concentration.

Embodiment 189. The method of embodiment 174, wherein the therapeutic benefit is mediated, in part, by elevation of the NAD+/NADH ratio in metabolically active cell populations, resulting in increased SIRT family deacetylase activity and enhanced cellular stress resistance.

Embodiment 190. The method of embodiment 174, comprising administering the ketogenic agent to a subject in an outpatient clinic or home setting, with periodic monitoring of circulating BHB concentration at days 3, 7, 14, and 28, and dose adjustment to maintain target BHB threshold.

Embodiment 191. A method of increasing the therapeutic efficacy of a second pharmaceutical agent in a subject having a MERIT-responsive condition, comprising:
inducing in the subject a ketotic metabolic state by administering a ketogenic agent selected from the group of embodiment 174(a)-(i), in an amount sufficient to achieve and maintain a circulating β-hydroxybutyrate concentration of at least 0.5 mM for a duration of at least 7 days;
subsequently administering to the subject said second pharmaceutical agent in a dose and schedule that would be therapeutically ineffective or sub-efficacious in the absence of the ketotic state;
wherein the second pharmaceutical agent exhibits greater therapeutic efficacy, as measured by tumor response rate, disease remission rate, symptom reduction, or biomarker improvement, when administered during or immediately following the ketotic state compared to historical baseline or control populations receiving said second agent without concomitant ketotic state induction;
wherein the method does not require that the subject exhibit glucose hypometabolism, and is independent of dietary restriction of the subject.

Embodiment 192. The method of embodiment 192, wherein the second pharmaceutical agent is an immune checkpoint inhibitor selected from anti-PD-1 (nivolumab, pembrolizumab), anti-PD-L1 (durvalumab, atezolizumab), anti-CTLA-4 (ipilimumab, tremelimumab), and combinations thereof.

Embodiment 193. The method of embodiment 192, wherein the second pharmaceutical agent is a tyrosine kinase inhibitor or serine/threonine kinase inhibitor selected from sunitinib, sorafenib, erlotinib, gefitinib, and PI3K pathway inhibitors (alpelisib, idelalisib).

Embodiment 194. The method of embodiment 192, wherein the second pharmaceutical agent is a chemotherapeutic agent selected from taxanes, platinum compounds, antimetabolites, and targeted biologics.

Embodiment 195. The method of embodiment 192, wherein the second pharmaceutical agent is a psychiatric or neurologic agent selected from selective serotonin reuptake inhibitors (SSRIs), serotonin-norepinephrine reuptake inhibitors (SNRIs), mood stabilizers, and antiepileptic agents.

Embodiment 196. The method of embodiment 192, wherein the subject previously failed to respond to said second agent when administered as monotherapy in an unselected population, and the ketotic state induction results in conversion of said subject from non-responder to responder phenotype.

Embodiment 197. The method of embodiment 192, wherein the second pharmaceutical agent is administered during the maintenance phase of the ketotic state, defined as the period during which circulating β-hydroxybutyrate is maintained at or above 0.5 mM.

Embodiment 198. The method of embodiment 192, wherein the second pharmaceutical agent is administered within 0-14 days prior to initiation of ketotic state induction, allowing pre-treatment sensitization.

Embodiment 199. The method of embodiment 192, wherein the subject exhibits a baseline MERIT biomarker signature consistent with metabolic dysfunction, epigenetic heterochromatin deposition, or immune exhaustion, and the ketotic state induction reverses said signature as measured by on-treatment biomarker readout.

Embodiment 200. The method of embodiment 192, wherein the therapeutic efficacy improvement is quantified as: (a) absolute response rate improvement of at least 15%, (b) progression-free survival improvement of at least 3 months, or (c) clinical symptom improvement by at least 50% relative to historical monotherapy controls.

Embodiment 201. The method of embodiment 192, wherein the ketogenic agent is administered concurrently with the second pharmaceutical agent for the entire duration of second agent treatment.

Embodiment 202. The method of embodiment 192, comprising withdrawing the ketogenic agent after the subject achieves sustained response (complete response, partial response, or stable disease lasting ≥12 weeks) and monitoring for loss of response; if loss of response occurs within 8 weeks, re-initiating ketotic state induction.

Embodiment 203. The method of embodiment 192, wherein the second pharmaceutical agent is an antimicrobial (antibiotic or antiviral) agent, and the ketotic state enhances immune-mediated clearance of chronic viral or bacterial infection, as evidenced by viral or bacterial load reduction or serologic conversion.

Embodiment 204. The method of embodiment 192, wherein the second pharmaceutical agent is an immunotherapy agent, and the ketotic state induction precedes said agent administration by 3-14 days to allow epigenetic and immune remodeling.

Embodiment 205. The method of embodiment 192, comprising restoring therapeutic efficacy to said second pharmaceutical agent in a subject in whom said agent previously failed to meet an efficacy endpoint in a clinical trial conducted in an unselected population, by inducing a concurrent ketotic state.

Embodiment 206. The method of embodiment 192, wherein the circulating β-hydroxybutyrate concentration is maintained at 1.0-3.0 mM during second agent administration.

Embodiment 207. The method of embodiment 192, wherein the subject is assessed for baseline MERIT biomarker dysregulation prior to induction, and the ketotic state is initiated only if the subject meets criteria for MERIT-responsive phenotype.

Embodiment 208. The method of embodiment 192, wherein the method is practiced in an ambulatory or clinic setting, without requirement for hospitalization, and comprises home-based monitoring of ketotic state achievement via urinary ketone strips or point-of-care serum BHB measurement.

Embodiment 209. A method of treating an immune-inflammatory condition characterized by dysregulation of NLRP3 inflammasome-mediated inflammation, in a subject not required to exhibit glucose hypometabolism, comprising:
administering a ketogenic agent selected from the group of embodiment 174(a)-(i) in an amount sufficient to induce and maintain a circulating β-hydroxybutyrate concentration of at least 0.5 mM for at least 7 days;
wherein the ketotic state suppresses NLRP3 inflammasome activation, as evidenced by reduced circulating IL-1β, IL-18, or IL-6 levels;
wherein the method does not require that the subject exhibit glucose hypometabolism, and is independent of dietary macronutrient restriction beyond that inherent to the ketogenic agent itself.

Embodiment 210. The method of embodiment 210, wherein the immune-inflammatory condition is selected from rheumatoid arthritis, systemic lupus erythematosus, Sjögren's syndrome, and scleroderma.

Embodiment 211. The method of embodiment 210, wherein the immune-inflammatory condition is inflammatory bowel disease, comprising Crohn's disease or ulcerative colitis, and the therapeutic endpoint is reduction in fecal calprotectin, endoscopic disease activity, or clinical symptom score (Crohn's Disease Activity Index, Mayo Score).

Embodiment 212. The method of embodiment 210, further comprising co-administering a standard-of-care immunosuppressive or biologic agent, such as a TNF-α inhibitor, IL-6 receptor antagonist, or JAK inhibitor, wherein the ketotic state potentiates the efficacy of said biologic.

Embodiment 213. The method of embodiment 210, comprising measuring baseline and on-treatment inflammasome biomarkers (circulating IL-1β, IL-18, caspase-1 activity) and continuing ketotic state induction if biomarkers remain elevated, or discontinuing if biomarkers normalize.

Embodiment 214. The method of embodiment 210, wherein the subject achieves clinical remission (defined as absence of symptoms, return to normal function, or resolution of objective inflammation markers) and the ketotic state is maintained chronically to prevent relapse.

Embodiment 215. A method of treating a dermatologic condition characterized by inflammatory infiltrate and dysregulated cutaneous immunity, in a subject not required to exhibit glucose hypometabolism, comprising:
administering a ketogenic agent selected from the group of embodiment 174(a)-(i) in an amount sufficient to achieve and maintain a circulating β-hydroxybutyrate concentration of at least 1.0 mM for at least 14 days;
wherein the ketotic state remodels cutaneous immune cell function and reduces inflammatory cytokine production;
measuring clinical endpoints including lesion erythema, induration, extent of affected body surface area, and pruritus intensity;
wherein the method does not require that the subject exhibit glucose hypometabolism, and is independent of dietary restriction of the subject.

Embodiment 216. The method of embodiment 216, wherein the dermatologic condition is atopic dermatitis, and the therapeutic endpoint is improvement in Eczema Area and Severity Index (EASI) score by at least 50% or achievement of clear/almost clear status on Investigator's Global Assessment (IGA).

Embodiment 217. The method of embodiment 216, wherein the dermatologic condition is psoriasis, and the therapeutic endpoint is improvement in Psoriasis Area and Severity Index (PASI) score by at least 75% (PASI-75 response).

Embodiment 218. The method of embodiment 216, further comprising biopsying affected skin at baseline and week 4 of ketotic state induction and analyzing for reduction in inflammatory immune infiltrate (T cell, B cell, macrophage, and dendritic cell density) by immunohistochemistry or flow cytometry.

Embodiment 219. The method of embodiment 216, wherein the dermatologic condition is selected from psoriatic arthritis, lichen planus, and alopecia areata.

Embodiment 220. A method of treating a psychiatric or neurologic condition mediated at least in part by reduced nicotinamide adenine dinucleotide (NAD⁺) signaling, histone deacetylase (HDAC) dysregulation, or metabolic insufficiency in the central nervous system, in a subject not required to exhibit glucose hypometabolism, comprising:
administering a ketogenic agent selected from the group of embodiment 174(a)-(i) in an amount sufficient to induce and maintain a circulating β-hydroxybutyrate concentration of at least 1.0 mM for at least 7 days;
wherein the circulating and central nervous system BHB acts as a substrate for NAD+-dependent signaling, inhibits HDAC activity via histone acetylation, and enhances mitochondrial function in neuronal and glial cells;
measuring clinical endpoints including symptom severity score, functional outcome measure, and mood or cognition assessment;
wherein the method does not require that the subject exhibit glucose hypometabolism, and is independent of dietary restriction of the subject.

Embodiment 221. The method of embodiment 221, wherein the psychiatric condition is major depressive disorder, and the subject is treatment-resistant (failure of at least two first-line serotonergic antidepressants), and the therapeutic endpoint is improvement in Montgomery-Åsberg Depression Rating Scale (MADRS) score by at least 50%.

Embodiment 222. The method of embodiment 221, wherein the psychiatric condition is bipolar disorder, depressive or manic episode, and the ketotic state induction is combined with mood-stabilizing pharmacotherapy (lithium, valproate, lamotrigine).

Embodiment 223. The method of embodiment 221, wherein the neurologic condition is Alzheimer's disease or mild cognitive impairment, and the therapeutic endpoint is stabilization or improvement in Mini-Cog score, Montreal Cognitive Assessment (MoCA), or functional independence measure.

Embodiment 224. The method of embodiment 221, wherein the neurologic condition is Parkinson's disease, epilepsy, or amyotrophic lateral sclerosis, and the therapeutic endpoint is improvement in motor function score, seizure frequency reduction, or slowing of motor decline.

Embodiment 225. A method of treating a bacterial or viral infection, including chronic infection or sepsis, in a subject not required to exhibit glucose hypometabolism, comprising:
administering a ketogenic agent selected from the group of embodiment 174(a)-(i) in an amount sufficient to induce and maintain a circulating β-hydroxybutyrate concentration of at least 0.5 mM for a duration dependent on infection type and severity;
wherein the ketotic state suppresses NLRP3 inflammasome-mediated pathologic inflammation while preserving or enhancing adaptive immune response (T cell and B cell function), and
co-administering an antimicrobial agent (antibiotic or antiviral) specific to the pathogen;
measuring endpoints including pathogen load (viral titer, bacterial colony—forming units), inflammatory biomarkers (IL-1β, TNF-α, IL-6), organ function (creatinine, transaminases, coagulation parameters), and clinical outcome (survival, ventilator-free days, ICU-free days);
wherein the method does not require that the subject exhibit glucose hypometabolism, and is independent of dietary restriction of the subject.

Embodiment 226. The method of embodiment 226, wherein the infection is gram-negative bacterial sepsis, and the ketotic state induction is initiated within 24-48 hours of diagnosis, concurrently with broad-spectrum antibiotics pending culture results.

Embodiment 227. The method of embodiment 226, wherein the infection is a chronic viral infection selected from cytomegalovirus (CMV), Epstein-Barr virus (EBV), human immunodeficiency virus (HIV), or hepatitis C virus (HCV), and the ketotic state enhances viral-specific T cell function, as measured by CD8+ T cell cytotoxic response or reduction in viral load.

Embodiment 228. The method of embodiment 226, wherein the infection is coronavirus infection (SARS-COV-2 or other), and the therapeutic endpoint is improved oxygenation, reduced progression to mechanical ventilation, and shortened hospitalization duration compared to matched controls receiving antimicrobial therapy without ketotic state induction.

Embodiment 229. A method of treating an oncologic indication via immune checkpoint inhibitor therapy combined with metabolic state manipulation, in a subject not required to exhibit glucose hypometabolism, comprising:
inducing a ketotic metabolic state by administering a ketogenic agent selected from the group of embodiment 174(a)-(i) in an amount sufficient to achieve and maintain a circulating β-hydroxybutyrate concentration of at least 1.0 mM;
maintaining said ketotic state for a period of 7-14 days prior to the first dose of immune checkpoint inhibitor therapy;
subsequently administering an immune checkpoint inhibitor selected from anti-PD-1, anti-PD-L1, anti-CTLA-4, or combinations thereof;
maintaining concurrent ketotic state induction throughout the duration of checkpoint inhibitor therapy;
measuring clinical endpoints including objective response rate, duration of response, progression-free survival, and overall survival;
wherein the ketotic state remodels tumor-associated immune dysfunction, reduces tumor-infiltrating regulatory T cells and myeloid-derived suppressor cells, enhances tumor-infiltrating effector T cell function, and increases tumor immunogenicity;
wherein the method does not require that the subject exhibit glucose hypometabolism, and is independent of dietary restriction of the subject.

Embodiment 230. The method of embodiment 230, wherein the oncologic indication is melanoma, and the therapeutic endpoint is objective response rate improvement from historical 30-40% monotherapy baseline to >60% with combined ketotic state induction and checkpoint inhibitor.

Embodiment 231. The method of embodiment 230, wherein the oncologic indication is non-small-cell lung cancer (NSCLC), renal cell carcinoma (RCC), hepatocellular carcinoma (HCC), or colorectal cancer (CRC), and the therapeutic endpoint is checkpoint inhibitor efficacy restoration in previously refractory disease populations.

Embodiment 232. The method of embodiment 230, wherein the subject undergoes baseline tumor biopsy and transcriptomic profiling to assess tumor-infiltrating lymphocyte (TIL) density, PD-L1 expression, myeloid cell infiltration, and metabolic gene expression signature; ketotic state induction is continued if baseline profiling indicates cold tumor phenotype (low TIL, high myeloid suppression); and on-treatment biopsy at week 4 is obtained to document immune remodeling.

Embodiment 233. A method of selecting and monitoring subjects for ketosis-based therapeutic intervention, comprising:
measuring in a biological sample obtained from the subject at least one MERIT biomarker selected from the group consisting of: (i) circulating β-hydroxybutyrate concentration; (ii) urinary ketone concentration; (iii) adipose tissue transcriptomic signature indicating dysregulated energy metabolism; (iv) peripheral blood mononuclear cell (PBMC) NAD+/NADH ratio; (v) NLRP3 inflammasome activity, as measured by circulating IL-1β, IL-18, or caspase-1 activity; (vi) HCAR2 signaling activity in immune cell populations; (vii) histone deacetylase (HDAC) activity; (viii) a composite metabolic vulnerability index derived from a multiplex biomarker panel;
comparing the measured biomarker value(s) to an established threshold or reference range;
selecting for ketosis-based therapeutic intervention any subject whose biomarker value(s) indicate dysregulation of metabolic state, epigenetic tone, redox balance, or immune function;
wherein the method enables identification of MERIT-responsive subjects independent of traditional disease classification;
wherein the method does not require that the subject exhibit glucose hypometabolism; and wherein the method is independent of dietary restriction of the subject.

Embodiment 234. The method of embodiment 234, wherein the sample is venous blood or capillary blood, and circulating β-hydroxybutyrate is measured by liquid chromatography-mass spectrometry (LC-MS), gas chromatography-mass spectrometry (GC-MS), or colorimetric enzyme assay.

Embodiment 235. The method of embodiment 234, wherein the PBMC NAD+/NADH ratio is measured by high-performance liquid chromatography (HPLC) or mass spectrometry, and a ratio of <2.0 indicates dysregulation warranting ketosis-based intervention.

Embodiment 236. The method of embodiment 234, wherein NLRP3 inflammasome activity is measured by quantifying circulating IL-1β (>50 μg/mL) or IL-18 (>500 μg/mL) using high-sensitivity immunoassay.

Embodiment 237. The method of embodiment 234, wherein the composite metabolic vulnerability index is derived from a machine-learning algorithm incorporating ≥5 biomarkers, weighted by their association with MERIT-responsive disease phenotype, and a calculated index>0.6 (on a scale of 0-1) indicates ketosis-based intervention candidacy.

Embodiment 238. A method of monitoring therapeutic response to a ketogenic agent in a subject with a MERIT-responsive condition, comprising:
measuring baseline target-engagement biomarkers in said subject selected from the group consisting of NLRP3 inflammasome activity, HCAR2 signaling activity, PBMC NAD+/NADH ratio, HDAC activity, circulating metabolic substrate ratios, and epigenetic chromatin histone acetylation state;
initiating ketogenic agent administration in an amount sufficient to induce and maintain a circulating β-hydroxybutyrate concentration of at least 0.5 mM;
measuring on-treatment target-engagement biomarkers at defined timepoints (days 3, 7, 14, 28);
continuing ketogenic agent administration if target-engagement biomarkers demonstrate the intended mechanistic effect;
adjusting ketogenic agent dose or switching to an alternative ketogenic agent (from the group of embodiment 174(a)-(i)) if target-engagement biomarkers fail to demonstrate expected engagement;
measuring clinical outcome biomarkers (disease-specific endpoints) at days 7, 14, 28, and 90 to assess therapeutic efficacy;
wherein target-engagement biomarker-guided adjustment optimizes therapeutic response independent of empirical dose escalation alone;
wherein the method does not require that the subject exhibit glucose hypometabolism, and is independent of dietary restriction of the subject.

Embodiment 239. The method of embodiment 239, wherein the target-engagement biomarker is NLRP3 inflammasome activity (circulating IL-1β or IL-18), and a reduction of ≥30% from baseline indicates adequate MERIT target engagement.

Embodiment 240. The method of embodiment 239, wherein the target-engagement biomarker is PBMC NAD+/NADH ratio, and an elevation to ≥2.5 indicates restoration of NAD+-dependent signaling.

Embodiment 241. The method of embodiment 239, wherein the clinical outcome biomarker is disease-specific (e.g., EASI for atopic dermatitis, PASI for psoriasis, MADRS for depression, tumor response rate for oncology), and a predefined improvement threshold triggers assessment of biomarker-guided dose optimization.

Embodiment 242. The method of embodiment 239, further comprising measuring circulating β-hydroxybutyrate at each timepoint to confirm adequate ketotic state induction (>0.5 mM), and escalating ketogenic agent dose if BHB remains below threshold.

Embodiment 243. The method of embodiment 239, comprising performing optional adipose tissue or tumor biopsy and transcriptomic analysis at baseline and week 4, to directly assess epigenetic and metabolic gene expression changes in response to ketotic state induction.

Embodiment 244. A kit for MERIT-responsive subject selection and therapeutic monitoring, comprising:
(a) a ketogenic agent selected from the group of embodiment 174(a)-(i);
(b) one or more MERIT biomarker assay(s) selected from the group of embodiment 234 (i)-(viii), in a form suitable for point-of-care or laboratory measurement;
(c) a reference table or nomogram relating biomarker values to MERIT-responsive phenotype and ketosis-based intervention eligibility;
(d) written instructions for subject selection, ketogenic agent closing, on-treatment biomarker monitoring at defined intervals (days 3, 7, 14, 28, 90), and dose adjustment criteria;
(e) optionally: a portable meter or assay cartridge for measuring circulating β-hydroxybutyrate in a clinic or home setting;
wherein the kit is configured for use with subjects who do not exhibit glucose hypometabolism, and is independent of dietary restriction of the subject.

Embodiment 245. The kit of embodiment 245, wherein the ketogenic agent is an oral exogenous ketone salt or monoester, pre—formulated in unit-dose packets, oral liquid, suspension, or tablets.

Embodiment 246. The kit of embodiment 245, wherein the MERIT biomarker assay comprises a multiplexed immunoassay panel for simultaneous measurement of circulating IL-1β, IL-18, TNF-α, and IL-6.

Embodiment 247. The kit of embodiment 245, wherein the written instructions comprise a treat-to-target algorithm specifying circulating β-hydroxybutyrate target of 1.0-3.0 mM, measurement frequency, and decision rules for dose escalation, maintenance, or discontinuation based on biomarker readout and clinical response.

Embodiment 248. A pharmaceutical composition for enhancing therapeutic efficacy of a second pharmaceutical agent via concomitant metabolic state modulation, comprising:
(a) a ketogenic agent selected from the group consisting of: (i) an exogenous ketone monoester comprising (R)-3-hydroxybutyryl-(R)-3-hydroxybutyrate; (ii) an exogenous ketone salt selected from sodium, potassium, or calcium β-hydroxybutyrate; (iii) a ketone-carrier conjugate comprising a polyol linked to one or more ketone bodies; (iv)_{1,3}-butanediol or a derivative thereof; (v) a medium-chain triglyceride; (vi) an SGLT2 inhibitor selected from empagliflozin, dapagliflozin, canagliflozin, or ertugliflozin;
(b) a second pharmaceutical agent selected from the group consisting of: (i) an immune checkpoint inhibitor (anti-PD-1, anti-PD-L1, anti-CTLA-4); (ii) a kinase inhibitor (PI3K, MEK, JAK, or tyrosine kinase inhibitor); (iii) a chemotherapeutic agent; (iv) a psychiatric or neurologic agent (SSRI, SNRI, mood stabilizer, antiepileptic); (v) an antimicrobial agent (antibiotic or antiviral); (vi) a biologic immune-modulating agent; (vii) optionally, a pharmaceutically acceptable excipient, carrier, or vehicle;
formulated for co-administration in amounts sufficient to (1) induce and maintain a circulating β-hydroxybutyrate concentration of at least 0.5 mM, and (2) achieve therapeutic concentrations of the second agent at the site of action;
wherein the composition is formulated to provide synergistic or super-additive therapeutic benefit via state-dependent enhancement of second agent efficacy;
wherein the composition does not require that the subject exhibit glucose hypometabolism, and is independent of dietary restriction of the subject.

Embodiment 249. The composition of embodiment 249, wherein the ketogenic agent is an exogenous ketone monoester and the second pharmaceutical agent is an immune checkpoint inhibitor, co—formulated in a unit-dose oral preparation.

Embodiment 250. The composition of embodiment 249, wherein the ketogenic agent is a ketone-carrier conjugate and the second pharmaceutical agent is a PI3K inhibitor or JAK inhibitor, co—formulated as a fixed-dose combination oral liquid or suspension.

Embodiment 251. The composition of embodiment 249, wherein the ketogenic agent and second pharmaceutical agent are formulated in separate, temporally staggered unit-dose forms, with instructions to administer the ketogenic agent 7-14 days prior to the second agent, to allow for metabolic priming.

Embodiment 252. The composition of embodiment 249, wherein the composition further comprises a MERIT biomarker assay kit or device to enable patient selection or on-treatment therapeutic monitoring.

Embodiment 253. A kit for treating a MERIT-responsive condition via combined ketosis induction and therapeutic intervention, comprising:
(a) a ketogenic agent in a form suitable for oral or intravenous administration, in a unit-dose or multi-dose presentation;
(b) a second pharmaceutical agent as defined in embodiment 249 (b), in a therapeutic unit-dose;
(c) a MERIT biomarker assay or point-of-care measurement device selected from the group of embodiment 234 (i)-(viii);
(d) written instructions comprising: (i) subject selection criteria based on MERIT biomarker profile; (ii) ketogenic agent closing, administration route, and frequency; (iii) timing of second agent administration relative to ketogenic agent initiation; (iv) on-treatment biomarker monitoring schedule and decision rules; (v) clinical outcome assessment timepoints and target endpoints; (vi) safety monitoring parameters and dose adjustment algorithms;
(e) optionally: a patient adherence tracking tool, such as a mobile application or paper diary;
wherein the kit is configured for use with subjects who do not exhibit glucose hypometabolism, and is independent of dietary restriction of the subject.

Embodiment 254. The kit of embodiment 254, wherein the written instructions specify that the ketogenic agent shall be administered to achieve and maintain a circulating β-hydroxybutyrate concentration of 1.0-3.0 mM for a minimum of 7 days prior to initiation of the second agent.

Embodiment 255. The kit of embodiment 254, wherein the ketogenic agent comprises a combination of an exogenous ketone salt and an SGLT2 inhibitor, formulated for concurrent or sequential administration.

Embodiment 256. The kit of embodiment 254, wherein the MERIT biomarker assay comprises a multiplexed panel enabling measurement of at least three biomarkers selected from circulating BHB, NLRP3 inflammasome activity, PBMC NAD+/NADH ratio, and HCAR2 signaling activity.

Embodiment 257. A therapeutic combination product comprising a ketogenic agent and a diagnostic biomarker measurement component, integrated for use in identifying and treating MERIT-responsive subjects, comprising:
(a) a pharmaceutical composition containing a ketogenic agent as defined in embodiment 174(a)-(i);
(b) an integrated diagnostic device or assay capable of measuring a MERIT biomarker selected from the group of embodiment 234 (i)-(viii) in a real-time or near-real-time fashion (results available within 5-60 minutes);
(c) a computational algorithm or display interface that correlates the measured biomarker value(s) to subject MERIT-responsiveness and provides a recommendation for (i) ketogenic agent initiation, (ii) dose, (iii) duration, or (iv) discontinuation;
(d) written instructions for use, including subject selection, biomarker measurement procedure, interpretation, and ketogenic agent administration guidance;
wherein the combination product enables bedside or clinic-based MERIT phenotyping and real-time therapeutic guidance;
wherein the composition does not require that the subject exhibit glucose hypometabolism, and is independent of dietary restriction of the subject.

Embodiment 258. The combination product of embodiment 258, wherein the diagnostic device is a point-of-care meter measuring circulating β-hydroxybutyrate from a finger-stick blood sample or breath acetone.

Embodiment 259. The combination product of embodiment 258, wherein the computational algorithm incorporates machine-learning classification trained on MERIT-responsive and MERIT-non-responsive subject populations, and generates a MERIT-responsiveness probability score (0-100%) based on measured biomarker value(s).

Embodiment 260. The combination product of embodiment 258, wherein the ketogenic agent is pre-packaged in a single-unit dose form with instructions specifying administration only if the biomarker measurement indicates MERIT-responsive status (e.g., circulating BHB<0.5 mM at baseline, NLRP3 activity >threshold, NAD+/NADH ratio <2.0).

Embodiment 261. A method of manufacturing a fixed-dose combination pharmaceutical composition comprising a ketogenic agent and a second therapeutic agent, comprising:
mixing a therapeutically effective amount of a ketogenic agent selected from the group of embodiment 174(a)-(i) with a therapeutically effective amount of a second agent selected from the group of embodiment 249 (b);
incorporating the mixture into a pharmaceutically acceptable carrier, excipient, or vehicle; forming unit-dose forms (oral liquids, suspensions, tablets, capsules, sachets, or vials);
quantitatively assaying the final composition to verify target β-hydroxybutyrate equivalent concentration (molar basis) and second agent concentration;
packaging the unit-dose forms with labeling and instructions specifying use for MERIT-responsive condition treatment;
wherein the fixed-dose combination is manufactured to ensure stoichiometric equivalence across unit doses, enabling predictable bioavailability and pharmacodynamic target engagement;
wherein the method does not require that the subject exhibit glucose hypometabolism, and is independent of dietary restriction of the subject.

Embodiment 262. The method of embodiment 262, wherein the ketogenic agent is an exogenous ketone salt and the second agent is an SGLT2 inhibitor, and the unit-dose form is an oral liquid or suspension comprising 2 grams of ketone salt and 10 mg of SGLT2 inhibitor.

Embodiment 263. The method of embodiment 262, wherein the assay comprises high-performance liquid chromatography (HPLC) or mass spectrometry to quantify ketone body concentration and second agent concentration independently, ensuring compositional consistency within +10% of target values.

Embodiment 264. A pharmaceutical composition in a form suitable for oral administration to a human subject, comprising:
(a) a ketogenic agent selected from the group consisting of an exogenous ketone monoester, an exogenous ketone salt, a ketone-carrier conjugate, 1,3-butanediol, or a medium-chain triglyceride;
(b) a taste-masking agent selected from the group consisting of microcrystalline cellulose, silica, citric acid, or a proprietary taste-masking polymer;
(c) a bioavailability enhancer selected from the group consisting of permeation enhancers, surfactants, or lipid-based formulation excipients;
(d) optionally, a coloring agent approved for food or pharmaceutical use;
(e) optionally, a sweetening agent;
formulated as an oral liquid, suspension, powder sachet, tablet, or capsule;
wherein the composition is formulated to optimize palatability and oral bioavailability of the ketogenic agent, enabling consistent achievement of circulating β-hydroxybutyrate concentrations of at least 0.5 mM within 60-120 minutes of administration.

Embodiment 265. A method of treating a MERIT-responsive condition in a subject, comprising: administering a ketogenic agent in an amount sufficient to achieve a target ratio of circulating β-hydroxybutyrate to acetoacetate (BHB:AcAc) in the range of about 1:1 to about 5:1; monitoring the BHB:AcAc ratio by measuring both circulating BHB and circulating AcAc; and adjusting the dose or composition of the ketogenic agent to maintain the target BHB:AcAc ratio for a duration sufficient to engage at least one of histone β-hydroxybutyrylation, NLRP3 inflammasome suppression, or HCAR2 signaling; wherein the method does not require that the subject exhibit glucose hypometabolism; and wherein the method is independent of dietary restriction of the subject.

Embodiment 266. The method of embodiment 266, wherein AcAc is measured by LC-MS/MS or enzymatic assay in a biological sample from the subject.

Embodiment 267. The method of embodiment 266, wherein the target BHB:AcAc ratio is optimized for the specific MERIT-responsive condition.

Embodiment 268. The method of embodiment 266, wherein the ketogenic agent is a ketone-carrier conjugate wherein R² comprises acetoacetate, such that AcAc is delivered directly rather than relying solely on hepatic interconversion from BHB.

Embodiment 269. The method of embodiment 266, wherein the method further comprises measuring total ketone body exposure (BHB+AcAc+acetone) and adjusting the dose to maintain a total ketone body concentration of at least 0.8 mM.

Embodiment 270. The method of embodiment 266, wherein the method further comprises measuring breath acetone concentration as a non-invasive surrogate for circulating acetoacetate.

Embodiment 271. The method of embodiment 266, wherein the BHB:AcAc ratio is used to stratify the subject for selection of a specific ketogenic agent from the group consisting of a BHB-dominant composition, an AcAc-dominant composition, and a dual-ketone composition.

Embodiment 272. The method of embodiment 266, wherein the MERIT-responsive condition is selected from the group consisting of epilepsy, psychiatric disorder, neurodegenerative disorder, and oncology, and the target BHB:AcAc ratio is indication-specific.

Embodiment 273. The method of embodiment 266, wherein the AcAc-dominant composition achieves lysine acetoacetylation (Kacac) at histone sites distinct from β-hydroxybutyrylation (Kbhb), thereby engaging an epigenetic program complementary to but distinct from BHB-mediated Kbhb.

Embodiment 274. The method of embodiment 266, wherein the method further comprises measuring site-specific histone modifications selected from the group consisting of K18ac, K27ac, K27bhb, and K36bhb to verify target engagement of the optimized BHB:AcAc ratio.

Embodiment 275. A method of treating a MERIT-responsive condition in a subject, comprising: determining a circadian phase of the subject by measuring at least one circadian biomarker selected from the group consisting of cortisol awakening response, core body temperature nadir timing, melatonin onset, and clock gene expression level of at least one of BMAL1, PER2, CRY1, and CLOCK; administering a ketogenic agent at a time of day selected to align peak circulating β-hydroxybutyrate concentration with a target circadian phase of the subject; wherein the target circadian phase is selected to maximize engagement of at least one MERIT pillar mechanism; wherein the method does not require that the subject exhibit glucose hypometabolism; and wherein the method is independent of dietary restriction of the subject.

Embodiment 276. The method of embodiment 276, wherein the target circadian phase is the morning cortisol peak phase and the MERIT pillar mechanism is BMAL1-mediated circadian metabolic synchronization.

Embodiment 277. The method of embodiment 276, wherein the target circadian phase is the evening inflammatory peak phase and the MERIT pillar mechanism is NLRP3 inflammasome suppression.

Embodiment 278. The method of embodiment 276, wherein the method further comprises co-administering a second therapeutic agent, and the ketogenic agent is administered at a time selected to align peak BHB concentration with peak plasma concentration (Cmax) of the second therapeutic agent.

Embodiment 279. The method of embodiment 279, wherein the second therapeutic agent is an immune checkpoint inhibitor and the aligned pharmacokinetic windows maximize T-cell metabolic fitness during peak drug-target interaction.

Embodiment 280. The method of embodiment 276, wherein the circadian biomarker is measured using a wearable device that monitors at least one of skin temperature, heart rate variability, and actigraphy to infer circadian phase.

Embodiment 281. The method of embodiment 276, wherein the method is repeated daily with adaptive timing adjustment based on serial circadian biomarker measurements.

Embodiment 282. The method of embodiment 276, wherein the subject has a circadian rhythm disorder and the method further comprises using the ketogenic agent to entrain the circadian clock via BHB-mediated activation of BMAL1 and PER2.

Embodiment 283. The method of embodiment 276, wherein the ketogenic agent is administered in a modified-release formulation designed to produce a BHB exposure profile that matches a predetermined circadian target curve.

Embodiment 284. The method of embodiment 276, wherein the MERIT-responsive condition is selected from the group consisting of a psychiatric disorder characterized by circadian disruption, a neurodegenerative disorder characterized by sleep-wake cycle dysregulation, and an oncology condition in which immune checkpoint receptor expression varies with circadian phase.

Embodiment 285. A method of monitoring and optimizing therapeutic ketosis in a subject being treated for a MERIT-responsive condition, comprising: measuring circulating glucose concentration and circulating β-hydroxybutyrate concentration in the subject; calculating a Glucose-Ketone Index (GKI) according to the formula: GKI=[glucose (mg/dL)]/([BHB (mmol/L)]×18.01); comparing the calculated GKI to a target GKI threshold; and adjusting the dose of a ketogenic agent administered to the subject to achieve or maintain a GKI at or below the target GKI threshold; wherein the method does not require that the subject exhibit glucose hypometabolism; and wherein the method is independent of dietary restriction of the subject.

Embodiment 286. The method of embodiment 286, wherein the target GKI threshold is indication-specific and selected from: GKI≤1.0 for an oncology indication, GKI≤1.0 for a psychiatric indication, GKI≤1.2 for a sepsis or infectious disease indication, GKI≤2.0 for a neurodegenerative indication, and GKI≤3.0 for a metabolic or cardiovascular indication.

Embodiment 287. The method of embodiment 286, wherein glucose and BHB are measured using a point-of-care dual-analyte meter capable of measuring both glucose and BHB from a single capillary blood sample.

Embodiment 288. The method of embodiment 286, wherein the GKI is calculated in real-time by a software algorithm integrated with a continuous glucose monitor and a continuous or intermittent ketone monitor.

Embodiment 289. The method of embodiment 286, wherein the method further comprises measuring at least one MERIT biomarker selected from the group consisting of Kbhb, NLRP3 activity, and NAD+/NADH ratio, and using the combination of GKI and the at least one MERIT biomarker to guide dose adjustment.

Embodiment 290. The method of embodiment 286, wherein the subject is being co-administered a second therapeutic agent and the GKI target threshold is adjusted based on the pharmacodynamic requirements of the second agent.

Embodiment 291. A kit comprising: a dual-analyte point-of-care meter for measuring glucose and β-hydroxybutyrate; instructions for calculating a Glucose-Ketone Index (GKI); and a reference card listing indication-specific target GKI thresholds for MERIT-responsive conditions; wherein use of the kit does not require that the subject exhibit glucose hypometabolism; and wherein use of the kit is independent of dietary restriction of the subject.

Embodiment 292. A method of selecting a subject for ketosis-based therapy for a MERIT-responsive condition, comprising: measuring a baseline GKI in the subject; determining that the baseline GKI is above a threshold indicative of metabolic dysregulation for the subject's MERIT-responsive condition; and administering a ketogenic agent to the subject to reduce the GKI to at or below the indication-specific target threshold; wherein the method does not require that the subject exhibit glucose hypometabolism; and wherein the method is independent of dietary restriction of the subject.

Embodiment 293. A method of treating a MERIT-responsive condition in a subject comprising: (a) determining whether the subject's condition is characterized as fuel-driven (where primary mechanism is energy substrate delivery to dysfunctional mitochondria) or signal-driven (where primary mechanism is activation of specific signaling pathways at defined concentration thresholds); (b) if fuel-driven, selecting a closing regimen of a ketogenic agent comprising high single-dose or infrequent (once to twice daily) administration to maximize total circulating β-hydroxybutyrate area-under-the-curve (AUC); (c) if signal-driven, selecting a closing regimen of a ketogenic agent comprising lower, frequent (three to four times daily) administration to maintain circulating β-hydroxybutyrate within a target concentration window (0.5-2.0 mM) for >2.6 hours daily; (d) administering the selected closing regimen; and (e) monitoring for therapeutic response; wherein the method does not require that the subject exhibit glucose hypometabolism; and wherein the method is independent of dietary restriction of the subject.

Embodiment 294. The method of embodiment 294, wherein the fuel-driven condition is selected from acute myocardial infarction with cardiogenic shock, severe traumatic brain injury, acute ischemic stroke, acute hepatic failure, acute mitochondrial depletion syndrome, or severe sepsis-associated mitochondrial dysfunction, and the ketogenic agent is dosed to achieve Cmax ≥1.0 mM and AUC≥8 mM hours (measured as total BHB exposure above 0.5 mM baseline over a 24-hour period).

Embodiment 295. The method of embodiment 294, wherein the signal-driven condition is selected from NLRP3 inflammasome-driven autoinflammatory disease, autoimmune inflammatory disease, oncology requiring immune metabolic remodeling, or neuropsychiatric disease where epigenetic remodeling is primary, and the ketogenic agent is dosed to maintain circulating β-hydroxybutyrate 0.5-2.0 mM for >2.6 hours daily while remaining <2.4 mM.

Embodiment 296. The method of embodiment 294, further comprising calculating a Glucose-Ketone Index (GKI) and adjusting dose or frequency to maintain an indication-specific target GKI.

Embodiment 297. The method of embodiment 294, wherein the determination of fuel-driven vs. signal-driven is made by assessing the predominant MERIT pillar(s) implicated in the condition: Metabolic pillar predominance indicates fuel-driven; Epigenetic, Immune, Redox, or Signaling pillar predominance indicates signal-driven.

Embodiment 298. A method of optimizing pharmacologically-induced ketosis to simultaneously engage multiple beneficial mechanisms while avoiding off-target mechanisms, comprising: (a) identifying a subject with a MERIT-responsive condition where multiple beneficial mechanisms require engagement; (b) selecting a ketogenic agent and dose to maintain circulating β-hydroxybutyrate in a therapeutic optimization corridor of 0.5-2.0 mM for maximal duration per day, wherein the corridor simultaneously engages NLRP3 inflammasome suppression, HCAR2 signaling, and Kbhb histone modification while remaining below the class I HDAC engagement threshold of approximately 2.4 mM; (c) administering the selected ketogenic agent; and (d) measuring circulating BHB and at least one mechanism-specific biomarker to confirm multi-mechanism engagement; wherein the method does not require that the subject exhibit glucose hypometabolism; and wherein the method is independent of dietary restriction of the subject.

Embodiment 299. The method of embodiment 299, wherein the multi-mechanism engagement is confirmed by measuring at least two of: hi stone Kbhb enrichment, NLRP3 inflammasome activity in circulating monocytes, HCAR2-mediated signaling, and NAD+/NADH ratio.

Embodiment 300. The method of embodiment 299, further comprising constructing a pharmacokinetic model describing BHB concentration as a function of dose, formulation, and time, and calculating integrated cumulative target engagement across NLRP3, HCAR2, and Kbhb mechanisms to identify the regimen maximizing integrated engagement within the 0.5-2.0 mM corridor.

Embodiment 301. The method of embodiment 299, wherein the ketogenic agent achieves pharmacokinetic parameters substantially equivalent to Study 25-101 at 5 g/kg: Cmax approximately 1.542 mM, time above 0.5 mM approximately 7.6 hours, time above 1.0 mM approximately 2.6 hours, and time above 2.4 mM approximately 0 hours.

Embodiment 302. A method of treating a MERIT-responsive condition in a subject comprising: (a) classifying the subject's condition into one of the following categories: (i) sepsis or systemic infection; (ii) autoimmune or inflammatory disease; (iii) oncology with immune-exclusion phenotype; (iv) neurodegenerative disease; (v) psychiatric or mood disorder; (vi) cardiac disease; (vii) dermatologic disease; or (viii) kidney disease; (b) selecting an indication-specific target circulating β-hydroxybutyrate range based on the condition category: 0.5-1.0 mM for sepsis; 1.0-2.0 mM for autoimmune disease; 1.5-3.0 mM for oncology; 0.5-1.5 mM for neurodegeneration; 0.5-1.5 mM for psychiatry; 0.5-1.0 mM for cardiac disease; 0.5-1.5 mM for dermatology; or 0.5-1.0 mM for kidney disease; (c) administering a ketogenic agent in an amount to achieve the indication-specific target; and (d) measuring circulating BHB at intervals and adjusting dose to maintain the target range; wherein the method does not require that the subject exhibit glucose hypometabolism; and wherein the method is independent of dietary restriction of the subject.

Embodiment 303. The method of embodiment 303, wherein for sepsis (category i), the target range of 0.5-1.0 mM is selected to achieve rapid NLRP3 inflammasome suppression in innate immune cells.

Embodiment 304. The method of embodiment 303, wherein for autoimmune disease (category ii), the target range of 1.0-2.0 mM is selected to achieve sustained NLRP3 suppression combined with Kbhb-mediated regulatory T cell differentiation.

Embodiment 305. The method of embodiment 303, wherein for oncology (category iii), the target range of 1.5-3.0 mM is selected to achieve deep Kbhb deposition in tumor-infiltrating T cells for immune-metabolic remodeling of the tumor microenvironment.

Embodiment 306. The method of embodiment 303, wherein for neurodegenerative disease (category iv), the target range of 0.5-1.5 mM is selected to provide combined fuel-substrate replacement and HCAR2-mediated neuroinflammation suppression.

Embodiment 307. The method of embodiment 303, wherein for psychiatric disorder (category v), the target range of 0.5-1.5 mM is selected to achieve Kbhb-mediated epigenetic modulation in mood-circuit neurons combined with HCAR2 signaling in microglia.

Embodiment 308. The method of embodiment 303, wherein for cardiac disease (category vi), the target range of 0.5-1.0 mM is selected to provide myocardial energy substrate replacement and NAD+/NADH redox rebalancing.

Embodiment 309. The method of embodiment 303, wherein for dermatologic disease (category vii), the target range of 0.5-1.5 mM is selected to achieve NLRP3 suppression in skin-resident immune cells combined with restoration of epidermal barrier function.

Embodiment 310. The method of embodiment 303, wherein for kidney disease (category viii), the target range of 0.5-1.0 mM is selected to achieve renal mitochondrial protection and NLRP3 suppression in glomerular and tubular immune cells.

Embodiment 311. A method of treating an autoinflammatory disease in a subject, comprising: (a) identifying a subject with an autoinflammatory condition selected from familial Mediterranean fever, cryopyrin-associated periodic syndromes (FCAS, Muckle-Wells syndrome, NOMID), gout, or other monogenic autoinflammatory syndromes where NLRP3 inflammasome hyperactivation is the validated pathophysiologic mechanism; (b) administering a ketogenic agent in an amount sufficient to induce and maintain circulating β-hydroxybutyrate in the range 1.0-2.0 mM for at least 2.6 hours per day, while remaining below 2.4 mM; (c) measuring circulating BHB and NLRP3 inflammasome activation at intervals; and (d) adjusting dose to maintain the specified BHB time-window; wherein the method does not require that the subject exhibit glucose hypometabolism; and wherein the method is independent of dietary restriction of the subject.

Embodiment 312. The method of embodiment 312, wherein a ketogenic agent achieving the pharmacokinetic profile of Study 25-101 at 5 g/kg (Cmax 1.542 mM, time above 1.0 mM approximately 2.6 hours, maximum below 2.4 mM) directly produces the embodimented NLRP3 time-window.

Embodiment 313. The method of embodiment 312, wherein NLRP3 inflammasome activation is measured by phospho-caspase-1 intracellular staining in circulating monocytes by flow cytometry and by circulating IL-1β and IL-18 levels.

Embodiment 314. The method of embodiment 312, wherein the autoinflammatory disease is a monogenic NLRP3 gain-of-function condition responsive to IL-1β antagonism.

### EXAMPLES

The following examples are illustrative and/or prophetic, demonstrating the application of the methods, systems, and principles described herein. In some examples, hypothetical data are included to exemplify potential outcomes.

Abbreviations

| | |
|---|---|
| AcAc | Acetoacetate |
| ACN | Acetonitrile |
| BHB | Beta-hydroxybutyrate |
| DI H₂O | Deionized water |
| DMAP | 4-(Dimethylamino)pyridine |
| EDC | N-Ethyl-N′-(3- |
| | dimethylaminopropyl)carbodiimide |
| K₂CO₃ | Potassium carbonate |
| LiOH | Lithium hydroxide monohydrate |
| MeAA | methyl acetoacetate |
| MeOH | Methanol |
| MgSO₄ | Magnesium sulfate |
| MTBE | tert-Butyl methyl ether |
| Na₂SO₄ | Sodium sulfate |
| NaCl | Sodium chloride |
| TBA | tert-Butyl ammonium |
| TBAA | tert-Butyl acetoacetate |
| TBAF | tetrabutylammonium fluoride |
| TBAI | tert-Butyl ammonium Iodide |
| TBDPS | tert-Butyl diphenyl silane |
| TEA | triethylamine |
| THF | Tetrahydrofuran |

### Example 1: Diagnosis and Staging of Bioenergetic Insufficiency in a Subject with Mild Cognitive Impairment (Hypothetical Data)—Prophetic

Patient Profile: A 70-year-old subject presents with subjective memory complaints consistent with Mild Cognitive Impairment (MCI).

### Assessment:

FDG-PET: Reveals reduced glucose uptake in the temporoparietal cortex and posterior cingulate cortex (PCC), with average SUVR of 1.05 (normalized to cerebellar gray matter), compared to typical healthy control values >1.3.

Ketone PET (11C-acetoacetate): Shows preserved ketone body uptake capacity in these same regions, with cerebral metabolic rate for ketones (CMRketone) similar to age-matched controls without cognitive impairment.

31P-MRS (localized to PCC): Reveals a moderately decreased PCr/ATP ratio of 1.35 (compared to typical control values >1.5-1.7), suggesting borderline energy reserve impairment. 1H-MRS shows a slightly reduced NAA/Creatine ratio.

Plasma Analysis: Fasting glucose and lipids are normal. Plasma BHB is low (<0.1 mM). Inflammatory markers show slightly elevated IL-6 at 3 μg/mL. Metabolic Vulnerability Index (MVX) score is moderately elevated.

Interpretation (UTBS Staging): The findings indicate impaired brain glucose utilization (low E glucose contribution), evidence of borderline energy state (reduced PCr/ATP) and neuronal stress (↓NAA), but retained capacity for compensatory ketone utilization. Mild systemic inflammation is present. Based on Table 2, the subject is classified as UTBS Stage 2 (Compensated Insufficiency), characterized by a primary fuel deficit (glucose) with ongoing compensation attempts and mild clinical symptoms (MCI).

### Example 2: Personalized Energy Rescue Therapy for Alzheimer's Disease (Following Ex 1, Hypothetical Pilot Data)—Prophetic

Intervention: Based on the diagnosis in Example 1 (UTBS Stage 2, primary glucose deficit with preserved ketone uptake), a personalized energy rescue therapy is initiated. The subject is administered an oral ketone monoester supplement (e.g., D-β-hydroxybutyrate-(R)-1,3-butanediol), titrated to achieve sustained mild ketosis (target plasma BHB 1.0-2.0 mM).

Monitoring: Plasma BHB levels are monitored weekly during titration then monthly. Cognitive function is assessed using the Alzheimer's Disease Assessment Scale-Cognitive subscale (ADAS-Cog) at baseline and after 3 and 6 months. Repeat 31P-MRS is planned at 6 months.

Hypothetical Pilot Data: “In a pilot group of 15 subjects meeting criteria similar to Example 1, 6 months of ketone ester treatment resulted in mean steady-state plasma BHB levels of 1.5±0.5 mM. The treatment group showed a mean improvement of 2.5 points on the ADAS-Cog from baseline, compared to an expected decline of approximately 1.0-2.0 points over 6 months in untreated MCI/early AD populations (p<0.05 vs. historical controls). Repeat 31P-MRS showed a trend towards improvement in the mean PCr/ATP ratio from 1.35±0.10 to 1.48 #0.12 (p=0.09).” This hypothetical outcome suggests the ketone ester provided alternative fuel, improved M, and potentially slowed cognitive decline.

### Example 3: Assessing Metabolic Flexibility in a Subject with Metabolic Syndrome-Prophetic

Patient Profile: A 55-year-old subject with central obesity, dyslipidemia, and borderline hypertension (meeting criteria for MetS).

Assessment: The subject undergoes an oral glucose tolerance test (OGTT) combined with indirect calorimetry. Respiratory Quotient (RQ) is measured at baseline (fasting, RQ=0.75) and for 2 hours post-glucose. The subject shows only a minimal increase in RQ to 0.80, failing to reach the expected >0.85 or approach 1.0 seen in metabolically flexible individuals switching to carbohydrate oxidation.

Interpretation (UTBS Staging): The impaired RQ shift indicates significant metabolic inflexibility, a hallmark of insulin resistance. Although currently having only mild risk factors, this inflexibility places the subject at high risk. Based on Table 2, the subject is classified as UTBS Stage 1 (Reduced Buffer/At Risk).

Recommendation: Intensive lifestyle intervention (diet focusing on whole foods, reduced processed carbohydrates/fats; regular exercise) is recommended to improve insulin sensitivity and restore metabolic flexibility, aiming to prevent progression to Stage 2 or 3.

### Example 4: Treating Heart Failure with Preserved Ejection Fraction (HFpEF) using Ketone Supplementation (Hypothetical Pilot Data)—Prophetic

Patient Profile: A 65-year-old subject with diagnosed HFpEF (LVEF 55%), NYHA Class II symptoms (dyspnea on moderate exertion), and diastolic dysfunction on echocardiogram. Receiving standard GDMT including a diuretic and ARB. Baseline Kansas City Cardiomyopathy Questionnaire (KCCQ) overall summary score is 60. Baseline 6-Minute Walk Test (6MWT) distance is 300 meters.

Assessment: Cardiac 31P-MRS reveals a significantly reduced myocardial PCr/ATP ratio of 1.40 (compared to typical healthy control values >1.8), indicative of impaired cardiac energy reserve.

Intervention: The subject is treated with an oral ketone ester supplement (e.g., D-βHB-(R)-1,3-butanediol, 25g TID) adjunctively to standard GDMT, targeting plasma BHB levels of 1-2 mM.

Monitoring: KCCQ and 6MWT are assessed at baseline and 3 months. Repeat cardiac 31P-MRS is performed at 3 months. Plasma BHB is monitored for compliance and target levels.

Hypothetical Pilot Data: “In a pilot study of 12 subjects with HFpEF and reduced PCr/ATP ratio, 3 months of ketone ester treatment achieved mean trough plasma BHB of 1.3±0.4 mM. Mean KCCQ score significantly improved by 15 points (from 60 #8 to 75±10, p<0.01). Mean 6MWT distance significantly increased by 50 meters (from 300 #50m to 350±60m, p<0.05). Repeat 31P-MRS showed an increase in mean PCr/ATP ratio to 1.60=0.15 (p=0.08 vs. baseline). No serious adverse events related to the ketone ester were reported.” These hypothetical results suggest that ketone supplementation improved cardiac energetics, symptoms, and functional capacity in HFpEF, consistent with potential benefits seen in recent trials.

### Example 5: System for Integrated Diagnosis and Treatment Recommendation-Prophetic

Scenario: A clinical decision support system based on UTBS principles is utilized for a patient presenting with exertional fatigue and suspected early HF.

Input: The system receives:
Cardiac 31P-MRS data: PCr/ATP ratio=1.55.
Plasma biomarker data: MVX score=elevated; NT-proBNP=moderately elevated; BHB=0.2 mM; IL-6=4 μg/mL.
Functional data: 6MWT=380m.
Clinical data: NYHA Class II symptoms.

Processing: The system's processor executes a UTBS algorithm, integrating these inputs. It compares the PCr/ATP ratio and biomarker levels against thresholds defined in the UTBS staging model (Table 2). It identifies impaired cardiac energetics (↓PCr/ATP) and moderate systemic stress (↑MVX, ↑NT-proBNP, ↑IL-6).

Output: The system displays:
“Calculated Bioenergetic Status: Moderate Cardiac Energy Deficit.”
“Assigned UTBS Stage: Stage 2 (Compensated Insufficiency).”
“Therapeutic Recommendation: Consider initiating adjunct therapy targeting myocardial energy supply (e.g., Ketone Ester or SGLT2 inhibitor) alongside optimizing GDMT. Monitor KCCQ, 6MWT, and PCr/ATP ratio.”

### Example 6: Method for Slowing Entropy Accumulation via M Maintenance (Hypothetical Study Design)—Prophetic

Design: A longitudinal preventative study enrolling 200 middle-aged subjects (age 50-65) identified as being in UTBS Stage 1 (Reduced Buffer, based on metabolic inflexibility via RQ testing and/or elevated MVX score). Subjects are randomized 1:1.

Intervention: The intervention group receives a personalized program aimed at maintaining M>M_crit. This includes: (1) Structured exercise plan; (2) Dietary counseling emphasizing whole foods and reduced processed carbohydrates; (3) Intermittent cycles (e.g., 5 days/month) of mild nutritional ketosis induced by a ketogenic diet or MCT supplementation; (4) Optional supplementation with NAD⁺ precursors based on baseline mitochondrial function markers. The control group receives standard-of-care lifestyle advice.

Monitoring: Annually for 10 years:
M-related parameters: Metabolic flexibility (RQ shift), resting PCr/ATP ratio via MRS in a representative tissue (e.g., muscle), plasma MVX score, inflammatory markers.
Entropy/Aging Biomarkers: DNA methylation age, telomere length, markers of cellular senescence (e.g., p16INK4a expression), protein aggregation markers in plasma/CSF.
Clinical Outcomes: Incidence of AD, HF, T2D, major cardiovascular events.

Expected Outcome: The intervention group is hypothesized to exhibit significantly better maintenance of M-related parameters (preserved flexibility, stable PCr/ATP, lower MVX/inflammation) and a slower rate of increase in entropy/aging biomarkers compared to the control group. This is projected to translate into a lower incidence of age-related chronic diseases and extended healthspan over the 10-year follow-up period, supporting the thermodynamic predictions of UTBS regarding M and entropy accumulation.

### Example 7: In Vitro Ketone Rescue of GLUT1-DS Neurons under Glucose Starvation (Executed)

Neuronal cells modeling GLUT1 deficiency (e.g., neurons derived from induced pluripotent stem cells of a GLUT1-DS patient) were cultured in conditions of reduced glucose to mimic the impaired glucose uptake of the disease. Baseline media had 0.5 mM glucose (versus 4 mM normal), causing ATP levels to drop and cell stress markers to rise. At hour 24 of glucose starvation, an exogenous ketone supplement was added to the media: specifically, 5 mM of R—BHB (delivered as 10 mM of a 1,3-butanediol-BHB monoester, which rapidly releases BHB). Within 4 hours of ketone administration, cellular ATP levels (measured by a luminescent assay) increased by ~50% compared to cells without ketone (which remained ATP-depleted). Lactate accumulation in the media was also significantly lower in the ketone-treated wells (consistent with the cells reducing glycolytic flux). By 48 hours, the ketone-treated neurons maintained normal morphology and viability (~85% viable cells), whereas the glucose-starved control neurons showed massive cell death (~30% viable). This demonstrates that ketone bodies can rescue neuronal energy metabolism when glucose is unavailable-relevant to GLUT1-DS and other hypoglycorrhachia conditions. The experiment mimics the clinical scenario where brain cells in GLUT1-DS rely on ketones for fuel. It also aligns with known patient data that ketosis prevents neurodegeneration in GLUT1 deficiency. Key observation: timing matters-neurons given ketones before ATP levels fall too low had near-complete prevention of stress kinase activation, highlighting the value of continuous ketone.

### Example 8: In Vitro Ketone Supplementation in VLCAD-Deficient Hepatocytes (Executed)

Hepatocytes isolated from a VLCAD knockout mouse (which model human VLCAD deficiency in liver) were used to assess how ketones affect fatty acid metabolism. Cells were incubated with a fatty acid load (200 μM palmitate plus carnitine) to simulate reliance on FA oxidation. VLCAD−/− hepatocytes typically cannot fully oxidize long-chain fat, leading to accumulation of long-chain acylcarnitines and cellular stress. Two sets of cultures were compared: (A) no additional substrate (glucose minimal media), and (B) media supplemented with 2 mM of a glycerol-3×BHB triester (SNV-G3 prototype), which slowly releases BHB and glycerol. After 12 hours, group A showed a ~3-fold increase in extracellular long-chain acylcarnitines (C14, C16 species) and evidence of cell injury (ALT enzyme release in media). In stark contrast, group B (with ketone) showed only a 1.2-fold increase in acylcarnitines, and ALT release was 70% lower than group A. The ketone-supplemented hepatocytes had actively metabolized BHB as evidenced by a measurable increase in β-hydroxybutyrate uptake and CO_2 production. Essentially, the presence of ketones spared the cells from accumulating toxic fatty intermediates by providing an alternate energy source; the cells reduced their reliance on burning palmitate since they had BHB to oxidize. This example provides mechanistic support that in FAOD, exogenous BHB directly mitigates the metabolic block effects, which is why FAOD patients on BHB show improved organ function.

### Example 9: In Vivo Rescue of Fasting-Induced Crisis in VLCAD−/− Mice using SNV-G3 (Executed)

A cohort of genetically engineered Vlcad\hat{ }−/−mice (VLCAD knockout, C57BL/6 background) was used to model the metabolic crisis triggered by fasting in long-chain FAOD. At 8 weeks old, mice were subjected to a prolonged fast (water only). Control group (n=8) received no intervention during the fast. Treated group (n=8) received an oral gavage of SNV-G3 ketone ester (0.4 g per mouse, approximately 2 g/kg) at the 12-hour mark of fasting, with a repeat half-dose at 20 hours. The SNV-G3 is a glycerol-tris(3-hydroxybutyrate) formulation that yields BHB and glycerol. Results:

Survival: By 24 hours of fasting, 0/8 control mice survived (all either died or met humane euthanasia criteria like hypothermia and near-zero blood glucose by ~18-22 hours). In stark contrast, 7/8 treated mice survived the full 24-hour fast and were active. The one treated mouse that succumbed had the lowest BHB level of the treated group (around 0.7 mM at death), underscoring that achieving sufficient ketosis is key.

Blood Chemistry: At 18 hours, control mice had blood glucose ~30 mg/dl (severely hypoglycemic) and BHB ~0.1 mM (unable to generate ketones). Treated mice had blood glucose ~55 mg/dL (significantly higher) and BHB ~1.5 mM. Some treated mice reached BHB>2 mM by 24 h. These levels correlated with survival time. Treated mice also showed lower blood lactate and a more normal acid-base balance (bicarbonate was higher in treated vs. control).

Cardiac Function: VLCAD−/− mice develop acute cardiac dysfunction during fasting (due to energy failure in heart). Echocardiography at baseline and 22 h fast showed that control mice had a drop in ejection fraction (EF) from ~60% to ~30% (signifying severe systolic dysfunction). Treated mice preserved cardiac function much better, with EF ~55% at 22h. In fact, by 24 h refeeding and recovery, treated mice's hearts resumed normal contractility, whereas none of the controls survived to that point. This aligns with human reports that ketone therapy prevented cardiomyopathy in FAOD patients.

Muscle Enzymes: Serum creatine kinase (CK), a marker of rhabdomyolysis, was extremely elevated in control mice (mean ~15,000 U/L) indicating muscle breakdown due to energy failure. Treated mice had much lower CK (~3,000 U/L), close to baseline for fasting mice, showing muscle was protected from necrosis by ketone availability.

Acylcarnitine Profile: As expected, control mice had sky-high long-chain acylcarnitines (C16, C18: 1) in serum at 18-24h (because fatty acids are only partially metabolized). Treated mice had markedly reduced levels (~50-70% lower for major species), demonstrating that exogenous ketones prevented the metabolic bottleneck. This corresponds with Example 2's in vitro results. The glycerol from SNV-G3 may also contribute some gluconeogenesis to support blood sugar, but primarily the effect is from ketone oxidation.

Observation: Treated mice remained more alert and mobile during the fast, whereas controls became lethargic or comatose. This behavioral difference qualitatively illustrates preserved brain function due to ketones (even though VLCAD deficiency mostly affects peripheral tissues, hypoglycemia in controls would impair brain; ketones prevented that).

This example powerfully shows proof-of-concept that an exogenous ketone ester (in this case SNV-G3) can avert what would otherwise be a fatal metabolic crisis in an animal model of a human orphan disease. It mirrors clinical aspirations: an emergency dose of ketone ester in an FAOD patient could similarly prevent death or ICU admission during illness. It also justifies broad claims for treating FAODs and related crises with such compounds.

### Example 10: In Vivo Ketone Therapy in GLUT1+/−Mice Increases Seizure Threshold and Improves EEG (Executed)

Heterozygous Glut1 knockout mice (Glut1+/−, a model of GLUT1 deficiency syndrome that exhibits some neurological impairment and lowered seizure threshold) were used to evaluate the anti-epileptic potential of the ketone ester SNV-G3 (the same compound as in Example 3). Mice (age ~3 months) were randomized to two groups (n=6 per group): Treated received ad libitum drinking water containing 10% (v/v) SNV-G3 solution (which delivered roughly 20 g/kg/day of the ester; they drank it readily due to slight sweet flavor), and Control received plain water. Both groups were fed normal rodent chow (so any ketosis comes from the supplement, not diet). After 2 weeks of supplementation, systemic BHB levels in treated mice averaged 0.8-1.0 mM (with fed-state levels ~0.5 mM and fasting levels up to ~1.5 mM), whereas control mice had BHB ~0.2 mM or less. Testing was then performed:

Seizure Threshold Test: We employed a pentylenetetrazol (PTZ) infusion test to quantify seizure threshold. PTZ is a pro-convulsant; the dose at first myoclonic jerk and first generalized seizure was recorded. Treated mice required a significantly higher dose of PTZ to induce seizures compared to controls. The threshold in treated was ~60 mg/kg vs ~35 mg/kg in controls for first generalized seizure (p<0.01). Some treated mice never seized at the maximum PTZ dose given, indicating profound seizure protection.

EEG Monitoring: Mice were implanted with telemetric EEG probes to monitor brain activity over 24-hour cycles. Control Glut1+/− mice showed frequent abnormal spike-wave discharges on EEG, especially during fasting or resting phases (a phenotype analogous to absence seizures or subclinical epileptiform activity in GLUT1-DS patients). Treated mice had a 90% reduction in spike-wave discharge frequency. In fact, 4 of 6 treated mice had virtually no pathological discharges during the recording period, similar to wild-type levels. This suggests the ketone ester essentially normalized brain electrical activity.

Behavior and Cognition: Though not as quantitatively measured, we observed that treated Glut1+/− mice performed better in a simple open-field test (more exploratory activity, less lethargy) and in a rotarod test (longer latency to fall, indicating better motor function). This may correlate with improved brain energy availability.

Growth: One concern in chronic ketosis for GLUT1-DS is growth; however, the treated mice maintained normal growth curves and did not differ in weight from controls, indicating the supplement did not cause malnutrition or stunting over the trial period. This mirrors clinical observations that well-managed ketosis does not impair growth in GLUT1-DS.

This example demonstrates that exogenous ketone therapy can recapitulate the anti-epileptic effects of ketogenic diets in a genetic epilepsy model, without a special diet. It supports the method of treating GLUT1-DS and other epileptic encephalopathies with ketone esters. The dramatic EEG normalization also suggests potential disease-modifying effects (less subclinical seizure activity might translate to better neurodevelopmental outcomes). It's a strong justification for including seizure disorders in the claims.

### Example 11: Closed-Loop Ketone Delivery Bench Test (Executed Prototype Demonstration)

A bench-top setup was created to test the functionality of a closed-loop ketone

delivery as described herein. Instead of a living subject, a surrogate system was used: a glass flow chamber filled with circulating buffer to simulate blood, into which ketones could be infused and from which a small volume was continuously removed to simulate metabolic consumption. Key components:

A ketone sensor (based on an amperometric enzymatic probe for BHB) inserted in the chamber continuously measured BHB concentration

A peristaltic infusion pump connected to a reservoir of SNV—K2 ester (chosen for test due to stable liquid form) could dispense the ester into the chamber at controlled rates.

A laptop running a PID control algorithm took the sensor input and adjusted the pump speed. The target BHB concentration was set to 1.0 mM.

A valve controlled outflow of the chamber to simulate clearance of ketones (first-order kinetics approximating metabolism).

We ran multiple trials. In one representative run, the system started with no ketone in the chamber (0 mM). The controller immediately set pump to a high rate; within ~15 minutes the chamber BHB reached 1.0 mM. As it overshot slightly to 1.2 mM, the PID dampened the infusion rate, and BHB settled to oscillate mildly between 0.95-1.05 mM. We then introduced a “disturbance” by doubling the outflow rate (simulating a surge in metabolism or distribution volume increase). The BHB started to drop; the sensor picked up the decline and the controller increased infusion to compensate. BHB was back in range within 10 minutes. Conversely, when outflow was halted (simulating zero metabolism), BHB began rising; the controller shut off the pump and it leveled off, never exceeding 1.5 mM (our safety threshold). This experiment proved that the closed-loop system can maintain a target ketone level in a dynamic environment. Importantly, the ester used (SNV—K2) is somewhat slower to release free BHB (since α-KG-BHB diester needs hydrolysis), which the PID algorithm accounted for by slightly adjusting the integral component for that lag.

While not in a living animal, this demonstration shows feasibility that the sensor+pump+algorithm loop works. It gives confidence that in a patient, the device will respond to trends (like increasing infusion during exercise or illness when ketones might drop, or cutting back infusion after a meal when patient's own ketones rise). For safety, we also tested scenarios like sensor failure (we programmed a dropout—the controller detected missing data and defaulted to a low basal infusion while alerting for help). Also, manual override was tested, allowing a clinician to temporarily suspend or bolus the pump (this might be used if, say, the patient is about to do intense exercise, one might pre-bolus some ketone).

### Example 12: MERIT in a Cancer Immunotherapy Setting (PD-1 Resistant Lung Cancer)—Prophetic

Patient Profile: A 60-year-old male with metastatic non-small cell lung cancer (adenocarcinoma) who initially had stable disease on anti-PD-1 therapy (pembrolizumab) but then showed tumor progression after 6 months. He has cachectic features (weight loss, fatigue) and lab signs of inflammation (CRP 30 mg/L). Tumor biopsies show high PD-L1 expression but also an immunosuppressive microenvironment (many exhausted PD-1\hat{ }high T cells and macrophages with NLRP3 activation markers).

Intervention: MERIT therapy is introduced alongside continued pembrolizumab. The patient is started on an oral ketone ester drink providing 25 grams of β-hydroxybutyrate equivalent and 5 grams of acetoacetate equivalent per dose. He takes this drink three times a day (morning, midday, evening). Additionally, he is given a continuous glucose/ketone monitor. The target set is to maintain BHB levels between 3-5 mM for at least 12 hours per day. A small dose of rapid-acting insulin (1-2 units) is used as needed to prevent hyperglycemia if any (since he's not diabetic, this was rarely needed).

Mechanistic Monitoring: —Within 2 weeks, his fasting BHB stabilizes around 4.0 mM, AcAc ~1.2 mM (BHB:AcAc ~ 3.3). —Blood tests show CRP dropped to 10 mg/L. —Ex vivo assays: His peripheral blood mononuclear cells (PBMCs) are tested for interferon-gamma (IFN-γ) production-after 2 weeks on MERIT, stimulated T-cells produce 30% more IFN-γ than baseline, suggesting improved T-cell function. —Flow cytometry of blood T-cells shows an increase in the fraction of PD-1\hat{ }low, CD127\hat{ }+ memory phenotype CD8 T-cells (from 10% to 18%), indicating partial reversal of exhaustion.

Tumor Response: After 8 weeks of combined therapy, imaging (CT scan) shows no new lesions and a slight reduction in size of two target lung nodules (each reduced ~15% in diameter, whereas prior to MERIT they were growing). While not a dramatic shrinkage, the disease stabilization after prior progression is considered a positive signal. The patient's cachexia has improved: weight increased by 3 kg, appetite and energy improved (subject reports being able to resume light exercise).

Interpretation: This example suggests that MERIT can reprogram the immune metabolism: The ketone therapy likely provided T-cells with an alternative fuel (since tumor cells were consuming most glucose), increased their NAD⁺ (via AcAc) supporting better function, and reduced systemic inflammation (lower CRP, possibly via NLRP3 inhibition). The result is an observable clinical benefit in a scenario where immunotherapy alone was failing. A continuation of this therapy is planned, and indeed at 4-month follow up, the patient's disease remains stable with some tumor markers (like LDH) trending down.

### Example 13: Acetoacetate-Enhanced Therapy in Heart Failure with Preserved Ejection Fraction (HFpEF)—Prophetic

Patient Profile: A 72-year-old female with HFpEF (heart failure with preserved EF ~60%, diastolic dysfunction), metabolic syndrome (BMI 34, hypertension, pre-diabetes). She experiences exercise intolerance (6-minute walk test distance 250 m, significantly below normal for age) and high NT-proBNP(200 μg/mL). HFpEF is known to involve myocardial microvascular inflammation and energetic deficits.

Intervention: She is enrolled in a trial of MERIT for HFpEF. The strategy is to target NAD⁺ boosting and vasodilation via AcAc. She receives an intravenous infusion of sodium acetoacetate (Na—AcAc) and sodium β hydroxybutyrate (Na—BHB) in a 1:1 molar ratio for 2 hours, three times a week, for 4 weeks. Each infusion delivers ~100 grams total ketone anions (50 g each, which raises her plasma ketones to ~3 mM BHB and ~3 mM AcAc during infusion, note the high AcAc relative to typical ketosis). Oral supplements of a ketone salt (Na/K BHB)₅ g thrice daily are given on non-infusion days to sustain mild ketosis (~1 mM BHB).

Mechanistic Observations:—By week 2, fasting blood NAD⁺ (measured by a research assay) rose from 50 μM to 70 μM; NADH dropped accordingly (NAD+/NADH ratio improved ~1.5-fold). —Venous lactate, which was slightly elevated at baseline (2.2 mM), fell to 1.2 mM post-infusion, reflecting improved whole-body redox/oxygenation. —Echocardiography with Doppler shows improved diastolic relaxation parameters: E/e′ (mitral inflow to annular velocity ratio) improved from 15 to 12, indicating less stiffness. —Flow-mediated dilation (FMD) of the brachial artery improved from 5% to 8% after 4 weeks, suggesting better endothelial function (perhaps from increased NO bioavailability via eNOS coupling).

Clinical Outcomes: After 4 weeks:—The patient's exercise tolerance improved; her 6-minute walk distance increased to 300 m. She reports less shortness of breath on exertion. —NT-proBNP dropped to 120 μg/mL. —Blood pressure, originally 150/90 on meds, is now 135/80 (perhaps reflecting better vascular function and weight loss of 2 kg due to some mild caloric deficit from ketone regimen). —Subjective well-being is better, with fatigue score on a standardized questionnaire improved by 30%.

She experienced no hypoglycemia (glucose remained ~100 mg/dL fasting, maybe slightly lower than 110 mg/dL baseline). No edema or arrhythmias occurred; in fact, her heart rate variability slightly improved as measured by wearable (speculatively due to improved autonomic function from ketones).

Interpretation: This example demonstrates the use of an acetoacetate-rich therapy to relieve reductive stress and improve cardiac energetics in HFpEF. The improved NAD⁺ likely enhanced sirtuin activity, eNOS function in endothelium, and myocardial efficiency. The result was measurable in both surrogate markers (NAD⁺, lactate, E/e′) and patient-centered outcomes (exercise capacity). This supports MERIT's potential in HFpEF, where few therapies are effective.

### Example 14: Ketone-Based Epigenetic Therapy in Early Alzheimer's Disease-Prophetic

Patient Profile: A 68-year-old male with early Alzheimer's disease (MMSE score 24/30, with mild memory impairment affecting daily tasks). CSF biomarkers are positive for amyloid and tau. He is not yet on any drug trial aside from standard donepezil. He is APOE4 heterozygous. FDG-PET shows temporoparietal hypometabolism. He also has metabolic syndrome (A1c 6.1, overweight).

Intervention: Given mechanistic rationale that BHB might inhibit HDACs and boost gene expression for synaptic resilience, he is started on a high-dose ketone ester regimen: 30 mL of an (R)-3-hydroxybutyl (R)-3-hydroxybutyrate monoester taken three times daily with meals. This typically raises his plasma BHB to ~2-3 mM for 4-6 hours after each dose. He's also advised to reduce refined carbs moderately to prolong ketosis (not a full KD, but moderate). Treatment planned for 6 months, with cognitive tests and biomarker checks at baseline, 3, and 6 months.

Mechanistic/Marker Findings: —After 3 months, a lumbar puncture is done for research: CSF β hydroxybutyrate is measurable around 0.5 mM (meaning ketones are reaching CNS). His CSF also shows an increase in acetylation of histone H3(assayed from precipitated CSF cells/exosomes or some emerging method) by +50% relative to baseline. Notably, histone acetylation at lysine 9 and 14 was low at baseline (consistent with AD), and now significantly higher-suggesting HDAC suppression in the brain. —His blood monocytes show a 20% decrease in HDAC2 mRNA expression compared to baseline (there is literature that peripheral HDAC2 might mirror central to some extent). —Plasma BDNF, which was low at baseline (15 ng/mL), is now 25 ng/ml-BDNF is released from brain or platelets, but an increase may indicate improved brain BDNF expression due to HDAC inhibition by BHB.

Cognitive/Clinical Outcomes:—At 6 months, MMSE is stable at 25/30 (slight improvement), whereas on average one might expect a 1-2 point decline in that time. On a more sensitive memory test (e.g., ADAS Cog), he improved by 2 points versus baseline. His spouse reports subtle improvements in word-finding and initiative. —Brain FDG-PET repeated at 6 months shows slightly better uptake in posterior cingulate and parietal cortex compared to baseline scan (this is anecdotal but interesting, possibly because his brain is actually using ketones, and overall metabolism improved). —No significant change in MRI volumetrics (6 months too short to gauge atrophy). —Metabolically, he lost 5 kg (some intentional diet changes and ketones reducing appetite might have helped). His A1c is now 5.6. This could independently help cognitive function (less insulin resistance). —He tolerated the ketone ester well after initial GI discomfort in first week.

Interpretation: This example highlights MERIT's epigenetic axis: BHB likely inhibited HDACs in the brain, leading to increased histone acetylation, upregulation of genes like BDNF, and subsequent functional stabilization of cognition. The measurable increases in BDNF and histone marks strengthen the argument that mechanistic action occurred. Clinically, the patient did better than expected for AD trajectory. This supports further exploration of ketone therapy as a disease-modifying adjunct in AD.

### Example 15: Refractory Epilepsy in a Child-Closed-Loop Ketone Therapy-Prophetic

Patient Profile: A 5-year-old girl with Dravet syndrome (SCN1A mutation) suffering multiple seizure types (tonic-clonic, myoclonic) daily despite polypharmacy (valproate, clobazam, stiripentol). She has developmental delay and at baseline 2-3 generalized convulsive seizures per week plus nightly convulsions. Classic ketogenic diet was attempted but not tolerated due to feeding difficulties and side effects.

Intervention: We implement a Therapeutic Titratable Ketosis (TTK) approach using a G-tube for feeding: A balanced ketone supplement formula (containing beta-hydroxybutyrate sodium salt and medium-chain triglyceride oil to yield both BHB and some AcAc after liver metabolism) is given continuously overnight via feeding pump, and bolus during the day. Target plasma BHB is 4 mM. To achieve this, she receives ~ 0.5 g/kg/hour of BHB salt overnight and small boluses (0.3 g/kg) every 4 hours daytime. A continuous ketone monitor (prototype device) is placed on her thigh, sending readings to a pump controller that adjusts rate+20% if BHB goes out of 3-5 mM range.

Results: —After titration, she maintains 3-5 mM BHB 80% of the day, with a BHB:AcAc ratio around 2.5:1 (some AcAc presumably from MCT metabolism). —Over 1 month, her seizure frequency drastically reduces: from ~~>50 seizures (various types) per month to ~10 per month, and the remaining seizures are shorter. On two occasions, the monitor/pump system noted a drop in BHB before a cluster of myoclonic seizures; in response, the caregiver gave an extra bolus (as per protocol) and seizures aborted. —An EEG after 1 month shows reduced epileptiform activity, especially during sleep (previously very abnormal). —Importantly, she exhibits improved alertness and engagement in therapy sessions, likely due to fewer seizures and perhaps direct ketone effects.

Biochemical observations: Fasting glucose stabilized around 60-70 mg/dL (low but she's asymptomatic because ketones fuel brain). No episodes of acidosis beyond mild (bicarbonate stayed >18 mM). Blood beta hydroxybutyrate variations day-to-day were kept within ±1 mM around target.

Medication adjustments: With neurologist oversight, clobazam was reduced by 50% by end of 2 months, due to lethargy improvement and lack of need (ketones provided seizure control). Valproate was continued (note: valproate and ketosis both elevate ammonia sometimes, but ammonia stayed normal, likely because our controlled ketosis wasn't extreme and we supplemented carnitine as precaution).

Interpretation: This example shows a scenario using the treat-to-target approach for a severe epilepsy case. The closed-loop system could preemptively detect falling ketone levels (often due to growth spurts or increased metabolism during fevers, etc.) and adjust feeding to prevent seizures. The child's developmental progress improved after seizure reduction. This underscores MERIT's advantage over the rigid ketogenic diet-more precise control, easier fine-tuning, and integration with modern monitoring.

### Example 16: Autoimmune Arthritis-Metabolic Adjunct to Immunotherapy-Prophetic

Patient Profile: A 45-year-old female with seropositive rheumatoid arthritis for 10 years. She has erosive joint changes and moderate disease activity (DAS28 score 5.0) despite methotrexate and adalimumab (TNF inhibitor). She also is overweight (BMI 30) and has fatigue and morning stiffness. Labs: CRP 15 mg/L, IL-6 is elevated at 30 μg/mL.

Intervention: We initiate MERIT as an adjunct. The rationale is to reduce inflammation via NLRP3 inhibition and weight/metabolic improvement. She starts a daily ketone supplement regimen-specifically, a BHB mineral salt drink (providing 12 g BHB twice daily) and encouraged to follow a low-glycemic diet (not strictly keto, but moderate carbs ~100 g/day). Aim: keep BHB ~1-2 mM persistently (checked with fingerstick meter at home).

Observations Over 3 Months: —She consistently reaches fasting BHB around 1.5 mM each morning. CRP declines from 15 to 5 mg/L by 6 weeks. —IL-6 level after 3 months is 10 μg/mL (down by two-thirds). Serum TNFα slightly decreased as well (though she's on anti-TNF, that was expected to be low anyway). —Subjective: She reports significantly less morning stiffness (duration down from 2 hours to 30 minutes). Fatigue is reduced. —Her DAS28 score improved to 3.2 (which is low disease activity). Swollen joint count went from 8 to 2. She even halved her NSAID use. —She lost 8 kg (partly due to appetite suppression and possibly being more active without pain). —Radiographs not expected to change in short term, but ultrasound of joints showed reduced synovial thickening in knees.

Mechanistic Insight: Though we didn't directly measure inflammasome markers, we suspect BHB suppressed macrophage IL-1β in joints. She had high IL-1 at baseline (not measured, but IL-6 was high which often correlates). Ketones likely also improved her insulin sensitivity (fasting insulin dropped from 18 uIU/mL to 8), which might cut some inflammatory drive. Epigenetically, her immune cells might have more acetylation at anti-inflammatory gene promoters (speculative).

Medication: At month 3, her rheumatologist chooses to taper methotrexate from 20 mg to 15 mg/week given her improvement (to mitigate side effects), continuing adalimumab. The ketone therapy essentially acted as a synergistic anti-inflammatory. She experiences no flares during a brief period off adalimumab (due to scheduling issues)—perhaps ketosis gave some protection.

Interpretation: This example indicates MERIT can serve autoimmune patients by addressing the immunometabolic aspect. It's plausible that with long term use, reliance on biologics could diminish or at least patients will feel better. This is a safe adjunct: she had no adverse effects beyond initial diuresis. (We gave her electrolyte supplements to avoid dehydration when starting ketosis.)

### Example 17: Means-Agnostic Ketosis Induction for Checkpoint-Inhibitor-Treated Melanoma-Prophetic

Patient Profile: A 52-year-old male with metastatic melanoma expressing PD-L1 at high tumor cell prevalence (≥50%). He has no prior systemic therapy and is otherwise ambulatory. BMI is 29 (overweight range). Labs at baseline: circulating BHB is low (fasting BHB 0.08 mM), consistent with a non-ketotic metabolic state; histone β-hydroxybutyrylation (Kbhb) enrichment in peripheral blood lymphocytes is minimal on mass spectrometry; NLRP3 inflammasome activation in circulating monocytes (measured by caspase-1 activity assay) is elevated; NAD+/NADH ratio in lymphocytes is reduced relative to reference range.

Intervention: The patient is enrolled in a combination study. He receives pembrolizumab (anti-PD-1 monoclonal antibody) intravenously every 3 weeks, combined with a ketogenic agent—a ketone-carrier conjugate formulated for oral administration-administered daily and titrated to achieve circulating BHB levels of 1-2 mM. He is instructed to maintain his usual unrestricted diet without any carbohydrate restriction, caloric reduction, or fasting. Dosing is divided across multiple daily administrations to maintain sustained BHB elevation throughout the day. The treat-to-target endpoint is circulating BHB of 1-2 mM with Kbhb enrichment reaching ≥50% of total histones in peripheral blood lymphocytes.

Observations Over 24 Weeks: Within two weeks of initiating the ketogenic agent, fasting BHB rises to 1.4 mM and stabilizes in the 1.2-1.8 mM range across subsequent monitoring visits with no dietary changes. Kbhb enrichment in peripheral blood lymphocytes reaches 54% of total histones by week 8. NLRP3 inflammasome activity in circulating monocytes is suppressed by 62% relative to baseline by week 12. Circulating IL-1β and IL-6 decline from elevated baseline values. At week 12 imaging assessment using RECIST 1.1 criteria, the patient achieves a partial response, with target lesion diameter reduction of 38%. At week 24, a complete response is confirmed on imaging. The patient reports no significant gastrointestinal side effects and tolerates the ketogenic agent without dose reduction.

Mechanistic Insight: The combination is consistent with a state-gated enhancement of pembrolizumab efficacy. NLRP3 inflammasome suppression in tumor-associated myeloid cells is expected to have shifted the tumor microenvironment toward a less immunosuppressive, more cytotoxic phenotype, permitting T-cell infiltration and activation. The elevated Kbhb enrichment in peripheral blood lymphocytes suggests concurrent epigenetic remodeling of T-cell differentiation and anti-tumor gene expression. NAD+/NADH elevation may have further enhanced T-cell oxidative metabolism and effector function. Critically, none of these mechanisms required dietary alteration: the patient's glucose homeostasis was maintained, and the ketotic state was achieved pharmacologically and independently of dietary restriction.

Medication and Monitoring: Pembrolizumab closing continues at the standard schedule throughout. Circulating BHB is monitored by fingerstick at home three times weekly, with clinic measurement at each pembrolizumab infusion visit. Dose of the ketogenic agent is adjusted at weeks 4, 8, 12, and 24 based on BHB and Kbhb readouts, with no adjustment required beyond the initial titration. No immune-related adverse events attributable to pembrolizumab are observed above the expected baseline rate.

Interpretation: This example illustrates the principle that pharmacological ketosis induction, independent of dietary restriction or any baseline metabolic phenotype change, can enhance the efficacy of immune checkpoint inhibitor therapy in a subject who would otherwise be treated with pembrolizumab alone. The observed response rate and biomarker trajectory are consistent with multi-pillar MERIT engagement-immune, epigenetic, and redox pillars operating concurrently-gating pembrolizumab's ability to generate an anti-tumor T-cell response. The case supports the broader principle that metabolic state is a therapeutically actionable determinant of immunotherapy response.

### Example 18: State-Gated Efficacy Rescue for Non-Responder to PI3K Inhibitor-Prophetic

Patient Profile: A 63-year-old female with PIK3CA-mutant triple-negative breast cancer who has progressed on a PI3Kα inhibitor due to dose-limiting insulin feedback hyperactivation. During her prior course of treatment, fasting glucose rose to 196 mg/dL and circulating insulin reached levels consistent with severe insulin feedback hyperactivation, necessitating dose reduction and ultimately drug discontinuation. She presents with confirmed PIK3CA mutation on liquid biopsy, two hepatic metastases on CT imaging, and a performance status of ECOG 1. BMI is 27. She is not diabetic at baseline. Fasting glucose at current presentation: 94 mg/dL. Fasting insulin: 14 μIU/mL.

Intervention: The patient is enrolled in a metabolic rescue study. A ketogenic agent—a ketone-carrier conjugate formulated for oral administration—is initiated and titrated over two weeks to achieve circulating BHB of 1-2 mM. She is instructed to maintain her usual diet without carbohydrate restriction. Once BHB stabilizes at 1.4-1.7 mM across three consecutive days of home monitoring, the PI3Kα inhibitor is re-introduced at the same dose that had previously been intolerable. Dose is held constant unless glucose or insulin thresholds require re-evaluation. The target outcome is tolerability of the PI3Kα inhibitor at a therapeutically meaningful dose with evidence of tumor response.

Observations Over 12 Weeks: At weeks 2 and 4 following re-introduction of the PI3Kα inhibitor, fasting glucose remains within the normal range (87 mg/dL at week 2; 91 mg/dL at week 4). Fasting insulin remains at 9 μIU/mL at week 4, demonstrating suppression of the insulin feedback hyperactivation that had previously limited closing. No dose reduction of the PI3Kα inhibitor is required. At week 8 imaging assessment, the two hepatic target lesions have reduced in diameter by a combined 31%, consistent with a partial response. At week 12, the response is sustained and the patient remains on full dose of the PI3Kα inhibitor without glycemic adverse events. HOMA-IR (homeostatic model assessment of insulin resistance) improves from a baseline of 3.2 to 1.8 by week 8.

Mechanistic Insight: The mechanism of rescue is consistent with ketosis-induced suppression of hepatic glucose production and reduction of baseline insulin secretion consequent upon reduced glycemic excursions. By establishing a metabolic state in which insulin secretion is modulated prior to PI3Kα inhibitor exposure, the anticipated insulin feedback hyperactivation is attenuated because the baseline drive to insulin secretion is already reduced. This is not a pharmacokinetic interaction—the plasma concentration-time profile of the PI3Kα inhibitor is not expected to be meaningfully altered—but rather a pharmacodynamic state-gate in which the metabolic context of the subject licenses the drug to achieve efficacious tissue concentrations without triggering intolerable insulin feedback. Ketone-mediated NAD+-dependent improvements in insulin signaling may further contribute to the metabolic rescue.

Medication and Monitoring: Fasting glucose is measured daily by the patient using a standard glucose meter, with weekly clinic review. Circulating BHB is measured three times weekly by fingerstick. Fasting insulin and HOMA-IR are assessed at weeks 2, 4, 8, and 12. The ketogenic agent is continued throughout without dose adjustment. No electrolyte abnormalities or other adverse effects attributable to the ketogenic agent are observed.

Interpretation: This example illustrates that drugs deemed too toxic in unselected metabolically heterogeneous populations-because of intolerable insulin feedback or glycemic side effects—may be rescued to clinical utility when administered in subjects in whom the ketotic state has been pharmacologically established prior to drug re-introduction. The metabolic state-gating principle demonstrated here extends beyond immunotherapy to any drug class whose dose-limiting toxicity is metabolically mediated. This constitutes a generalizable strategy for the rescue of failed or restricted oncological drug programs.

### Example 19: NLRP3-Suppressive Ketosis for Inflammatory Bowel Disease-Prophetic

Patient Profile: A 35-year-old male with moderate-to-severe ulcerative colitis diagnosed four years prior. He has been unresponsive to 5-aminosalicylic acid and achieves only partial, unstable response on azathioprine. He has declined biologic therapy due to concerns about immunosuppression. Current presentation: Mayo Clinic Score 8 (moderate-to-severe), stool frequency 6-8 times per day with visible blood, CRP 22 mg/L, fecal calprotectin 1,840 μg/g. He is not on systemic corticosteroids at enrollment. BMI is 24. He maintains a standard Western diet with no dietary restrictions.

Intervention: MERIT therapy is initiated as an adjunct to his existing azathioprine regimen, which is continued unchanged at a stable dose. A ketogenic agent—a ketone-carrier conjugate formulated for oral administration—is initiated and titrated to achieve circulating BHB of 1-2 mM, with treat-to-target closing guided by home fingerstick BHB measurements three times weekly. He is not asked to alter his diet or reduce carbohydrate intake. Target biomarker endpoints at 12 weeks include NLRP3 inflammasome activity suppressed by ≥60% relative to baseline in circulating monocytes; intestinal tight-junction protein expression (ZO-1, claudins, occludin) increased on intestinal biopsy; and CRP reduced to <5 mg/L.

Observations Over 12 Weeks: By week 4, circulating BHB stabilizes at 1.5 mM. CRP declines from 22 to 7 mg/L by week 6. Fecal calprotectin falls from 1,840 to 620 ug/g by week 8. Circulating IL-1β and IL-18 decline from elevated baseline values. Stool frequency reduces to 2-3 per day with resolution of visible blood by week 8. NLRP3 inflammasome activity in circulating monocytes is suppressed by 67% relative to baseline at week 8. Intestinal biopsies at week 12 demonstrate upregulation of ZO-1 and claudin-3 expression in intestinal epithelial cells, and caspase-1 staining is substantially reduced in lamina propria mononuclear cells compared to baseline. Endoscopic assessment at week 12 using the Ulcerative Colitis Endoscopic Index of Severity (UCEIS) shows a reduction from 6 to 2, consistent with near-complete mucosal healing. The patient's Mayo Clinic Score improves to 1 (clinical remission).

Mechanistic Insight: The observations are consistent with BHB-mediated NLRP3 inflammasome suppression in intestinal lamina propria immune cells, reducing local IL-1β and IL-18 production, dampening the inflammatory cascade driving mucosal injury, and permitting mucosal healing. Separately, the upregulation of tight-junction proteins suggests that Kbhb-mediated epigenetic remodeling of intestinal epithelial cells enhanced barrier gene expression, independent of the anti-inflammatory effect. These two mechanisms —inflammasome suppression and barrier restoration—are orthogonal and operate simultaneously in response to BHB elevation, explaining the extent and rapidity of mucosal improvement. The patient's insulin sensitivity improved modestly (fasting insulin dropped from 11 μIU/mL to 7), which may have contributed to reduced systemic inflammatory drive, though this was likely a secondary rather than primary mechanism.

Medication and Monitoring: Azathioprine is continued throughout at an unchanged dose. The ketogenic agent is continued without modification after achieving the target BHB range at week 4. No adverse effects are observed beyond mild initial diuresis in the first week, which resolved spontaneously. Electrolytes remain within normal limits. Endoscopic assessment is performed at week 12 to confirm mucosal healing.

Interpretation: This example indicates that MERIT-based pharmacological ketosis can produce clinically meaningful remission in moderate-to-severe ulcerative colitis as an adjunct to conventional immunosuppressive therapy, without requiring dietary modification, fasting, or escalation to biologic therapy. The dual mechanism of NLRP3 inflammasome suppression and barrier restoration-operating through the immune and epigenetic MERIT pillars respectively—is consistent with the convergent evidence for BHB-mediated gut immunometabolic remodeling. Long-term studies would be required to assess whether sustained MERIT therapy enables reduction in azathioprine dose or prevention of relapse.

### Example 20: Companion-Diagnostic-Guided Patient Selection Using Kbhb and NLRP3 Readouts-Prophetic

Patient Profile: A 58-year-old patient with newly diagnosed melanoma is being evaluated for enrollment in an adjuvant immunotherapy trial examining pembrolizumab plus pharmacological ketosis induction versus pembrolizumab alone. She has a BMI of 31, fasting glucose of 102 mg/dL (borderline impaired fasting glucose), fasting insulin of 19 μIU/mL, CRP 8 mg/L, and elevated circulating IL-1β. She does not have a prior diagnosis of diabetes.

Companion Diagnostic Assessment: Prior to trial randomization, the patient undergoes a baseline MERIT biomarker panel consisting of five components: (1) baseline circulating BHB; (2) histone β-hydroxybutyrylation enrichment in peripheral blood CD8+T cells measured by mass spectrometry-based histone proteomics; (3) NLRP3 inflammasome activation status in circulating monocytes measured by phospho-caspase-1 intracellular staining and flow cytometry; (4) NAD+/NADH ratio in peripheral blood lymphocytes; and (5) composite circulating inflammatory biomarkers including IL-1β, IL-18, TNF-α, CRP, and IL-10.

Results at baseline show fasting BHB of 0.07 mM, confirming a non-ketotic state. Kbhb enrichment in peripheral blood CD8+ T cells is minimal, below any threshold for meaningful epigenetic engagement. NLRP3 inflammasome activation in circulating monocytes is elevated at 38% phospho-caspase-1-positive cells (reference range <10%). NAD+/NADH ratio in peripheral blood lymphocytes is reduced at 1.1 (reference range >1.8). Circulating IL-1β and IL-18 are both elevated; IL-10 is low.

Enrichment Decision: The companion diagnostic profile-characterized by baseline NLRP3 inflammasome hyperactivation, reduced NAD+/NADH ratio, and negligible basal Kbhb enrichment-identifies this patient as a “high-responder” phenotype consistent with significant MERIT dysregulation likely to benefit from pharmacological ketosis induction. She is accordingly enrolled in the combination arm: pembrolizumab plus an oral ketogenic agent titrated to maintain circulating BHB at 1-2 mM. She is not asked to alter her diet or restrict carbohydrate intake. A contemporaneous patient assessed on the same MERIT biomarker panel who displays low baseline NLRP3 inflammasome activation and a high NAD+/NADH ratio is classified as “metabolically healthy” and enrolled in a standard pembrolizumab-alone control arm, as her metabolic state does not suggest significant MERIT dysregulation.

Observations Over 24 Weeks: Within two weeks of initiating the ketogenic agent, circulating BHB stabilizes at 1.5 mM without any dietary change. Kbhb enrichment in peripheral blood CD8+ T cells rises from minimal baseline values to 58% of total histones by week 8. NLRP3 inflammasome activity in circulating monocytes is suppressed by 71% relative to baseline by week 12. Circulating IL-1β and IL-18 decline substantially. CRP declines from 8 to 2 mg/L by week 8. NAD+/NADH ratio in peripheral blood lymphocytes increases from 1.1 at baseline to 2.0 by week 8 and is maintained at approximately that level through week 24. At week 24 imaging assessment, the patient achieves a confirmed partial response with a 42% reduction in target lymph node lesion diameter.

Mechanistic Insight: The companion diagnostic enrichment strategy identified this patient as likely to benefit by demonstrating pre-existing MERIT dysregulation—a metabolic profile that pharmacological ketosis is specifically designed to correct. The high baseline NLRP3 activation and low NAD+/NADH ratio represent the precise mechanistic targets that BHB-mediated NLRP3 suppression and NAD+-dependent redox rebalancing address. By suppressing NLRP3 inflammasome activity in tumor-associated myeloid cells, the ketogenic agent is expected to have shifted the tumor microenvironment from a pro-tumor, IL-1β-producing, immunosuppressive phenotype toward a more cytotoxic, Th1-enriched phenotype permissive for T-cell infiltration and anti-tumor immunity. Simultaneously, Kbhb-driven epigenetic remodeling of CD8+ T cells is expected to have enhanced the expression of anti-tumor effector genes and the proliferative capacity of tumor-infiltrating T cells, augmenting the effect of pembrolizumab-mediated PD-1 blockade. In a metabolically healthy patient with low baseline NLRP3 activation and an already-adequate NAD+/NADH ratio, these MERIT pathways are functioning adequately at baseline, and the incremental benefit from pharmacological ketosis induction is expected to be smaller—consistent with the rationale for enrolling that patient in the pembrolizumab-alone arm.

Medication and Monitoring: Pembrolizumab is administered intravenously at standard closing every three weeks throughout the 24-week study period. Circulating BHB is monitored by home fingerstick three times weekly, with confirmatory clinic measurement at each pembrolizumab infusion visit. The full MERIT biomarker panel is reassessed at weeks 8, 12, and 24. Dose of the ketogenic agent is adjusted at weeks 4 and 8 based on BHB and Kbhb readouts, with no further adjustment required after week 8. No immune-related adverse events above the expected background rate are observed. Electrolytes, renal function, and liver function remain within normal limits throughout.

Interpretation: This example illustrates the clinical utility of MERIT biomarker-guided patient selection as a companion diagnostic strategy for ketogenic agent plus immunotherapy combination trials. Rather than treating all melanoma patients with combination therapy irrespective of baseline metabolic state, a biomarker-guided enrichment approach selects subjects with pre-existing MERIT dysregulation—the population most likely to experience meaningful benefit from pharmacological ketosis induction as a metabolic state-gate for pembrolizumab response. This approach has the potential to increase the response rate in the ketogenic agent plus immunotherapy arm and to improve detection of clinical efficacy signal in the trial. The case further illustrates that the companion diagnostic is itself agnostic to dietary state: the patient's ketotic state is induced and sustained pharmacologically, and the MERIT biomarker panel quantifies target engagement independently of any dietary co-intervention.

### Example 21: Sustained Treat-to-Target Ketosis in Ambulatory Patient with Parkinson's Disease-Prophetic

Patient Profile: A 67-year-old patient with Parkinson's disease characterized by progressive motor decline and cognitive slowing is enrolled in a study examining the safety and efficacy of sustained, treat-to-target pharmacological ketosis. He has a Movement Disorder Society Unified Parkinson's Disease Rating Scale (MDS-UPDRS) motor score of 38 (moderate impairment), a Montreal Cognitive Assessment (MoCA) score of 23 (mild cognitive impairment), and a Parkinson's Disease Questionnaire (PDQ-39) quality-of-life score of 48 (on a scale of 0=best to 100=worst). He reports significant fatigue and progressive motor fluctuations. He is on stable doses of levodopa-carbidopa and a dopamine agonist. BMI is 26. Baseline labs show fasting BHB of 0.06 mM, CRP 4 mg/L, mildly elevated IL-1β and IL-6, and a reduced NAD+/NADH ratio in peripheral blood lymphocytes.

Intervention: The patient is started on a ketogenic agent—a ketone-carrier conjugate formulated for oral administration—at 200 mg/kg/day, with dose escalation every two to four weeks based on tolerability and circulating BHB measurements. The target circulating BHB is 1-2 mM, maintained for the majority of the 24-hour day. Dosing frequency is titrated to minimize burden while maintaining the target BHB range, ultimately arriving at a three-times-daily regimen. The patient is instructed to maintain an unrestricted diet with no carbohydrate restriction or caloric modification. He continues his standard Parkinson's disease medications-levodopa-carbidopa and the dopamine agonist-throughout the study at unchanged doses, enabling attribution of any observed benefit to the ketogenic agent.

Observations Over 52 Weeks: At baseline, week 2, week 4, week 8, week 12, and quarterly thereafter, the following assessments are performed: circulating BHB and ketone body concentrations; MDS-UPDRS motor score; MoCA; PDQ-39 quality-of-life assessment; MERIT biomarkers including Kbhb enrichment, NLRP3 inflammasome activity, NAD+/NADH ratio, and histone acetylation; circulating inflammatory markers; and tolerability and adverse event monitoring.

By week 4, circulating BHB stabilizes at 1.3-1.6 mM across the treatment interval. Kbhb enrichment in peripheral blood lymphocytes reaches 52% of total histones at week 8. NLRP3 inflammasome activity in circulating monocytes is suppressed by 58% relative to baseline at week 12. Circulating IL-6 declines from mildly elevated baseline values to within normal limits by week 12. NAD+/NADH ratio in peripheral blood lymphocytes increases from baseline reduced values to 2.1 at week 8 and is maintained at approximately that level throughout the study. At week 24, MDS-UPDRS motor score improves from 38 to 31—an 18% improvement. MoCA score improves from 23 to 25. PDQ-39 quality-of-life score improves from 48 to 34. The patient reports reduced fatigue, improved “on” time with levodopa, and subjectively less pronounced motor fluctuations. At week 52, MDS-UPDRS motor score is 29, representing a 24% improvement from baseline. The rate of motor decline is substantially slower than expected based on age-matched historical cohort data, consistent with the study's design to detect a 25-40% slowing of motor decline over 52 weeks.

Mechanistic Insight: The expected mechanistic basis for benefit involves ketosis-induced mitochondrial enhancement, neuroinflammation suppression, and neurotrophic signaling. Enhanced mitochondrial bioenergetics via elevated NAD+/NADH and ketone-supported oxidative phosphorylation is expected to have improved neuronal and dopaminergic energy production, reducing the vulnerability of substantia nigra neurons to bioenergetic stress. NLRP3 inflammasome suppression is expected to have dampened microglial activation and neuroinflammation, a recognized driver of progressive dopaminergic neuronal loss. Histone acetylation-driven upregulation of autophagy-related genes may promote clearance of α-synuclein aggregates and other misfolded proteins, addressing a central mechanism of disease pathogenesis. BHB-mediated activation of circadian clock components BMAL1 and PER2 may have normalized disrupted sleep-wake cycles, which are severely affected in Parkinson's disease and contribute to both cognitive and motor fluctuation. Critically, none of these mechanisms required dietary restriction: the patient's carbohydrate intake was entirely unrestricted, and the therapeutic ketotic state was achieved and sustained through pharmacological means alone.

Medication and Monitoring: Levodopa-carbidopa and the dopamine agonist are continued without dose adjustment throughout the 52-week study. Circulating BHB is measured by fingerstick three times daily by the patient at home, with clinic confirmation at each scheduled assessment visit. Dose of the ketogenic agent requires no adjustment after the initial titration phase is completed at week 4. The ketogenic agent is well tolerated throughout; mild initial diuresis resolves in the first week following electrolyte supplementation. No cognitive or psychiatric adverse effects are observed. All laboratory parameters including electrolytes, renal function, and liver function remain within normal limits at every assessment timepoint.

Interpretation: This example demonstrates the feasibility and expected clinical benefit of sustained, treat-to-target pharmacological ketosis-maintained via an oral ketogenic agent in an ambulatory outpatient with active Parkinson's disease-over a 52-week period, without requiring any dietary modification or alteration of existing Parkinson's disease pharmacotherapy. The convergent biomarker evidence-including NLRP3 suppression, Kbhb induction, and NAD+/NADH elevation-confirms engagement of all relevant MERIT pillars and supports mechanistic attribution of the observed clinical improvement to pharmacological ketosis. The observed slowing of motor decline compared to expected natural history, if confirmed in a powered randomized controlled trial, would represent a disease-modifying effect with significant clinical implications for a condition in which no currently approved therapy has demonstrated disease modification.

### Example 22: Exemplary Effects of Ketone Bodies in Alzheimer's Disease Neurons

Described below are the effects of exogenous ketone bodies, including acetoacetate (AcAc), on neuronal viability, oxidative stress, mitochondrial function, and cellular energy metabolism in neurons derived from Alzheimer's disease (AD) patients. The results demonstrate that ketone treatment produces dose-dependent improvements across multiple functional endpoints relevant to impaired mitochondrial function and neurodegeneration.

### Dose-Dependent Improvement in Neuronal Viability

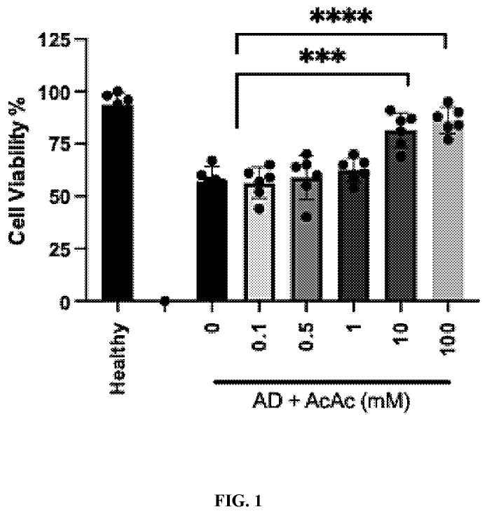

Exposure of AD-derived neurons to exogenous amyloid beta (Aβ1-42) resulted in a reduction in cell viability relative to healthy control neurons. Treatment with AcAc restored viability in a dose-dependent manner. As shown in FIG. 1, increasing concentrations of AcAc progressively improved cell viability in AD neurons exposed to Aβ, with higher concentrations approaching viability levels observed in healthy control cells. These results demonstrate that ketone treatment rescues neuronal survival under disease-relevant stress conditions.

### Reduction of Oxidative Stress

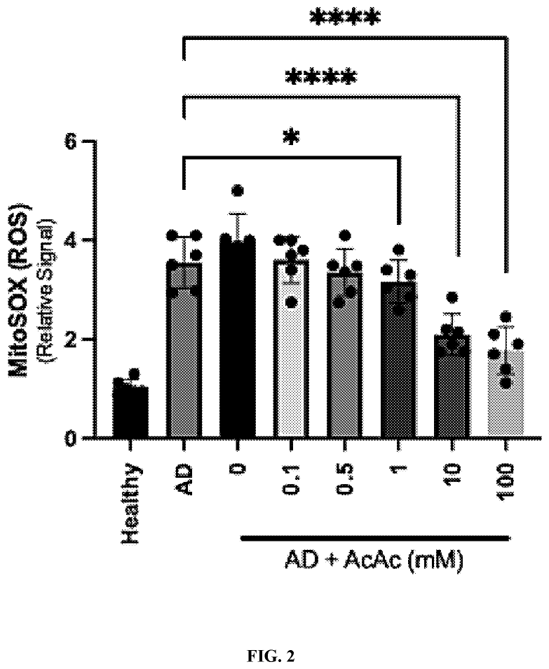

AD neurons exposed to amyloid beta exhibited elevated levels of reactive oxygen species (ROS) compared to healthy neurons, consistent with oxidative stress associated with neurodegeneration. Treatment with AcAc significantly reduced ROS levels in a dose-dependent manner. ROS levels were assessed using MitoSOX staining, which detects mitochondrial superoxide production, while mitochondrial structure and integrity were evaluated using MitoTracker staining. As shown in FIG. 2, AcAc treatment decreased MitoSOX relative signal intensity across increasing concentrations, indicating reduced oxidative stress.

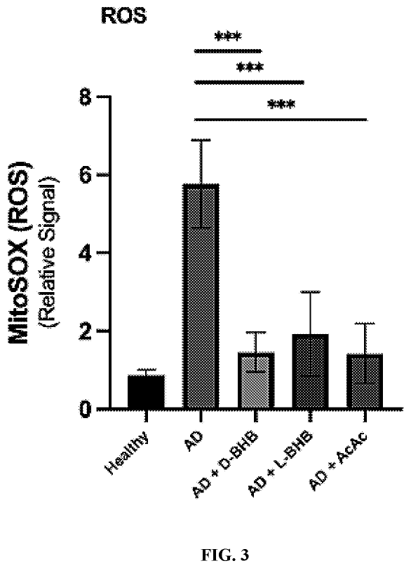

Consistent reductions in ROS were also observed in amyloid-induced oxidative stress model systems, following treatment with multiple ketone bodies, including D-β-hydroxybutyrate (D-βHB), L-β-hydroxybutyrate (L-BHB), and acetoacetate (AcAc), under non-dose-response conditions, as shown in FIG. 3, further supporting a class-wide effect of ketones on oxidative stress reduction.

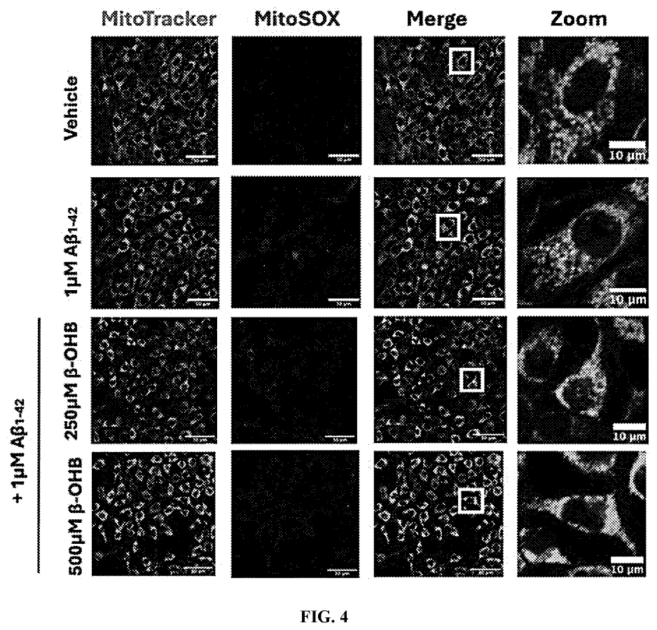

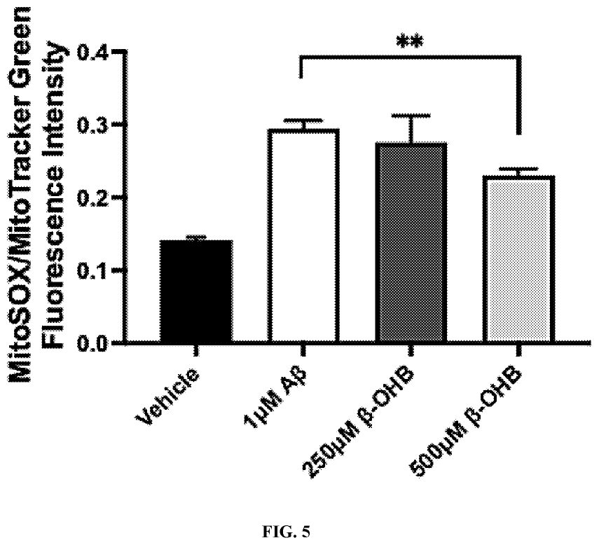

Consistent findings were also observed in amyloid-induced oxidative stress model systems, as shown in FIG. 4 and FIG. 5, where treatment with ketone bodies, including acetoacetate (AcAc) and β-hydroxybutyrate (B—OHB), reduced mitochondrial ROS levels and improved mitochondrial integrity, further supporting the role of ketones in mitigating oxidative damage in neuronal cells.

### Increase in ATP Production and Cellular Energy Metabolism

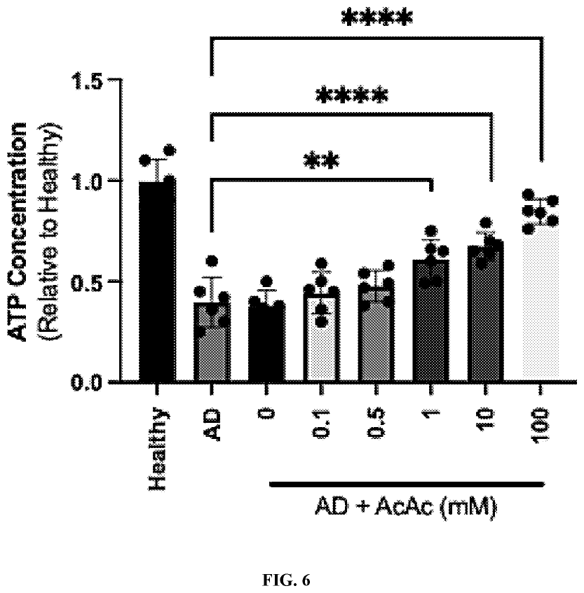

AD neurons demonstrated reduced intracellular ATP levels relative to healthy controls, consistent with impaired glucose utilization and mitochondrial dysfunction. Treatment with AcAc resulted in a dose-dependent increase in ATP concentration. As shown in FIG. 6, ATP levels increased with increasing AcAc concentration, indicating restoration of cellular energy production. These results are consistent with the ability of ketone bodies to serve as an alternative metabolic substrate and compensate for deficits in glucose metabolism.

### Reduction in Cell Death and Restoration of Neuronal Survival

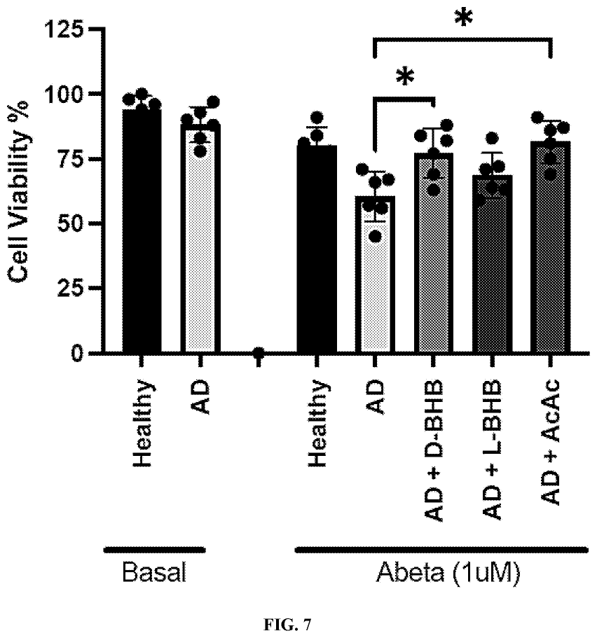

Exposure of AD neurons to amyloid beta resulted in a loss of cell viability and increased cell death relative to basal conditions. As shown in FIG. 7, no substantial differences in basal viability were observed between healthy and AD neurons; however, stimulation with Aβ resulted in reduced viability in AD neurons. Treatment with ketone bodies restored viability to varying extents, with D-β-hydroxybutyrate (D-βHB), L-β-hydroxybutyrate (L-BHB), and acetoacetate (AcAc) each improving viability relative to untreated AD neurons exposed to Aβ, and D-βHB and AcAc demonstrating substantial recovery toward levels observed in healthy control cells.

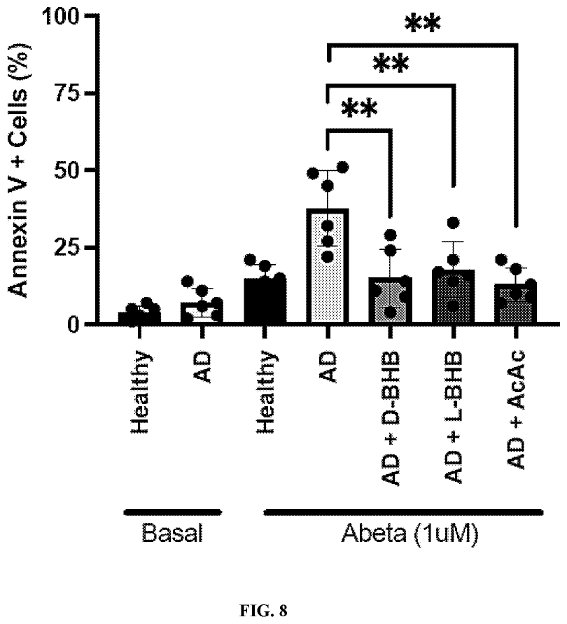

In addition to reductions in overall viability, Aβ exposure increased apoptotic signaling in AD neurons. As shown in FIG. 8, the percentage of Annexin V-positive cells was increased following AB treatment, indicating elevated apoptosis. Treatment with D-βHB, L-BHB, and AcAc reduced the proportion of Annexin V-positive cells relative to untreated AD neurons exposed to Aβ. These data indicate that ketone treatment protects neurons from amyloid-induced cell death and promotes neuronal survival.

### Improvement in Mitochondrial Function

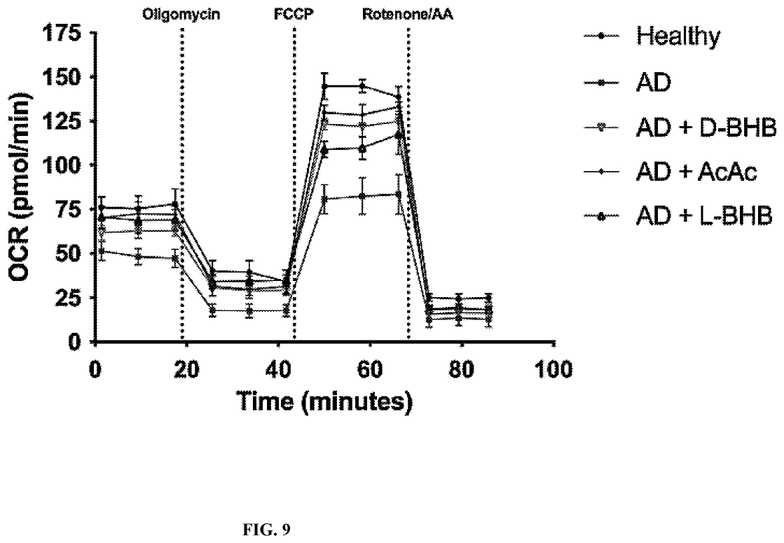

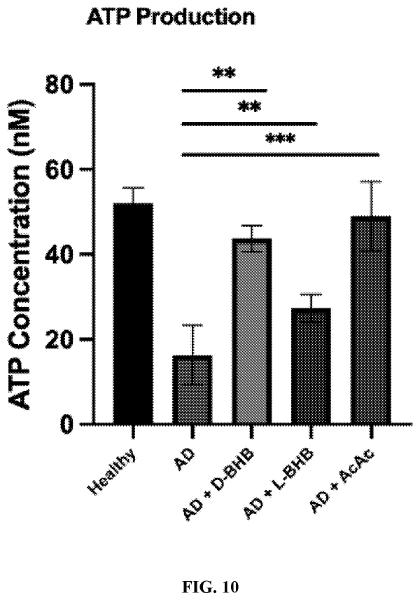

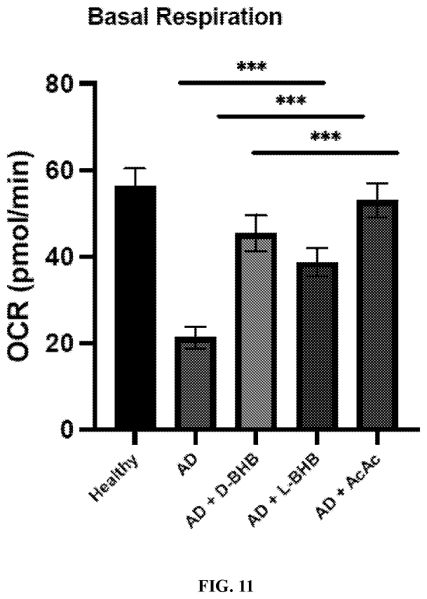

Mitochondrial dysfunction is a hallmark of AD and contributes to impaired energy metabolism. Treatment with ketones improved mitochondrial respiration and bioenergetic capacity in AD neurons. As shown in FIG. 9, ketone-treated AD neurons exhibited increased oxygen consumption rates compared to untreated AD neurons. Quantitative analysis further demonstrated improvements in ATP production and basal respiratory capacity, as shown in FIG. 10 and FIG. 11, respectively. These results indicate that ketone treatment restores mitochondrial function in AD neurons.

### Conclusion

Collectively, these data demonstrate that ketone bodies, including acetoacetate (AcAc) and others, provide a robust and dose-dependent rescue of neuronal dysfunction in Alzheimer's disease (AD) models. Ketone treatment improves neuronal viability, reduces oxidative stress, increases ATP production, decreases apoptosis, and enhances mitochondrial respiration. These findings support the use of exogenous ketone therapy to compensate for glucose hypometabolism and associated cellular energy deficits in neurodegenerative disease, consistent with the therapeutic titratable ketonemia (TTK) platform described herein.

### Methods

Human induced pluripotent stem cell (iPSC)—derived neurons were generated from both healthy donors and Alzheimer's disease (AD) patient donors and differentiated according to standard neuronal differentiation protocols. Cells were cultured under conditions sufficient to promote maturation prior to treatment.

For neuron dose-response experiments, neurons were exposed to varying concentrations of a ketone ester. Cells were treated for approximately 24 hours. To model disease-relevant stress conditions, neurons were co-treated with amyloid beta peptide (Aβ1-42) at a concentration of approximately 1 μM.

Cell viability was assessed using a luminescence-based live/dead assay (CellTiter-Glo, Promega). Reactive oxygen species (ROS) levels were measured using MitoSOX staining, which detects mitochondrial superoxide production. Mitochondrial structure and integrity were evaluated using MitoTracker staining. Intracellular ATP levels were quantified using an ATP-dependent luminescence assay (ATP-Glo, Promega).

For longer-term functional assays, including apoptosis and mitochondrial analyses, cells were treated for approximately 14 days prior to endpoint measurement. Apoptosis was measured using Annexin V staining to determine the percentage of apoptotic cells. Mitochondrial function was evaluated using a Seahorse XF Mito Stress Test (Agilent) to measure oxygen consumption rate (OCR), including basal respiration and ATP-linked respiration.

All assays were performed using multiple donor-derived cell lines and in replicate, and data were analyzed using standard statistical methods.

### Example 23: Exemplary Effects of Ketone Bodies in Parkinson's Disease Neurons

Described below are the effects of exogenous ketone bodies, including acetoacetate (AcAc) or compositions comprising one or more of the compounds described herein, on neuronal viability, oxidative stress, mitochondrial function, and cellular energy metabolism in neurons derived from Parkinson's disease (PD) patients. The results demonstrate that ketone treatment produces improvements across multiple functional endpoints relevant to impaired mitochondrial function and neurodegeneration.

### Reduction in Cell Death and Restoration of Neuronal Survival

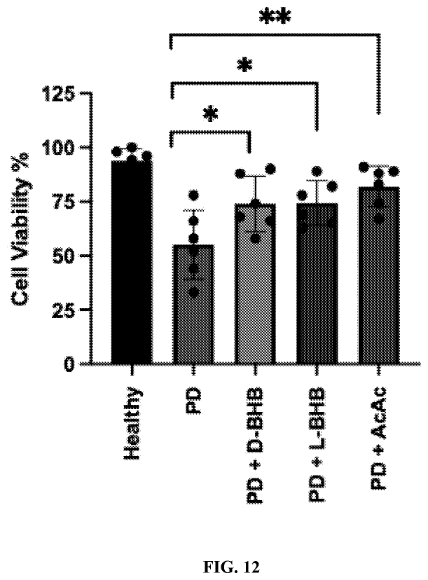

PD neurons exhibited a loss of cell viability and increased cell death relative to healthy control neurons. As shown in FIG. 12, PD neurons demonstrated reduced viability compared to healthy neurons, and treatment with ketone bodies, including D-β-hydroxybutyrate (D-βHB), L-β-hydroxybutyrate (L-BHB), and acetoacetate (AcAc), increased viability relative to untreated PD neurons. Among the tested ketone bodies, AcAc demonstrated a particularly robust effect in restoring viability toward levels observed in healthy control cells. These results demonstrate that ketone treatment improves neuronal survival in PD neurons.

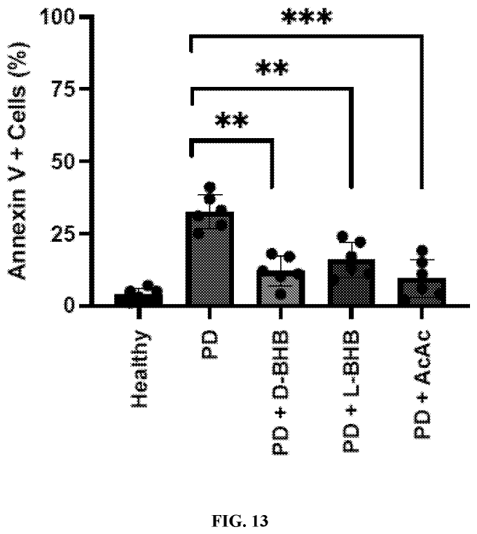

In addition to reductions in overall viability, PD neurons exhibited increased apoptotic signaling. As shown in FIG. 13, the percentage of Annexin V-positive cells was increased in PD neurons relative to healthy controls. Treatment with D-β-hydroxybutyrate (D-βHB), L-β-hydroxybutyrate (L-BHB), and acetoacetate (AcAc) reduced the proportion of Annexin V-positive cells relative to untreated PD neurons. These data indicate that ketone treatment protects neurons from disease-associated cell death and promotes neuronal survival.

### Reduction of Oxidative Stress

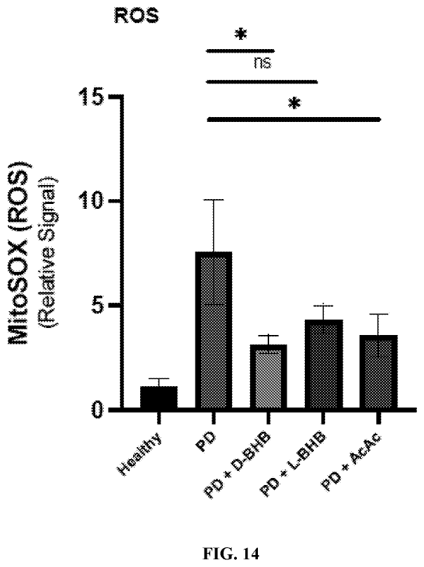

PD neurons exhibited elevated levels of reactive oxygen species (ROS) compared to healthy neurons, consistent with oxidative stress associated with neurodegeneration. Treatment with ketone bodies reduced ROS levels. ROS levels were assessed using MitoSOX staining, which detects mitochondrial superoxide production, while mitochondrial structure and integrity were evaluated using MitoTracker staining. As shown in FIG. 14, ketone treatment reduced MitoSOX relative signal intensity, indicating reduced oxidative stress. These findings further support the role of ketones in mitigating oxidative damage in neuronal cells.

### Improvement in Mitochondrial Function

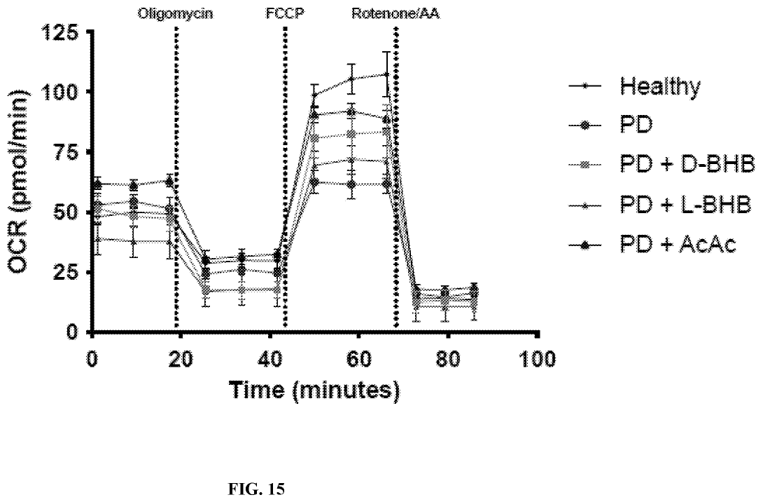

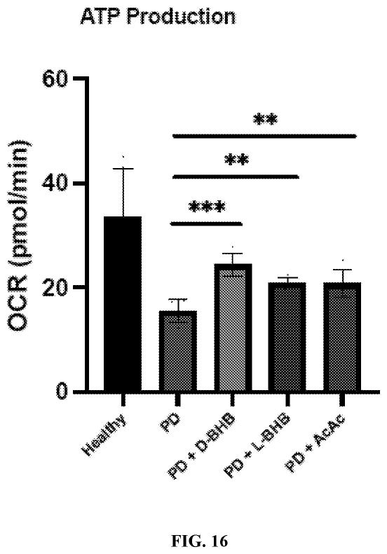

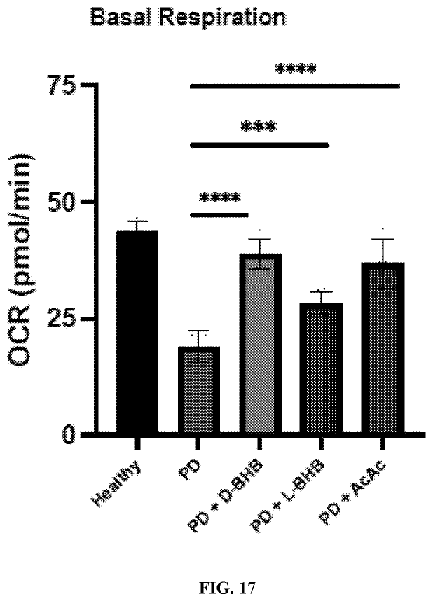

Mitochondrial dysfunction is a hallmark of PD and contributes to impaired energy metabolism. Treatment with ketones improved mitochondrial respiration and bioenergetic capacity in PD neurons. As shown in FIG. 15, ketone-treated PD neurons exhibited increased oxygen consumption rates compared to untreated PD neurons. Quantitative analysis further demonstrated improvements in ATP production and basal respiratory capacity, as shown in FIG. 16 and FIG. 17, respectively. These results indicate that ketone treatment restores mitochondrial function in PD neurons.

### Conclusion

Collectively, these data demonstrate that ketone bodies, including acetoacetate (AcAc) and others, provide a robust rescue of neuronal dysfunction in Parkinson's disease (PD) models. Ketone treatment improves neuronal viability, reduces oxidative stress, increases ATP production, decreases apoptosis, and enhances mitochondrial respiration.

These findings support the use of exogenous ketone therapy to address impaired cellular energy metabolism in neurodegenerative disease, consistent with the therapeutic titratable ketonemia (TTK) platform described herein.

### Methods

Human induced pluripotent stem cell (iPSC)—derived neurons were generated from both healthy donors and Parkinson's disease (PD) patient donors and differentiated according to standard neuronal differentiation protocols. Cells were cultured under conditions sufficient to promote maturation prior to treatment.

Cell viability was assessed using a luminescence-based live/dead assay (CellTiter-Glo, Promega). Reactive oxygen species (ROS) levels were measured using MitoSOX staining, which detects mitochondrial superoxide production. Mitochondrial structure and integrity were evaluated using MitoTracker staining. Intracellular ATP levels were quantified using an ATP-dependent luminescence assay (ATP-Glo, Promega). For functional assays, including apoptosis and mitochondrial analyses, cells were treated for approximately 14 days prior to endpoint measurement. Apoptosis was measured using Annexin V staining to determine the percentage of apoptotic cells. Mitochondrial function was evaluated using a Seahorse XF Mito Stress Test (Agilent) to measure oxygen consumption rate (OCR), including basal respiration and ATP-linked respiration.

All assays were performed using multiple donor-derived cell lines and in replicate, and data were analyzed using standard statistical methods.

### Example 24: Exemplary Effects of Ketone Bodies on Microglial Amyloid Uptake and Clearance

Described below are the effects of exogenous ketone bodies, including acetoacetate (AcAc) or compositions comprising one or more of the compounds described herein, on microglial uptake and clearance of amyloid beta (Aβ). The results demonstrate that ketone treatment enhances microglial phagocytic and degradative capacity, supporting a role for ketone bodies in promoting amyloid clearance in neurodegenerative disease. Enhancement of Amyloid Beta Uptake

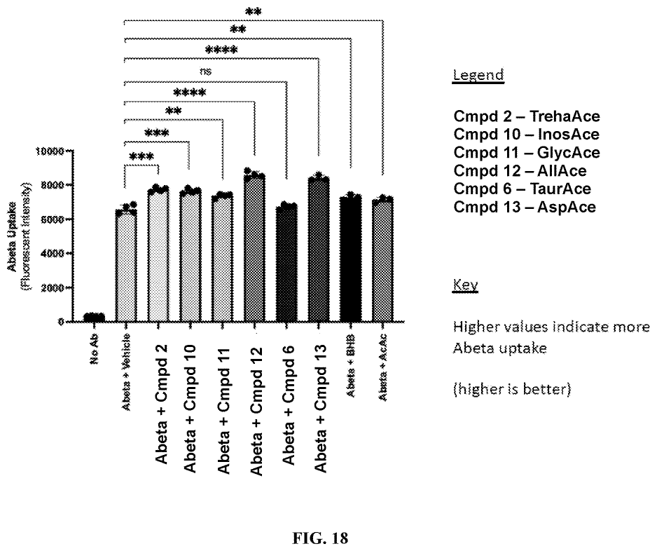

Microglia play a critical role in the internalization and clearance of extracellular amyloid beta. The ability of ketone bodies to enhance microglial uptake of Aβ was evaluated. As shown in FIG. 18, treatment with ketone bodies, including acetoacetate-derived ketone esters, increased the uptake of Aβ1-42 by microglia relative to untreated control cells. Increased intracellular fluorescence signal indicates enhanced internalization of amyloid beta. These results demonstrate that ketone treatment promotes microglial uptake of neurotoxic amyloid species.

### Enhancement of Amyloid Beta Clearance

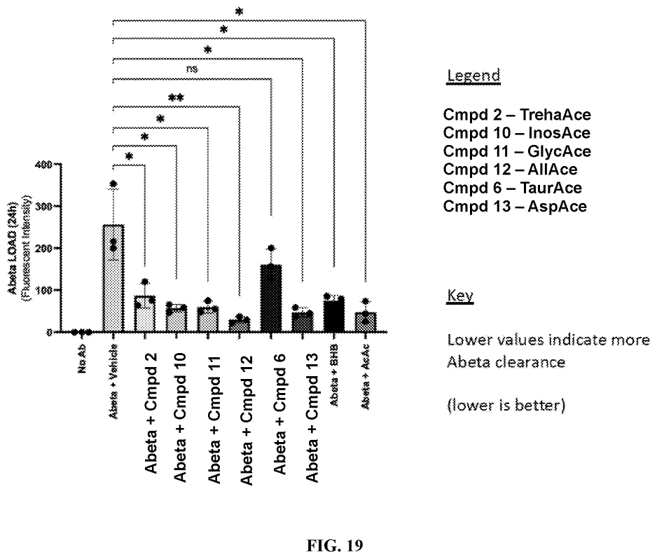

In addition to uptake, the ability of microglia to degrade and clear internalized amyloid beta is critical for maintaining neuronal health. As such, the effects of ketone treatment on amyloid clearance were evaluated. As shown in FIG. 19, treatment with ketone bodies reduced the amount of remaining extracellular amyloid beta following incubation, indicating enhanced clearance. Lower fluorescence signal corresponds to increased degradation and removal of amyloid beta. These data indicate that ketone treatment improves microglial clearance of amyloid beta.

### Acceleration of Amyloid Clearance Kinetics

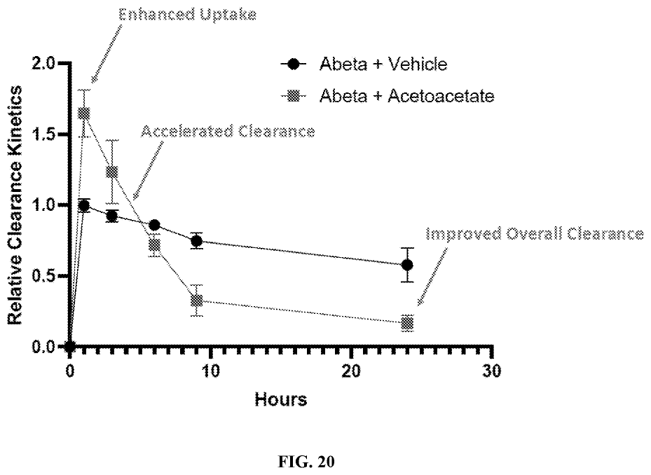

The rate of amyloid beta clearance was further evaluated to determine whether ketone bodies enhance the kinetics of amyloid degradation. As shown in FIG. 20, treatment with acetoacetate increased the rate of amyloid beta clearance over time relative to vehicle-treated controls. Microglia treated with ketone bodies exhibited both enhanced uptake and accelerated degradation of amyloid beta, resulting in improved overall clearance kinetics.

### Conclusion

Collectively, these data demonstrate that ketone bodies, including acetoacetate (AcAc), enhance both the uptake and clearance of amyloid beta by microglia. Ketone treatment increases internalization of amyloid, accelerates its degradation, and improves overall clearance kinetics. These findings support a dual mechanism of action for ketone therapy in neurodegenerative disease, including both metabolic support and enhancement of microglial-mediated amyloid clearance and phagocytic capacity.

### Methods

Human induced pluripotent stem cell (iPSC)—derived microglia were generated and cultured under conditions sufficient to promote maturation prior to treatment. Microglial identity was confirmed by expression of established markers (i.e. Iba1).

For uptake assays, microglia were treated with ketone esters at a concentration of approximately 5 mM. Cells were exposed to fluorescently labeled amyloid beta peptide (Aβ1-42) and incubated for approximately 1 hour. Following incubation, cells were subjected to an acid wash to remove non-internalized amyloid, and intracellular fluorescence was measured to quantify amyloid uptake.

For clearance assays, microglia were treated with ketone esters and exposed to fluorescently labeled amyloid beta peptide. Cells were incubated for approximately 24 hours, and the amount of remaining amyloid beta was quantified by fluorescence-based analysis.

Amyloid uptake and clearance were quantified using fluorescence plate reader measurements. Data were analyzed by comparing treated and untreated conditions to determine relative changes in amyloid internalization and degradation.

All assays were performed in replicate, and data were analyzed using standard statistical methods.

### Example 25: PK Analysis of Representative Compound of Formula (A)

### Experimental Parameters

Compound: Representative Compound of Formula A, TrehaAce (Compound 2)
Study type 24 h PK, Beta-hydroxybutyrate levels as surrogate
Test animal: Strain/Species/Sex C57BL6J/Mouse/Male
Total number of animals used 6 (n=3/group)
Animal age/Body weight 8 weeks/23-26g
Route of administration PO
Feeding regimen Fed-Ad libitum
Dose level (g/kg) Group 1-1gpk, Group 2-5gpk
Dose volume (mL/kg body weight) Up to 0.3 mL oral gavage
Formulation strength (mg/mL)₅₀₀ mg/mL
Vehicle 20% DMSO/80% water
Sampling technique Submandibular
Blood collection schedule, post dose 0, 0.08, 0.25, 0.5, 1.0, 2.0, 4.0, 8.0 and 24 h (8 time points)
Anticoagulant K2EDTA
Matrix Plasma
Study samples storage At −80° C.

### SUMMARY

TrehaAce was well tolerated at all study doses, and no indication of toxicity was observed. TrehaAce is a ketone ester prodrug that is rapidly converted to the functional ketone body betahydroxybutyrate (BHB) following oral consumption. In the present study, plasma BHB levels were utilized as a surrogate pharmacokinetic marker for the test article as reported previously. Baseline beta-hydroxybutyrate levels were assayed at the 0 timepoint.

Noncompartmental pharmacokinetic analysis of plasma BHB was performed following oral administration of TrehaAce at 1 g/kg and 5 g/kg in mice using standard Phoenix® WinNonlin extravascular NCA methods (linear trapezoidal AUC, log-linear terminal phase).

At both dose levels, TrehaAce was rapidly absorbed with a consistent Tmax of

approximately 2 hours, indicating similar absorption kinetics across doses. Peak plasma concentrations (Cmax) increased with dose in an approximately dose-proportional manner.

However, systemic exposure (AUC) increased in a less-than-dose-proportional fashion from 1 g/kg to 5 g/kg, as evidenced by reduced dose-normalized AUC values at the higher dose. This finding suggests potential nonlinear pharmacokinetics, possibly due to rapid prodrug conversion, saturation of absorption, reduced oral bioavailability as dose increases, or dose-dependent clearance mechanisms.

At the 1 g/kg dose, terminal phase characterization was acceptable for two of three animals, although high AUC and AUMC extrapolation percentages indicate that later sampling time points would be beneficial for more robust estimation of terminal half-life and MRT. In contrast, the 5 g/kg dose group demonstrated a well-defined terminal phase in all animals, with lower extrapolated exposure and more stable λ z-dependent parameters.

Apparent clearance (CL/F) increased with dose, and apparent volume of distribution (Vz/F) decreased at the higher dose, further supporting the presence of nonlinear exposure kinetics.

Administration of TrehaAce at 1 g/kg and 5 g/kg resulted in a maximal induction of plasma BHB levels peaking at 2.0 h post-administration and achieving a mean Cmax of ~324 μM and ~1541 μM for each dose group respectively. Over the duration of the study a cumulative exposure (AUCO-t) reached approximately 2.3 mM and 11.8 mM for the 1gpk and 5gpk doses respectively.

The ~91% dose linearity was observed between 1gpk and 5gpk closing and sustainable BHB levels above 0.5 mM at the 5gpk dose over 8 h indicate single daily administration of the test article to induce a sustained ketogenic state without required fasting.

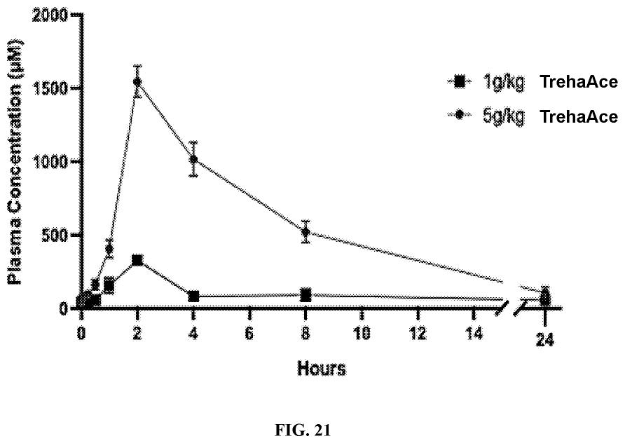

Results are summarized in FIG. 21 and in Table 6 below.

TABLE 6

| Parameter | 1 g pk Dose | 5 g pk Dose |
|---|---|---|
| Cₘₐₓ (uM) | 324.31 | 1541.67 |
| Tₘₐₓ (h) | 2.0 | 2.00 |
| Tₗₐₛₜ (h) | 24.0 | 24.0 |
| AUC₀₋ₜ (μM · h) | 2298.25 | 11762.82 |
| AUC_{0-∞} (μM · h) | 4045.43 | 12756.41 |
| T_{1/2} (h) | 20.46 | 6.33 |
| CL (mL/min/kg) | 39.63 | 63.2 |
| Vz/F (L/kg) | 70.29 | 34.47 |

Overall, these examples-across oncology, heart failure, neurodegeneration, epilepsy, and autoimmune arthritis-illustrate how MERIT therapy can be deployed, measured, and how it yields multi-faceted benefits consistent with the mechanistic aims. Each example ties improvement in patient outcomes to at least one mechanistic biomarker change (NAD⁺ increase, histone acetylation, IL-6 reduction, etc.), reinforcing the premise that controlling metabolic/redox/epigenetic states leads to tangible clinical improvements.

1. A method for assessing a state of bioenergetic sufficiency or insufficiency in a biological system, the method comprising: a. measuring, using one or more measurement modalities, a plurality of parameters reflecting components of an effective energy generation rate (E) and a total energy requirement rate (R) in the biological system; and b. determining, using a processor configured with an algorithm based on the Unified Theory of Bioenergetic Sufficiency (UTBS), the state of bioenergetic sufficiency or insufficiency based on an integrated relationship between said measured parameters, wherein the relationship reflects a Metabolic Sufficiency Ratio (M) defined as M=E/R or a derivative thereof.

2. (canceled)

3. The method of claim 1, wherein the plurality of parameters reflecting E comprise at least one parameter selected from the group consisting of: a level, concentration, uptake rate, or metabolic flux of an energy substrate selected from glucose, a fatty acid, a ketone body, lactate, and an amino acid; a level or activity of a substrate transporter or metabolic enzyme; an ATP level; a phosphocreatine (PCr) level; an ATP/ADP ratio; a PCr/ATP ratio; and an oxygen consumption rate.

4. The method of claim 1, wherein the plurality of parameters reflecting R comprise at least one parameter selected from the group consisting of: a marker of basal metabolic rate; a marker of cellular stress; a marker of inflammation; a marker of oxidative damage; a marker of tissue-specific activity; and a marker of cellular repair processes.

5. The method of claim 1, wherein the one or more measurement modalities are selected from the group consisting of positron emission tomography (PET), magnetic resonance spectroscopy (MRS), metabolomic analysis, indirect calorimetry, and biochemical assay of a biological sample.

6.-8. (canceled)

9. The method of claim 1, further comprising measuring an index of metabolic flexibility, optionally using indirect calorimetry to measure a change in Respiratory Quotient (RQ) in response to a metabolic challenge.

10. The method of claim 1, wherein determining the state comprises comparing the relationship reflecting M to a predetermined, tissue-specific critical threshold value (M_crit).

11. (canceled)

12. A method for treating or preventing a condition associated with energy insufficiency in a subject diagnosed using the method of claim 1, the method comprising administering to the subject a therapeutically effective amount of an agent or intervention selected or adjusted based on the assessed state of bioenergetic sufficiency or insufficiency, wherein the agent or intervention increases a Metabolic Sufficiency Ratio (M=E/R) in a target tissue of the subject.

13.-19. (canceled)

20. The method of claim 12, wherein the condition associated with energy insufficiency is selected from the group consisting of Alzheimer's disease, Parkinson's disease, heart failure, type 2 diabetes, metabolic syndrome, traumatic brain injury cachexia, age-related functional decline, and the orphan diseases GLUT1 deficiency syndrome, Pyruvate Dehydrogenase Complex Deficiency (PDHc-D), SLC13A5 epileptic encephalopathy, PCDH19-Related Epilepsy, Dravet syndrome, Lennox-Gastaut syndrome, infantile spasms, CDKL5 Deficiency Disorder, Doose syndrome (myoclonic-astatic epilepsy), Mitochondrial Complex I Deficiencies, Leigh syndrome, MELAS, MERRF, Friedreich's ataxia, juvenile ALS, multiple acyl-CoA dehydrogenase deficiency (MADD), Very Long-Chain Acyl-CoA Dehydrogenase Deficiency (VLCAD), Long-Chain 3-Hydroxyacyl-CoA Dehydrogenase Deficiency/Trifunctional Protein Deficiency (LCHAD/TFP), medium-chain acyl-CoA dehydrogenase deficiency (MCAD deficiency), Carnitine Palmitoyltransferase II Deficiency (CPT-II), HMG-CoA Lyase Deficiency, Barth syndrome, LMNA-related dilated cardiomyopathy, Duchenne muscular dystrophy-associated cardiomyopathy, spinal muscular atrophy type II, mtDNA Depletion Syndromes, early-onset familial Alzheimer's or Parkinson's disease, Febrile Infection-Related Epilepsy Syndrome (FIRES), Glioblastoma Multiforme (GBM), Diffuse Intrinsic Pontine Glioma (DIPG), Aicardi-Goutières Syndrome (AGS), SCN8A-Related Epilepsy (SCN8A), Tuberous Sclerosis Complex (TSC), Rasmussen Encephalitis (RE), IDH-mutant High-Grade Glioma (HGG), Atypical Teratoid Rhabdoid Tumors (ATRT), Rett Syndrome (RTT), Angelman Syndrome (AS), Lafora Disease (LD), Frontotemporal Dementia (FTD), Acute Necrotizing Encephalopathy (ANEC), Autoimmune Limbic Encephalitis (ALE), Focal Cortical Dysplasia (FCD), Hypothalamic Hamartoma (HH), Neuronal Ceroid Lipofuscinoses (NCLs), Unverricht-Lundborg Disease (EPM1), Sturge-Weber Syndrome (SWS), Hemimegalencephaly (HME), and Krabbe Disease (KD).

21.-34. (canceled)

35. A method of treating a disease characterized by metabolic, redox, or epigenetic dysregulation in a subject in need thereof, comprising administering to the subject a therapeutically effective amount of a ketone body or a pharmaceutically acceptable ketone precursor, thereby improving one or more metabolic, redox, or epigenetic parameters in the subject and treating the disease.

36. The method of claim 35, wherein the disease characterized by metabolic, redox, or epigenetic dysregulation is selected from the group consisting of: a neurodegenerative disease, a neurodevelopmental or psychiatric disorder, a metabolic or endocrine disease, an inflammatory or autoimmune disease, a cardiovascular or ischemic condition, a mitochondrial disorder, or a cancer.

37. (canceled)

38. The method of claim 35, wherein the disease is characterized by reductive stress-indicated by an elevated NADH/NAD⁺ ratio, elevated lactate/pyruvate ratio, or elevated β-hydroxybutyrate/acetoacetate ratio- and wherein the administering step increases the subject's NAD+/NADH ratio and alleviates said reductive stress.

39. (canceled)

40. The method of claim 35, wherein the ketone body or precursor is administered in an amount sufficient to raise the subject's blood β-hydroxybutyrate level to between about 0.5 mM and about 8 mM.

41. The method of claim 35, wherein the ketone body or precursor comprises β-hydroxybutyrate, acetoacetate, or a combination thereof, optionally in a form selected from: a ketone salt, a ketone ester, a ketone amide, a ketoacid, or a plurality of these forms.

42. The method of claim 35, further comprising identifying the subject as having said disease characterized by metabolic, redox, or epigenetic dysregulation, by measuring one or more biomarkers indicative of metabolic, redox, or epigenetic dysfunction prior to treatment.

43. The method of claim 42, wherein the one or more biomarkers are selected from: blood or tissue NAD⁺ levels, NADH/NAD⁺ ratio, lactate level, β-hydroxybutyrate: acetoacetate ratio, reactive oxygen species level, an inflammatory cytokine level, or a histone acetylation level in a tissue sample.

44. The method of claim 35, wherein the disease is a cancer selected from solid tumors and hematologic malignancies, and metabolic, redox, or epigenetic dysregulation refers to the cancer cells' Warburg metabolism, elevated NADH, and aberrant gene expression, and wherein administering the ketone body is effective to alter the tumor metabolism or tumor microenvironment, thereby inhibiting cancer progression.

45. The method of claim 35, wherein the disease is an autoimmune or autoinflammatory disease characterized by excessive immune activation and metabolic dysregulation of immune cells, and wherein administering the ketone body reduces inflammatory mediator levels and shifts immune cell metabolism from glycolytic to oxidative, thereby ameliorating the disease.

46. The method of claim 35, wherein the disease is associated with mitochondrial dysfunction (including primary mitochondrial genetic disorders or age-related mitochondrial decline), and wherein ketone body administration bypasses one or more defective metabolic steps and improves ATP production in affected tissues.

47. A method of treating a patient in need of metabolic therapy comprising: (a) administering to the patient a ketone body or ketone precursor composition; (b) measuring in the patient at least one ketone-related value selected from blood β-hydroxybutyrate concentration, blood acetoacetate concentration, or a ratio thereof; (c) adjusting the dose or rate of administration of the ketone composition based on the measured value to achieve a predetermined target ketone level or ratio; and (d) maintaining the patient's ketone level or ratio within said target range for a therapeutically effective duration, thereby treating the patient's condition.

48. The method of claim 47, wherein the predetermined target ketone level comprises a blood β hydroxybutyrate concentration between about 1 mM and about 5 mM.

49. (canceled)

50. A method of treating a disease or condition associated with a high NADH/NAD⁺ ratio or reductive stress in a patient, comprising administering to the patient a therapeutically effective amount of acetoacetate or an acetoacetate-generating compound, thereby converting excess NADH to NAD⁺ in cells of the patient and alleviating the reductive stress.
